SEQUENCE LISTING (185 sequences)

SEQ ID NO: 1 - protein, 107 aa, Homo sapiens [sequence omitted]

SEQ ID NO: 2 - protein, 1337 aa, Homo sapiens
EICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 3 - DNA, 393 bp, Homo sapiens
gaagttcaattgttagagtctggtggcggtcttgttcagcctggtggttctttacgtctttcttgcgctgcttccggattcactttctctccttactctatgctttgggttcgccaagctcctggtaaaggtttggagtgggtttcttctatcggttcttctggtggctctactcgttatgctgactccgttaaaggtcgcttcactatctctagagacaactctaagaatactctctacttgcagatgaacagcttaagggctgaggacaccgccatgtattactgtgcacgggtacgggggatccttcattacgatattttgattggtagaaatctctactactactacatggacgtctggggcaaagggaccacggtcaccgtctcaagc

SEQ ID NO: 4 - protein, 131 aa, Homo sapiens
EVQLLESGGGLVQPGGSLRLSCAASGFTFSPYSMLWVRQAPGKGLEWVSSIGSSGGSTRYADSVKGRFTISRDNSKNTLYLQMNSLRAEDTAMYYCARVRGILHYDILIGRNLYYYYMDVWGKGTTVTVSS

SEQ ID NO: 5 - protein, 5 aa, Homo sapiens
PYSML

SEQ ID NO: 6 - protein, 17 aa, Homo sapiens
SIGSSGGSTRYADSVKG

SEQ ID NO: 7 - protein, 22 aa, Homo sapiens
VRGILHYDILIGRNLYYYYMDV

SEQ ID NO: 8 - DNA, 390 bp, Homo sapiens
gaagttcaattgttagagtctggtggcggtcttgttcagcctggtggttctttacgtctttcttgcgctgcttccggattcactttctctaagtacactatgcattgggttcgccaagctcctggtaaaggtttggagtgggtttcttctatcgtttcttctggtggctggactgattatgctgactccgttaaaggtcgcttcactatctctagagacaactctaagaatactctctacttgcagatgaacagcttaagggctgaggacacggccgtgtattactgtgcgagagatcggagtatagcagcagctggtaccggttggtctgtgagttttgtggactggttcgacccctggggccagggaaccctggtcaccgtctcaagc

SEQ ID NO: 9 - protein, 130 aa, Homo sapiens
EVQLLESGGGLVQPGGSLRLSCAASGFTFSKYTMHWVRQAPGKGLEWVSSIVSSGGWTDYADSVKGRFTISRDNSKNTLYLQMNSLRAEDTAVYYCARDRSIAAAGTGWSVSFVDWFDPWGQGTLVTVSS

SEQ ID NO: 10 - protein, 5 aa, Homo sapiens
KYTMH

SEQ ID NO: 11 - protein, 17 aa, Homo sapiens
SIVSSGGWTDYADSVKG

SEQ ID NO: 12 - protein, 21 aa, Homo sapiens
DRSIAAAGTGWSVSFVDWFDP

SEQ ID NO: 13 - DNA, 360 bp, Homo sapiens
gaagttcaattgttagagtctggtggcggtcttgttcagcctggtggttctttacgtctttcttgcgctgcttccggattcactttctctatttaccgtatgcagtgggttcgccaagctcctggtaaaggtttggagtgggtttctggtatctctccttctggtggcactacttggtatgctgactccgttaaaggtcgcttcactatctctagagacaactctaagaatactctctacttgcagatgaacagcttaagggctgaggacacggccgtgtattactgtgcgagatggagcgggggttcgggctatgcttttgatatctggggccaagggacaatggtcaccgtctcaagc

SEQ ID NO: 14 - protein, 120 aa, Homo sapiens
EVQLLESGGGLVQPGGSLRLSCAASGFTFSIYRMQWVRQAPGKGLEWVSGISPSGGTTWYADSVKGRFTISRDNSKNTLYLQMNSLRAEDTAVYYCARWSGGSGYAFDIWGQGTMVTVSS

SEQ ID NO: 15 - protein, 5 aa, Homo sapiens
IYRMQ

SEQ ID NO: 16 - protein, 17 aa, Homo sapiens
GISPSGGTTWYADSVKG

SEQ ID NO: 17 - protein, 11 aa, Homo sapiens
WSGGSGYAFDI

SEQ ID NO: 18 - DNA, 360 bp, Homo sapiens
gaggtccagctgttggagtccggcggtggcctggtgcagcctggggggtccctgagactctcctgcgcagctagcggcttcaccttcagcatttaccgtatgcagtgggtgcgccaggctcctggaaaggggctggagtgggtttccggtatctctccctctggtggcacgacgtggtatgctgactccgtgaagggccggttcacaatctccagagacaattccaagaacactctgtacctgcaaatgaacagcctgagagctgaggatactgcagtgtactactgcgccagatggtccgggggctccggatacgccttcgacatctggggacagggaaccatggtcaccgtctcaagc

SEQ ID NO: 19 - DNA, 363 bp, Homo sapiens
gaagttcaattgttagagtctggtggcggtcttgttcagcctggtggttctttacgtctttcttgcgctgcttccggattcactttctctaattaccatatggcttgggttcgccaagctcctggtaaaggtttggagtgggtttctgttatctctcctactggtggccgtactacttatgctgactccgttaaaggtcgcttcactatctctagagacaactctaagaatactctctacttgcagatgaacagcttaagggctgaggacacagccacatattactgtgcgagagcggggtacagctatggttatggctactttgactactggggccagggaaccctggtcaccgtctcaagc

SEQ ID NO: 20 - protein, 121 aa, Homo sapiens
EVQLLESGGGLVQPGGSLRLSCAASGFTFSNYHMAWVRQAPGKGLEWVSVISPTGGRTTYADSVKGRFTISRDNSKNTLYLQMNSLRAEDTATYYCARAGYSYGYGYFDYWGQGTLVTVSS

SEQ ID NO: 21 - protein, 5 aa, Homo sapiens
NYHMA

SEQ ID NO: 22 - protein, 17 aa, Homo sapiens
VISPTGGRTTYADSVKG

SEQ ID NO: 23 - protein, 12 aa, Homo sapiens
AGYSYGYGYFDY

SEQ ID NO: 24 - DNA, 363 bp, Homo sapiens
gaggtccagctgttggagtccggcggtggcctggtgcagcctggggggtccctgagactctcctgcgcagctagcggcttcaccttcagcaattaccacatggcctgggtgcgccaggctcctggaaaggggctggagtgggtttccgtgatctctcctaccggtggcaggaccacttacgctgactccgtgaagggccggttcacaatctccagagacaattccaagaacactctgtacctgcaaatgaacagcctgagagctgaggatactgcaacatactactgcgccagagccgggtactcctacggctacggatacttcgactactggggacagggaaccctggtcaccgtctcaagc

SEQ ID NO: 25 - DNA, 357 bp, Homo sapiens
gaagttcaattgttagagtctggtggcggtcttgttcagcctggtggttctttacgtctttcttgcgctgcttccggattcactttctctaagtacatgatgtcttgggttcgccaagctcctggtaaaggtttggagtgggtttcttatatctctccttctggtggccttacttggtatgctgactccgttaaaggtcgcttcactatctctagagacaactctaagaatactctctacttgcagatgaacagcttaagggctgaggacacggccgtgtattactgtgcgagagatggagctagaggctacggtatggacgtctggggccaagggaccacggtcaccgtctcaagc

SEQ ID NO: 26 - protein, 119 aa, Homo sapiens
EVQLLESGGGLVQPGGSLRLSCAASGFTFSKYMMSWVRQAPGKGLEWVSYISPSGGLTWYADSVKGRFTISRDNSKNTLYLQMNSLRAEDTAVYYCARDGARGYGMDVWGQGTTVTVSS

SEQ ID NO: 27 - protein, 5 aa, Homo sapiens
KYMMS

SEQ ID NO: 28 - protein, 17 aa, Homo sapiens
YISPSGGLTWYADSVKG

SEQ ID NO: 29 - protein, 10 aa, Homo sapiens
DGARGYGMDV

SEQ ID NO: 30 - DNA, 357 bp, Homo sapiens
gaggtccagctgttggagtccggcggtggcctggtgcagcctggggggtccctgagactctcctgcgcagctagcggcttcaccttcagcaagtacatgatgtcttgggtgcgccaggctcctggaaaggggctggagtgggtttcctatatctctccctctggtggcctgacgtggtatgctgactccgtgaagggccggttcacaatctccagagacaattccaagaacactctgtacctgcaaatgaacagcctgagagctgaggatactgcagtgtactactgcgccagagatggggctagaggatacggaatggacgtctggggacagggaaccaccgtcaccgtctcaagc

SEQ ID NO: 31 - DNA, 372 bp, Homo sapiens
gaagttcaattgttagagtctggtggcggtcttgttcagcctggtggttctttacgtctttcttgcgctgcttccggattcactttctctaattaccctatgtattgggttcgccaagctcctggtaaaggtttggagtgggtttctcgtatctcttcttctggtggccgtactgtttatgctgactccgttaaaggtcgcttcactatctctagagacaactctaagaatactctctacttgcagatgaacagcttaagggctgaggacacggccgtgtattactgtgcgagagatcgatggtccagatctgcagctgaatatgggttgggtggctactggggccagggaaccctggtcaccgtctcaagc

SEQ ID NO: 32 - protein, 124 aa, Homo sapiens
EVQLLESGGGLVQPGGSLRLSCAASGFTFSNYPMYWVRQAPGKGLEWVSRISSSGGRTVYADSVKGRFTISRDNSKNTLYLQMNSLRAEDTAVYYCARDRWSRSAAEYGLGGYWGQGTLVTVSS

SEQ ID NO: 33 - protein, 5 aa, Homo sapiens
NYPMY

SEQ ID NO: 34 - protein, 17 aa, Homo sapiens
RISSSGGRTVYADSVKG

SEQ ID NO: 35 - protein, 15 aa, Homo sapiens
DRWSRSAAEYGLGGY

SEQ ID NO: 36 - DNA, 372 bp, Homo sapiens
gaggtccagctgttggagtccggcggtggcctggtgcagcctggggggtccctgagactctcctgcgcagctagcggcttcaccttcagcaattaccccatgtactgggtgcgccaggctcctggaaaggggctggagtgggtttccaggatctctagcagcggtggcaggaccgtgtacgctgactccgtgaagggccggttcacaatctccagagacaattccaagaacactctgtacctgcaaatgaacagcctgagagctgaggatactgcagtgtactactgcgccagagataggtggtccagatctgcagccgagtacggactggggggctactggggacagggaaccctggtcaccgtctcaagc

SEQ ID NO: 37 - DNA, 369 bp, Homo sapiens
caggttcagctgcagcagtctggacctgagctagtgaagcctggggcttcagtgaagatgtcctgcaaggcttctggaaacacattcactgactatgttataaactgggtgaagcagagaactggacagggccttgagtggattggagagatttatcctggaaatgaaaatacttattacaatgagaagttcaagggcaaggccacactgactgcagacaaatcctccaacacagcctacatgcagctcagtagcctgacatctgaggactctgcggtctatttctgtgcaagagggatttattactacggtagtaggacgaggactatggactactggggtcaaggaacctcagtcaccgtctcctca

SEQ ID NO: 38 - protein, 123 aa, Homo sapiens
QVQLQQSGPELVKPGASVKMSCKASGNTFTDYVINWVKQRTGQGLEWIGEIYPGNENTYYNEKFKGKATLTADKSSNTAYMQLSSLTSEDSAVYFCARGIYYYGSRTRTMDYWGQGTSVTVSS

SEQ ID NO: 39 - protein, 5 aa, Homo sapiens
DYVIN

SEQ ID NO: 40 - protein, 16 aa, Homo sapiens
IYPGNENTYYNEKFKG

SEQ ID NO: 41 - protein, 14 aa, Homo sapiens
GIYYYGSRTRTMDY

SEQ ID NO: 42 - DNA, 366 bp, Homo sapiens
gacgtccaactgcaggagtctggacctgacctggtgaaaccttctcagtcactttcactcacctgcactgtcactggctactccatcaccagtggttatagctggcactggatccggcagtttccaggaaacaaactggaatggatgggctacatacactacagtggtggcactaactacaacccatctctcaaaagtcgaatctctatcactcgagacacatccaagaaccagttcttcctccagttgaattctgtgactactgaggacacagccacatattactgtgcaagatcggggtacggctacaggagtgcgtactattttgactactggggccaagggaccacggtcaccgtctcctca

SEQ ID NO: 43 - protein, 122 aa, Homo sapiens
DVQLQESGPDLVKPSQSLSLTCTVTGYSITSGYSWHWIRQFPGNKLEWMGYIHYSGGTNYNPSLKSRISITRDTSKNQFFLQLNSVTTEDTATYYCARSGYGYRSAYYFDYWGQGTTVTVSS

SEQ ID NO: 44 - protein, 6 aa, Homo sapiens
SGYSWH

SEQ ID NO: 45 - protein, 16 aa, Homo sapiens
YIHYSGGTNYNPSLKS

SEQ ID NO: 46 - protein, 13 aa, Homo sapiens
SGYGYRSAYYFDY

SEQ ID NO: 47 - DNA, 363 bp, Homo sapiens
caaatacagttggttcagagcggacctgagctgaagaagcctggagagacagtcaagatctcctgcaaggcttctgggtataccttcacaaaccatggaatgaactgggtgaagcaggctccaggaaagggtttaaagtggatgggctggataaacacctccactggagagccaacatatgctgatgacttcaagggacgttttgccttctctttggaaacctctgccagcactgcctttttgcagatcaacaacctcaaaaatgaggacacggcttcatatttctgtgcaagtcccctctactatatgtacgggcggtatatcgatgtctggggcgcagggaccgcggtcaccgtctcctca

SEQ ID NO: 48 - protein, 120 aa, Homo sapiens
QIQLVQSGPELKKPGETVKISCKASGYTFTNHGMNWVKQAPGKGLKWMGWNTSTGEPTYADDFKGRFAFSLETSASTAFLQINNLKNEDTASYFCASPLYYMYGRYIDVWGAGTAVTVSS

SEQ ID NO: 49 - protein, 5 aa, Homo sapiens
NHGMN

SEQ ID NO: 50 - protein, 15 aa, Homo sapiens
NTSTGEPTYADDFKG

SEQ ID NO: 51 - protein, 12 aa, Homo sapiens
PLYYMYGRYIDV

SEQ ID NO: 52 - DNA, 315 bp, Homo sapiens
acgtccaactgcaggagtctggacctgacctggtgaaaccttctcagtcactttcactcacctgcactgtcactggctactccatcaccagtggttatagctggcactggatccggcagtttccaggaaacaaactggaatggatgggctacatacactacagtggtggcactaactacaacccatctctcaaaagtcgaatctctatcactcgagacacatccaagaaccagttcttcctccagttgaattctgtgactactgaggacacagccacatattactgtgcaagatcggggtacggctacaggagtg

SEQ ID NO: 53 - protein, 122 aa, Homo sapiens
DVQLQESGPDLVKPSQSLSLTCTVTGYSITSGYSWHWIRQFPGNKLEWMGYIHYSGGTNYNPSLKSRISITRDTSKNQFFLQLNSVTTEDTATYYCARSGYGYRSAYYFDYWGQGTTLTVSS

SEQ ID NO: 54 - protein, 6 aa, Homo sapiens
SGYSWH

SEQ ID NO: 55 - protein, 16 aa, Homo sapiens
YIHYSGGTNYNPSLKS

SEQ ID NO: 56 - protein, 13 aa, Homo sapiens
SGYGYRSAYYFDY

SEQ ID NO: 57 - DNA, 360 bp, Homo sapiens
cagatccagttggtgcagtctggacctgacctgaagaagcctggagagacagtcaagatctcctgcaaggcttctgggtataccttcacaaaccatggaatgaactgggtgaagcaggctccaggaaaggatttaaagtggatgggctggataaacaccaacactggagagccaacatatgctgatgacttcaagggacggtttgccttctctttggaaacctctgccagcactgcctatttgcagatcaacaacctcaaaaatgaggacacggctacatatttctgtgcaagtcccctctactataggaacgggcgatacttcgatgtctggggcgcagggaccacggtcaccgtctcc

SEQ ID NO: 58 - protein, 121 aa, Homo sapiens
QIQLVQSGPDLKKPGETVKISCKASGYTFTNHGMNWVKQAPGKDLKWMGWINTNTGEPTYADDFKGRFAFSLETSASTAYLQINNLKNEDTATYFCASPLYYRNGRYFDVWGAGTTVTVSS

SEQ ID NO: 59 - protein, 5 aa, Homo sapiens
NHGMN

SEQ ID NO: 60 - protein, 17 aa, Homo sapiens
WINTNTGEPTYADDFKG

SEQ ID NO: 61 - protein, 12 aa, Homo sapiens
PLYYRNGRYFDV

SEQ ID NO: 62 - DNA, 354 bp, Homo sapiens
caggtccaactgcagcagcctggggctgaactggtgaagcctggggcttcagtgaagctgtcctgtaaggcttctggctacaccttcaccagctactggatgcactgggtgaagcagaggcctggacaaggccttgagtggattggagagattaatcctacctacggtcgtagtaattacaatgagaagttcaagagtaaggccacactgactgtagacaaatcctccagcacagcctacatgcaactcagcagcctgacatctgaggactctgcggtctattactgtgcaagattagtacgcctacggtacttcgatgtctggggcgcagggaccacggtcaccgtctcctca

SEQ ID NO: 63 - protein, 118 aa, Homo sapiens
QVQLQQPGAELVKPGASVKLSCKASGYTFTSYWMHWVKQRPGQGLEWIGEINPTYGRSNYNEKFKSKATLTVDKSSSTAYMQLSSLTSEDSAVYYCARLVRLRYFDVWGAGTTVTVSS

SEQ ID NO: 64 - protein, 5 aa, Homo sapiens
SYWMH

SEQ ID NO: 65 - protein, 17 aa, Homo sapiens
EINPTYGRSNYNEKFKS

SEQ ID NO: 66 - protein, 9 aa, Homo sapiens
LVRLRYFDV

SEQ ID NO: 67 - DNA, 330 bp, Homo sapiens
cagtacgaattgactcagccgccctcggtgtctgaggccccccggcagagggtcaccatctcctgttctggaagcagctccaacatcggaaataatgctataaactggtaccagcaactcccaggaaagcctcccaaactcctcatctattatgatgatctgttgccctcaggggtctctgaccgattctctggctccaagtctggcacctcaggctccctggccatcagtgggctgcagtctgaggatgaggctgattattactgtgcagcatgggatgacaacctgaatggtgtgattttcggcggagggaccaagctgaccgtccta

SEQ ID NO: 68 - protein, 110 aa, Homo sapiens
QYELTQPPSVSEAPRQRVTISCSGSSSNIGNNAINWYQQLPGKPPKLLIYYDDLLPSGVSDRFSGSKSGTSGSLAISGLQSEDEADYYCAAWDDNLNGVIFGGGTKLTVL

SEQ ID NO: 69 - protein, 13 aa, Homo sapiens
SGSSSNIGNNAIN

SEQ ID NO: 70 - protein, 7 aa, Homo sapiens
YDDLLPS

SEQ ID NO: 71 - protein, 11 aa, Homo sapiens
AAWDDNLNGVI

SEQ ID NO: 72 - DNA, 324 bp, Homo sapiens
gacatccagatgacccagtctccactctccctgtctgcatctgtaggagacagagtcaccatcacttgccgggcaagtcagagcattaacggctacttaaattggtatcagcagaaaccagggaaagcccctaacctcctgatctacgctacatccagtttgcaaagtggggtcccatcaaggttcagtggcagtggatctgggacagatttcactctcaccatcagcagtctgcaacctgaagattttgcaacttactactgtcaacagagttacagtacccccccgtacacttttggccaggggaccaagctggagatcaaa

SEQ ID NO: 73 - protein, 108 aa, Homo sapiens
DIQMTQSPLSLSASVGDRVTITCRASQSINGYLNWYQQKPGKAPNLLIYATSSLQSGVPSRFSGSGSGTDFTLTISSLQPEDFATYYCQQSYSTPPYTFGQGTKLEIK

SEQ ID NO: 74 - protein, 11 aa, Homo sapiens
RASQSINGYLN

SEQ ID NO: 75 - protein, 7 aa, Homo sapiens
ATSSLQS

SEQ ID NO: 76 - protein, 10 aa, Homo sapiens
QQSYSTPPYT

SEQ ID NO: 77 - DNA, 321 bp, Homo sapiens
gacatccagatgacccagtctccactctccctgtctgcatctgtaggagacagagtcaccatcacttgccaggcgagtcgggacattagaaactatttaaattggtatcaacaaaaaccagggaaagccccgaagctcctgatctacgatgcatccagtttgcaaacaggggtcccatcaaggttcggtggcagtggatctgggacagactttagtttcaccatcggcagcctgcagcctgaagatattgcaacatattactgtcaacagtttgatagtctccctcacacttttggccaggggaccaaactggagatcaaa

SEQ ID NO: 78 - protein, 107 aa, Homo sapiens
DIQMTQSPLSLSASVGDRVTITCQASRDIRNYLNWYQQKPGKAPKLLIYDASSLQTGVPSRFGGSGSGTDFSFTIGSLQPEDIATYYCQQFDSLPHTFGQGTKLEIK

SEQ ID NO: 79 - protein, 11 aa, Homo sapiens
QASRDIRNYLN

SEQ ID NO: 80 - protein, 7 aa, Homo sapiens
DASSLQT

SEQ ID NO: 81 - protein, 9 aa, Homo sapiens
QQFDSLPHT

SEQ ID NO: 82 - DNA, 324 bp, Homo sapiens
gacatccagatgacccagtttccagccaccctgtctgtgtctccaggggaaagagccaccctctcctgcagggccagtcagagtgttatgaggaacttagcctggtaccagcagaaacctggccagcctcccaggctcctcatctatggtgcatccaaaagggccactggcatcccagccaggttcagtggcagtgggtctgggacagccttcactctcaccatcagcaacctagagcctgaagattttgcagtttattactgtcaccaacgtagcacctggcctctggggactttcggccctgggaccaaactggaggccaaa

SEQ ID NO: 83 - protein, 108 aa, Homo sapiens
DIQMTQFPATLSVSPGERATLSCRASQSVMRNLAWYQQKPGQPPRLLIYGASKRATGIPARFSGSGSGTAFTLTISNLEPEDFAVYYCHQRSTWPLGTFGPGTKLEAK

SEQ ID NO: 84 - protein, 11 aa, Homo sapiens
RASQSVMRNLA

SEQ ID NO: 85 - protein, 7 aa, Homo sapiens
GASKRAT

SEQ ID NO: 86 - protein, 10 aa, Homo sapiens
HQRSTWPLGT

SEQ ID NO: 87 - DNA, 327 bp, Homo sapiens
gacatccagatgacccagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggaggtcactttcggccctgggaccaaagtggatatcaaa

SEQ ID NO: 88 - protein, 109 aa, Homo sapiens
DIQMTQSPATLSLSPGERATLSCRASQSVSSYLAWYQQKPGQAPRLLIYDASNRATGIPARFSGSGSGTDFTLTISSLEPEDFAVYYCQQRSNWPPEVTFGPGTKVDIK

SEQ ID NO: 89 - protein, 11 aa, Homo sapiens
RASQSVSSYLA

SEQ ID NO: 90 - protein, 7 aa, Homo sapiens
DASNRAT

SEQ ID NO: 91 - protein, 11 aa, Homo sapiens
QQRSNWPPEVT

SEQ ID NO: 92 - DNA, 336 bp, Homo sapiens
gacatccagatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttacttggcatctacccgggaatccggggtccctgaccgattcagtggcagcgggtctgggacagatttcactctcaccatcagcagcctgcaggctgaagatgtggcagtttattactgtcagcaatattatagtacttggacgttcggccaagggaccaaggtggaaatcaaa

SEQ ID NO: 93 - protein, 112 aa, Homo sapiens
DIQMTQSPDSLAVSLGERATINCKSSQSVLYSSNNKNYLAWYQQKPGQPPKLLIYLASTRESGVPDRFSGSGSGTDFTLTISSLQAEDVAVYYCQQYYSTWTFGQGTKVEIK

SEQ ID NO: 94 - protein, 17 aa, Homo sapiens
KSSQSVLYSSNNKNYLA

SEQ ID NO: 95 - protein, 7 aa, Homo sapiens
LASTRES

SEQ ID NO: 96 - protein, 8 aa, Homo sapiens
QQYYSTWT

SEQ ID NO: 97 - DNA, 324 bp, Homo sapiens
gaagttgtgctcacccagtctccaaccgccatggctgcatctcccggggagaagatcactatcacctgcagtgccagctcaactttaagttccaattacttgcattggtatcagcagaagccaggattctcccctaaactcttgatttataggacatccaatctggcctctggagtcccaggtcgcttcagtggcagtgggtctgggacctcttactctctcacaattggcaccatggaggctgaagatgttgccacttactactgccagcagggtagtagtataccgctcacgttcggtgctgggaccaagctggagctgaag

SEQ ID NO: 98 - protein, 108 aa, Homo sapiens
EVVLTQSPTAMAASPGEKITITCSASSTLSSNYLHWYQQKPGFSPKLLIYRTSNLASGVPGRFSGSGSGTSYSLTIGTMEAEDVATYYCQQGSSIPLTFGAGTKLELK

SEQ ID NO: 99 - protein, 12 aa, Homo sapiens
SASSTLSSNYLH

SEQ ID NO: 100 - protein, 7 aa, Homo sapiens
RTSNLAS

SEQ ID NO: 101 - protein, 9 aa, Homo sapiens
QQGSSIPLT

SEQ ID NO: 102 - DNA, 330 bp, Homo sapiens
gacattgtgctgacacagtctcctgcttccttagctgtatctctggggcagagggccaccatctcatgcagggccagcaaaagtgtcagtacatctgcctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgtatacgttcggaggggggaccaagctggaaatc

SEQ ID NO: 103 - protein, 111 aa, Homo sapiens
DIVLTQSPASLAVSLGQRATISCRASKSVSTSAYSYMHWYQQKPGQPPKLLIYLASNLESGVPARFSGSGSGTDFTLNIHPVEEEDAATYYCQHSRELPYTFGGGTKLEIK

SEQ ID NO: 104 - protein, 15 aa, Homo sapiens
RASKSVSTSAYSYMH

SEQ ID NO: 105 - protein, 7 aa, Homo sapiens
LASNLES

SEQ ID NO: 106 - protein, 9 aa, Homo sapiens
QHSRELPYT

SEQ ID NO: 107 - DNA, 321 bp, Homo sapiens
gatatccagatgacacagactacatcctccctatctgcctctctgggagacagagtcaccatcagttgcagggcaagtcaggacattagcaattatttaaactggtatcagcagaaaccagatggaactattaaactcctgatctactacacatcaagattacactcaggagtcccatcaaggttcagtggcagtgggtctggaacagattattctctcaccattagcaacctggaacaagaagattttgccacttacttttgccaacagggtaaaacgcttccgtggacgttcggtggaggcaccaagctggaaatcaaa

SEQ ID NO: 108 - protein, 107 aa, Homo sapiens
DIQMTQTTSSLSASLGDRVTISCRASQDISNYLNWYQQKPDGTIKLLIYYTSRLHSGVPSRFSGSGSGTDYSLTISNLEQEDFATYFCQQGKTLPWTFGGGTKLEIK

SEQ ID NO: 109 - protein, 11 aa, Homo sapiens
RASQDISNYLN

SEQ ID NO: 110 - protein, 6 aa, Homo sapiens
TSRLHS

SEQ ID NO: 111 - protein, 9 aa, Homo sapiens
QQGKTLPWT

SEQ ID NO: 112 - DNA, 321 bp, Homo sapiens
gatatccagatgacacagactacatcctccctgtctgcctctctgggagacagagtcaccatcagttgcagggcaagtcaggacattagtaattatttaaattggtatcagcagaaaccagatggatctgttaaactcctgatctactacacatcaagattacactcaggagtcccatcaaggttcagtggcagtgggtctggaacagattattctctcaccattagcaacctggaacaagaagatattgccacttacttttgccaacagggaaagacgcttccgtggacgttcggtggaggcaccaagctggaaatcaaa

SEQ ID NO: 113 - protein, 107 aa, Homo sapiens
DIQMTQTTSSLSASLGDRVTISCRASQDISNYLNWYQQKPDGSVKLLIYYTSRLHSGVPSRFSGSGSGTDYSLTISNLEQEDIATYFCQQGKTLPWTFGGGTKLEIK

SEQ ID NO: 114 - protein, 11 aa, Homo sapiens
RASQDISNYLN

SEQ ID NO: 115 - protein, 5 aa, Homo sapiens
TSRLH

SEQ ID NO: 116 - protein, 9 aa, Homo sapiens
QQGKTLPWT

SEQ ID NO: 117 - DNA, 336 bp, Homo sapiens
gatattgtgatgacgcaggctgcattctccaatccagtcactcttggaacatcagcttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttccgtacacgttcggaggggggaccaagctggaaatcaaa

SEQ ID NO: 118 - protein, 112 aa, Homo sapiens
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPYTFGGGTKLEIK

SEQ ID NO: 119 - protein, 16 aa, Homo sapiens
RSSKSLLHSNGITYLY

SEQ ID NO: 120 - protein, 7 aa, Homo sapiens
QMSNLAS

SEQ ID NO: 121 - protein, 9 aa, Homo sapiens
AQNLELPYT

SEQ ID NO: 122 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSAYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 123 - DNA, 816 bp, Artificial Sequence
  Synthetic oligonucleotide
gacatccagatgacccagtctccactctccctgtctgcatctgtaggagacagagtcaccatcacttgccaggcgagtcgggacattagaaactatttaaattggtatcaacaaaaaccagggaaagccccgaagctcctgatctacgatgcatccagtttgcaaacaggggtcccatcaaggttcggtggcagtggatctgggacagactttagtttcaccatcggcagcctgcagcctgaagatattgcaacatattactgtcaacagtttgatagtctccctcacacttttggccaggggaccaaactggagatcaaaggtgggggcggatctgggggcggcgggtccggtggtggtggtagtgaagttcaattgttagagtctggtggcggtcttgttcagcctggtggttctttacgtctttcttgcgctgcttccggattcactttctctatttaccgtatgcagtgggttcgccaagctcctggtaaaggtttggagtgggtttctggtatctctccttctggtggcactacttggtatgctgactccgttaaaggtcgcttcactatctctagagacaactctaagaatactctctacttgcagatgaacagcttaagggctgaggacacggccgtgtattactgtgcgagatggagcgggggttcgggctatgcttttgatatctggggccaagggacaatggtcaccgtctcaagcgacgacgacgacaaaagctttctagaacaaaaactcatctcagaagaggatctgaatagcgccgtcgaccatcatcatcatcatcattga

SEQ ID NO: 124 - protein, 242 aa, Artificial Sequence
  Synthetic peptide
DIQMTQSPLSLSASVGDRVTITCQASRDIRNYLNWYQQKPGKAPKLLIYDASSLQTGVPSRFGGSGSGTDFSFTIGSLQPEDIATYYCQQFDSLPHTFGQGTKLEIKGGGGSGGGGSGGGGSEVQLLESGGGLVQPGGSLRLSCAASGFTFSIYRMQWVRQAPGKGLEWVSGISPSGGTTWYADSVKGRFTISRDNSKNTLYLQMNSLRAEDTAVYYCARWSGGSGYAFDIWGQGTMVTVSS

SEQ ID NO: 125 - DNA, 816 bp, Artificial Sequence
  Synthetic oligonucleotide
gaagttcaattgttagagtctggtggcggtcttgttcagcctggtggttctttacgtctttcttgcgctgcttccggattcactttctctatttaccgtatgcagtgggttcgccaagctcctggtaaaggtttggagtgggtttctggtatctctccttctggtggcactacttggtatgctgactccgttaaaggtcgcttcactatctctagagacaactctaagaatactctctacttgcagatgaacagcttaagggctgaggacacggccgtgtattactgtgcgagatggagcgggggttcgggctatgcttttgatatctggggccaagggacaatggtcaccgtctcaagcggtgggggcggatctgggggcggcgggtccggtggtggtggtagtgacatccagatgacccagtctccactctccctgtctgcatctgtaggagacagagtcaccatcacttgccaggcgagtcgggacattagaaactatttaaattggtatcaacaaaaaccagggaaagccccgaagctcctgatctacgatgcatccagtttgcaaacaggggtcccatcaaggttcggtggcagtggatctgggacagactttagtttcaccatcggcagcctgcagcctgaagatattgcaacatattactgtcaacagtttgatagtctccctcacacttttggccaggggaccaaactggagatcaaagacgacgacgacaaaagctttctagaacaaaaactcatctcagaagaggatctgaatagcgccgtcgaccatcatcatcatcatcattga

SEQ ID NO: 126 - protein, 242 aa, Artificial Sequence
  Synthetic peptide
EVQLLESGGGLVQPGGSLRLSCAASGFTFSIYRMQWVRQAPGKGLEWVSGISPSGGTTWYADSVKGRFTISRDNSKNTLYLQMNSLRAEDTAVYYCARWSGGSGYAFDIWGQGTMVTVSSGGGGSGGGGSGGGGSDIQMTQSPLSLSASVGDRVTITCQASRDIRNYLNWYQQKPGKAPKLLIYDASSLQTGVPSRFGGSGSGTDFSFTIGSLQPEDIATYYCQQFDSLPHTFGQGTKLEIK

SEQ ID NO: 127 - DNA, 816 bp, Artificial Sequence
  Synthetic oligonucleotide
gacatccagatgacccagtctccactctccctgtctgcatctgtaggagacagagtcaccatcacttgccaggcgagtcgggacattagaaactatttaaattggtatcaacaaaaaccagggaaagccccgaagctcctgatctacgatgcatccagtttgcaaacaggggtcccatcaaggttcggtggcagtggatctgggacagactttagtttcaccatcggcagcctgcagcctgaagatgaggcaacatattactgtcaacagtttgatagtctccctcacacttttggccaggggaccaaactggagatcaaaggtgggggcggatctgggggcggcgggtccggtggtggtggtagtgaagttcaattgttagagtctggtggcggtcttgttcagcctggtggttctttacgtctttcttgcgctgcttccggattcactttctctatttaccgtatgcagtgggttcgccaagctcctggtaaaggtttggagtgggtttctggtatctctccttctggtggcactacttggtatgctgactccgttaaaggtcgcttcactatctctagagacaactctaagaatactctctacttgcagatgaacagcttaagggctgaggacacggccgtgtattactgtgcgagatggagcgggggttcgggctatgcttttgatatctggggccaagggacaatggtcaccgtctcaagcgacgacgacgacaaaagctttctagaacaaaaactcatctcagaagaggatctgaatagcgccgtcgaccatcatcatcatcatcattga

SEQ ID NO: 128 - protein, 242 aa, Artificial Sequence
  Synthetic peptide
DIQMTQSPLSLSASVGDRVTITCQASRDIRNYLNWYQQKPGKAPKLLIYDASSLQTGVPSRFGGSGSGTDFSFTIGSLQPEDEATYYCQQFDSLPHTFGQGTKLEIKGGGGSGGGGSGGGGSEVQLLESGGGLVQPGGSLRLSCAASGFTFSIYRMQWVRQAPGKGLEWVSGISPSGGTTWYADSVKGRFTISRDNSKNTLYLQMNSLRAEDTAVYYCARWSGGSGYAFDIWGQGTMVTVSS

SEQ ID NO: 129 - DNA, 726 bp, Artificial Sequence
  Synthetic oligonucleotide
atggacatgagggtccccgctcagctcctggggctccttctgctctggctcccaggtgccagatgtgacatccagatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttacttggcatctacccgggaatccggggtccctgaccgattcagtggcagcgggtctgggacagatttcactctcaccatcagcagcctgcaggctgaagatgtggcagtttattactgtcagcaatattatagtacttggacgttcggccaaggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 130 - protein, 219 aa, Artificial Sequence
  Synthetic peptide
DIQMTQSPDSLAVSLGERATINCKSSQSVLYSSNNKNYLAWYQQKPGQPPKLLIYLASTRESGVPDRFSGSGSGTDFTLTISSLQAEDVAVYYCQQYYSTWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 22 aa, Artificial Sequence
  Synthetic peptide
MDMRVPAQLLGLLLLWLPGARC

SEQ ID NO: 132 - DNA, 2220 bp, Artificial Sequence
  Synthetic oligonucleotide
atgggttggagcctcatcttgctcttccttgtcgctgttgctacgcgtgtcctgtccgacatccagatgacccagtctccactctccctgtctgcatctgtaggagacagagtcaccatcacttgccaggcgagtcgggacattagaaactatttaaattggtatcaacaaaaaccagggaaagccccgaagctcctgatctacgatgcatccagtttgcaaacaggggtcccatcaaggttcggtggcagtggatctgggacagactttagtttcaccatcggcagcctgcagcctgaagatgaggcaacatattactgtcaacagtttgatagtctccctcacacttttggccaggggaccaaactggagatcaaaggtgggggcggatctgggggcggcgggtccggtggtggtggtagtgaagttcaattgttagagtctggtggcggtcttgttcagcctggtggttctttacgtctttcttgcgctgcttccggattcactttctctatttaccgtatgcagtgggttcgccaagctcctggtaaaggtttggagtgggtttctggtatctctccttctggtggcactacttggtatgctgactccgttaaaggtcgcttcactatctctagagacaactctaagaatactctctacttgcagatgaacagcttaagggctgaggacacggccgtgtattactgtgcgagatggagcgggggttcgggctatgcttttgatatctggggccaagggacaatggtcaccgtctcaagcggcggtggagggtccggtggagggggctctggagggggcggttcagggggcggtggatccgggggaggtggctccgaggtccagctgttggagtccggcggtggcctggtgcagcctggggggtccctgagactctcctgcgcagctagcggcttcaccttcagcaattaccccatgtactgggtgcgccaggctcctggaaaggggctggagtgggtttccaggatctctagcagcggtggcaggaccgtgtacgctgactccgtgaagggccggttcacaatctccagagacaattccaagaacactctgtacctgcaaatgaacagcctgagagctgaggatactgcagtgtactactgcgccagagataggtggtccagatctgcagccgagtacggactggggggctactggggacagggaaccctggtcaccgtctcaagcgcctcaacgaaggggcccagcgtgttccccctggcccccagcagcaagagcaccagcggcggcaccgccgccctgggctgcctggtgaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 133 - protein, 720 aa, Artificial Sequence
  Synthetic peptide
DIQMTQSPLSLSASVGDRVTITCQASRDIRNYLNWYQQKPGKAPKLLIYDASSLQTGVPSRFGGSGSGTDFSFTIGSLQPEDEATYYCQQFDSLPHTFGQGTKLEIKGGGGSGGGGSGGGGSEVQLLESGGGLVQPGGSLRLSCAASGFTFSIYRMQWVRQAPGKGLEWVSGISPSGGTTWYADSVKGRFTISRDNSKNTLYLQMNSLRAEDTAVYYCARWSGGSGYAFDIWGQGTMVTVSSGGGGSGGGGSGGGGSGGGGSGGGGSEVQLLESGGGLVQPGGSLRLSCAASGFTFSNYPMYWVRQAPGKGLEWVSRISSSGGRTVYADSVKGRFTISRDNSKNTLYLQMNSLRAEDTAVYYCARDRWSRSAAEYGLGGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 134 - protein, 19 aa, Artificial Sequence
  Synthetic peptide
MGWSLILLFLVAVATRVLS

SEQ ID NO: 135 - protein, 20 aa, Artificial Sequence
  Synthetic peptide
GGGGSGGGGSGGGGSGGGGS

SEQ ID NO: 136 - DNA, 2196 bp, Artificial Sequence
  Synthetic oligonucleotide
atgggttggagcctcatcttgctcttccttgtcgctgttgctacgcgtgtcctgtccgaggtccagctgttggagtccggcggtggcctggtgcagcctggggggtccctgagactctcctgcgcagctagcggcttcaccttcagcaattaccccatgtactgggtgcgccaggctcctggaaaggggctggagtgggtttccaggatctctagcagcggtggcaggaccgtgtacgctgactccgtgaagggccggttcacaatctccagagacaattccaagaacactctgtacctgcaaatgaacagcctgagagctgaggatactgcagtgtactactgcgccagagataggtggtccagatctgcagccgagtacggactggggggctactggggacagggaaccctggtcaccgtctcaagcgcctcaacgaaggggcccagcgtgttccccctggcccccagcagcaagagcaccagcggcggcaccgccgccctgggctgcctggtgaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccggcgggggtggatccggtggagggggctccggcggtggcgggtccgacatccagatgacccagtctccactctccctgtctgcatctgtaggagacagagtcaccatcacttgccaggcgagtcgggacattagaaactatttaaattggtatcaacaaaaaccagggaaagccccgaagctcctgatctacgatgcatccagtttgcaaacaggggtcccatcaaggttcggtggcagtggatctgggacagactttagtttcaccatcggcagcctgcagcctgaagatgaggcaacatattactgtcaacagtttgatagtctccctcacacttttggccaggggaccaaactggagatcaaaggtgggggcggatctgggggcggcgggtccggtggtggtggtagtgaagttcaattgttagagtctggtggcggtcttgttcagcctggtggttctttacgtctttcttgcgctgcttccggattcactttctctatttaccgtatgcagtgggttcgccaagctcctggtaaaggtttggagtgggtttctggtatctctccttctggtggcactacttggtatgctgactccgttaaaggtcgcttcactatctctagagacaactctaagaatactctctacttgcagatgaacagcttaagggctgaggacacggccgtgtattactgtgcgagatggagcgggggttcgggctatgcttttgatatctggggccaagggacaatggtcaccgtctcaagctga

SEQ ID NO: 137 - protein, 712 aa, Artificial Sequence
  Synthetic peptide
EVQLLESGGGLVQPGGSLRLSCAASGFTFSNYPMYWVRQAPGKGLEWVSRISSSGGRTVYADSVKGRFTISRDNSKNTLYLQMNSLRAEDTAVYYCARDRWSRSAAEYGLGGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSGGGGSGGGGSDIQMTQSPLSLSASVGDRVTITCQASRDIRNYLNWYQQKPGKAPKLLIYDASSLQTGVPSRFGGSGSGTDFSFTIGSLQPEDEATYYCQQFDSLPHTFGQGTKLEIKGGGGSGGGGSGGGGSEVQLLESGGGLVQPGGSLRLSCAASGFTFSIYRMQWVRQAPGKGLEWVSGISPSGGTTWYADSVKGRFTISRDNSKNTLYLQMNSLRAEDTAVYYCARWSGGSGYAFDIWGQGTMVTVSS

SEQ ID NO: 138 - protein, 16 aa, Artificial Sequence
  Synthetic peptide
SGGGGSGGGGSGGGGS

SEQ ID NO: 139 - DNA, 711 bp, Artificial Sequence
  Synthetic oligonucleotide
atggacatgagggtccccgctcagctcctggggctccttctgctctggctcccaggtgccagatgtgacatccagatgacccagtctccactctccctgtctgcatctgtaggagacagagtcaccatcacttgccaggcgagtcgggacattagaaactatttaaattggtatcaacaaaaaccagggaaagccccgaagctcctgatctacgatgcatccagtttgcaaacaggggtcccatcaaggttcggtggcagtggatctgggacagactttagtttcaccatcggcagcctgcagcctgaagatattgcaacatattactgtcaacagtttgatagtctccctcacacttttggccaggggaccaaactggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 140 - protein, 214 aa, Artificial Sequence
  Synthetic peptide
DIQMTQSPLSLSASVGDRVTITCQASRDIRNYLNWYQQKPGKAPKLLIYDASSLQTGVPSRFGGSGSGTDFSFTIGSLQPEDIATYYCQQFDSLPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - DNA, 2250 bp, Artificial Sequence
  Synthetic oligonucleotide
atgggttggagcctcatcttgctcttccttgtcgctgttgctacgcgtgtcctgtccgacatccagatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttacttggcatctacccgggaatccggggtccctgaccgattcagtggcagcgggtctgggacagatttcactctcaccatcagcagcctgcaggctgaagatgtggcagtttattactgtcagcaatattatagtacttggacgttcggccaagggaccaaggtggaaatcaaaggcggtggagggtccggtgggggcggatctgggggcggcgggtccggtggtggtggtagtgaggtccagctgttggagtccggcggtggcctggtgcagcctggggggtccctgagactctcctgcgcagctagcggcttcaccttcagcaattaccccatgtactgggtgcgccaggctcctggaaaggggctggagtgggtttccaggatctctagcagcggtggcaggaccgtgtacgctgactccgtgaagggccggttcacaatctccagagacaattccaagaacactctgtacctgcaaatgaacagcctgagagctgaggatactgcagtgtactactgcgccagagataggtggtccagatctgcagccgagtacggactggggggctactggggacagggaaccctggtcaccgtctcaagcggcggtggagggtccggtggagggggctctggagggggcggttcagggggcggtggatccgggggaggtggctccgaggtccagctgttggagtccggcggtggcctggtgcagcctggggggtccctgagactctcctgcgcagctagcggcttcaccttcagcatttaccgtatgcagtgggtgcgccaggctcctggaaaggggctggagtgggtttccggtatctctccctctggtggcacgacgtggtatgctgactccgtgaagggccggttcacaatctccagagacaattccaagaacactctgtacctgcaaatgaacagcctgagagctgaggatactgcagtgtactactgcgccagatggtccgggggctccggatacgccttcgacatctggggacagggaaccatggtcaccgtctcaagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttga

SEQ ID NO: 142 - protein, 730 aa, Artificial Sequence
  Synthetic peptide
DIQMTQSPDSLAVSLGERATINCKSSQSVLYSSNNKNYLAWYQQKPGQPPKLLIYLASTRESGVPDRFSGSGSGTDFTLTISSLQAEDVAVYYCQQYYSTWTFGQGTKVEIKGGGGSGGGGSGGGGSGGGGSEVQLLESGGGLVQPGGSLRLSCAASGFTFSNYPMYWVRQAPGKGLEWVSRISSSGGRTVYADSVKGRFTISRDNSKNTLYLQMNSLRAEDTAVYYCARDRWSRSAAEYGLGGYWGQGTLVTVSSGGGGSGGGGSGGGGSGGGGSGGGGSEVQLLESGGGLVQPGGSLRLSCAASGFTFSIYRMQWVRQAPGKGLEWVSGISPSGGTTWYADSVKGRFTISRDNSKNTLYLQMNSLRAEDTAVYYCARWSGGSGYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 143 - DNA, 2226 bp, Artificial Sequence
  Synthetic oligonucleotide
atgggttggagcctcatcttgctcttccttgtcgctgttgctacgcgtgtcctgtccgaggtccagctgttggagtccggcggtggcctggtgcagcctggggggtccctgagactctcctgcgcagctagcggcttcaccttcagcatttaccgtatgcagtgggtgcgccaggctcctggaaaggggctggagtgggtttccggtatctctccctctggtggcacgacgtggtatgctgactccgtgaagggccggttcacaatctccagagacaattccaagaacactctgtacctgcaaatgaacagcctgagagctgaggatactgcagtgtactactgcgccagatggtccgggggctccggatacgccttcgacatctggggacagggaaccatggtcaccgtctcaagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccggcgggggtggatccggtggagggggctccggcggtggcgggtccgacatccagatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttacttggcatctacccgggaatccggggtccctgaccgattcagtggcagcgggtctgggacagatttcactctcaccatcagcagcctgcaggctgaagatgtggcagtttattactgtcagcaatattatagtacttggacgttcggccaagggaccaaggtggaaatcaaaggcggtggagggtccggtgggggcggatctgggggcggcgggtccggtggtggtggtagtgaggtccagctgttggagtccggcggtggcctggtgcagcctggggggtccctgagactctcctgcgcagctagcggcttcaccttcagcaattaccccatgtactgggtgcgccaggctcctggaaaggggctggagtgggtttccaggatctctagcagcggtggcaggaccgtgtacgctgactccgtgaagggccggttcacaatctccagagacaattccaagaacactctgtacctgcaaatgaacagcctgagagctgaggatactgcagtgtactactgcgccagagataggtggtccagatctgcagccgagtacggactggggggctactggggacagggaaccctggtcaccgtctcaagctga

SEQ ID NO: 144 - protein, 722 aa, Artificial Sequence
  Synthetic peptide
EVQLLESGGGLVQPGGSLRLSCAASGFTFSIYRMQWVRQAPGKGLEWVSGISPSGGTTWYADSVKGRFTISRDNSKNTLYLQMNSLRAEDTAVYYCARWSGGSGYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSGGGGSGGGGSDIQMTQSPDSLAVSLGERATINCKSSQSVLYSSNNKNYLAWYQQKPGQPPKLLIYLASTRESGVPDRFSGSGSGTDFTLTISSLQAEDVAVYYCQQYYSTWTFGQGTKVEIKGGGGSGGGGSGGGGSGGGGSEVQLLESGGGLVQPGGSLRLSCAASGFTFSNYPMYWVRQAPGKGLEWVSRISSSGGRTVYADSVKGRFTISRDNSKNTLYLQMNSLRAEDTAVYYCARDRWSRSAAEYGLGGYWGQGTLVTVSS

SEQ ID NO: 145 - DNA, 57 bp, Artificial Sequence
  Synthetic primer
gaattacatatgaaaaaactgctgttcgcgattccgctggtggtgccgttctatagc

SEQ ID NO: 146 - DNA, 59 bp, Artificial Sequence
  Synthetic primer
atgatggtcgacggcgctattcagatcctcttctgagatgagtttttgttctagaaagc

SEQ ID NO: 147 - DNA, 51 bp, Artificial Sequence
  Synthetic primer
gctggtggtgccgttctatagccatagtgaagttcaattgttagagtctgg

SEQ ID NO: 148 - DNA, 56 bp, Artificial Sequence
  Synthetic primer
tctgggggcggcgggtccggtggtggtggtagtgacatccagatgacccagtctcc

SEQ ID NO: 149 - DNA, 60 bp, Artificial Sequence
  Synthetic primer
gagtttttgttctagaaagcttttgtcgtcgtcgtctttgatctccagtttggtcccctg

SEQ ID NO: 150 - DNA, 54 bp, Artificial Sequence
  Synthetic primer
ccggacccgccgcccccagatccgcccccaccgcttgagacggtgaccattgtc

SEQ ID NO: 151 - DNA, 62 bp, Artificial Sequence
  Synthetic primer
gggcggatctgggggcggcgggtccggtggtggtggtagtgacatccagatgacccagtctc

SEQ ID NO: 152 - DNA, 64 bp, Artificial Sequence
  Synthetic primer
cccgccgcccccagatccgcccccaccggaccctccaccgccgcttgagacggtgaccattgtc

SEQ ID NO: 153 - DNA, 50 bp, Artificial Sequence
  Synthetic primer
gctggtggtgccgttctatagccatagtgacatccagatgacccagtctc

SEQ ID NO: 154 - DNA, 60 bp, Artificial Sequence
  Synthetic primer
atctgggggcggcgggtccggtggtggtggtagtgaagttcaattgttagagtctggtgg

SEQ ID NO: 155 - DNA, 58 bp, Artificial Sequence
  Synthetic primer
gagtttttgttctagaaagcttttgtcgtcgtcgtcgcttgagacggtgaccattgtc

SEQ ID NO: 156 - DNA, 57 bp, Artificial Sequence
  Synthetic primer
accggacccgccgcccccagatccgcccccacctttgatctccagtttggtcccctg

SEQ ID NO: 157 - DNA, 62 bp, Artificial Sequence
  Synthetic primer
gggcggatctgggggcggcgggtccggtggtggtggtagtgaagttcaattgttagagtctg

SEQ ID NO: 158 - DNA, 65 bp, Artificial Sequence
  Synthetic primer
ccgccgcccccagatccgcccccaccggaccctccaccgcctttgatctccagtttggtcccctg

SEQ ID NO: 159 - DNA, 33 bp, Artificial Sequence
  Synthetic primer
catagtgacatccagctgacccagtctccactc

SEQ ID NO: 160 - DNA, 34 bp, Artificial Sequence
  Synthetic primer
ggtaaaggtttggagwtcgtttctggtatctctc

SEQ ID NO: 161 - DNA, 34 bp, Artificial Sequence
  Synthetic primer
ctccctgtctgcatctvncggagacagagtcacc

SEQ ID NO: 162 - DNA, 35 bp, Artificial Sequence
  Synthetic primer
gtcaccatcacttgcrvggcgagtcgggacattag

SEQ ID NO: 163 - DNA, 33 bp, Artificial Sequence
  Synthetic primer
gtaggagacagagtccgcatcacttgccaggcg

SEQ ID NO: 164 - DNA, 33 bp, Artificial Sequence
  Synthetic primer
gcgagtcgggacattamcaactatttaaattgg

SEQ ID NO: 165 - DNA, 32 bp, Artificial Sequence
  Synthetic primer
ggttctttacgtcttgaatgcgctgcttccgg

SEQ ID NO: 166 - DNA, 34 bp, Artificial Sequence
  Synthetic primer
ctcctgatctacgatggctccagtttgcaaacag

SEQ ID NO: 167 - DNA, 32 bp, Artificial Sequence
  Synthetic primer
ctgggacagactttwmcttcaccatcggcagc

SEQ ID NO: 168 - DNA, 34 bp, Artificial Sequence
  Synthetic primer
ctgcagcctgaagatnnkgcaacatattactgtc

SEQ ID NO: 169 - DNA, 34 bp, Artificial Sequence; identical to SEQ ID NO: 168
  Synthetic primer

SEQ ID NO: 170 - DNA, 33 bp, Artificial Sequence
  Synthetic primer
caagggacaatggtcgtggtctcaagcgacgac

SEQ ID NO: 171 - DNA, 41 bp, Artificial Sequence
  Synthetic primer
gttgctacgcgtgtcctgtccgacatccagatgacccagtc

SEQ ID NO: 172 - DNA, 105 bp, Artificial Sequence
  Synthetic primer
gaagccgctagctgcgcaggagagtctcagggaccccccaggctgcaccaggccaccgccggactccaacagctggacctcggagccacctcccccggatccacc

SEQ ID NO: 173 - DNA, 89 bp, Artificial Sequence
  Synthetic primer
acctcccccggatccaccgccccctgaaccgccccctccagagccccctccaccggaccctccaccgccgcttgagacggtgaccattg

SEQ ID NO: 174 - DNA, 62 bp, Artificial Sequence
  Synthetic primer
gggggtggatccggtggagggggctccggcggtggcgggtccgacatccagatgacccagtc

SEQ ID NO: 175 - DNA, 38 bp, Artificial Sequence
  Synthetic primer
ggatctcactgggtgtcagcttgagacggtgaccattg

SEQ ID NO: 176 - DNA, 22 bp, Artificial Sequence
  Synthetic primer
cttccccgaaccggtgacggtg

SEQ ID NO: 177 - DNA, 68 bp, Artificial Sequence
  Synthetic primer
gggcagagatctcactgggtgtcagccgggatccacccccgccggatttacccggagacagggagagg

SEQ ID NO: 178 - DNA, 33 bp, Artificial Sequence
  Synthetic primer
gaggtccagctgttgcagtccggcggtggcctg

SEQ ID NO: 179 - DNA, 33 bp, Artificial Sequence
  Synthetic primer
gggctggagtgggttgscaggatctctagcagc

SEQ ID NO: 180 - DNA, 35 bp, Artificial Sequence
  Synthetic primer
aagctgctcatttacnnkgcatctacccgggaatc

SEQ ID NO: 181 - DNA, 34 bp, Artificial Sequence
  Synthetic primer
ctgcaggctgaagatkwkgcagtttattactgtc

SEQ ID NO: 182 - protein, 5 aa, Artificial Sequence
  Synthetic peptide
GGGGS

SEQ ID NO: 183 - protein, 6 aa, Artificial Sequence
  Synthetic peptide
SGGGGS

SEQ ID NO: 184 - protein, 25 aa, Artificial Sequence
  Synthetic peptide
GGGGSGGGGSGGGGSGGGGSGGGGS

SEQ ID NO: 185 - protein, 15 aa, Artificial Sequence
  Synthetic peptide
GGGGSGGGGSGGGGS

(19) United States
(12) Patent Application Publication
(10) Pub. No.: US 2009/0130105 A1
(43) Pub. Date: May 21, 2009

(54) COMPOSITIONS THAT BIND MULTIPLE EPITOPES OF IGF-1R

(75) Inventors: Scott Glaser, San Diego, CA (US); Stephen Demarest, San Diego, CA (US); Brian Robert Miller, San Diego, CA (US); Kandasamy Hariharan, San Diego, CA (US); Steffan Ho, San Diego, CA (US); Jianying Dong, San Diego, CA (US); Alexey Alexandrovich Lugovskoy, Woburn, MA (US)

Correspondence Address: LAHIVE & COCKFIELD, LLP / BIOGEN IDEC;FLOOR 30, SUITE 3000, ONE POST OFFICE SQUARE, BOSTON, MA, 02109-2127, US

(73) Assignee: Biogen Idec MA Inc., Cambridge, MA (US)

(21) Appl. No.: 12/200,816

(22) Filed: Aug. 28, 2008

Related U.S. Application Data
(60) Provisional application No. 60/966,475, filed on Aug. 28, 2007.

(51) Int. Cl. A61K 39/395; C07K 16/00; C07H 21/00; C12N 15/63; C12N 5/10; C12P 21/00; A61P 35/00

(52) U.S. Cl. 424/136.1; 424/130.1; 530/389.2; 530/387.3; 536/23.1; 435/320.1; 435/325; 435/69.6

(57) ABSTRACT

The instant invention is based, at least in part on the finding that binding molecules which bind to different epitopes within IGF-1R result in improved IGF-1 and/or IGF-2 blocking capabilities when compared to binding molecules that bind to a single IGF-1R epitope. The instant invention provides compositions that bind to multiple epitopes of IGF-1R, for example, combinations of monospecific binding molecules or multispecific binding molecules (e.g., bispecific molecules). Methods of making the subject binding molecules and methods of using the binding molecules of the invention to antagonize IGF-1R signaling are also provided.

### CROSS-REFERENCES TO RELATED APPLICATIONS

This application claims benefit under § 119(e) of U.S. Provisional Application No. 60/966,475 filed Aug. 28, 2007, entitled, “Compositions that Bind Multiple Epitopes of IGF-IR”. This application is related U.S. Ser. No. ______, filed Aug. 28, 2008, which claims benefit under § 119(e) of U.S. Provisional Application No. ______, filed Aug. 28, 2007, entitled, “Anti-IGF-IR Antibodies and Uses Thereof”. This application is also related to U.S. patent application Ser. No. 11/727,887, filed on Mar. 28, 2007, which claims benefit under 35 U.S.C. § 119(e) of U.S. Provisional Application No. 60/786,347, filed on Mar. 28, 2006 and of U.S. Provisional Application No. 60/876,554 filed on Dec. 22, 2006. Each of the above-referenced patent applications are hereby incorporated by reference in its entirety.

### BACKGROUND OF THE INVENTION

In a cancer cell, receptor tyrosine kinases (TK) play important role in connecting the extra-cellular tumor microenvironment to the intracellular signaling pathways that control diverse cellular functions, such as, cell division cycle, survival, apoptosis, gene expression, cytoskeletal architecture, cell adhesion, and cell migration. As the mechanisms controlling cell signaling are better understood, therapeutic strategies of disrupting one or more of these cellular functions could be developed by targeting at the level of ligand binding, receptor expression/recycling, receptor activation and the proteins involved in the signaling events (Hanahan and Weinberg, Cell 2000. 100:57-70).

The type I insulin like growth factor receptor (IGF-1R, CD221) belongs to receptor tyrosine kinase (RTK) family, (Ullrich et al., Cell. 1990., 61:203-12). IGF-1 and IGF-2 are the two activating ligands of IGF-1R. Together with insulin like growth factor receptor 2 (IGF-2R; CD222) and associated IGF binding proteins (IGFBP-1 to IGFBP-6), these proteins collectively form an IGF system that has been shown to play a significant role in pre- and post-natal development, growth hormone responsiveness, cell transformation, survival, and the acquisition of an invasive and metastatic tumor phenotype (Baserga, Cell. 1994. 79:927-30; Baserga et al., Exp. Cell Res. 1999. 253:1-6, Baserga et al., Int J. Cancer. 2003. 107:873-77).

Several studies have shown that a number of human tumors express high levels of IGF-1R. IGF-1R expressing tumors receive both paracrine receptor activation signals from IGF-1 in the circulation (liver produced) and autocrine receptor activation signals from IGF-2 made by the tumor itself. Recent data from early clinical trials suggest that inhibition of the IGF-1R pathway can lead to clinical responses in sensitive tumors. However, it has been noted that antibody-induced downregulation of IGF-1R expression often leads to increased systemic levels of IGF-1 in patients. As a result, complete inhibition of the IGF-1R pathway is often not feasible. Therefore, there is a need in the art for therapeutic methods and compositions which can more effectively block the IGF-1R mediated pathway of cell survival and growth in neoplastic diseases, including cancer and metastases thereof.

### SUMMARY OF THE INVENTION

The instant invention is based, at least in part on the finding that binding molecules which recognize different epitopes on IGF-1R result in improved IGF-1 and/or IGF-2 blocking capabilities when compared to binding molecules that bind to a single IGF-1R epitope. The instant invention provides compositions that bind to multiple epitopes of IGF-1R, for example, combinations of monospecific binding molecules or multispecific binding molecules (e.g., bispecific molecules). Methods of making the subject binding molecules and methods of using the binding molecules of the invention to antagonize IGF-1R signaling are also provided.

In one aspect, the invention pertains to a method of inhibiting proliferation of a tumor cell expressing IGF-1R comprising contacting the tumor cell with a first binding moiety that binds to a first epitope of IGF-1R and blocks the binding of at least one of IGF-1 and IGF-2 to IGF-1R and a second binding moiety that binds to a second, different epitope of IGF-1R and blocks the binding of at least one of IGF-1 and IGF-2 to IGF-1R, wherein the binding of the first and second moiety to IGF-1R block IGF-1R-mediated signaling to a greater extent than the binding of the first or second moiety alone, to thereby inhibit survival or growth of a tumor cell expressing IGF-1R.

In one embodiment, the first and the second binding moiety block the binding of at least one of IGF-1 and IGF-2 to IGF-1R by different mechanisms.

In one embodiment, the first and the second binding moiety are present in the same binding molecule. In another embodiment, the first and the second binding moiety are present in separate binding molecules.

In one embodiment, the first and the second binding moiety do not compete for binding to IGF-1R.

In one aspect, the invention pertains to a multispecific IGF-1R binding molecule comprising a first IGF-1R binding moiety that binds to a first epitope of IGF-1R and blocks the binding of at least one of IGF-1 and IGF-2 to IGF-1R and a second binding moiety that binds to a second, different epitope of IGF-1R and blocks the binding of at least one of IGF-1 and IGF-2 to IGF-1R.

In one aspect, the invention pertains to a multispecific IGF-1R binding molecule said molecule comprising: a) at least a first allosteric IGF-1R binding moiety which specifically binds a first allosteric IGF-1R epitope thereby allosterically blocking binding of IGF-1 and IGF-2 to IGF-1R; and b) at least a second IGF-1R binding moiety wherein said second binding moiety specifically binds (i) a competitive IGF-1R epitope thereby competitively blocking binding of IGF-1 and IGF-2 to IGF-1R; or (ii) a second allosteric IGF-1R epitope thereby allosterically blocking binding of IGF-1 and not IGF-2 to IGF-1R.

In one embodiment, the first allosteric epitope is located within a region spanning the FnIII-1 domain of IGF-1R and comprising amino acids 437-586 of IGF-1R. In another embodiment, the first allosteric epitope comprises at least 3 contiguous or non contiguous amino acids wherein at least one of the amino acids of the epitope is selected from the group consisting of amino acid positions 437, 438, 459, 460, 461, 462, 464, 466, 467, 469, 470, 471, 472, 474, 476, 477, 478, 479, 480, 482, 483, 488, 490, 492, 493, 495, 496, 509, 513, 514, 515, 53, 544, 545, 546, 547, 548, 551, 564, 565, 568, 570, 571, 572, 573, 577, 578, 579, 582, 584, 585, 586, and 587 of IGF-1R. In another embodiment, the first allosteric epitope comprises at least one of amino acids 461, 462, and 464 of IGF-1R.

In one embodiment, the competitive epitope is located within a region encompassing a portion of the CRR domain and which region encompasses amino acid residues 248-303 of IGF-1R. In another embodiment, the competitive epitope comprises at least 3 contiguous or non-contiguous amino acids wherein at least one of the amino acids of the epitope is selected from the group consisting of amino acids 248, 250, 254, 257, 259, 260, 263, 265, 301, and 303 of IGF-1R. In another embodiment, the competitive epitope comprises amino acids 248, 250, and 254 of IGF-1R.

In one embodiment, the second allosteric epitope is located within a region that includes both the CRR and L2 domains of IGF-1R and which region encompasses residues 241-379 of IGF-1R. In another embodiment, the second allosteric epitope comprises at least 3 contiguous or non-contiguous amino acids wherein at least one of the amino acids is selected from the group consisting of amino acids 241, 248, 250, 251, 254, 257, 263, 265, 266, 301, 303, 308, 327, and 379 of IGF-1R. In another embodiment, the second allosteric epitope comprises at least one of amino acids 241, 242, 251, 257, 265, and 266 of IGF-1R.

In one embodiment, said first allosteric binding moiety is derived from a M13-C06 antibody (ATCC Accession No. PTA-7444) or a M14-C03 antibody (ATCC Accession No. PTA-7445). In another embodiment, the first allosteric binding moiety is an antigen binding site comprising CDRs 1-6 of the M13-C06 antibody (ATCC Accession No. PTA-7444) or the M14-C03 antibody (ATCC Accession No. PTA-7445). In another embodiment, said first allosteric binding moiety competes for binding to IGF-1R with a M13-C06 antibody (ATCC Accession No. PTA-7444) or a M14-C03 antibody (ATCC Accession No. PTA-7445).

In one embodiment, said competitive binding moiety is derived from a M14-G11 antibody (ATCC Accession No. PTA-7855). In another embodiment, the competitive binding moiety is an antigen binding site comprising CDRs 1-6 of the M14-G11 antibody (ATCC Accession No. PTA-7855). In another embodiment, said competitive binding moiety competes for binding to IGF-1R with a M14-G11 antibody (ATCC Accession No. PTA-7855).

In one embodiment, said second allosteric binding moiety is derived from a P1E2 antibody (ATCC Accession No. PTA-7730) or a αIR3 antibody. In another embodiment, second allosteric binding moiety is an antigen binding site comprising CDRs 1-6 of the P1E2 antibody (ATCC Accession No. PTA-7730) or the αIR3 antibody. In another embodiment, said second allosteric binding moiety is derived from an antibody which competes with a P1E2 antibody (ATCC Accession No. PTA-7730) or a αIR3 antibody for binding to IGF-1R.

In one aspect, the invention pertains to a binding molecule of the invention, which is bispecific. In one embodiment, the binding molecule is multivalent for the first binding specificity. In another embodiment, the binding molecule is multivalent for the second binding specificity.

In one embodiment, the binding molecule comprises four binding moieties.

In one embodiment, at least one of the binding moieties is an scFv molecule.

In one embodiment, the binding molecule is a tetravalent antibody molecule comprising two or more scFv molecules. Said scFv molecules may be independently selected from any one of the scFv molecules disclosed herein.

In one embodiment, said scFv molecules are fused to the C-termini of the heavy chains of the tetravalent antibody molecule. In another embodiment, said scFv molecules are fused to the N-termini of heavy chains of the tetravalent antibody molecule. In another embodiment, said scFv molecules are fused to the N-termini of light chains of the tetravalent antibody molecule.

In one embodiment, the binding molecule comprises a stabilized scFv molecule.

In one embodiment, the binding molecule is fully human. In another embodiment, the binding molecule comprises a humanized variable region. In another embodiment, the binding molecule comprises a chimeric variable region.

In one embodiment, the binding molecule comprises a heavy chain constant region or fragment thereof. In another embodiment, said heavy chain constant region or fragment thereof is human IgG4. In one embodiment, said IgG4 constant region lacks glycosylation. In one embodiment, said IgG4 constant regions comprises a S228P and T299A mutation as compared a to a wild-type IgG4 constant region, numbering according to the EU numbering system.

In yet another aspect, the invention pertains to a bispecific IGF-1R antibody molecule comprising two allosteric binding moieties (e.g., any two of the allosteric binding moieties disclosed herein, e.g., allosteric binding moieties derived from a M13-C06 antibody (ATCC Accession No. PTA-7444)) and two competitive binding moieties (e.g., any two of the competitive binding moieties disclosed herein, e.g., competitive binding moieties derived from a M14-G11 antibody (ATCC Accession No. PTA-7855)).

In one embodiment, said competitive binding moieties are provided by an IgG antibody and said allosteric binding moieties are provided by two scFv molecules that are linked or fused to said IgG antibody. In certain embodiments, said scFv molecules are independently selected from any one of the CO6 scFv molecules disclosed herein.

In one embodiment, said IgG antibody comprises the light chain (VL) and heavy chain (VH) variable domains from the M14-G11 antibody. In one embodiment, said VL domain of said IgG antibody comprises the amino acid sequence of SEQ ID NO:93 and said VH domain of said IgG antibody comprises the amino acid sequence of SEQ ID NO:32.

In one embodiment, one or both of said scFv molecules of said allosteric binding moieties comprise a light chain (VL) and a heavy chain (VH) variable domain derived from the M13-C06 antibody.

In one embodiment, one or both of said scFv molecules is a stabilized C06 scFv molecule having a T50 of greater than 60-61° C. In one embodiment, one or both of said scFv molecules is a stabilized scFv molecule having a T50 that is at least 2° C.-10° C. higher than that of a conventional C06 scFv molecule (pWXU092 or pWXU090).

In one embodiment, the variable light domain (VL) of said stabilized scFv is identical to the VL domain of the M13-CO6 antibody (SEQ ID NO:78) but for the presence of one or more stabilizing mutations at amino acid positions within the VL domain selected from the group consisting of: (i) M4, (ii) L11; (iii) V15, (iv) T20, (v) Q24, (vi) R30, (vii) T47, (viii) A51, (ix) G63, (x) D70, (xi) S72, (xii) T74, (xiii) S77 and (xiv) 183 (Kabat numbering convention).

In one embodiment, said stabilizing mutations are selected from the group consisting of: M4L, L11G, V15A, V15D, V15E, V15G, V15I, V15N, V15P, V15R, V15S, T20R, Q24K, R30N, R30T, R30Y, A51G, G63S, D70E, S72N, S72Y, T74S, S77G, I83D, I83E, I83G, I83M, I83R, I83S and I83V.

In one embodiment, the variable heavy domain (VH) of said stabilized scFv is identical to the VH domain of the M13-CO6 antibody (SEQ ID NO:14) but for the presence one or more stabilizing mutations at amino acid positions selected from the group consisting of: (i) S21, (ii) W47, (iii) R83 and (iv) T110 (Kabat numbering convention).

In one embodiment, said stabilizing mutations are selected from the group consisting of: S21E, W47F, R83K, R83T and T110V. In another embodiment, said stabilized scFv molecule comprises the following combination of mutations VL L15S: VH T110V. In another embodiment, said stabilized scFv molecule comprises the following combination of mutations VL S77G: VL 183Q.

In one embodiment, one or both of said stabilized scFv molecule(s) are stabilized CO6 scFv molecule independently selected from selected from the group consisting of MJF-014, MJF-015, MJF-016, MJF-017, MJF-018, MJF-019, MJF-020, MJF-021, MJF-022, MJF-023, MJF-024, MJF-025, MJF-026, MJF-027, MJF-028, MJF-029, MJF-030, MJF-031, MJF-032, MJF-033, MJF-034, MJF-035, MJF-036, MJF-037, MJF-038, MJF-039, MJF-040, MJF-041, MJF-042, MJF-043, MJF-044, MJF-045, MJF-046, MJF-047, MJF-048, MJF-049, MJF-050 and MJF-051.

In one embodiment, said stabilized scFv molecule is a stabilized CO6 VH/VL (183E) scFv molecule comprising the amino acid sequence of MJF-045 (SEQ ID NO:128).

In one embodiment, one or both of said scFv molecules is linked to said IgG antibody by a Gly/Ser linker. In another embodiment, said Gly/Ser linker is a (Gly₄Ser)₅ or Ser(Gly₄Ser)₃ linker.

In one embodiment, said scFv molecules are linked or fused to said IgG antibody via the VL domain of said scFv molecules. In one embodiment, the scFv molecule is of the orientation VH→(Gly4Ser)ₙ linker →VL, and wherein n is 3, 4, 5, or 6. In another embodiment, said scFv molecules are linked or fused to said IgG antibody via the VH domain of said scFv molecules. In one embodiment, the scFv molecule is of the orientation VL→(Gly4Ser)ₙ linker →VH, and wherein n is 3, 4, 5 or 6.

In one embodiment, one or both of said scFv molecules is linked or fused to a heavy chain of said IgG antibody to form a heavy chain of said bispecific antibody. In one embodiment, one of said scFv molecules is linked or fused to a first heavy chain of said IgG antibody and one of said scFv molecules is linked or fused to a second heavy chain of said IgG antibody. In another embodiment, said scFv molecules are linked or fused to the N-terminus of said first and second heavy chains of said IgG antibody.

In one embodiment, the light chains of said IgG antibody comprise the G11 light chain sequence of SEQ ID NO: 130 (pXWU118); and wherein the heavy chains of said bispecific antibody comprise the amino acid sequence of SEQ ID NO:133 (pXWU136).

In one embodiment, said binding molecule is produced by the cell line deposited as ATCC Deposit No. XXX.

In one embodiment, said scFv molecules are linked or fused to the C-terminus of said first and second heavy chains of said IgG antibody to form the heavy chains of said bispecific antibody molecule.

In one embodiment, the light chains of said IgG antibody comprise the G11 light chain sequence of SEQ ID NO: 130 (pXWU118) and wherein the scFv molecule when linked to the N-terminus of said heavy chain comprises the sequence of SEQ ID NO:137 (pXWU135).

In one embodiment, said binding molecule is produced by the cell line deposited as ATCC Deposit No. XXX.

In one embodiment, one or both of said scFv molecules is linked or fused to a light chain of said IgG antibody.

In one embodiment, one of said scFv molecules is linked or fused to a first light chain of said IgG antibody and one of said scFv molecules is linked or fused to a second light chain of said IgG antibody. In one embodiment, said scFv molecules are linked or fused to the N-terminus of said first and second light chains of said IgG antibody.

In one embodiment, said allosteric binding moieties are provided by a IgG antibody and said competitive binding moieties are provided by two scFv molecules that are linked or fused to said IgG antibody.

In one embodiment, said IgG antibody comprises the light chain (VL) and heavy chain (VH) variable domains from the M13-C06 antibody.

In one embodiment, said VL domain of said IgG antibody comprises the amino acid sequence of SEQ ID NO:78 and said VH domain of said IgG antibody comprises the amino acid sequence of SEQ ID NO:14.

In one embodiment, one or both of said scFv molecules comprise a light chain (VL) and a heavy chain (VH) variable domain derived from the M14-G11 antibody.

In one embodiment, one or both of said scFv molecules is a stabilized G1 scFv molecule having a T50 of greater than 50-51° C. one or both of said scFv molecules is a stabilized scFv molecule having a T50 that is at least 2° C.-10° C. higher than that of a conventional G11 (VL/GS4/VH) scFv molecule (pMJF060).

In one embodiment, the variable light domain (VL) of said stabilized scFv is identical to the VL domain of the M14-G11 antibody (SEQ ID NO:93) but for the presence of one or more stabilizing mutations at amino acid positions L50 and/or V83 (Kabat numbering convention).

In one embodiment, said stabilizing mutations are selected from the group consisting of: L50G, L50M, L50N and V83E.

In one embodiment, the variable heavy domain (VH) of said stabilized scFv is identical to the VH domain of the M14-G11 antibody (SEQ ID NO:32) but for the presence one or more stabilizing mutations at amino acid positions E6 and/or S49 (Kabat numbering convention).

In one embodiment, said stabilizing mutations are selected from the group consisting of: E6Q, S49A and S49G.

In one embodiment, said stabilized scFv molecule comprises the following combination of mutations VL L50N: VH E6Q.

In one embodiment, said stabilized scFv molecule comprises the following combination of mutations VL V83E: VH E6Q.

In one embodiment, said stabilized scFv molecule is a stabilized G11 scFv molecule is selected from the group consisting of MJF-060, MJF-084, MJF-085, MJF-086, MJF-087, MJF-091, MJF-092 and MJF-097.

In one embodiment, one or both of said scFv molecules is linked to said IgG antibody by a Gly/Ser linker.

In one embodiment, said Gly/Ser linker is a (Gly₄Ser)₅ or Ser(Gly₄Ser)₃ linker.

In one embodiment, said scFv molecules are linked or fused to said IgG antibody via the VL domain of said scFv molecules.

In one embodiment, the scFv molecule is of the orientation VH→(Gly4Ser)ₙ linker →VL, and wherein n is 3, 4, 5, or 6.

In one embodiment, said scFv molecules are linked or fused to said IgG antibody via the VH domain of said scFv molecules.

In one embodiment, the scFv molecule is of the orientation VL→(Gly4Ser)ₙ linker →VH, and wherein n is 3, 4, 5 or 6.

In one embodiment, one or both of said scFv molecules is linked or fused to a heavy chain of said IgG antibody.

In one embodiment, one of said scFv molecules is linked or fused to a first heavy chain of said IgG antibody and one of said scFv molecules is linked or fused to a second heavy chain of said IgG antibody.

In one embodiment, said scFv molecules are linked or fused to the N-terminus of said first and second heavy chains of said IgG antibody.

In one embodiment, the light chains of said IgG antibody comprise the CO6 light chain sequence of SEQ ID NO:140 and wherein the scFv molecule when linked to the N-terminus of said heavy chain comprises the sequence of SEQ ID NO:144.

In one embodiment, said binding molecule is produced by the cell line deposited as ATCC Deposit No. XXX.

In one embodiment, said scFv molecules are linked or fused to the C-terminus of said first and second heavy chains of said IgG antibody.

In one embodiment, the light chains of said IgG antibody comprise the CO6 light chain sequence of SEQ ID NO:140 and wherein the scFv molecule when linked to the N-terminus of said heavy chain comprises the sequence of SEQ ID NO:144.

said binding molecule is produced by the cell line deposited as ATCC Deposit No. XXX.

In one embodiment, one or both of said scFv molecules is linked or fused to a light chain of said IgG antibody. In one embodiment, one of said scFv molecules is linked or fused to a first light chain of said IgG antibody and one of said scFv molecules is linked or fused to a second light chain of said IgG antibody. In one embodiment, said scFv molecules are linked or fused to the N-terminus of said first and second light chains of said IgG antibody.

In one embodiment, said IgG antibody comprises heavy chain constant domains of the human IgG4 isotype. In another embodiment, said IgG antibody comprises heavy chain constant domains of the human IgG1 isotype.

In one embodiment, said IgG antibody is a chimeric of heavy chain constant domain portions from two or more human antibody isotypes.

In one embodiment, the IgG antibody comprises a Fc region wherein residues 233-236 and 327-331 (EU numbering convention) of the Fc region are from a human IgG2 antibody and the remaining residues of the Fc region are from a human IgG4 antibody.

In one embodiment, the heavy chain constant regions of said IgG antibody lack glycosylation.

In one embodiment, said IgG antibody comprises a S228P in the hinge domain of said whole antibody and/or a T299A mutation in a CH2 domain of said whole antibody, wherein said mutations are relative to a wild-type human IgG antibody (EU numbering system).

In one embodiment, the binding molecule is essentially resistant to aggregation when produced at commercial scale.

In one embodiment, a binding molecule of the invention inhibits IGF-1R-mediated cell proliferation. In one embodiment, a binding molecule of the invention inhibits IGF-1 or IGF-2-mediated IGF-1R phosphorylation. In one embodiment, a binding molecule of the invention inhibits IGF-1 or IGF-2-mediated AKT phosphorylation. In one embodiment, a binding molecule of the invention inhibits AKT mediated survival signaling. In one embodiment, a binding molecule of the invention inhibits tumor growth in vivo. In one embodiment, a binding molecule of the invention inhibits IGF-1R internalization.

In one embodiment, a binding molecule of the invention said binding molecule is conjugated to an agent selected from the group consisting of cytotoxic agent, a therapeutic agent, cytostatic agent, a biological toxin, a prodrug, a peptide, a protein, an enzyme, a virus, a lipid, a biological response modifier, pharmaceutical agent, a lymphokine, a heterologous antibody or fragment thereof, a detectable label, polyethylene glycol (PEG), and a combination of two or more of any said agents.

In one embodiment, a binding molecule of the invention said cytotoxic agent is selected from the group consisting of a radionuclide, a biotoxin, an enzymatically active toxin, a cytostatic or cytotoxic therapeutic agent, a prodrugs, an immunologically active ligand, a biological response modifier, or a combination of two or more of any said cytotoxic agents.

In one embodiment, the invention pertains to a binding molecule of the invention and a carrier.

In one aspect, the invention pertains to a method of treating a subject suffering from a hyperproliferative disorder comprising administering a binding molecule of the invention to the subject such that treatment occurs.

In one embodiment, a binding molecule of the invention said hyperproliferative disorder is selected from group consisting of cancer, a neoplasm, a tumor, a malignancy, or a metastasis thereof. In one embodiment, the hyperproliferative disorder is cancer, said cancer selected from the group consisting of: sarcomas, lung cancer, breast cancer, colorectal cancer, melanoma, leukemia, stomach cancer, brain cancer, pancreatic cancer, cervical cancer, ovarian cancer, uterine cancer, liver cancer, bladder cancer, renal cancer, prostate cancer, testicular cancer, thyroid cancer, head and neck cancer, squamous cell cancer, multiple myeloma, lymphoma and leukemia.

In one embodiment, the invention pertains to a nucleic acid molecule encoding the binding molecule of the invention or a heavy chain or a light chain thereof. In one embodiment, the nucleic acid molecule is in a vector. In one embodiment, the invention pertains to a host cell comprising a vector of the invention.

In one embodiment, the invention pertains to a method of producing a binding molecule of the invention, comprising culturing the host cell of the invention such that the binding molecule is secreted in host cell culture media and (ii) isolating the binding molecule from the media.

In yet another aspect, the invention pertains to a stabilized scFv molecule wherein the stabilized scFv molecule has a T50 that is at least 2° C.-10° C. higher than that of a conventional scFv molecule. In certain embodiments, the stabilized scFv molecule of the invention has binding specificity for IGF-1R,

In one embodiment, said scFv molecule has a T50 of greater than 50° C. In another embodiment, the scFv molecule of the invention has a T50 of greater than 60° C.

In another aspect, a binding molecule of the invention comprises one or more stabilizing mutations as compared to a conventional scFv molecule, wherein said mutations are present at VL amino acid positions selected from the group of VL amino acid positions consisting of: (i) 4, (ii) 11; (iii) 15, (iv) 20, (v) 24, (vi) 30, (vii) 47, (viii) 50, (ix) 51, (x) 63, (xi) 70, (xii) 72, (xiii) 74, (xiv) 77 and (xv) 83 (Kabat numbering convention).

In one embodiment, said stabilizing mutations are selected from the group consisting of: 4L, 11G, 15A, 15D, 15E, 15G, 15I, 15N, 15P, 15R, 15S, 20R, 24K, 30N, 30T, 30Y, 50G, 50M, 50N, 51G, 63S, 70E, 72N, 72Y, 74S, 77G, 83D, 83E, 83G, 83M, 83R, 83S and 83V.

In one embodiment, a binding molecule of the invention comprises one or more stabilizing mutations as compared to a conventional scFv molecule, wherein said mutations are present at VH amino acid positions selected from the group of VH amino acid positions consisting of: (i) 6, (ii) 21, (iii) 47, (iv) 49 and (v) 110 (Kabat numbering convention).

In one embodiment, said stabilizing mutations are selected from the group consisting of: 6Q, 21E, 47F, 49A, 49G, 83K, 83T and 110V.

In one embodiment, a binding molecule of the invention comprises one or more stabilizing mutations as compared to a conventional scFv molecule, wherein said mutations are present at amino acid positions selected consisting of: (i) VL amino acid position 50, (ii) VL amino acid position 83; (iii) VH amino acid position 6 and (iv) VH amino acid position 49 (Kabat numbering convention).

In one embodiment, a binding molecule of the invention comprises stabilizing mutations as compared to a conventional scFv molecule, wherein said mutations are present at: (i) VL amino acid position 50, (ii) VL amino acid position 83; (iii) VH amino acid position 6 and (iv) VH amino acid position 49 (Kabat numbering convention).

In one embodiment, said stabilizing mutations are selected from the group consisting of: VL 50G, VL 50M, VL 50N, VL 83D, VL 83E, VL 83G, VL 83M, VL 83R, VL 83S, VL 83V, VH 6Q, VH 49A and VH 49G.

In one embodiment, a binding molecule of the invention said stabilized scFv molecule has a T50 that is at least 2° C.-10° C. higher than that of a conventional C06 scFv molecule (pWXU092 or pWXU090).

In one embodiment, the variable light domain (VL) of said stabilized scFv is identical to the VL domain of the M13-CO6 antibody (SEQ ID NO:78) but for the presence of one or more stabilizing mutations at amino acid positions within the VL domain selected from the group consisting of: (i) M4, (ii) L11; (iii) V15, (iv) T20, (v) Q24, (vi) R30, (vii) T47, (viii) A51, (ix) G63, (x) D70, (xi) S72, (xii) T74, (xiii) S77 and (xiv) 183 (Kabat numbering convention).

In one embodiment, said stabilizing mutations are selected from the group consisting of: M4L, L11G, V15A, V15D, V15E, V15G, V15I, V15N, V15P, V15R, V15S, T20R, Q24K, R30N, R30T, R30Y, A51G, G63S, D70E, S72N, S72Y, T74S, S77G, I83D, I83E, I83G, I83M, I83R, I83S and I83V.

In one embodiment, the variable heavy domain (VH) of said stabilized scFv is identical to the VH domain of the M13-CO6 antibody (SEQ ID NO:14) but for the presence one or more stabilizing mutations at amino acid positions selected from the group consisting of: (i) S21, (ii) W47, (iii) R83 and (iv) T110 (Kabat numbering convention).

In one embodiment, said stabilizing mutations are selected from the group consisting of: S21E, W47F, R83K, R83T and T110V. In one embodiment, said stabilized scFv molecule comprises the following combination of mutations VL L15S: VH T110V. In one embodiment, said stabilized scFv molecule comprises the following combination of mutations VL S77G: VL I83Q.

In one embodiment, said stabilized scFv molecule is a stabilized CO6 scFv molecule is selected from the group consisting of MJF-014, MJF-015, MJF-016, MJF-017, MJF-018, MJF-019, MJF-020, MJF-021, MJF-022, MJF-023, MJF-024, MJF-025, MJF-026, MJF-027, MJF-028, MJF-029, MJF-030, MJF-031, MJF-032, MJF-033, MJF-034, MJF-035, MJF-036, MJF-037, MJF-038, MJF-039, MJF-040, MJF-041, MJF-042, MJF-043, MJF-044, MJF-045, MJF-046, MJF-047, MJF-048, MJF-049, MJF-050 and MJF-051.

In one embodiment, a binding molecule of the invention is a stabilized scFv molecule having a T50 that is at least 2° C.-10° C. higher than that of a conventional G11 (VL/GS4/VH) scFv molecule (pMJF060).

In one embodiment, the variable light domain (VL) of said stabilized scFv is identical to the VL domain of the M14-G11 antibody (SEQ ID NO:93) but for the presence of one or more stabilizing mutations at amino acid positions L50 and/or V83 (Kabat numbering convention).

In one embodiment, said stabilizing mutations are selected from the group consisting of: L50G, L50M, L50N and V83E.

In one embodiment, the variable heavy domain (VH) of said stabilized scFv is identical to the VH domain of the M14-G11 antibody (SEQ ID NO:32) but for the presence one or more stabilizing mutations at amino acid positions E6 and/or S49 (Kabat numbering convention).

In one embodiment, said stabilizing mutations are selected from the group consisting of: E6Q, S49A and S49G.

In one embodiment, said stabilized scFv molecule comprises the following combination of mutations VL L50N: VH E6Q. In one embodiment, said stabilized scFv molecule comprises the following combination of mutations VL V83E: VH E6Q.

In one embodiment, said stabilized scFv molecule is a stabilized G11 scFv molecule is selected from the group consisting of MJF-060, MJF-084, MJF-085, MJF-086, MJF-087, MJF-091, MJF-092 and MJF-097.

In one embodiment, the invention pertains to a multivalent binding molecule comprising the stabilized scFv molecule of the invention.

In one embodiment, a binding molecule of the invention is essentially free of aggregates when produced at a commercial scale.

In one embodiment, a binding molecule of the invention is essentially free of aggregates following incubation in a buffering system (e.g., PBS) for at least 3 months.

In one embodiment, a binding molecule of the invention has a melting temperature (Tm) of at least 60° C.

In another aspect, the invention pertains to a method of making a stabilized multivalent binding molecule, the method comprising genetically fusing a stabilized scFv molecule of the invention to an amino terminus or a carboxy terminus of a light or heavy chain of an antibody molecule.

In one aspect, the invention pertains to a nucleic acid molecule comprising a nucleotide sequence which encodes the stabilized scFv molecule of the invention or the multivalent binding molecule of the invention.

In one embodiment, the invention pertains to a method of producing a stabilized binding molecule, comprising culturing the host cell of the invention under conditions such that the stabilized binding molecule is produced.

In one embodiment, the host cell is cultured at commercial scale (e.g., 50 L) and wherein at least 5 mg of the stabilized binding molecule is produced for every liter of the host cell culture medium.

In one embodiment, the host cell is cultured at commercial scale (e.g., 50 L) and wherein at least 50 mg of the stabilized binding molecule is produced for every liter of the host cell culture medium

In one embodiment, the host cell is cultured at commercial scale and wherein not more than 10% of the binding molecule is present in aggregate form.

In still another aspect, the invention pertains to a multi specific IGF-1R binding molecule said molecule comprising: a) at least a first IGF-1R binding moiety which specifically binds a first IGF-1R epitope; and b) at least a second IGF-1R binding moiety wherein said second binding moiety specifically binds a second IGF-1R epitope that is non-overlapping with said first epitope; wherein binding of the multispecific IGF-1R binding molecule to IGF-1R inhibits IGF-1R mediated tumor cell growth in vitro to a greater extent than a (i) a first monospecific binding molecule comprising said first binding moiety, (ii) a second monospecific binding molecule comprising said second moiety, or (iii) a combination of said first and second monospecific binding molecules.

In still another aspect, the invention pertains to a multi specific IGF-1R binding molecule said molecule comprising: at least a first IGF-1R binding moiety which specifically binds a first IGF-1R epitope; and at least a second IGF-1R binding moiety wherein said second binding moiety specifically binds a second IGF-1R epitope that is non-overlapping with said first epitope; wherein binding of the multispecific IGF-1R binding molecule to IGF-1R inhibits IGF-1R mediated tumor cell growth in to a greater extent than a (i) a first monospecific binding molecule comprising said first binding moiety, (ii) a second monospecific binding molecule comprising said second moiety, or (iii) a combination of said first and second monospecific binding molecules.

In still another aspect, the invention pertains to a multi specific IGF-1R binding molecule said molecule comprising: at least a first IGF-1R binding moiety which specifically binds a first IGF-1R epitope; and at least a second IGF-1R binding moiety wherein said second binding moiety specifically binds a second IGF-1R epitope that is non-overlapping with said first epitope; wherein binding of the multispecific IGF-1R binding molecule to IGF-1R blocks IGF-1R-mediated signaling to a greater extent than a (i) a first monospecific binding molecule comprising said first binding moiety, (ii) a second monospecific binding molecule comprising said second moiety, or (iii) a combination of said first and second monospecific binding molecules.

In still another aspect, the invention pertains to a multi specific IGF-1R binding molecule said molecule comprising: at least a first IGF-1R binding moiety which specifically binds a first IGF-1R epitope; and at least a second IGF-1R binding moiety wherein said second binding moiety specifically binds a second IGF-1R epitope that is non-overlapping with said first epitope; wherein the multispecific IGF-1R binding molecule binds to IGF-1R with a higher binding affinity than a (i) a first monospecific binding molecule comprising said first binding moiety, (ii) a second monospecific binding molecule comprising said second moiety, or (iii) a combination of said first and second monospecific binding molecules.

In still another aspect, the invention pertains to a multi specific IGF-1R binding molecule said molecule comprising: at least a first IGF-1R binding moiety which specifically binds a first IGF-1R epitope; and at least a second IGF-1R binding moiety wherein said second binding moiety specifically binds a second IGF-1R epitope that is non-overlapping with said first epitope; wherein binding of the multispecific IGF-1R binding molecule to IGF-1R blocks binding of IGF-1 and/or IGF-2 to IGF-1R to a greater extent than a (i) a first monospecific binding molecule comprising said first binding moiety, (ii) a second monospecific binding molecule comprising said second moiety, or (iii) a combination of said first and second monospecific binding molecules.

In still another aspect, the invention pertains to a multi specific IGF-1R binding molecule said molecule comprising: at least a first IGF-1R binding moiety which specifically binds a first IGF-1R epitope; and at least a second IGF-1R binding moiety wherein said second binding moiety specifically binds a second IGF-1R epitope that is non-overlapping with said first epitope; wherein the multispecific IGF-1R binding molecule has a longer serum half-life than a (i) a first monospecific binding molecule comprising said first binding moiety, (ii) a second monospecific binding molecule comprising said second moiety, or (iii) a combination of said first and second monospecific binding molecules.

In still another aspect, the invention pertains to a multi specific IGF-1R binding molecule said molecule comprising: at least a first IGF-1R binding moiety which specifically binds a first IGF-1R epitope; and at least a second IGF-1R binding moiety wherein said second binding moiety specifically binds a second IGF-1R epitope that is non-overlapping with said first epitope; wherein binding of the multispecific IGF-1R binding molecule to IGF-1R inhibits IGF-1 or IGF-2-mediated IGF-1R phosphorylation to a greater extent than a (i) a first monospecific binding molecule comprising said first binding moiety, (ii) a second monospecific binding molecule comprising said second moiety, or (iii) a combination of said first and second monospecific binding molecules.

In still another aspect, the invention pertains to a multi specific IGF-1R binding molecule said molecule comprising: at least a first IGF-1R binding moiety which specifically binds a first IGF-1R epitope; and at least a second IGF-1R binding moiety wherein said second binding moiety specifically binds a second IGF-1R epitope that is non-overlapping with said first epitope; wherein binding of the multispecific IGF-1R binding molecule to IGF-1R inhibits IGF-1 or IGF-2-mediated AKT and/or MAPK phosphorylation to a greater extent than a (i) a first monospecific binding molecule comprising said first binding moiety, (ii) a second monospecific binding molecule comprising said second moiety, or (iii) a combination of said first and second monospecific binding molecules.

In still another aspect, the invention pertains to a multispecific IGF-1R binding molecule said molecule comprising: at least a first IGF-1R binding moiety which specifically binds a first IGF-1R epitope; and at least a second IGF-1R binding moiety wherein said second binding moiety specifically binds a second IGF-1R epitope that is non-overlapping with said first epitope; wherein the multispecific IGF-1R binding molecule cross-links IGF-1R receptors to a greater extent than a (i) a first monospecific binding molecule comprising said first binding moiety, (ii) a second monospecific binding molecule comprising said second moiety, or (iii) a combination of said first and second monospecific binding molecules.

In still another aspect, the invention pertains to a multispecific IGF-1R binding molecule said molecule comprising: at least a first IGF-1R binding moiety which specifically binds a first IGF-1R epitope; and at least a second IGF-1R binding moiety wherein said second binding moiety specifically binds a second IGF-1R epitope that is non-overlapping with said first epitope; wherein binding of the multispecific IGF-1R binding molecule to IGF-1R induces IGF-1R receptor internalization to a greater extent than a (i) a first monospecific binding molecule comprising said first binding moiety, (ii) a second monospecific binding molecule comprising said second moiety, or (iii) a combination of said first and second monospecific binding molecules.

In still another aspect, the invention pertains to a multispecific IGF-1R binding molecule said molecule comprising: at least a first IGF-1R binding moiety which specifically binds a first IGF-1R epitope; and at least a second IGF-1R binding moiety wherein said second binding moiety specifically binds a second IGF-1R epitope that is non-overlapping with said first epitope; wherein binding of the multispecific IGF-1R binding molecule to IGF-1R induces tumor cell cycle arrest to a greater extent than a (i) a first monospecific binding molecule comprising said first binding moiety, (ii) a second monospecific binding molecule comprising said second moiety, or (iii) a combination of said first and second monospecific binding molecules.

In still another aspect, the invention pertains to a multispecific IGF-1R binding molecule said molecule comprising: at least a first IGF-1R binding moiety which specifically binds a first IGF-1R epitope; and at least a second IGF-1R binding moiety wherein said second binding moiety specifically binds a second IGF-1R epitope that is non-overlapping with said first epitope; wherein binding of the multispecific IGF-1R binding molecule to IGF-1R inhibits IGF-1R mediated tumor cell growth to a greater extent than a (i) a first monospecific binding molecule comprising said first binding moiety, (ii) a second monospecific binding molecule comprising said second moiety, or (iii) a combination of said first and second monospecific binding molecules.

### BRIEF DESCRIPTION OF THE DRAWINGS

FIG. 1: Schematic diagram of the structure of IGF-1R. The FnIII-2 domain contains loop structure that is proteolytically processed in vivo as shown by a zig-zag line. The transmembrane region is shown as a helical loop that traverses a schematic of a phospholipid bilayer. The location of the IGF-1/IGF-2 binding site within IGF-1R is shown by a star. It has been demonstrated that only one IGF-1/IGF-2 molecule binds to each IGF-1R heterodimeric molecule.

FIG. 2: The mature polypeptide sequence of IGF-1R (SEQ ID NO: 2).

FIG. 3: The nucleotide and the amino acid sequence of the original, unmodified VH and VL regions of M13-C06. (a) (SEQ ID NO:13) shows the single-stranded DNA sequence of the VH region of M13-C06. (b) (SEQ ID NO:77) shows the single-stranded DNA sequence of the VL region of M13-C06. (c) (SEQ ID NO:14) shows the amino acid sequence of the VH region of M13-C06. (d) (SEQ ID NO:78) shows the amino acid sequence of the VL region of M13-C06.

FIG. 4: The nucleotide and the amino acid sequence of the optimized VH regions of M13-C06. (a) (SEQ ID NO:18) shows the single-stranded DNA sequence of sequence optimized VH regions of M13-C06. (b) (SEQ ID NO:14) shows the amino acid sequence of the optimized VH region M13-C06.

FIG. 5: The nucleotide and the amino acid sequence of the original, unmodified versions of the VH and VL regions of M14-C03. (a) (SEQ ID NO:25) shows the single-stranded DNA sequence of heavy chain variable region (VH) of M14-C03. (b) (SEQ ID NO:87) shows the single-stranded DNA sequence of light chain variable region (VL) of M14-C03. (c) (SEQ ID NO:26) shows the amino acid sequence of heavy chain variable region (VH) of M14-C03. (d) (SEQ ID NO:88) shows the amino acid sequence of light chain variable region (VL) of M14-C03.

FIG. 6: The nucleotide and the amino acid sequence of the optimized VH region of M14-C03.

(a) (SEQ ID NO:30) shows the single-stranded DNA sequence of sequence optimized VH region of M14-C03. (b) (SEQ ID NO:26) shows the amino acid sequence of sequence optimized VH region of M14-C03.

FIG. 7: The nucleotide and the amino acid sequence of the original, unmodified versions of the VH and VL regions of M14-G11: (a) (SEQ ID NO:31) shows the single-stranded DNA sequence of heavy chain variable region (VH) of M14-G11. (b) (SEQ ID NO:92) shows the single-stranded DNA sequence of light chain variable region (VL) of M14-G11. (c) (SEQ ID NO:32) shows the amino acid sequence of heavy chain variable region (VH) of M14-G11. (d) (SEQ ID NO:93) shows the amino acid sequence of light chain variable region (VL) of M14-G11.

FIG. 8: The nucleotide and the amino acid sequence of the optimized heavy chain variable region (VH) of M14-G11. (a) (SEQ ID NO:36) shows the single-stranded DNA sequence of the optimized VH region of M14-G11. (b) (SEQ ID NO:32) shows the amino acid sequence of sequence optimized VH region of M14-G11.

FIG. 9: The nucleotide and the amino acid sequence of the unmodified versions of VH and VL regions of P1E2.3B12. (a) (SEQ ID NO:62) shows the single-stranded DNA sequence of the VH region of P1E2.3B12. (b) (SEQ ID NO:117) shows the single-stranded DNA sequence of the VL region P1E2.3B12. (c) (SEQ ID NO:63) shows the amino acid sequence of the VH region of P1E2.3B12. (d) (SEQ ID NO:118) shows the amino acid sequence of the VL region of P1E2.3B12.

FIG. 10: The amino acid sequences of constant domains employed in binding molecules of the invention. (a) (SEQ ID NO:1) shows the amino acid sequence of light chain constant domain. (b) (SEQ ID NO:122) shows the amino acid sequence of heavy chain aglyIgG4.P constant domains.

FIG. 11: Cross-competition binding analysis of IGF-1R antibody binding epitopes. +++++=antibody binding competition relative to itself (90-100%). ++++=70-90% competition. +++=50-70% competition. ++=30-50% competition. +=10-30% competition. +/−=0-10% competition. N/A=results not available.

FIG. 12: Examples of M13.C06 antibody binding to hIGF-1R-Fc (FIG. 12A) and mIGF-1R-Fc (FIG. 12B) controls in the SPR assay compared to antibody binding to IGF-1R mutant proteins SD006 (FIG. 12C; binding positive) and SD015 (FIG. 12D; binding negative).

FIG. 13: Inability of M13-C06 and M14-G11 to cross-block one another in an SPR-based Competition Assay. Soluble M14-G11 and M13-C06 was titrated into a solution of hIGF-1R-His prior to injection over sensorchip surfaces containing immobilized M13-C06 (FIG. 13A) or M14-G11 (FIG. 13B). The reduction in the SPR signal of IGF-1R binding to M13-C06 and M14-G11 sensorchip surfaces in the presence of (a) IGF-1 and (b) IGF-2 are depicted in FIGS. 13C and 13D, respectively.

FIG. 14: Inhibition of human IGF-1 His (FIG. 14A) or human IGF-2 His (FIG. 14B) binding to biotinylated hIGF-1R-Fc by antibodies M13-C06, M14-C03, M14-G11, P1E2, and/or αIR3.

FIG. 15: ELISA assays for detecting human IGF-1 H is binding to biotinylated hIGF-1R (FIG. 15A; Human IGF-1His was serially diluted in PBST (circles) and PBST containing 2 μM M13-C06 (squares)) as well as IGF-1 (FIG. 15B) or IGF-2 (FIG. 15C) blocking properties of antibody combinations in comparison to single monoclonal antibodies.

FIG. 16: Residues whose mutation affected the binding of M13-C06 to hIGF-1R-Fc were mapped to the structure of the homologous 1R ectodomain. Mutation of IGF-1R amino acid residues 415, 427, 468, 478 and 532 had no detectable affect on M13-C06 antibody binding. Mutation of IGF-1R amino acid residues 466, 467, 533, 564 and 565 had a weak negative affect on M13-C06 antibody binding. Mutation of IGF-1R amino acid residues 459, 460, 461, 462, 464, 480, 482, 483, 490, 570 and 571 had a strong negative affect on M13-C06 antibody binding. See, Table 7 for a compilation of mutation analysis results.

FIG. 17: Residues whose mutation affected the binding of M14-G11 to hIGF-1R-Fc were mapped to the structure of the first three ectodomains of human IGF-1R. Mutation of IGF-1R amino acid residues 28, 227, 237, 285, 286, 301, 327 and 412 had no detectable affect on M14-G11 antibody binding. Mutation of IGF-1R amino acid residues 257, 259, 260, 263 and 265 had a weak negative affect on M14-G11 antibody binding. Mutation of IGF-1R amino acid residue 254 had a moderate negative affect on M14-G11 antibody binding. Mutation of IGF-1R amino acid residues 248 and 250 had a strong negative affect on M14-G11 antibody binding. See, Table 7 for a compilation of mutation analysis results.

FIG. 18: Residues whose mutation affected the binding of αIR3 and P1E2 to hIGF-1R-Fc were mapped to the structure of the first three ectodomains of human IGF-1R. Mutation of IGF-1R amino acid residues 28, 227, 237, 250, 259, 260, 264, 285, 286, 306 and 412 had no detectable affect on antibody binding. Mutation of IGF-1R amino acid residues 257, 263, 301, 303, 308, 327 and 389 had a weak negative affect on antibody binding. Mutation of IGF-1R amino acid residue 248 and 254 had a moderate negative affect on M14-G11 antibody binding. Mutation of IGF-1R amino acid residue 265 had a strong negative affect on antibody binding. See, Table 7 for a compilation of mutation analysis results.

FIG. 19: A model of synergistic Anti-IGF-1R Inhibition. Binding of individual antibodies to multiple epitopes (D) leads to synergistic inhibition of IGF-1 and IGF-2 mediated signaling, relative to binding of a single epitope (B and C).

FIG. 20: Enhanced inhibition of tumor cell growth stimulated by IGF-1/IGF-2 through combined targeting of distinct IGF-1R epitopes. Enhanced inhibition of BXPC3 cell growth was observed under serum-free conditions with equimolar doses of C06 and G1 antibodies (100, 10 and 1 nM (FIG. 20A) and 1 uM to 0.15 nM (FIG. 20B)). Enhanced inhibition of H322M cell growth was also observed in 10% serum augmented with IGF-1/IGF-2 (FIG. 20C).

FIG. 21: An exemplary tetravalent bispecific binding molecule of the invention comprising scFv molecules with a first binding specificity fused to a bivalent IgG antibody with a different binding specificity. scFv molecules may be linked or fused to the C-terminus of the heavy chain or the N-terminus of the light or heavy chain of the bivalent antibody to create a bispecific binding molecule. In preferred embodiments, the scFv molecule is a stabilized molecule.

FIG. 22: A model of synergistic Anti-IGF-1R inhibition following binding of an exemplary bispecific binding molecule of the invention. Binding of a bispecific antibody to multiple epitopes (B) leads to synergistic inhibition of IGF-1 and IGF-2 mediated signaling, relative to binding of a single epitope (A).

FIG. 23: Schematic diagram of IgG-like N- and C-bispecific antibodies. A stability-engineered anti-Ep-1 scFv is genetically tethered to the amino- or carboxyl-terminal of the full-length heavy chain using either a 25- or 16-amino acid flexible Gly/Ser linker, respectively. The full-length antibody has specificity to Ep-2. In preferred embodiments, at least one of the scFv molecules is a stabilized scFv molecule. In certain embodiments, scFv molecules may be fused or linked to either the C-terminus or N-terminus of the heavy chain or to the N-terminus of the antibody light chain. Ep=epitope.

FIG. 24: shows a schematic representation of the steps and PCR products used for assembly of C06 scFvs as described in Example 1.

FIG. 25: The single-stranded DNA sequence (SEQ ID NO: 123, FIG. 25A) and amino acid sequence (SEQ ID NO: 124; FIG. 25B) of a conventional C06 (VL/GS3VH) scFv (pXWU092). The Myc and His tag sequence DDDKSFLEQKLISEEDLNSAVDHHHHHH was appended to the C-terminus of the scFv to facilitate purification.

FIG. 26: The single-stranded DNA sequence (SEQ ID NO: 125, FIG. 26A) and amino acid sequence (SEQ ID NO: 126; FIG. 26B) of a conventional C06 (VH/GS3/VL) scFv (pXWU090). The Myc and His tag sequence DDDKSFLEQKLISEEDLNSAVDHHHHHH was appended to the C-terminus of the scFv to facilitate purification.

FIG. 27 depicts the results of a thermal challenge assay in which the thermal stabilities of a conventional C06 (VH/GS3/VL) scFv containing a (Gly₄Ser)₃ linker (), a conventional C06 (VH/GS4/VL) scFv containing a (Gly₄Ser)₄ linker (◯),a conventional C06 (VL/GS3/VH) scFv containing a (Gly₄Ser)₃ linker (▪), and a conventional C06 (VL/GS4/VH) scFv containing a (Gly₄Ser)₄ linker (□) are compared. The temperatures (° C.) at which 50% of the scFv molecules retain their binding activity to IGF-1R (T₅₀) are indicated in the figure.

FIG. 28 shows the single-stranded DNA sequence (SEQ ID NO: 127, FIG. 28A) and amino acid sequence (SEQ ID NO: 128; FIG. 28B) of a stabilized anti-IGF-1R C06 (I83E) scFv. The Myc and His tag sequence DDDKSFLEQKLISEEDLNSAVDHHHHHH was appended to the C-terminus of the scFv to facilitate purification.

FIG. 29 shows the single-stranded DNA sequence (SEQ ID NO: 129, FIG. 29A) and amino acid sequence (SEQ ID NO: 130; FIG. 29B) of an anti-IGF-1R G11 light chain. The italicized sequence within FIG. 29A denotes DNA sequence encoding the signal peptide MDMRVPAQLLGLLLLWLPGARC (SEQ ID NO:131).

FIG. 30 shows the single-stranded DNA sequence (SEQ ID NO: 132, FIG. 30A) and amino acid sequence (SEQ ID NO: 133; FIG. 30B) of the heavy chain of an N-anti-IGF-1R bispecific antibody (pXWU136). The italicized sequence within FIG. 30A denotes DNA sequence encoding the signal peptide: MGWSLILLFLVAVATRVLS (SEQ ID NO: 134). The stability-engineered anti-IGF-1R scFv (MJF-045) is shown in the V_{L}→V_{H} orientation and is appended to the N-terminus of the anti-IGF-1R G11 heavy chain through a (GlyGlyGlyGlySer)₄ (SEQ ID NO:135) linker.

FIG. 31 shows the single-stranded DNA sequence (SEQ ID NO: 136, FIG. 31A) and amino acid sequence (SEQ ID NO: 137; FIG. 31B) of the heavy chain of a C-anti-IGF-1R bispecific antibody (pXWU135). The italicized sequence within FIG. 31A denotes DNA sequence encoding the signal peptide: MGWSLILLFLVAVATRVLS (SEQ ID NO:134). The stability-engineered anti-IGF-1R scFv (MJF-045) is shown in the V_{L}→V_{H} orientation and is appended to the C-terminus of the anti-IGF-1R G1 heavy chain through a Ser(GlyGlyGlyGlySer)₃ (SEQ ID NO:138) linker.

FIG. 32 shows the single-stranded DNA sequence (SEQ ID NO: 139, FIG. 32A) and amino acid sequence (SEQ ID NO: 140; FIG. 32B) of an anti-IGF-1R C06 light chain. The italicized sequence within FIG. 32A denotes DNA sequence encoding the signal peptide: MDMRVPAQLLGLLLLWLPGARC (SEQ ID NO: 131).

FIG. 33 shows the single-stranded DNA sequence (SEQ ID NO: 141, FIG. 33A) and amino acid sequence (SEQ ID NO: 142; FIG. 33B) of the heavy chain of an N-anti-IGF-1R bispecific antibody. The italicized sequence within FIG. 33A denotes DNA sequence encoding the signal peptide: MGWSLILLFLVAVATRVLS (SEQ ID NO:134). The anti-IGF-1R G11 scFv is shown in the V_{L}→V_{H} orientation and is appended to the N-terminus of the anti-IGF-1R C06 heavy chain through a (GlyGlyGlyGlySer)₄ (SEQ ID NO:135) linker.

FIG. 34 shows the single-stranded DNA sequence (SEQ ID NO: 143, FIG. 34A) and amino acid sequence (SEQ ID NO: 144; FIG. 34B) of the heavy chain of a C-anti-IGF-1R bispecific antibody. The italicized sequence within FIG. 34A denotes DNA sequence encoding the signal peptide: MGWSLILLFLVAVATRVLS (SEQ ID NO:134). The anti-IGF-1R G11 scFv is shown in the V_{L}→V_{H} orientation and is appended to the C-terminus of the anti-IGF-1R C06 heavy chain through a Ser(GlyGlyGlyGlySer)₃ (SEQ ID NO:138) linker.

FIG. 35 shows an SDS-PAGE gel (FIG. 35A) and an analytical SEC elution profile (FIG. 35B) of purified stability-engineered C-anti-IGF-1R bispecific antibody (pXWU135/pXWU118).

FIG. 36 shows an SDS-PAGE gel (FIG. 36A) and an analytical SEC elution profile (FIG. 36B) of purified stability-engineered N-anti-IGF-1R bispecific antibody (pXWU136/pXWU118).

FIG. 37: Schematic diagrams of the N- and C-terminal anti-IGF-1R bispecific antibodies (also denoted N- and C-term. IGF-1R bispecific antibodies). The scFv was derived from the C06 MAb and the IgG1 antibody was derived from the G11 antibody.

FIG. 38: SDS PAGE and analytical size exclusion chromatography (SEC) of N- and C-term. IGF-1R bispecific antibodies. Purified N-Term. (FIG. 38A) and C-term. (FIG. 38B) IGF-1R bispecific antibody proteins run on a 4-20% Tris-Glycine Novex® Gel under both non-reducing and reducing conditions. N- and C-term. IGF-1R̂ (30 □g each) were also passed over an analytical SEC column (FIG. 38C). The BsAbs eluted at the expected molecular weight of ˜200 kDa based on protein molecular weight standards.

FIG. 39: Near UV (FIG. 39A) and Far UV (FIG. 39B) CD spectra at 10° C. and DSC scans (FIG. 39C) of the N- and C-terminal bispecific antibodies and the G1 IgG1 control antibody. The generate an understanding of the signal-to-noise and potential drift due to very low sensitivity of the near UV CD region, a second non-protein PBS baseline was run and baseline corrected using the first PBS baseline and displayed on the plot (FIGS. 39A and B). The GIl IgG1 demonstrates the classical 3 transitions common for human IgG1s. Both the N- and C-terminal BsAbs also exhibit the 3 transitions for the C_{H}2, C_{H}3, and Fab domains plus one extra transition arising from the unfolding of the stabilized C06 scFv domains.

FIG. 40: ITC demonstrates the ability of the C06 and G1 antibodies, as well as N- and C-terminal IGF-1Rbispecific antibodies, to co-engage IGF-1R (FIG. 40A and. FIG. 40A: Raw plot of the heat capacity in the ITC cell as first injections of the C06 MAb are made followed by injections of G11 MAb. FIG. 40B: Conversion of the raw data from FIG. 40A into enthalpies of binding for the MAb titrations. FIG. 40C: Raw plot of the heat capacity in the ITC cell as injections N-term. IGF-1R bispecific antibody (above) and C-term. IGF-1R bispecific antibody are made into a solution containing sIGF-1R(1-903). FIG. 40D. Conversion of the raw data from FIG. 40C into enthalpies of binding for the BsAb titrations.

FIG. 41: Equilibrium solution-binding experiments between sIGF-1R(1-903) and the N- and C-terminal IGF-1R bispecific antibodies. C06 and G11 MAbs and Fabs were used as controls in the experiment. FIG. 41A: Solution binding experiments using C06 as the capture reagent in the Biacore3000. FIG. 41B: Solution binding experiments using G11 as the capture reagent in the Biacore3000.

FIG. 42: IGF-1R ligand blocking ELISAs using antibodies C06 and G11 and the N- and C-terminal IGF-1R bispecific antibodies. FIG. 42A: IGF-1 blocking ELISA. FIG. 42B: IGF-2 blocking ELISA.

FIG. 43: Discriminating the allosteric versus competitive IGF-1 and IGF-2 blocking properties of inhibitory anti-IGF-1R antibodies C06 and G11. FIG. 43A: Results of adding the competitive inhibitor, G11, into the IGF-1 blocking assay performed at various IGF-1 concentrations. FIG. 43B: Results of adding the allosteric inhibitor, C06, into the IGF-1 blocking assay performed at various IGF-1 concentrations.

FIG. 44: Ligand blocking properties of the N- and C-terminal IGF-1R bispecific proteins at multiple IGF-1 and IGF-2 concentrations using the inhibitory ELISA assays. FIG. 44A: IGF-1 blocking with the C-term. IGF-1R bispecific antibodies. FIG. 44B: IGF-2 blocking with the C-term. IGF-1R bispecific antibodies. FIG. 44C. IGF-1 blocking with the N-term. IGF-1R bispecific antibody. FIG. 44D. IGF-2 blocking with the N-term. IGF-1R bispecific antibody.

FIG. 45: Molecular weight determination by size exclusion chromatography (SEC) and static light scattering of the complexes formed between sIGF-1R(1-903) and the C06 and G11 MAbs (FIG. 45A) or the N- and C-terminal IGF-1R bispecific antibodies (FIG. 45B).

FIG. 46: IGF-1R bispecific antibody inhibits IGF-1R phosphorylation (FIG. 46A) and induces IGF-1R degradation over a 24 hr period (FIG. 46B) in H322M NSCLC cells.

FIG. 47: IGF-1R bispecific antibodies induce IGF-1R internalization over a 24 hr period (FIG. 47A) and inhibit p-ERK (FIG. 47B).

FIG. 48: IGF-1R bispecific antibodies inhibits p-AKT in H322M NSCLC cells (FIG. 48A); A549 NSCLC cells (FIG. 48B); and in BxPC3 cells (FIG. 48C).

FIG. 49: IGF-1R bispecific antibodies inhibit IGF-driven cell growth of: BxPC3 pancreatic cancer cells (FIG. 49A); H322M NSCLC cells (FIG. 49B); A431 cancer cells (FIG. 49C); and A549 NSCLC cells (FIG. 49D) in serum free medium (SFM).

FIG. 50: IGF-1R bispecific antibodies inhibit IGF-driven cell growth of: BxPC3 pancreatic cancer cells (FIG. 50A); A549 NSCLC cells (FIG. 50B); SJSA-1 osteosarcoma cells (FIG. 50C); and HT-29 colon cancer cells (FIG. 50D) in 10% FBS.

FIG. 51: IGF-1R bispecific antibodies inhibit serum-driven cell growth of A549 NSCLC cells (FIG. 51A) and H322M NSCLC cells (FIG. 51B) in 10% FBS.

FIG. 52: IGF-1R bispecific antibodies inhibit IGF-induced cell cycling of BxPC3 pancreatic cancer cells with no IGF stimulation (FIG. 52A) or with 100 ng/ml of IGF-1 and IGF-2 (FIG. 52B).

FIG. 53: IGF-1R bispecific antibodies do not elicit ADCC activity (FIG. 53A) but inhibit colony formation of A549 NSCLC cells (FIG. 53B).

FIG. 54: Combination of C06 and G11 led to enhanced inhibition of tumor growth in osteosarcoma SJSA-1 model.

FIG. 55: Cell-based flow cytometric analysis of antibody binding to the H322M non-small cell lung cancer cell line. Flow cytometry was performed using either an anti-human Fab (FIG. 55A) or an anti-human Fcgamma (FIG. 55B) antibody as a PE-conjugated secondary reagent to detect antibody binding.

FIG. 56: Serum concentration-Time profiles of C06, G11, C-IGF-1R bispecific antibodies (FIG. 56A) and N-IGF-1R bispecific antibodies (FIG. 56B) in non tumor bearing female CB17 SCID mice after a single intra-peritoneal (IP) administration.

FIG. 57: Equilibrium solution-binding experiments between sIGF-1R(1-903) and the N- and C-terminal IGF-1R bispecific antibodies diluted from serum. FIGS. 57A and B depict solution binding experiments using the C06 MAb as the capture reagent and C-term. IGF-1R bispecific antibodies (FIG. 57A) or N-term. IGF-1R bispecific antibody (FIG. 57B) diluted from serum. FIGS. 57C and D depict solution binding experiments using the G1 MAb as the capture reagent and C-term. IGF-1R bispecific antibody (FIG. 57C) or N-term. IGF-1R bispecific antibody (FIG. 57D) diluted from serum.

### DETAILED DESCRIPTION OF THE INVENTION

### I. Definitions

It is to be noted that the term “a” or “an” entity refers to one or more of that entity; for example, “an IGF-1R antibody,” is understood to represent one or more IGF-1R antibodies. As such, the terms “a” (or “an”), “one or more,” and “at least one” can be used interchangeably herein.

As used herein, the term “binding molecule” refers to a molecule which binds (e.g., specifically binds or preferentially binds) to a target molecule of interest, e.g., an antigen. In particular embodiments, a binding molecule of the invention is a polypeptide comprising a binding site which specifically or preferentially binds to at least one epitope of IGF-1R. Binding molecules within the scope of the invention also include small molecules, nucleic acids, peptides, peptidomimetics, dendrimers, and other molecules with binding specificity for an IGF-1R epitope described herein. In other embodiments, binding molecules of the invention comprise a binding moiety which is a polypeptide, small molecule, nucleic acid, peptide, peptidomimetic, dendrimer, or other molecule with binding specificity for an IGF-1R epitope.

As used herein, the term “polypeptide” is intended to encompass a singular “polypeptide” as well as plural “polypeptides,” (e.g., in the case of dimeric or multimeric polypeptides) and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protectingiblocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue.

An “isolated” polypeptide or a fragment, variant, or derivative thereof refers to a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique. Preferably, the polypeptides of the invention are isolated.

As used herein the term “derived from” a designated protein refers to the origin of the polypeptide. In one embodiment, the polypeptide or amino acid sequence which is derived from a particular starting polypeptide is a variable region sequence (e.g. a VH or VL) or sequence related thereto (e.g. a CDR or framework region). In one embodiment, the amino acid sequence which is derived from a particular starting polypeptide is not contiguous. For example, in one embodiment, one, two, three, four, five, or six CDRs are derived from a starting antibody. In one embodiment, the polypeptide or amino acid sequence that is derived from a particular starting polypeptide or amino acid sequence has an amino acid sequence that is essentially identical to that of the starting sequence or a portion thereof, wherein the portion consists of at least 3-5 amino acids, 5-10 amino acids, at least 10-20 amino acids, at least 20-30 amino acids, or at least 30-50 amino acids, or which is otherwise identifiable to one of ordinary skill in the art as having its origin in the starting sequence.

Also included as polypeptides of the present invention are fragments, derivatives, analogs, or variants of the foregoing polypeptides, and any combination thereof. The terms “fragment,” “variant,” “derivative” and “analog” when referring to binding molecules of the present invention include any polypeptides which retain at least some of the binding properties of the corresponding molecule. Fragments of polypeptides of the present invention include proteolytic fragments, as well as deletion fragments, in addition to specific antibody fragments discussed elsewhere herein. Variants of binding molecules of the present invention include fragments as described above, and also polypeptides with altered amino acid sequences due to amino acid substitutions, deletions, or insertions. Variants may occur naturally or be non-naturally occurring. Non-naturally occurring variants may be produced using art-known mutagenesis techniques. Variant polypeptides may comprise conservative or non-conservative amino acid substitutions, deletions or additions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, an amino acid residue in a polypeptide may be replaced with another amino acid residue from the same side chain family. In another embodiment, a string of amino acids can be replaced with a structurally similar string that differs in order and/or composition of side chain family members. Alternatively, in another embodiment, mutations may be introduced randomly along all or part of the polypeptide.

The polypeptides of the invention are binding molecules that comprise at least one binding site or moiety that specifically binds to a target molecule (e.g., IGF-1R). For example, in one embodiment, a binding molecule of the invention comprises an immunoglobulin antigen binding site or the portion of a receptor molecule responsible for ligand binding. The invention pertains to these binding molecules or the nucleic acid molecules which encode them. In one embodiment, the binding molecules comprise at least two binding sites. In one embodiment, the binding molecules comprise two binding sites. In one embodiment, the binding molecules comprise three binding sites. In another embodiment, the binding molecules comprise four binding sites. In another embodiment, the binding molecules comprise five binding sites. In another embodiment, the binding molecules comprise six binding sites.

In one embodiment, the binding molecules of the invention are monomers. In another embodiment, the binding molecules of the invention are multimers. For example, in one embodiment, the binding molecules of the invention are dimers. In one embodiment, the dimers of the invention are homodimers, comprising two identical monomeric subunits. In another embodiment, the dimers of the invention are heterodimers, comprising at least two non-identical monomeric subunits. The subunits of the dimer may comprise one or more polypeptide chains. For example, in one embodiment, the dimers comprise at least two polypeptide chains. In one embodiment, the dimers comprise two polypeptide chains. In another embodiment, the dimers comprise three polypeptide chains. In another embodiment, the dimers comprise four polypeptide chains (e.g., as in the case of antibody molecules). In another embodiment, the dimers comprise five polypeptide chains. In another embodiment, the dimers comprise six polypeptide chains.

In one embodiment, the binding molecules of the invention are monovalent, i.e., comprise one target binding site (e.g., as in the case of a scFv molecule). In the case of monovalent binding molecules, the compositions of the invention that bind to at least two different epitopes of IGF-1R comprise at least two such binding molecules, each having specificity for a different epitope of IGF1R. In one embodiment, the binding molecules of the invention are multivalent, i.e., comprise more than one target binding site. In another embodiment, the binding molecules comprise at least two binding sites. In one embodiment, the binding molecules comprise two binding sites. In one embodiment, the binding molecules comprise three binding sites. In another embodiment, the binding molecules comprise four binding sites. In another embodiment, the binding molecules comprise greater than four binding sites.

As used herein the term “valency” refers to the number of potential binding sites in a binding molecule. A binding molecule may be “monovalent” and have a single binding site or a binding molecule may be “multivalent” (e.g., bivalent, trivalent, tetravalent, or greater valency). Each binding site specifically binds one target molecule or specific site on a target molecule (e.g., an epitope). When a binding molecule comprises more than one target binding site (i.e. a multivalent binding molecule), each target binding site may specifically bind the same or different molecules (e.g., may bind to different IGF-1R molecules or to different epitopes on the same IGF-1R molecule).

As used herein, the term “binding moiety”, “binding site”, or “binding domain” refers to the portion of a binding molecule that specifically binds to a target molecule of interest (e.g., an IGF-1R). Exemplary binding domains include an antigen binding site of an antibody, an antibody variable domain (e.g., a VL or VH domain), a receptor binding domain of a ligand, a ligand binding domain of a receptor or an enzymatic domain. In one embodiment, the binding molecules have at least one binding site specific for IGF-1R. In certain embodiments, a binding site has a single IGF-1R binding specificity. In other embodiments, a binding site may have two or more binding specificities (e.g., wherein at least one binding specificity is an IGF-1R binding specificity). For example, a binding molecule may have a single binding site having dual specificity.

The term “binding specificity” or “specificity” refers to the ability of a binding molecule to specifically bind (e.g., immunoreact with) a given target molecule or epitope. In certain embodiments, the binding molecules of the invention comprise two or more binding specificities (i.e., they bind two or more different epitopes present on one or more different antigens at the same time). A binding molecule may be “monospecific” and have a single binding specificity or a binding molecule may be “multispecific” (e.g., bispecific or trispecific or of greater multispecificity) and have two or more binding specificities. In exemplary embodiments, the binding molecules of the invention are “bispecific” and comprise two binding specificities. Thus, whether an IGF-1R binding molecule is “monospecific” or “multispecific,” e.g., “bispecific,” refers to the number of different epitopes with which a binding molecule reacts. In exemplary embodiments, multispecific binding molecules of the invention may be specific for different epitopes on one or more IGF-1R molecule.

In one embodiment, the binding molecule may comprise a dual binding specificity. As used herein the term “dual binding specificity” or “dual specificity” refers to the ability of binding molecule to specifically bind to one or more different epitopes. For example, a binding molecule may comprise a binding specificity having at least one binding site which specifically binds two or more different epitopes (e.g., two or more non-overlapping or discontinuous epitopes) on a target molecule. Accordingly, a binding molecule having a dual binding specificity is said to cross-react with two or more epitopes.

A given binding molecule of the invention may be monovalent or multivalent for a particular binding specificity. For example, when an IGF-1R binding molecule is monospecific, the binding specificity may comprise a single binding site which specifically binds an epitope (i.e., a “monovalent monospecific” binding molecule) and such a binding molecule may be used in combination with a second binding molecule having at least one binding specificity for a different epitope of IGF-1R. In one embodiment, the monospecific IGF-1R binding molecule may comprise two binding domains which specifically bind the same epitope. Such a binding molecule is bivalent and monospecific. In other embodiments, where a binding molecule is multispecific, one or more of its binding specificities may comprise two or more binding domains which specifically bind the same epitope (i.e., a “multivalent binding specificity”). For example, a bispecific molecule may comprise a first binding specificity that is bivalent (ie. two binding sites which bind a first epitope) and a second binding specificity which is bivalent (i.e., two binding sites which bind a second, different epitope). In another embodiment, a bispecific molecule may comprise a first binding specificity that is monovalent (i.e, one binding site which binds a first epitope) and a second binding specificity which is bivalent or monovalent.

Binding molecules disclosed herein may be described or specified in terms of the epitope(s) or portion(s) of an antigen, e.g., a target polypeptide (e.g., IGF-1R) that they recognize or specifically bind. The portion of a target polypeptide which specifically interacts with the binding site or moiety of a binding molecule is an “epitope,” or an “antigenic determinant.” A target polypeptide may comprise a single epitope, but typically comprises at least two epitopes, and can include any number of epitopes, depending on the size, conformation, and type of antigen. Furthermore, it should be noted that an “epitope” on a target polypeptide may be or may include non-polypeptide elements, e.g., an “epitope may include a carbohydrate side chain. The minimum size of a peptide or polypeptide epitope for an antibody is thought to be about four to five amino acids. Peptide or polypeptide epitopes preferably contain at least seven, more preferably at least nine and most preferably between at least about 15 to about 30 amino acids. Since a CDR can recognize an antigenic peptide or polypeptide in its tertiary form, the amino acids comprising an epitope need not be contiguous, and in some cases, may not even be on the same peptide chain. In the present invention, peptide or polypeptide epitope recognized by IGF-1R antibodies of the present invention contains a sequence of at least 4, at least 5, at least 6, at least 7, more preferably at least 8, at least 9, at least 10, at least 15, at least 20, at least 25, or between about 15 to about 30 contiguous or non-contiguous amino acids of IGF-1R.

By “specifically binds,” it is generally meant that a binding molecule binds to an epitope via a binding site of the binding molecule (e.g., antigen binding domain), and that the binding entails some complementarity between that binding site and the epitope. According to this definition, a binding molecule is said to “specifically bind” to an epitope when it binds to that epitope, via the binding site, more readily than it would bind to an unrelated epitope. Where a binding molecule is multispecific, the binding molecule may specifically bind to a second epitope (ie., unrelated to the first epitope) via another binding site (e.g., antigen binding domain) of the binding molecule.

By “preferentially binds,” it is meant that the binding molecule specifically binds to an epitope via a binding site more readily than it would bind to a related, similar, homologous, or analogous epitope. Thus, an antibody which “preferentially binds” to a given epitope would more likely bind to that epitope than to a related epitope, even though such a binding molecule may cross-react with the related epitope.

As used herein, the term “cross-reactivity” refers to the ability of binding molecule, specific for one antigen or antibody, to react with a second antigen; a measure of relatedness between two different antigenic substances. Thus, an antibody is cross reactive if it binds to an epitope other than the one that induced its formation. The cross reactive epitope generally contains many of the same complementary structural features as the inducing epitope, and in some cases, may actually fit better than the original.

For example, certain binding molecules have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody may be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody may be deemed “highly specific” for a certain antigen or epitope, if it does not bind any other analog, ortholog, or homolog of that antigen or epitope.

As used herein, the term “affinity” refers to a measure of the strength of the binding of an individual epitope with the binding site of a binding molecule. See, e.g., Harlow et al., Antibodies: A Laboratory Manual, (Cold Spring Harbor Laboratory Press, 2nd ed. 1988) at pages 27-28. Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M 5×10⁻¹⁴ M, 10⁻¹⁴ M, 5×10⁻¹⁵ M, or 10⁻¹⁵ M.

As used herein, the term “avidity” refers to the overall stability of the complex between a population of binding molecules (e.g. antibodies) and an antigen, that is, the functional combining strength of a binding molecule mixture with the antigen. See, e.g, Harlow at pages 29-34. Avidity is related to both the affinity of individual binding molecules in the population with specific epitopes, and also the valencies of the binding molecules and the antigen. For example, the interaction between a bivalent monoclonal antibody and an antigen with a highly repeating epitope structure, such as a polymer, would be one of high avidity.

In certain embodiments, the binding site of a binding molecule of the invention is an antigen binding site. An antigen binding site is formed by variable regions that vary from one polypeptide to another. In one embodiment, the polypeptides of the invention comprise at least two antigen binding sites. As used herein, the term “antigen binding site” includes a site that specifically binds (immunoreacts with) an antigen (e.g., a cell surface or soluble form of an antigen). The antigen binding site includes an immunoglobulin heavy chain and light chain variable region and the binding site formed by these variable regions determines the specificity of the antibody. In one embodiment, an antigen binding site of the invention comprises at least one heavy or light chain CDR of an antibody molecule (e.g., the sequence of which is known in the art or described herein). In another embodiment, an antigen binding site of the invention comprises at least two CDRs from one or more antibody molecules. In another embodiment, an antigen binding site of the invention comprises at least three CDRs from one or more antibody molecules. In another embodiment, an antigen binding site of the invention comprises at least four CDRs from one or more antibody molecules. In another embodiment, an antigen binding site of the invention comprises at least five CDRs from one or more antibody molecules. In another embodiment, an antigen binding site of the invention comprises at least six CDRs from one or more antibody molecules. Exemplary binding sites comprising at least one CDR (e.g., CDRs 1-6) that can be included in the subject antigen binding molecules are known in the art and exemplary molecules are described herein.

Preferred binding molecules of the invention comprise framework and constant region amino acid sequences derived from a human amino acid sequence. However, binding polypeptides may comprise framework and/or constant region sequences derived from another mammalian species. For example, binding molecules comprising murine sequences may be appropriate for certain applications. In one embodiment, a primate framework region (e.g., non-human primate), heavy chain portion, and/or hinge portion may be included in the subject binding molecules. In one embodiment, one or more murine amino acids may be present in the framework region of a binding polypeptide, e.g., a human or non-human primate framework amino acid sequence may comprise one or more amino acid back mutations in which the corresponding murine amino acid residue is present and/or may comprise one or mutations to a different amino acid residue not found in the starting murine antibody. Preferred binding molecules of the invention are less immunogenic than murine antibodies.

A “fusion” or chimeric protein comprises a first amino acid sequence linked to a second amino acid sequence with which it is not naturally linked in nature. The amino acid sequences may normally exist in separate proteins that are brought together in the fusion polypeptide or they may normally exist in the same protein but are placed in a new arrangement in the fusion polypeptide. A fusion protein may be created, for example, by chemical synthesis, or by creating and translating a polynucleotide in which the peptide regions are encoded in the desired relationship.

The term “heterologous” as applied to a polynucleotide or a polypeptide, means that the polynucleotide or polypeptide is derived from a genotypically distinct entity from that of the entity to which it is being compared. For instance, a heterologous polynucleotide or antigen may be derived from a different species, different cell type of an individual, or the same or different type of cell of distinct individuals.

The term “receptor binding domain” or “receptor binding portion” as used herein refers to any native ligand or any region or derivative thereof retaining at least a qualitative receptor binding ability, and preferably the biological activity of a corresponding native ligand.

The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988).

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the instant invention. All immunoglobulin classes are clearly within the scope of the present invention, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains actually comprise the carboxy-terminus of the heavy and light chain, respectively.

As indicated above, the variable region allows the antibody to selectively recognize and specifically bind epitopes on antigens. That is, the VL domain and VH domain, or subset of the complementarity determining regions (CDRs), of an antibody (e.g., in some instances a CH3 domain) combine to form the variable region that defines a three dimensional antigen binding site. This quaternary antibody structure forms the antigen binding site present at the end of each arm of the Y. In one embodiment, the antigen binding site is defined by three CDRs on each of the VH and VL chains. In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

As used herein the term “variable region CDR amino acid residues” includes amino acids in a CDR or complementarity determining region as identified using sequence or structure based methods. As used herein, the term “CDR” or “complementarity determining region” refers to the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth in Table 1 for comparison. Preferably, the term “CDR” is a CDR as defined by Kabat based on sequence comparisons.

TABLE 1
CDR Definitions

CDR Definitions

| | Kabat¹ | Chothia² | MacCallum³ |
|---|---|---|---|
| V_{H}CDR1 | 31-35 | 26-32 | 30-35 |
| V_{H}CDR2 | 50-65 | 53-55 | 47-58 |
| V_{H}CDR3 | 95-102 | 96-101 | 93-101 |
| V_{L}CDR1 | 24-34 | 26-32 | 30-36 |
| V_{L}CDR2 | 50-56 | 50-52 | 46-55 |
| V_{L}CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

As used herein the term “variable region framework (FR) amino acid residues” refers to those amino acids in the framework region of an Ig chain. The term “framework region” or “FR region” as used herein, includes the amino acid residues that are part of the variable region, but are not part of the CDRs (e.g., using the Kabat definition of CDRs). Therefore, a variable region framework is between about 100-120 amino acids in length but includes only those amino acids outside of the CDRs. For the specific example of a heavy chain variable region and for the CDRs as defined by Kabat et al., framework region 1 corresponds to the domain of the variable region encompassing amino acids 1-30; framework region 2 corresponds to the domain of the variable region encompassing amino acids 36-49; framework region 3 corresponds to the domain of the variable region encompassing amino acids 66-94, and framework region 4 corresponds to the domain of the variable region from amino acids 103 to the end of the variable region. The framework regions for the light chain are similarly separated by each of the light chain variable region CDRs. Similarly, using the definition of CDRs by Chothia et al. or McCallum et al. the framework region boundaries are separated by the respective CDR termini as described above. In preferred embodiments, the CDRs are as defined by Kabat.

In naturally occurring antibodies, the six CDRs present on each monomeric antibody are short, non-contiguous sequences of amino acids that are specifically positioned to form the antigen binding site as the antibody assumes its three dimensional configuration in an aqueous environment. The remainder of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of the variable region of an IGF-1R antibody or antigen-binding fragment, variant, or derivative thereof of the present invention are according to the Kabat numbering system.

As used herein, the term “Fc domain” or “Fc region” refers to the portion of an immunoglobulin heavy chain beginning in the hinge region just upstream of the papain cleavage site (i.e. residue 216 in IgG, taking the first residue of heavy chain constant region to be 114) and ending at the C-terminus of the antibody. Accordingly, a complete Fc region comprises at least a hinge domain, a CH2 domain, and a CH3 domain. In certain embodiments, the Fc region is a dimer comprising at least two separate heavy chain portions. In other embodiments, the Fc region is a single chain Fc region (“scFc”) comprising at least two heavy chain portions that are fused or linked (e.g., via a Gly/Ser peptide or other peptide linker). ScFc regions are described in detail in International PCT Application No. PCT/US2008/006260, filed May 14, 2008, which is incorporated by reference herein in its entirety.

As used herein, the term “Fc domain portion” or “Fc portion” includes amino acid sequences derived from an Fc domain or Fc region. A polypeptide comprising a Fc portion comprises at least one of: a hinge (e.g., upper, middle, and/or lower hinge region) domain, a CH2 domain, a CH3 domain, a CH4 domain, or a variant or fragment thereof. In one embodiment, a polypeptide of the invention comprises at least one Fc region comprising at least a portion of a hinge domain, and a CH2 domain. In another embodiment, a polypeptide of the invention comprises at least one Fc region comprising a CH1 domain and a CH3 domain. In another embodiment, a polypeptide of the invention comprises at least one Fc region comprising a CH1 domain, at least a portion of a hinge domain, and a CH3 domain. In another embodiment, a polypeptide of the invention comprises at least one Fc region comprising a CH3 domain. In one embodiment, a polypeptide of the invention comprises at least one Fc region which lacks at least a portion of a CH2 domain (e.g., all or part of a CH2 domain). As set forth herein, it will be understood by one of ordinary skill in the art that any Fc region may be modified such that it varies in amino acid sequence from the native Fc region of a naturally occurring immunoglobulin molecule.

The Fc region of a polypeptide of the invention may be derived from different immunoglobulin molecules (e.g., two or more different human antibody isotypes). For example, an Fc region of a polypeptide may comprise a CH1 domain derived from an IgG1 molecule and a chimeric hinge region derived from an IgG3 molecule. In another example, an Fc region can comprise a hinge region derived, in part, from an IgG1 molecule and, in part, from an IgG3 molecule. In another example, an Fc region can comprise a chimeric hinge derived, in part, from an IgG1 molecule and, in part, from an IgG4 molecule. In another embodiment, the Fc region can comprise a hinge domain from a first antibody isotype (e.g., IgG1 or IgG2) and a CH2 domain from a different human antibody isotype (e.g., IgG4). In another embodiment, the Fc region can comprise a CH2 domain from a first antibody isotype (e.g., IgG1 or IgG2) and a CH3 domain from a different human antibody isotype (e.g., IgG4). In another embodiment, residues 233-236 and 327-331 of the Fc region are from a human IgG2 antibody and the remaining residues of the Fc region are from a human IgG4 antibody. Exemplary chimeric Fc regions are disclosed, for example, in PCT Publication No. WO/1999/58572, which is incorporated herein by reference in its entirety.

Amino acid positions in a heavy chain constant region, including amino acid positions in the CH1, hinge, CH2, and CH3 domains, are numbered herein according to the EU index numbering system (see Kabat et al., in “Sequences of Proteins of Immunological Interest”, U.S. Dept. Health and Human Services, 5^{th} edition, 1991). In contrast, amino acid positions in a light chain constant region (e.g. CL domains) are numbered herein according to the Kabat index numbering system (see Kabat et al., ibid).

Exemplary binding molecules include or may comprise, for example, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library, and anti-idiotypic (anti-Id) antibodies (including, e.g., anti-Id antibodies to IGF-1R antibodies disclosed herein). ScFv molecules are known in the art and are described, e.g., in U.S. Pat. No. 5,892,019. Binding molecules of the invention which comprise an Ig heavy chain may be of any type (e.g., IgG, IgE, IgM, IgD, IgA, and IgY), class (e.g., IgG1, IgG2, IgG3, IgG4, IgA1 and IgA2) or subclass of immunoglobulin molecule.

Binding molecules may comprise the variable region(s) alone or in combination with the entirety or a portion of the following: hinge region, CH1, CH2, and CH3 domains. Also included in the invention are antigen-binding fragments comprising any combination of variable region(s) with a hinge region, CH1, CH2, and CH3 domains. Binding molecules of the present invention may be or may be derived from antibodies of any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al.

The term “fragment” refers to a part or portion of a polypeptide (e.g., an antibody or an antibody chain) comprising fewer amino acid residues than an intact or complete polypeptide. The term “antigen-binding fragment” refers to a polypeptide fragment of an immunoglobulin or antibody that binds antigen or competes with intact antibody (i.e., with the intact antibody from which they were derived) for antigen binding (i.e., specific binding). As used herein, the term “fragment” of an antibody molecule includes antigen-binding fragments of antibodies, for example, an antibody light chain (VL), an antibody heavy chain (VH), a single chain antibody (scFv), a F(ab′)2 fragment, a Fab fragment, an Fd fragment, an Fv fragment, and a single domain antibody fragment (DAb). Fragments can be obtained, e.g., via chemical or enzymatic treatment of an intact or complete antibody or antibody chain or by recombinant means.

In one embodiment, a binding molecule of the invention comprises a constant region, e.g., a heavy chain constant region. In one embodiment, such a constant region is modified compared to a wild-type constant region. That is, the polypeptides of the invention disclosed herein may comprise alterations or modifications to one or more of the three heavy chain constant domains (CH1, CH2 or CH3) and/or to the light chain constant region domain (CL). Exemplary modifications include additions, deletions or substitutions of one or more amino acids in one or more domains. Other modified constants regions lack glycosylation or have altered glycan structures (e.g., afucosylated glycans). Such changes may be included to optimize or reduce or eliminate effector function, improve half-life, etc.

In certain embodiments, the binding molecules of the invention include a heavy chain portion which is linked to one or more of the binding sites of the binding molecule. As used herein, the term “heavy chain portion” includes amino acid sequences derived from a constant region of an immunoglobulin heavy chain. A polypeptide comprising a heavy chain portion comprises at least one of: a CH1 domain, a hinge (e.g., upper, middle, and/or lower hinge region) domain, a CH2 domain, a CH3 domain, or a variant or fragment thereof. For example, a binding polypeptide for use in the invention may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the invention comprises a polypeptide chain comprising a CH3 domain. Further, a binding polypeptide for use in the invention may lack at least a portion of a CH2 domain (e.g., all or part of a CH2 domain). As set forth above, it will be understood by one of ordinary skill in the art that these domains (e.g., the heavy chain portions) may be modified such that they vary in amino acid sequence from the naturally occurring immunoglobulin molecule. In certain embodiments where the binding molecule is a multimer, the heavy chain portions of one polypeptide chain of a multimer are identical to those on a second polypeptide chain of the multimer. Alternatively, in one embodiment, the heavy chain portion-containing monomers of the invention are not identical. For example, each monomer may comprise a different target binding site, forming, for example, a bispecific antibody.

The heavy chain portions of a binding polypeptide for use in the methods disclosed herein may be derived from different immunoglobulin molecules. For example, a heavy chain portion of a polypeptide may comprise a CH1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain. Preferably, the light chain portion comprises at least one of a VL or CL domain.

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein, the term “CH1 domain” includes the first (most amino terminal) constant region domain of an immunoglobulin heavy chain that extends, e.g., from about EU positions 118-215. The CH1 domain is adjacent to the VH domain and amino terminal to the hinge region of an immunoglobulin heavy chain molecule, and does not form a part of the Fc region of an immunoglobulin heavy chain. In one embodiment, a binding molecule of the invention comprises a CH1 domain derived from an immunoglobulin heavy chain molecule (e.g., a human IgG1 or IgG4 molecule).

As used herein, the term “CH2 domain” includes the portion of a heavy chain immunoglobulin molecule that extends, e.g., from about EU positions 231-340. The CH2 domain is unique in that it is not closely paired with another domain. Rather, two N-linked branched carbohydrate chains are interposed between the two CH2 domains of an intact native IgG molecule. In one embodiment, a binding molecule of the invention comprises a CH2 domain derived from an IgG1 molecule (e.g. a human IgG1 molecule). In another embodiment, an altered polypeptide of the invention comprises a CH2 domain derived from an IgG4 molecule (e.g., a human IgG4 molecule). In an exemplary embodiment, a polypeptide of the invention comprises a CH2 domain (EU positions 231-340), or a portion thereof.

As used herein, the term “CH3 domain” includes the portion of a heavy chain immunoglobulin molecule that extends approximately 110 residues from N-terminus of the CH2 domain, e.g., from about position 341-446b (EU numbering system). The CH3 domain typically forms the C-terminal portion of the antibody. In some immunoglobulins, however, additional domains may extend from CH3 domain to form the C-terminal portion of the molecule (e.g. the CH4 domain in the μ chain of IgM and the ε chain of IgE). In one embodiment, a binding molecule of the invention comprises a CH3 domain derived from an IgG1 molecule (e.g., a human IgG1 molecule). In another embodiment, a binding molecule of the invention comprises a CH3 domain derived from an IgG4 molecule (e.g., a human IgG4 molecule).

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., J. Immunol. 161:4083 (1998)).

As used herein, the term “effector function” refers to the functional ability of the Fc region or portion thereof to bind proteins and/or cells of the immune system and mediate various biological effects. Effector functions may be antigen-dependent or antigen-independent. A decrease in effector function refers to a decrease in one or more effector functions, while maintaining the antigen binding activity of the variable region of the antibody (or fragment thereof). Increase or decreases in effector function, e.g., Fc binding to an Fc receptor or complement protein, can be expressed in terms of fold change (e.g., changed by 1-fold, 2-fold, and the like) and can be calculated based on, e.g., the percent changes in binding activity determined using assays the are well-known in the art.

As used herein, the term “antigen-dependent effector function” refers to an effector function which is normally induced following the binding of an antibody to a corresponding antigen. Typical antigen-dependent effector functions include the ability to bind a complement protein (e.g. C1q). For example, binding of the C1 component of complement to the Fc region can activate the classical complement system leading to the opsonisation and lysis of cell pathogens, a process referred to as complement-dependent cytotoxicity (CDCC). The activation of complement also stimulates the inflammatory response and may also be involved in autoimmune hypersensitivity.

Other antigen-dependent effector functions are mediated by the binding of antibodies, via their Fc region, to certain Fc receptors (“FcRs”) on cells. There are a number of Fc receptors which are specific for different classes of antibody, including IgG (gamma receptors, or IgγRs), IgE (epsilon receptors, or IgεRs), IgA (alpha receptors, or IgαRs) and IgM (mu receptors, or IgμRs). Binding of antibody to Fc receptors on cell surfaces triggers a number of important and diverse biological responses including endocytosis of immune complexes, engulfment and destruction of antibody-coated particles or microorganisms (also called antibody-dependent phagocytosis, or ADCP), clearance of immune complexes, lysis of antibody-coated target cells by killer cells (called antibody-dependent cell-mediated cytotoxicity, or ADCC), release of inflammatory mediators, regulation of immune system cell activation, placental transfer and control of immunoglobulin production.

As used herein, the term “chimeric antibody” will be held to mean any antibody wherein the binding site or moiety (e.g., the variable region) is obtained or derived from a first species and the constant region (which may be intact, partial or modified in accordance with the instant invention) is obtained from a second species. In preferred embodiments the target binding region or site will be from a non-human source (e.g. mouse or primate) and the constant region is human.

As used herein the term “scFv molecule” includes binding molecules which consist of one light chain variable domain (VL) or portion thereof, and one heavy chain variable domain (VH) or portion thereof, wherein each variable domain (or portion thereof) is derived from the same or different antibodies. scFv molecules preferably comprise an scFv linker interposed between the VH domain and the VL domain. scFv molecules are known in the art and are described, e.g., in U.S. Pat. No. 5,892,019, Ho et al. 1989. Gene 77:51; Bird et al. 1988 Science 242:423; Pantoliano et al. 1991. Biochemistry 30:10117; Milenic et al. 1991. Cancer Research 51:6363; Takkinen et al. 1991. Protein Engineering 4:837. The VL and VH domains of an scFv molecule are derived from one or more antibody molecules. It will also be understood by one of ordinary skill in the art that the variable regions of the scFv molecules of the invention may be modified such that they vary in amino acid sequence from the antibody molecule from which they were derived. For example, in one embodiment, nucleotide or amino acid substitutions leading to conservative substitutions or changes at amino acid residues may be made (e.g., in CDR and/or framework residues). Alternatively or in addition, mutations may be made to CDR amino acid residues to optimize antigen binding using art recognized techniques. The binding molecules of the invention maintain the ability to bind to antigen.

A “scFv linker” as used herein refers to a moiety interposed between the VL and VH domains of the scFv. scFv linkers preferably maintain the scFv molecule in a antigen binding conformation. In one embodiment, an scFv linker comprises or consists of an scFv linker peptide. In certain embodiments, an scFv linker peptide comprises or consists of a gly-ser connecting peptide. In other embodiments, an scFv linker comprises a disulfide bond.

As used herein, the term “gly-ser connecting peptide” refers to a peptide that consists of glycine and serine residues. An exemplary gly/ser connecting peptide comprises the amino acid sequence (Gly₄ Ser)ₙ. In one embodiment, n=1. In one embodiment, n=2. In another embodiment, n=3. In a preferred embodiment, n=4, i.e., (Gly₄ Ser)₄. In another embodiment, n=5. In yet another embodiment, n=6. Another exemplary gly/ser connecting peptide comprises the amino acid sequence Ser(Gly₄Ser)ₙ. In one embodiment, n=1. In one embodiment, n=2. In a preferred embodiment, n=3. In another embodiment, n=4. In another embodiment, n=5. In yet another embodiment, n=6.

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by a disulfide bond and the two heavy chains are linked by two disulfide bonds at positions corresponding to 239 and 242 using the Kabat numbering system (position 226 or 229, EU numbering system).

As used herein the term “conventional scFv molecule” refers to an scFv molecules which is not a stabilized scFv molecule. For example, a typical conventional scFv molecule lacks stabilizing mutations and comprises a VH and a VL domain linked by a (G₄S)₃ linker.

A “stabilized scFv molecule” of the invention is an scFv molecule comprising at least one change or alteration as compared to a conventional scFv molecule which results in stabilization of the scFv molecule (i.e., as compared to the conventional scFv molecule). As used herein, the term “stabilizing mutation” includes a mutation which confers enhanced protein stability (e.g. thermal stability) to the scFv molecule and/or to a larger protein comprising said scFv molecule. In one embodiment, the stabilizing mutation comprises the substitution of a destabilizing amino acid with a replacement amino acid that confers enhanced protein stability (herein a “stabilizing amino acid”). In one embodiment, the stabilizing mutation is one in which the length of an scFv linker has been optimized. In one embodiment, a stabilized scFv molecule of the invention comprises one or more amino acid substitutions. For example, in one embodiment, a stabilizing mutation comprises a substitution of at least one amino acid residue which substitution results in an increase in stability of the VH and VL interface of an scFv molecule. In one embodiment, the amino acid is within the interface. In another embodiment, the amino acid is one which scaffolds the interaction between the VH and VL. In another embodiment, a stabilizing mutation comprises substituting at least one amino acid in the VH domain or VL domain that covaries with two or more amino acids at the interface between the VH and VL domains. In another embodiment, the stabilizing mutation is one in which at least one cysteine residue is introduced (i.e., is engineered into one or more of the VH or VL domain) such that the VH and VL domains are linked by at least one disulfide bond between an amino acid in the VH and an amino acid in the VL domain. In certain preferred embodiments, a stabilized scFv molecule of the invention is one in which both the length of the scFv linker is optimized and at least one amino acid residue is substituted and/or the VH and VL domains are linked by a disulfide bond between an amino acid in the VH and an amino acid in the VL domain. In one embodiment, one or more stabilizing mutations made to an scFv molecule simultaneously improves the thermal stability of both the VH and VL domains of the scFv molecule as compared to a conventional scFv molecule. In one embodiment, the stabilized scFv molecules of the population may comprise the same stabilizing mutation or a combination of stabilizing mutations. In other embodiments, the individual stabilized scFv molecules of the population comprise different stabilizing mutations. Exemplary stabilizing mutations are described in detail in U.S. patent application Ser. No. 11/725,970, which is incorporated by reference herein in its entirety.

As used herein the term “protein stability” refers to an art-recognized measure of the maintenance of one or more physical properties of a protein in response to an environmental condition (e.g. an elevated or lowered temperature). In one embodiment, the physical property is the maintenance of the covalent structure of the protein (e.g. the absence of proteolytic cleavage, unwanted oxidation or deamidation). In another embodiment, the physical property is the presence of the protein in a properly folded state (e.g. the absence of soluble or insoluble aggregates or precipitates). In one embodiment, stability of a protein is measured by assaying a biophysical property of the protein, for example thermal stability, pH unfolding profile, stable removal of glycosylation, solubility, biochemical function (e.g., ability to bind to a protein (e.g., a ligand, a receptor, an antigen, etc.) or chemical moiety, etc.), and/or combinations thereof. In another embodiment, biochemical function is demonstrated by the binding affinity of an interaction. In one embodiment, a measure of protein stability is thermal stability, i.e., resistance to thermal challenge. Stability can be measured using methods known in the art and/or described herein.

As used herein, the term “engineered antibody” refers to an antibody in which the variable domain in either the heavy and light chain or both is altered by at least partial replacement of one or more CDRs from an antibody of known specificity and, if necessary, by partial framework region replacement and sequence changing. Although the CDRs may be derived from an antibody of the same class or even subclass as the antibody from which the framework regions are derived, it is envisaged that the CDRs will be derived from an antibody of different class and preferably from an antibody from a different species. An engineered antibody in which one or more “donor” CDRs from a non-human antibody of known specificity is grafted into a human heavy or light chain framework region is referred to herein as a “humanized antibody.” It may not be necessary to replace all of the CDRs with the complete CDRs from the donor variable region to transfer the antigen binding capacity of one variable domain to another. Rather, it may only be necessary to transfer those residues that are necessary to maintain the activity of the target binding site. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein the term “properly folded polypeptide” includes polypeptides (e.g., IGF-1R scFv molecules) in which all of the functional domains comprising the polypeptide are distinctly active. As used herein, the term “improperly folded polypeptide” includes polypeptides in which at least one of the functional domains of the polypeptide is not active. In one embodiment, a properly folded polypeptide comprises polypeptide chains linked by at least one disulfide bond and, conversely, an improperly folded polypeptide comprises polypeptide chains not linked by at least one disulfide bond.

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding an IGF-1R binding molecule contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, polynucleotide or a nucleic acid may be or may include a regulatory element such as a promoter, ribosome binding site, or a transcription terminator.

As used herein, a “coding region” is a portion of nucleic acid molecule which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid of the invention may encode heterologous coding regions, either fused or unfused to a nucleic acid encoding an IGF-1R binding molecule or fragment, variant, or derivative thereof. Heterologous coding regions include without limitation specialized elements or motifs, such as a secretory signal peptide or a heterologous functional domain or tags that may facilitate identification or purification.

In certain embodiments, the polynucleotide or nucleic acid molecule is a DNA molecule. In the case of DNA, a polynucleotide comprising a nucleic acid which encodes a polypeptide normally may include a promoter and/or other transcription or translation control elements operably associated with one or more coding regions. In an operable association a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide of the present invention is an RNA molecule, for example, in the form of messenger RNA (mRNA).

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

As used herein the term “engineered” with reference to nucleic acid or polypeptide molecules refers to such molecules manipulated by synthetic means (e.g. by recombinant techniques, in vitro peptide synthesis, by enzymatic or chemical coupling of peptides or some combination of these techniques).

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments may be physically or spatially separated by, for example, in-frame linker sequence. For example, polynucleotides encoding the CDRs of an immunoglobulin variable region may be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (shRNA), small interfering RNA (siRNA) or any other RNA product, and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the terms “treat” or “treatment” refer to both therapeutic treatment and prophylactic or preventative measures, wherein the object is to prevent or slow down (lessen) an undesired physiological change or disorder, such as the development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on.

As used herein, phrases such as “a subject that would benefit from administration of a binding molecule” and “an animal in need of treatment” includes subjects, such as mammalian subjects, that would benefit from administration of a binding molecule used, e.g., for detection of an antigen recognized by a binding molecule (e.g., for a diagnostic procedure) and/or from treatment, i.e., palliation or prevention of a disease such as cancer, with a binding molecule which specifically binds a given target protein. As described in more detail herein, the binding molecule can be used in unconjugated form or can be conjugated, e.g., to a drug, prodrug, or an isotope.

By “hyperproliferative disease or disorder” is meant neoplastic cell growth or proliferation, whether malignant or benign, including transformed cells and tissues and all cancerous cells and tissues. Hyperproliferative diseases or disorders include, but are not limited to, precancerous lesions, abnormal cell growths, benign tumors, malignant tumors, and “cancer.” In certain embodiments of the present invention, the hyperproliferative disease or disorder, e.g., the precancerous lesion, abnormal cell growth, benign tumor, malignant tumor, or “cancer” comprises cells which express, over-express, or abnormally express IGF-1R.

Additional examples of hyperproliferative diseases, disorders, and/or conditions include, but are not limited to neoplasias, whether benign or malignant, located in the: prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital tract. Such neoplasias, in certain embodiments, express, over-express, or abnormally express IGF-1R.

Other hyperproliferative disorders include, but are not limited to: hypergammaglobulinemia, lymphoproliferative disorders, paraproteinemias, purpura, sarcoidosis, Sezary Syndrome, Waldenstron's macroglobulinemia, Gaucher's Disease, histiocytosis, and any other hyperproliferative disease, besides neoplasia, located in an organ system listed above. In certain embodiments of the present invention the diseases involve cells which express, over-express, or abnormally express IGF-1R.

As used herein, the terms “tumor” or “tumor tissue” refer to an abnormal mass of tissue that results from excessive cell division, in certain cases tissue comprising cells which express, over-express, or abnormally express IGF-1R. A tumor or tumor tissue comprises “tumor cells” which are neoplastic cells with abnormal growth properties and no useful bodily function. Tumors, tumor tissue and tumor cells may be benign or malignant. A tumor or tumor tissue may also comprise “tumor-associated non-tumor cells”, e.g., vascular cells which form blood vessels to supply the tumor or tumor tissue. Non-tumor cells may be induced to replicate and develop by tumor cells, for example, the induction of angiogenesis in a tumor or tumor tissue.

As used herein, the term “malignancy” refers to a non-benign tumor or a cancer. As used herein, the term “cancer” connotes a type of hyperproliferative disease which includes a malignancy characterized by deregulated or uncontrolled cell growth. Examples of cancer include, but are not limited to, carcinoma, lymphoma, blastoma, sarcoma, and leukemia or lymphoid malignancies. More particular examples of such cancers are noted below and include: squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). Cancers conducive to treatment methods of the present invention involves cells which express, over-express, or abnormally express IGF-1R.

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

The method of the present invention may be used to treat premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, Basic Pathology, 2d Ed., W.B. Saunders Co., Philadelphia, pp. 68-79 (1976). Such conditions in which cells begin to express, over-express, or abnormally express IGF-1R, are particularly treatable by the methods of the present invention.

Hyperplasia is a form of controlled cell proliferation, involving an increase in cell number in a tissue or organ, without significant alteration in structure or function. Hyperplastic disorders which can be treated by the method of the invention include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, focal epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

Metaplasia is a form of controlled cell growth in which one type of adult or fully differentiated cell substitutes for another type of adult cell. Metaplastic disorders which can be treated by the method of the invention include, but are not limited to, agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, and symptomatic myeloid metaplasia.

Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation. Dysplastic disorders which can be treated by the method of the invention include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, opthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders which can be treated by the method of the invention include, but are not limited to, benign dysproliferative disorders (e.g., benign tumors, fibrocystic conditions, tissue hypertrophy, intestinal polyps, colon polyps, and esophageal dysplasia), leukoplakia, keratoses, Bowen's disease, Farmer's Skin, solar cheilitis, and solar keratosis.

In preferred embodiments, the method of the invention is used to inhibit growth of hyperproliferative cells (e.g., proliferation of IGF-1R expressing tumor cells in vitro or in vivo), progression, and/or metastasis of cancers, in particular those listed above.

Additional hyperproliferative diseases, disorders, and/or conditions include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendroglioma, menangioma, melanoma, neuroblastoma, and retinoblastoma.

### II. The IGF System

The IGF system plays an important role in regulating cell proliferation, differentiation, apoptosis and transformation (Jones et al, Endocrinology Rev. 1995. 16:3-34) and tumor cells have been shown to produce one or more of the components of the IGF system. The IGF system comprises of two types of unrelated receptors, the insulin like growth factor receptor 1 (IGF-1R; CD221) and insulin like growth factor receptor 2 (IGF-2R; CD222); two ligands, insulin like growth factor 1 (IGF-1 and IGF-2); several IGF binding proteins (IGFBP-1 to IGFBP-6); and the proteins involved in intracellular signaling distal to IGF1R, which include members of the insulin-receptor substrate (IRS) family, AKT, target of rapamycin (TOR), and S6 kinase. In addition, a large group of IGFBP proteases (e.g.: caspases, metalloproteinases, prostate-specific antigen) hydrolyze IGF bound IGFBP to release free IGFs, which then interact with IGF-1R and IGF-2R. The IGF system is also intimately connected to insulin and insulin receptor (InsR) (Moschos et al. Oncology 2002. 63:317-32; Baserga et al., Int J. Cancer. 2003. 107:873-77; Pollak et al., Nature Reviews Cancer. 2004. 4:505-516).

### (a) IGF-1

IGF-1 has characteristics of both a circulating hormone and a tissue growth factor. Most IGF-1 found in the circulation is produced in the liver, but it is now recognized that IGF-1 is also synthesized in other organs where autocrine and paracrine mechanisms of action are also important. IGF-1 signaling stimulates proliferation and prolongs survival of cells. A number of epidemiological studies have shown that higher than normal circulating levels of IGF-1 are associated with increased risk for several common cancers, including breast (Hankinson et al, Lancet 1998.351:1393-6), prostate (Chan et al, Science. 1998. 279:563-6), lung (Yu et al, J. Natl. Cancer Inst. 1999. 91:151-6) and colorectal cancers (Ma et al, J. Natl. Cancer Inst. 1999. 91:620-5).

### (b) IGF-2

Elevated circulating levels of IGF-2 also have been shown to be associated with increases risk for endometrial cancer (Jonathan et al, Cancer Biomarker & Prevention. 2004. 13:748-52). IGF2 is also expressed in the liver and in extrahepatic sites. Although in vitro studies have indicated that tumors can produce IGF-1 or IGF-2, translational studies indicate that IGF-2 is the more relevant and commonly expressed IGF in the tumors. This is due to loss of imprinting (LOI) of the silenced IGF-2 allele in the tumor by epigenetic alterations, resulting in biallelic expression of the IGF-2 gene (Fienberg et al., Nat. Rev. Cancer 2004. 4:143-53; Giovannucci et al, Horm. Metab. Res. 2003. 35:694-04; De Souza et al, FASEB J. et al, 1997. 11:60-7). This in turn results in increased IGF-2 supply to cancer cells and to the microenvironment supporting tumor growth.

### (c) IGF-1R

Both IGF1 and IGF2 are ligands for IGF-1R, which is a cell-surface tyrosine kinase signaling molecule. IGF-1R is also known in the art by the names CD221 and JTK13. Following ligand binding to IGF-1R, intracellular signaling pathways that favor proliferation as well as cell survival are activated. Initial phosphorylation targets for IGF-1R include IRS proteins, and downstream signaling molecules include phosphatidylinositol 3-kinase, AKT, TOR, S6 kinase, and mitogen activated protein kinase (MAPK).

Structurally, IGF-1R is highly related to InsR (Pierre De Meyts and Whittaker, Nature Reviews Drug Discovery. 2002, 1: 769-83). IGF-1R contains 84% sequence identity to InsR at the kinase domain, whereas the juxta-membrane and the c-terminal regions share 61% and 44% sequence identity, respectively (Ulrich et al., EMBO J., 1986, 5:2503-12; Blakesley et al., Cytokine Growth Factor Rev., 1996. 7:153-56). Despite the high degree of homology between IGF-1R and InsR, evidence suggests that the two receptors have distinct biological roles; InsR is a key regulator of physiological functions such as glucose transport and biosynthesis of glycogen and fat, whereas the IGF-1R is a potent regulator of cell growth and differentiation. In contrast to InsR, IGF-1R is ubiquitously expressed in tissues where it plays a role in tissue growth, under the control of growth hormone (GH), which modulates IGF-1. Although IGF-1R activation has been shown to promote normal cell growth, experimental evidence suggests that IGF-1R is not an absolute requirement (Baserga et al, Exp Cell Res. 1999. 253:1-6; Baserga et al, Int. J. Cancer. 2003. 107:873-77). In a cancer cell, in addition to pro-survival and proliferative signaling, activation of IGF-1R has also been shown to be involved in motility and invasion (Ress et al., Oncogene 2001. 20:490-00, Nolan et al, Int. J. Cancer. 1997.72:828-34, Stracke et al, J. Biol. Chem. 1989. 264:21544-49; Jackson et al, Oncogene, 2001. 20:7318-25).

IGF-1R is expressed in a large number of tumor cells, including, but not limited to certain of the following: bladder tumors (Hum. Pathol. 34:803 (2003)); brain tumors (Clinical Cancer Res. 8:1822 (2002)); breast tumors (Eur. J. Cancer 30:307 (1994) and Hum Pathol. 36:448-449 (2005)); colon tumors, e.g., adenocarcinomas, metastases, and adenomas (Human Pathol. 30:1128 (1999), Virchows. Arc. 443:139 (2003), and Clinical Cancer Res. 10:843 (2004)); gastric tumors (Clin. Exp. Metastasis 21:755 (2004)); kidney tumors, e.g., clear cell, chromophobe and papillary RCC (Am. J. Clin. Pathol. 122:931-937 (2004)); lung tumors (Hum. Pathol. 34:803-808 (2003) and J. Cancer Res. Clinical Oncol. 119:665-668 (1993)); ovarian tumors (Hum. Pathol. 34:803-808 (2003)); pancreatic tumors, e.g., ductal adenocarcinoma (Digestive Diseases. Sci. 48:1972-1978 (2003) and Clinical Cancer Res. 11:3233-3242 (2005)); and prostate tumors (Cancer Res. 62:2942-2950 (2002)).

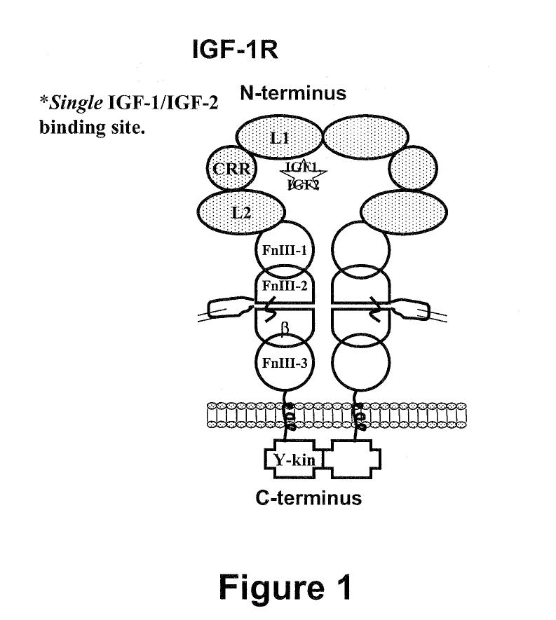

The molecular architecture of IGF-1R comprises, two extra-cellular α subunits (130-135 kD each) and two membrane spanning β subunits (95 kD each) that contain the cytoplasmic catalytic kinase domain. IGF-1R, like the insulin receptor (InsR), differs from other RTK family members by having covalent dimeric (α2β2) structures linked by disulfide bonds (Massagué, J. and Czech, M. P. J. Biol. Chem. 257:5038-5045 (1992)). The IGF-1R extracellular region consists of 6 protein domains which linked in series as follows: an N-terminal Leucine Rich Repeat Domain (L1); a Cysteine Rich Repeat (CRR); a second Leucine Rich Repeat domain (L2); and three Fibronectin Type III domains, denoted FnIII-1, FnIII-2, and FnIII-3 (see FIG. 1).

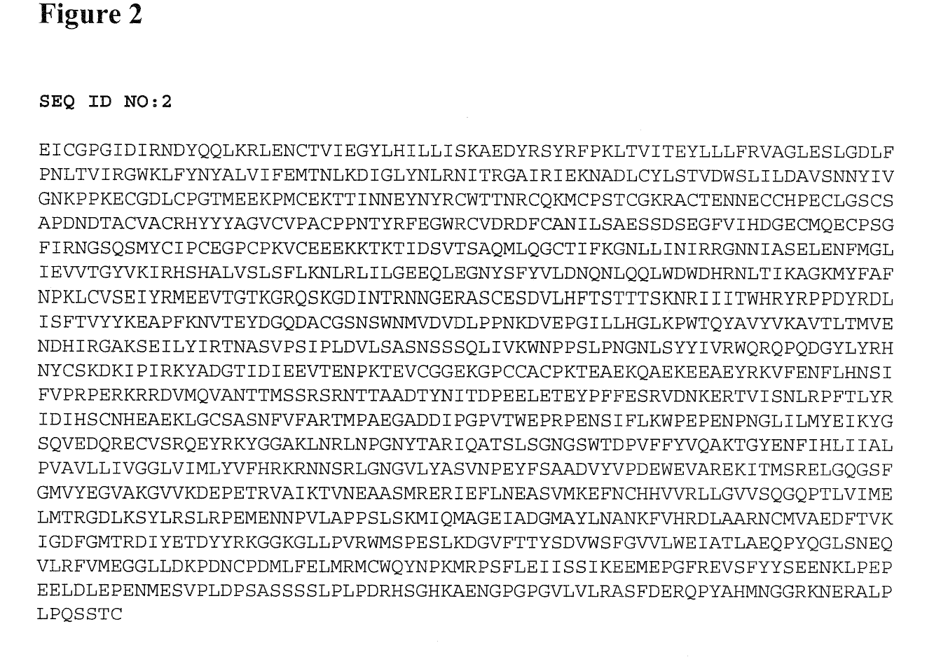

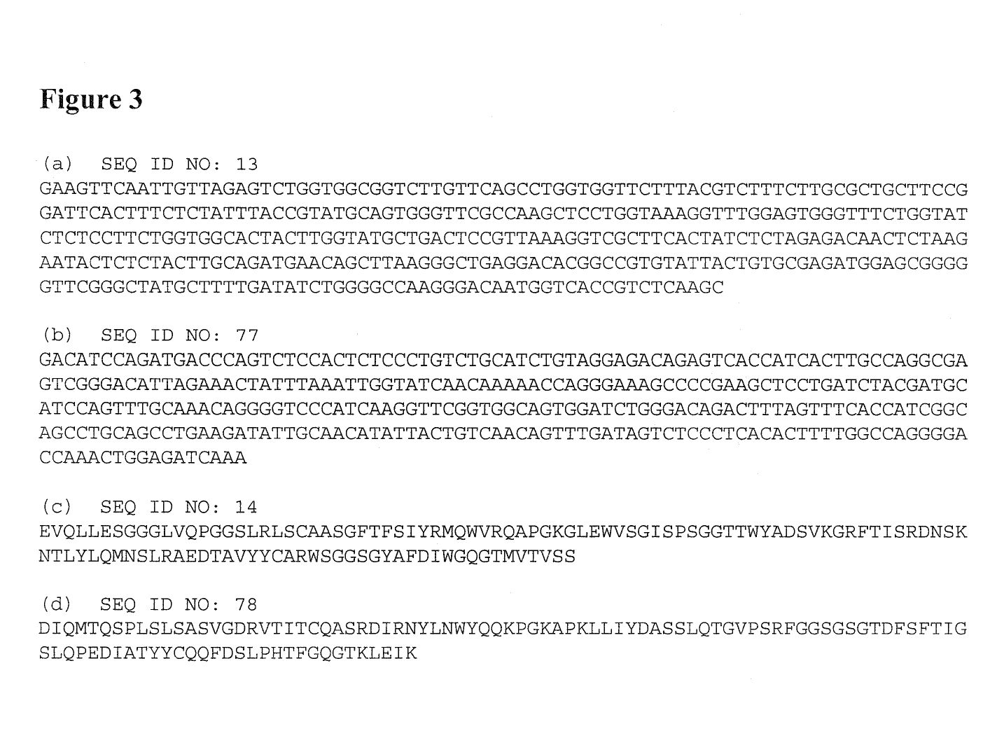

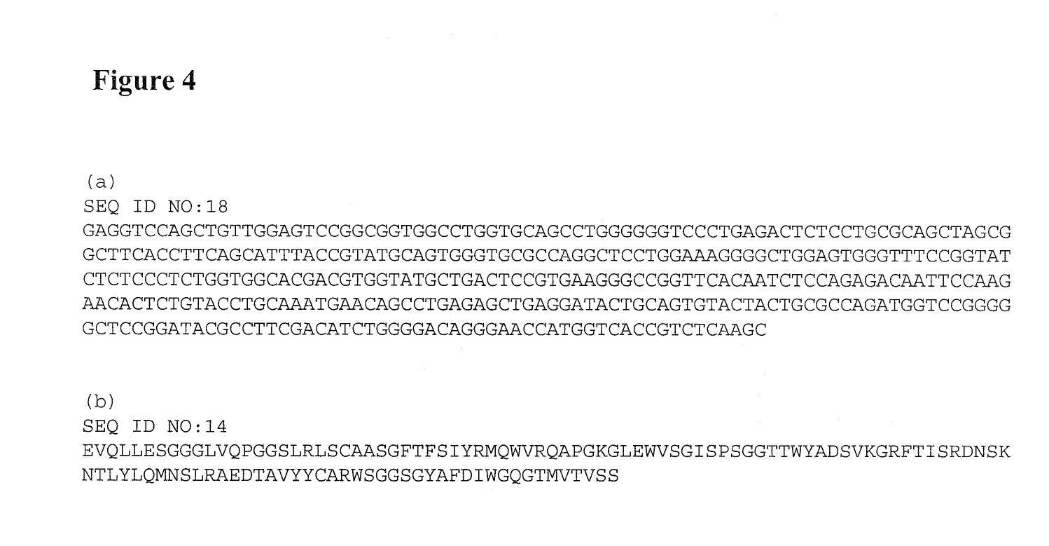

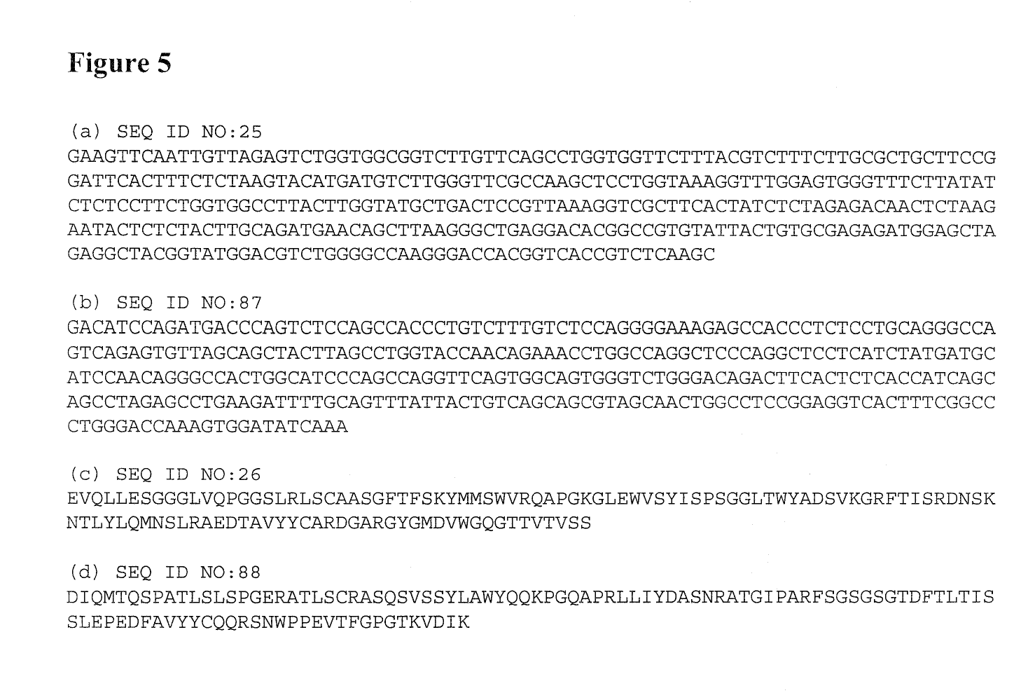

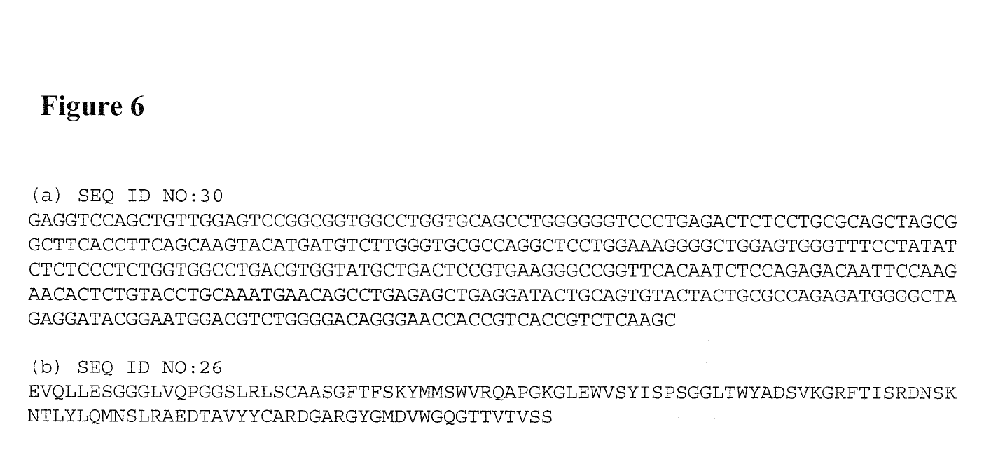

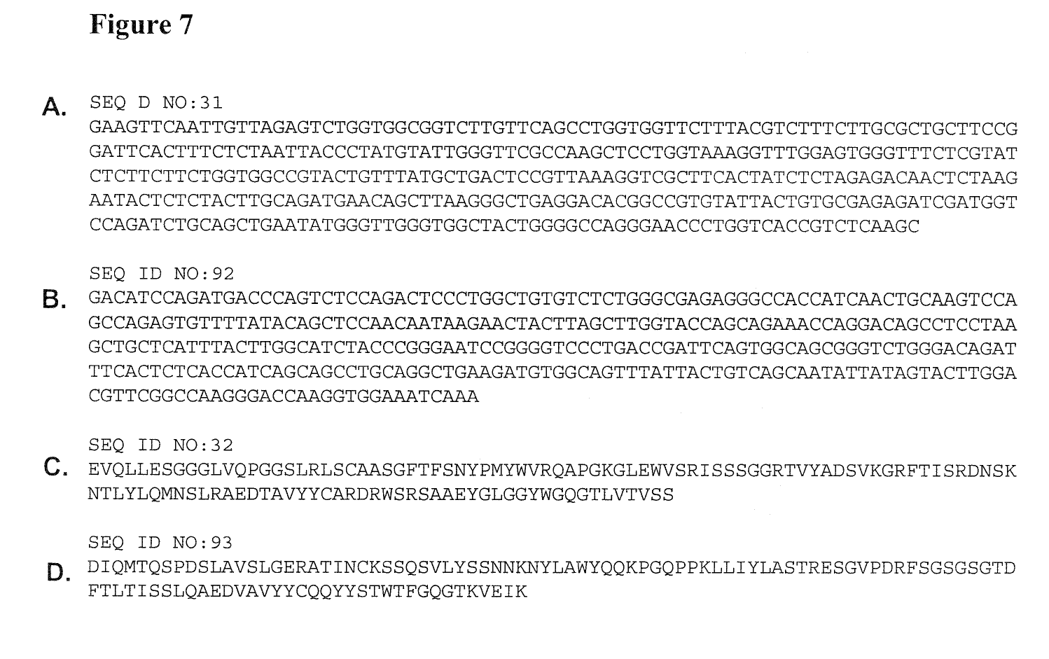

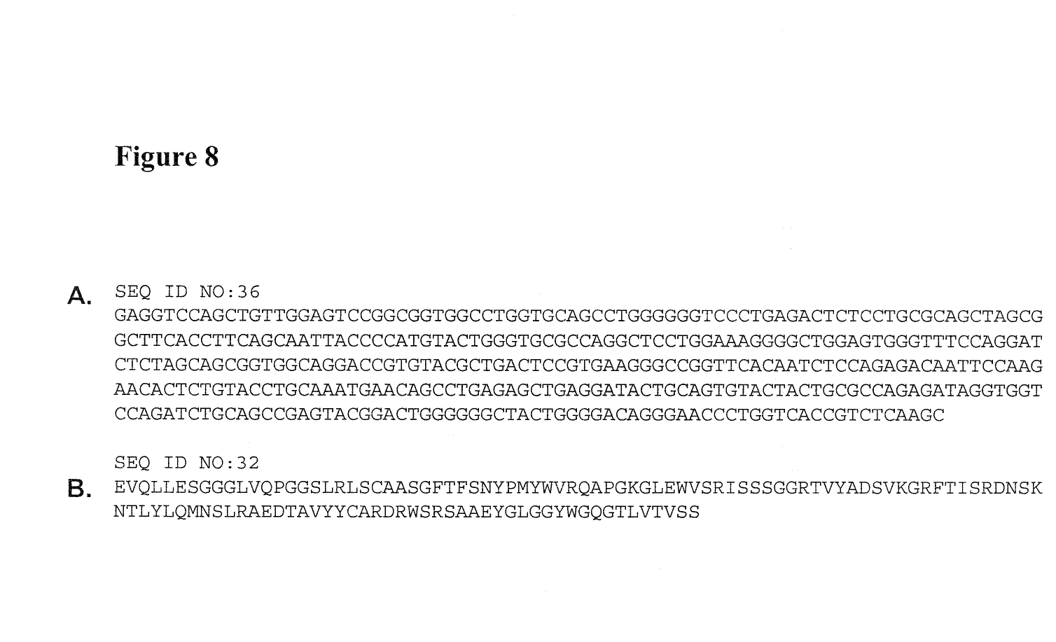

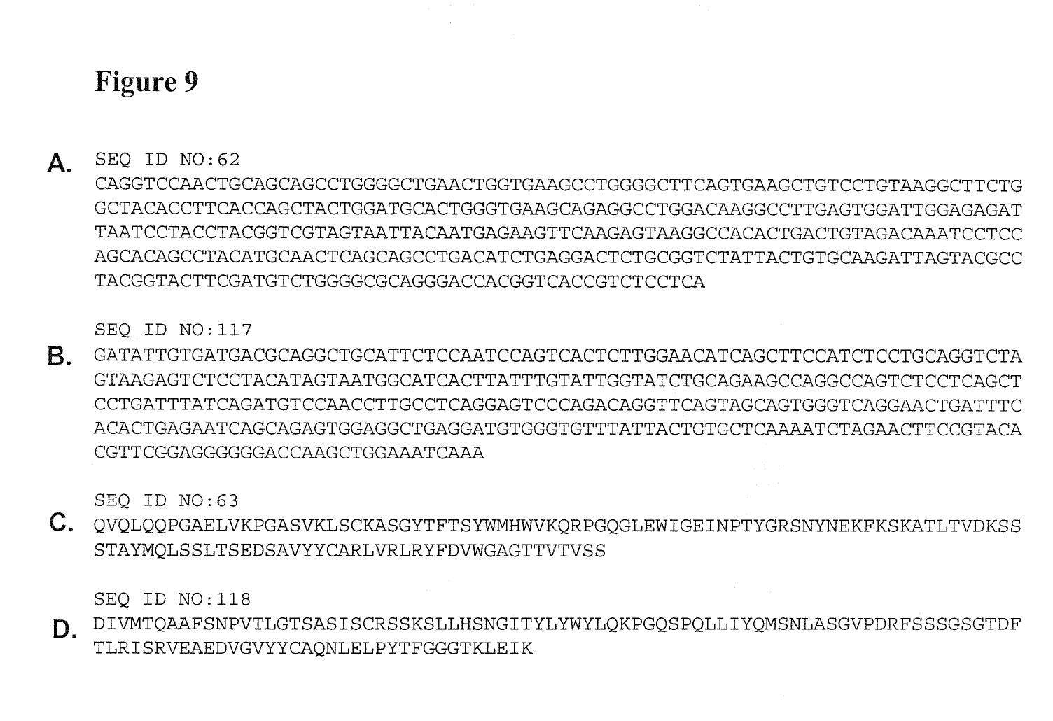

The nucleic acid sequence of the human IGF-1R mRNA is available under GenBank Accession Number NM_{—}000875 (gi|1119220593). The precursor polypeptide sequence is available under GenBank Accession Number NP_{—}000866 (gi 4557665). Amino acids 1 to 30 reported to encode the IGF-1R signal peptide, amino acids 31 to 740 are reported to encode the IGF-1R α-subunit, and amino acids 741 to 1367 are reported to encode the IGF-1R β-subunit. The mature IGF-1R polypeptide lacks the IGF1-R signal peptide. Therefore, numbering of IGF-1R amino acids in the instant application refers to the amino acid sequence of the mature form of human IGF-1R as shown in FIG. 2 (SEQ ID NO:2). Structural domains of this sequence are presented in Table 2.

TABLE 2

| SEQ ID | |
|---|---|
| NO: 2 | Feature |
| 1 to 796 | insulin-like growth factor 1 receptor alpha chain |
| 1 to 205 | Receptor L domain (L1) |
| 206 to 299 | Cysteine Repeat Region (CRR) |
| 300 to 457 | Receptor L domain (L2) |
| 458 to 575 | Fibronectin type 3 domain (FnIII-1) |
| 576 to 796 | Fibronectin type 3 domain (FnIII-2) |
| 797 to 1337 | insulin-like growth factor 1 receptor beta |
| 797 to 899 | Fibronectin type 3 domain (FnIII-3) |
| 900 to 925 | transmembrane region |
| 943 | Phosphorylation |
| 950 | Phosphorylation |
| 961 to 1238 | Tyrosine kinase, catalytic domain |
| 1131 | Phosphorylation |
| 1135 | Phosphorylation |
| 1136 | Phosphorylation |

### III. IGF-1R Binding Moieties

IGF-1R binding moieties of the binding molecules of the invention may comprise antigen recognition sites, entire variable regions, or one or more CDRs (e.g., six CDRs) derived from one or more starting or parental anti-IGF-1R antibodies. The parental antibodies can include naturally occurring antibodies or antibody fragments as well as antibodies or antibody fragments adapted from naturally occurring IGF-1R antibodies. Binding moieties may also be derived from anti-IGF-1R antibodies constructed de novo using sequences of IGF-1R antibodies or antibody fragments known to be specific for an IGF-1R target molecule. Sequences that may be derived from parental antibodies include heavy and/or light chain variable regions and/or CDRs, framework regions or other portions thereof.

In certain embodiments, an IGF-1R binding moiety specifically binds to at least one epitope of IGF-1R or fragment or variant, i.e., binds to such an epitope more readily than it would bind to an unrelated, or random epitope; binds preferentially to at least one epitope of IGF-1R or fragment or variant described above, i.e., binds to such an epitope more readily than it would bind to a related, similar, homologous, or analogous epitope; competitively inhibits binding of a reference antibody which itself binds specifically or preferentially to a certain epitope of IGF-1R or fragment or variant described above; or binds to at least one epitope of IGF-1R or fragment or variant described above with an affinity characterized by a dissociation constant K_{D} of less than about 5×10⁻² M, about 10⁻² M, about 5×10⁻³ M, about 10⁻³ M, about 5×10⁻⁴ M, about 10⁻⁴ M, about 5×10⁻⁵ M, about 10⁻⁵ M, about 5×10⁻⁶ M, about 10⁻⁶ M, about 5×10⁻⁷ M, about 10⁻⁷ M, about 5×10⁻⁸ M, about 10⁻⁸ M, about 5×10⁻⁹ M, about 10⁻⁹ M, about 5×10⁻¹⁰ M, about 10⁻¹⁰ M, about 5×10⁻¹¹ M, about 10⁻¹¹ M, about 5×10⁻¹² M, about 10⁻¹² M, about 5×10⁻¹³ M, about 10⁻¹³ M, about 5×10⁻¹⁴ M, about 10⁻¹⁴ M, about 5×10⁻¹⁵ M, or about 10⁻¹⁵ M.

In a particular aspect, the IGF-1R binding moiety preferentially binds to a human IGF-1R polypeptide or fragment thereof, relative to a murine IGF-1R polypeptide or fragment thereof. In another particular aspect, the IGF-1R binding moiety preferentially binds to one or more IGF-1R polypeptides or fragments thereof, e.g., one or more mammalian IGF-1R polypeptides, but does not bind to insulin receptor (InsR) polypeptides. In one embodiment, a binding moiety of a binding molecule of the invention does not cross react with InsR.

In specific embodiments, a binding moiety binds IGF-1R polypeptides or fragments or variants thereof with an off rate (k(off)) of less than or equal to 5×10⁻² sec⁻¹, 10⁻² sec⁻¹, 5×10⁻³ sec⁻¹ or 10⁻³ sec⁻¹. Alternatively, an IGF-1R binding moiety binds IGF-1R polypeptides or fragments or variants thereof with an off rate (k(off)) of less than or equal to 5×10⁻⁴ sec¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹. In other embodiments, an IGF-1R binding s moiety binds IGF-1R polypeptides or fragments or variants thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. Alternatively, an IGF-1R binding moiety binds IGF-1R polypeptides or fragments or variants thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×106 M⁻¹ sec⁻¹ or 10⁷ M⁻¹ sec⁻¹.

In various embodiments, an IGF-1R binding moiety acts to antagonize IGF-1R activity. In certain embodiments, for example, binding of an IGF-1R binding moiety to IGF-1R as expressed on a tumor cell has at least one of the following activities: inhibits binding of insulin growth factor, e.g., IGF-1, IGF-2, or both IGF-1 and IGF-2 to IGF-1R; promotes internalization of IGF-1R thereby inhibiting its signal transduction capability; inhibits phosphorylation of IGF-1R; inhibits phosphorylation of molecules downstream in the IGF-1R signal transduction pathway; e.g., Akt or p42/44 MAPK; inhibits tumor cell proliferation; inhibits tumor cell motility; and/or inhibits tumor cell metastasis.

In one embodiment, a binding moiety comprises at least one heavy or light chain CDR of an IGF-1R antibody molecule. In another embodiment, a binding moiety comprises at least two CDRs from one or more antibody molecules. In another embodiment, a binding moiety comprises at least three CDRs from one or more antibody molecules. In another embodiment, a binding moiety comprises at least four CDRs from one or more antibody molecules. In another embodiment, a binding moiety comprises at least five CDRs from one or more antibody molecules. In another embodiment, a binding moiety comprises at least six CDRs from one or more antibody molecules. Exemplary CDRs that can be included in the subject IGF-1R binding moieties (or binding molecules) of the invention are described herein (see, e.g., Tables 3 and 4).

Exemplary antibody molecules comprising at least one CDR that can be included in the subject IGF-1R binding molecules (or binding moieties) are also described herein. In certain embodiments, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to form a humanized binding specificity. The framework regions may be naturally occurring or consensus framework regions, and preferably human framework regions (see, e.g., Chothia et al., J. Mol. Biol. 278:457-479 (1998) for a listing of human framework regions). Preferably, the polynucleotide generated by the combination of the framework regions and CDRs encodes an antibody that specifically binds to at least one epitope of a desired polypeptide, e.g., IGF-1R. Preferably, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In one embodiment, the present invention provides an isolated polynucleotide encoding a binding molecule or binding moiety where the polynucleotide comprises, consists essentially of, or consists of a nucleic acid encoding an immunoglobulin heavy chain variable region (VH), where at least one of the CDRs of the heavy chain variable region or at least two of the VH-CDRs of the heavy chain variable region are at least 80%, 85%, 90%, 95%, or 100% identical to reference heavy chain VH-CDR1, VH-CDR2, or VH-CDR3 amino acid sequences from monoclonal IGF-1R antibodies disclosed herein. Thus, binding moieties (or binding molecules) of the invention may comprise a VH encoded by said polynucleotide. Alternatively, the VH-CDR1, VH-CDR2, and VH-CDR3 regions of the VH are at least 80%, 85%, 90%, 95%, or 100% identical to reference heavy chain VH-CDR1, VH-CDR2, and VH-CDR3 amino acid sequences from monoclonal IGF-1R antibodies disclosed herein. Thus, according to this embodiment a heavy chain variable region (e.g., of a binding molecule or binding moiety of the invention) has VH-CDR1, VH-CDR2, or VH-CDR3 polypeptide sequences related to the polypeptide sequences shown in Table 3:

TABLE 3
Reference VH-CDR1, VH-CDR2, and VH-CDR3 amino acid sequences*

| | VH SEQUENCE PN/PP | | | |
|---|---|---|---|---|
| | (VH-CDR1, VH-CDR2 and | | | |
| Antibody | VH-CDR3 underlined) | VH CDR1 | VH CDR2 | VH CDR3 |
| M12-E01 | GAAGTTCAATTGTTAGAGTCTGG | PYSML | SIGSSGG | VRGILH |
| | TGGCGGTCTTGTTCAGCCTGGTG | (SEQ ID NO:5) | STRYAD | YDILIGR |
| | GTTCTTTACGTCTTTCTTGCGCT | | SVKG | NLYYYY |
| | GCTTCCGGATTCACTTTCTCTCC | | (SEQ ID NO:6) | MDV |
| | TTACTCTATGCTTTGGGTTCGCC | | | (SEQ ID NO:7) |
| | AAGCTCCTGGTAAAGGTTTGGAG | | | |
| | TGGGTTTCTTCTATCGGTTCTTC | | | |
| | TGGTGGCTCTACTCGTTATGCTG | | | |
| | ACTCCGTTAAAGGTCGCTTCACT | | | |
| | ATCTCTAGAGACAACTCTAAGAA | | | |
| | TACTCTCTACTTGCAGATGAACA | | | |
| | GCTTAAGGGCTGAGGACACCGCC | | | |
| | ATGTATTACTGTGCACGGGTACG | | | |
| | GGGGATCCTTCATTACGATATTT | | | |
| | TGATTGGTAGAAATCTCTACTAC | | | |
| | TACTACATGGACGTCTGGGGCAA | | | |
| | AGGGACCACGGTCACCGTCTCAA | | | |
| | GC | | | |
| | (SEQ ID NO:3) | | | |
| | EVQLLESGGGLVQPGGSLRLSCA | | | |
| | ASGFTFSPYSMLWVRQAPGKGLE | | | |
| | WVSSIGSSGGSTRYADSVKGRFT | | | |
| | ISRDNSKNTLYLQMNSLRAEDTA | | | |
| | MYYCARVRGILHYDILIGRNLYY | | | |
| | YYMDVWGKGTTVTVSS | | | |
| | (SEQ ID NO:4) | | | |
| M12-G04 | GAAGTTCAATTGTTAGAGTCTGG | KYTMH | SIVSSGG | DRSIAAA |
| | TGGCGGTCTTGTTCAGCCTGGTG | (SEQ ID NO:10) | WTDYAD | GTGWSV |
| | GTTCTTTACGTCTTTCTTGCGCT | | SVKG | SFVDWF |
| | GCTTCCGGATTCACTTTCTCTAA | | (SEQ ID NO:11) | DP |
| | GTACACTATGCATTGGGTTCGCC | | | (SEQ ID NO:12) |
| | AAGCTCCTGGTAAAGGTTTGGAG | | | |
| | TGGGTTTCTTCTATCGTTTCTTC | | | |
| | TGGTGGCTGGACTGATTATGCTG | | | |
| | ACTCCGTTAAAGGTCGCTTCACT | | | |
| | ATCTCTAGAGACAACTCTAAGAA | | | |
| | TACTCTCTACTTGCAGATGAACA | | | |
| | GCTTAAGGGCTGAGGACACGGCC | | | |
| | GTGTATTACTGTGCGAGAGATCG | | | |
| | GAGTATAGCAGCAGCTGGTACCG | | | |
| | GTTGGTCTGTGAGTTTTGTGGAC | | | |
| | TGGTTGACCCCTGGGGCCAGGGA | | | |
| | ACCCGGTCACCGTCTCAAGC | | | |
| | (SEQ ID NO:8) | | | |
| | EVQLLESGGGLVQPGGSLRLSCA | | | |
| | ASGFTFSKYTMHWVRQAPGKGLE | | | |
| | WVSSIVSSGGWTDYADSVKGRFT | | | |
| | ISRDNSKNTLYLQMNSLRAEDTA | | | |
| | VYYCARDRSIAAAGTGWSVSFVD | | | |
| | WFDPWGQGTLVTVSS | | | |
| | (SEQ ID NO:9) | | | |
| M13-C06 | GAAGTTCAATTGTTAGAGTCTGG | IYRMQ | GISPSGG | WSGGSG |
| | TGGCGGTCTTGTTCAGCCTGGTG | (SEQ ID NO:15) | TTWYAD | YAFDI |
| | GTTCTTTACGTCTTTCTTGCGCT | | SVKG | (SEQ ID NO:17) |
| | GCTTCCGGATTCACTTTCTCTAT | | (SEQ ID NO:16) | |
| | TTACCGTATGCAGTGGGTTCGCC | | | |
| | AAGCTCCTGGTAAAGGTTTGGAG | | | |
| | TGGGTTTCTGGTATCTCTCCTTC | | | |
| | TGGTGGCACTACTTGGTATGCTG | | | |
| | ACTCCGTTAAAGGTCGCTTCACT | | | |
| | ATCTCTAGAGACAACTCTAAGAA | | | |
| | TACTCTCTACTTGCAGATGAACA | | | |
| | GCTTAAGGGCTGAGGACACGGCC | | | |
| | GTGTATTACTGTGCGAGATGGAG | | | |
| | CGGGGGTTCGGGCTATGCTTTTG | | | |
| | ATATCTGGGGCCAAGGGACAATG | | | |
| | GTCACCGTCTCAAGC | | | |
| | (SEQ ID NO:13) | | | |
| | EVQLLESGGGLVQPGGSLRLSCA | | | |
| | ASGFTFSIYRMQWVRQAPGKGLE | | | |
| | WVSGISPSGGTTWYADSVKGRFT | | | |
| | ISRDNSKNTLYLQMNSLRAEDTA | | | |
| | VYYCARWSGGSGYAFDIWGQGTM | | | |
| | VTVSS | | | |
| | (SEQ ID NO:14) | | | |
| M13-C06 | GAGGTCCAGCTGTTGGAGTCCGG | IYRMQ | GISPSGG | WSGGSG |
| Optimized | CGGTGGCCTGGTGCAGCCTGGGG | (SEQ ID NO:15) | TTWYAD | YAFDI |
| | GGTCCCTGAGACTCTCCTGCGCA | | SVKG | (SEQ ID NO:17) |
| | GCTAGCGGCTTCACCTTCAGCAT | | (SEQ ID NO:16) | |
| | TTACCGTATGCAGTGGGTGCGCC | | | |
| | AGGCTCCTGGAAAGGGGCTGGAG | | | |
| | GTGGGTTTCCGGTATCTCTCCCT | | | |
| | CTGGTGGCACGACGTGGTATGCT | | | |
| | GACTCCGTGAAGGGCCGGTTCAC | | | |
| | AATCTCCAGAGACAATTCCAAGA | | | |
| | ACACTCTGTACCTGCAAATGAAC | | | |
| | AGCCTGAGAGCTGAGGATACTGC | | | |
| | AGTGTACTACTGCGCCAGATGGT | | | |
| | CCGGGGGCTCCGGATACGCCTTC | | | |
| | GACATCTGGGGACAGGGAACCAT | | | |
| | GGTCACCGTCTCAAGC | | | |
| | (SEQ ID NO:18) | | | |
| | EVQLLESGGGLVQPGGSLRLSCA | | | |
| | ASGFTFSIYRMQWVRQAPGKGLE | | | |
| | WVSGISPSGGTTWYADSVKGRFT | | | |
| | ISRDNSKNTLYLQMNSLRAEDTA | | | |
| | VYYCARWSGGSGYAFDIWGQGTM | | | |
| | VTVSS | | | |
| | (SEQ ID NO:14) | | | |
| M14-B01 | GAAGTTCAATTGTTAGAGTCTGG | NYHMA | VISPTGG | AGYSYG |
| | TGGCGGTCTTGTTCAGCCTGGTG | (SEQ ID NO:21) | RTTYAD | YGYFDY |
| | GTTCTTTACGTCTTTCTTGCGCT | | SVKG | (SEQ ID NO:23) |
| | CTTCCGGATTCACTTTCTCTAAT | | (SEQ ID NO:22) | |
| | TACCATATGGCTTGGGTTCGCCA | | | |
| | AGCTCCTGGTAAAGGTTTGGAGT | | | |
| | GGGTTTCTGTTATCTCTCCTACT | | | |
| | GGTGGCCGTACTACTTATGCTGA | | | |
| | CTCCGTTAAAGGTCGCTTCACTA | | | |
| | TCTCTAGAGACAACTCTAAGAAT | | | |
| | ACTCTCTACTTGCAGATGAACAG | | | |
| | CTTAAGGGCTGAGGACACAGCCA | | | |
| | CATATTACTGTGCGAGAGCGGGG | | | |
| | TACAGCTATGGTTATGGCTACTT | | | |
| | TGACTACTGGGGCCAGGGAACCC | | | |
| | TGGTCACCGTCTCAAGC | | | |
| | (SEQ ID NO:19) | | | |
| | EVQLLESGGGLVQPGGSLRLSCA | | | |
| | ASGFTFSNYHMAWVRQAPGKGLE | | | |
| | WVSVISPTGGRTTYADSVKGRFT | | | |
| | ISRDNSKNTLYLQMNSLRAEDTA | | | |
| | TYYCARAGYSYGYGYFDYWGQGT | | | |
| | LVTVSS | | | |
| | (SEQ ID NO:20) | | | |
| M14-B01 | GAGGTCCAGCTGTTGGAGTCCGG | NYHMA | VISPTGG | AGYSYG |
| Optimized | CGGTGGCCTGGTGCAGCCTGGGG | (SEQ ID NO:21) | RTTYAD | YGYFDY |
| | GGTCCCTGAGACTCTCCTGCGCA | | SVKG | (SEQ ID NO:23) |
| | GCTAGCGGCTTCACCTTCAGCAA | | (SEQ ID NO:22) | |
| | TTACCACATGGCCTGGGTGCGCC | | | |
| | AGGCTCCTGGAAAGGGGCTGGAG | | | |
| | TGGGTTTCCGTGATCTCTCCTAC | | | |
| | CGGTGGCAGGACCACTTACGCTG | | | |
| | ACTCCGTGAAGGGCCGGTTCACA | | | |
| | ATCTCCAGAGACAATTCCAAGAA | | | |
| | CACTCTGTACCTGCAAATGAACA | | | |
| | GCCTGAGAGCTGAGGATACTGCA | | | |
| | ACATACTACTGCGCCAGAGCCGG | | | |
| | GTACTCCTACGGCTACGGATACT | | | |
| | TCGACTACTGGGGACAGGGAACC | | | |
| | CTGGTCACCGTCTCAAGC | | | |
| | (SEQ ID NO:24) | | | |
| | EVQLLESGGGLVQPGGSLRLSCA | | | |
| | ASGFTFSNYHMAWVRQAPGKGLE | | | |
| | WVSVISPTGGRTTYADSVKGRFT | | | |
| | ISRDNSKNTLYLQMNSLRAEDTA | | | |
| | TYYCARAGYSYGYGYFDYWGQGT | | | |
| | LVTVSS | | | |
| | (SEQ ID NO:20) | | | |
| M14-C03 | GAAGTTCAATTGTTAGAGTCTGG | KYMMS | YISPSGG | DGARGY |
| | TGGCGGTCTTGTTCAGCCTGGTG | (SEQ ID NO:27) | LTWYAD | GMDV |
| | GTTCTTTACGTCTTTCTTGCGCT | | SVKG | (SEQ ID NO:29) |
| | GCTTCCGGATTCACTTTCTCTAA | | (SEQ ID NO:28) | |
| | GTACATGATGTCTTGGGTTCGCC | | | |
| | AAGCTCCTGGTAAAGGTTTGGAG | | | |
| | TGGGTTTCTTATATCTCTCCTTC | | | |
| | TGGTGGCCTTACTTGGTATGCTG | | | |
| | ACTCCGTTAAAGGTCGCTTCACT | | | |
| | ATCTCTAGAGACAACTCTAAGAA | | | |
| | TACTCTCTACTTGCAGATGAACA | | | |
| | GCTTAAGGGCTGAGGACACGGCC | | | |
| | GTGTATTACTGTGCGAGAGATGG | | | |
| | AGCTAGAGGCTACGGTATGGACG | | | |
| | TCTGGGGCCAAGGGACCACGGTC | | | |
| | ACCGTCTCAAGC | | | |
| | (SEQ ID NO:25) | | | |
| | EVQLLESGGGLVQPGGSLRLSCA | | | |
| | ASGFTFSKYMMSWVRQAPGKGLE | | | |
| | WVSYISPSGGLTWYADSVKGRFT | | | |
| | ISRDNSKNTLYLQMNSLRAEDTA | | | |
| | VYYCARDGARGYGMDVWGQGTTV | | | |
| | TVSS | | | |
| | (SEQ ID NO:26) | | | |
| M14-C03 | GAGGTCCAGCTGTTGGAGTCCGG | KYMMS | YISPSGG | DGARGY |
| Optimized | CGGTGGCCTGGTGCAGCCTGGGG | (SEQ ID NO:27) | LTWYADS | GMDV |
| | GGTCCCTGAGACTCTCCTGCGCA | | VKG | (SEQ ID NO:29) |
| | GCTAGCGGCTTCACCTTCAGCAA | | (SEQ ID NO:28) | |
| | GTACATGATGTCTTGGGTGCGCC | | | |
| | AGGCTCCTGGAAAGGGGCTGGAG | | | |
| | TGGGTTTCCTATATCTCTCCCTC | | | |
| | TGGTGGCCTGACGTGGTATGCTG | | | |
| | ACTCCGTGAAGGGCCGGTTCACA | | | |
| | ATCTCCAGAGACAATTCCAAGAA | | | |
| | CACTCTGTACCTGCAAATGAACA | | | |
| | GCCTGAGAGCTGAGGATACTGCA | | | |
| | GTGTACTACTGCGCCAGAGATGG | | | |
| | GGCTAGAGGATACGGAATGGACG | | | |
| | TCTGGGGACAGGGAACCACCGTC | | | |
| | ACCGTCTCAAGC | | | |
| | (SEQ ID NO:30) | | | |
| | EVQLLESGGGLVQPGGSLRLSCA | | | |
| | ASGFTFSKYMMSWVRQAPGKGLE | | | |
| | WVSYISPSGGLTWYADSVKGRFT | | | |
| | ISRDNSKNTLYLQMNSLRAEDTA | | | |
| | VYYCARDGARGYGMDVWGQGTTV | | | |
| | TVSS | | | |
| | (SEQ ID NO:26) | | | |
| M14-G11 | GAAGTTCAATTGTTAGAGTCTGG | NYPMY | RISSSGG | DRWSRS |
| | TGGCGGTCTTGTTCAGCCTGGTG | (SEQ ID NO:33) | RTVYADS | AAEYGL |
| | GTTCTTTACGTCTTTCTTGCGCT | | VKG | GGY |
| | GCTTCCGGATTCACTTTCTCTAA | | (SEQ ID NO:34) | (SEQ ID NO:35) |
| | TTACCCTATGTATTGGGTTCGCC | | | |
| | AAGCTCCTGGTAAAGGTTTGGAG | | | |
| | TGGGTTTCTCGTATCTCTTCTTC | | | |
| | TGGTGGCCGTACTGTTTATGCTG | | | |
| | ACTCCGTTAAAGGTCGCTTCACT | | | |
| | ATCTCTAGAGACAACTCTAAGAA | | | |
| | TACTCTCTACTTGCAGATGAACA | | | |
| | GCTTAAGGGCTGAGGACACGGCC | | | |
| | GTGTATTACTGTGCGAGAGATCG | | | |
| | ATGGTCCAGATCTGCAGCTGAAT | | | |
| | ATGGGTTGGGTGGCTACTGGGGC | | | |
| | CAGGGAACCCTGGTCACCGTCTC | | | |
| | AAGC | | | |
| | (SEQ ID NO:31) | | | |
| | EVQLLESGGGLVQPGGSLRLSCA | | | |
| | ASGFTFSNYPMYWVRQAPGKGLE | | | |
| | WVSRISSSGGRTVYADSVKGRFT | | | |
| | ISRDNSKNTLYLQMNSLRAEDTA | | | |
| | VYYCARDRWSRSAAEYGLGGYWG | | | |
| | QGTLVTVSS | | | |
| | (SEQ ID NO:32) | | | |
| M14-G11 | GAGGTCCAGCTGTTGGAGTCCGG | NYPMY | RISSSGG | DRWSRS |
| Optimized | CGGTGGCCTGGTGCAGCCTGGGG | (SEQ ID NO:33) | RTVYADS | AEYGL |
| | GGTCCCTGAGACTCTCCTGCGCA | | VKG | GGY |
| | GCTAGCGGCTTCACCTTCAGCAA | | (SEQ ID NO:34) | (SEQ ID NO:35) |
| | TTACCCCATGTACTGGGTGCGCC | | | |
| | AGGCTCCTGGAAAGGGGCTGGAG | | | |
| | TGGGTTTCCAGGATCTCTAGCAG | | | |
| | CGGTGGCAGGACCGTGTACGCTG | | | |
| | ACTCCGTGAAGGGCCGGTTCACA | | | |
| | ATCTCCAGAGACAATTCCAAGAA | | | |
| | CACTCTGTACCTGCAAATGAACA | | | |
| | GCCTGAGAGCTGAGGATACTGCA | | | |
| | GTGTACTACTGCGCCAGAGATAG | | | |
| | GTGGTCCAGATCTGCAGCCGAGT | | | |
| | ACGGACTGGGGGGCTACTGGGGA | | | |
| | CAGGGAACCCTGGTCACCGTCTC | | | |
| | AAGC | | | |
| | (SEQ ID NO:36) | | | |
| | EVQLLESGGGLVQPGGSLRLSCA | | | |
| | ASGFTFSNYPMYWVRQAPGKGLE | | | |
| | WVSRISSSGGRTVYADSVKGRFT | | | |
| | ISRDNSKNTLYLQMNSLRAEDTA | | | |
| | VYYCARDRWSRSAAEYGLGGYWG | | | |
| | QGTLVTVSS | | | |
| | (SEQ ID NO:32) | | | |
| P2A7.3E11 | CAGGTTCAGCTGCAGCAGTCTGG | DYVIN | IYPGNEN | GIYYYG |
| | ACCTGAGCTAGTGAAGCCTGGGG | (SEQ ID NO:39) | TYYNEKF | SRTRTM |
| | CTTCAGTGAAGATGTCCTGCAAG | | KG | DY |
| | GCTTCTGGAAACACATTCACTGA | | (SEQ ID NO:40) | (SEQ ID NO:41) |
| | CTATGTTATAAACTGGGTGAAGC | | | |
| | AGAGAACTGGACAGGGCCTTGAG | | | |
| | TGGATTGGAGAGATTTATCCTGG | | | |
| | AAATGAAAATACTTATTACAATG | | | |
| | AGAAGTTCAAGGGCAAGGCCACA | | | |
| | CTGACTGCAGACAAATCCTCCAA | | | |
| | CACAGCCTACATGCAGCTCAGTA | | | |
| | GCCTGACATCTGAGGACTCTGCG | | | |
| | GTCTATTTCTGTGCAAGAGGGAT | | | |
| | TTATTACTACGGTAGTAGGACGA | | | |
| | GGACTATGGACTACTGGGGTCAA | | | |
| | GGAACCTCAGTCACCGTCTCCTC | | | |
| | A | | | |
| | (SEQ ID NO:37) | | | |
| | QVQLQQSGPELVKPGASVKMSCK | | | |
| | ASGNTFTDYVINWVKQRTGQGLE | | | |
| | WIGEIYPGNENTYYNEKFKGKAT | | | |
| | LTADKSSNTAYMQLSSLTSEDSA | | | |
| | VYFCARGIYYYGSRTRTMDYWGQ | | | |
| | GTSVTVSS | | | |
| | (SEQ ID NO:38) | | | |
| 20C8.3B8 | GACGTCCAACTGCAGGAGTCTGG | SGYSWH | YIHYSG | SGYGYR |
| | ACCTGACCTGGTGAAACCTTCTC | (SEQ ID NO:44) | GTNYNP | SAYYFD |
| | AGTCACTTTCACTCACCTGCACT | | SLKS | Y |
| | GTCACTGGCTACTCCATCACCAG | | (SEQ ID NO:45) | (SEQ ID NO:46) |
| | TGGTTATAGCTGGCACTGGATCC | | | |
| | GGCAGTTTCCAGGAAACAAACTG | | | |
| | GAATGGATGGGCTACATACACTA | | | |
| | CAGTGGTGGCACTAACTACAACC | | | |
| | CATCTCTCAAAAGTCGAATCTCT | | | |
| | ATCACTCGAGACACATCCAAGAA | | | |
| | CCAGTTCTTCCTCCAGTTGAATT | | | |
| | CTGTGACTACTGAGGACACAGCC | | | |
| | ACATATTACTGTGCAAGATCGGG | | | |
| | GTCGGCTACAGGAGTGCGTACTA | | | |
| | TTTTGACTACTGGGGCCAAGGGA | | | |
| | CACGGTCACCGTCTCCTCA | | | |
| | (SEQ ID NO:42) | | | |
| | DVQLQESGPDLVKPSQSLSLTCT | | | |
| | VTGYSITSGYSWHWIRQFPGNKL | | | |
| | EWMGYIHYSGGTNYNPSLKSRIS | | | |
| | ITRDTSKNQFFLQLNSVTTEDTA | | | |
| | TYYCARSGYGYRSAYYFDYWGQG | | | |
| | TTVTVSS | | | |
| | (SEQ ID NO:43) | | | |
| P1A2.2B11 | CAAATACAGTTGGTTCAGAGCGG | NHGMN | NTSTGEP | PLYYMY |
| | ACCTGAGCTGAAGAAGCCTGGAG | (SEQ ID NO:49) | TYADDFK | GRYIDV |
| | AGACAGTCAAGATCTCCTGCAAG | | G | (SEQ ID NO:51) |
| | GCTTCTGGGTATACCTTCACAAA | | (SEQ ID NO:50) | |
| | CCATGGAATGAACTGGGTGAAGC | | | |
| | AGGCTCCAGGAAAGGGTTTAAAG | | | |
| | TGGATGGGCTGGATAAACACCTC | | | |
| | CACTGGAGAGCCAACATATGCTG | | | |
| | ATGACTTCAAGGGACGTTTTGCC | | | |
| | TTCTCTTTGGAAACCTCTGCCAG | | | |
| | CACTGCCTTTTTGCAGATCAACA | | | |
| | ACCTCAAAAATGAGGACACGGCT | | | |
| | TATATTTCTGTGCAAGTCCCCTC | | | |
| | TGACTATATGTACGGGCGGTATA | | | |
| | TCGATGTCTGGGGCGCAGGGACC | | | |
| | GCGGTCACCGTCTCCTCA | | | |
| | (SEQ ID NO:47) | | | |
| | QIQLVQSGPELKKPGETVKISCK | | | |
| | ASGYTFTNHGMNWVKQAPGKGLK | | | |
| | WMGWNTSTGEPTYADDFKGRFAF | | | |
| | SLETSASTAFLQINNLKNEDTAS | | | |
| | YFCASPLYYMYGRYIDVWGAGTA | | | |
| | VTVSS | | | |
| | (SEQ ID NO:48) | | | |
| 20D8.24B11 | ACGTCCAACTGCAGGAGTCTGGA | SGYSWH | YIHYSG | SGYGYR |
| | CCTGACCTGGTGAAACCTTCTCA | (SEQ ID NO:54) | GTNYNP | SAYYFD |
| | GTCACTTTCACTCACCTGCACTG | | SLKS | Y |
| | TCACTGGCTACTCCATCACCAGT | | (SEQ ID NO:55) | (SEQ ID NO:56) |
| | GGTTATAGCTGGCACTGGATCCG | | | |
| | GCAGTTTCCAGGAAACAAACTGG | | | |
| | AATGGATGGGCTACATACACTAC | | | |
| | AGTGGTGGCACTAACTACAACCC | | | |
| | ATCTCTCAAAAGTCGAATCTCTA | | | |
| | TCACTCGAGACACATCCAAGAAC | | | |
| | CAGTTCTTCCTCCAGTTGAATTC | | | |
| | TGTGACTACTGAGGACACAGCCA | | | |
| | CATATTACTGTGCAAGATCGGGG | | | |
| | TACGGCTACAGGAGTG | | | |
| | (SEQ ID NO:52) | | | |
| | DVQLQESGPDLVKPSQSLSLTCT | | | |
| | VTGYSITSGYSWHWIRQFPGNKL | | | |
| | EWMGYIHYSGGTNYNPSLKSRIS | | | |
| | ITRDTSKNQFFLQLNSVTTEDTA | | | |
| | TYYCARSGYGYRSAYYFDYWGQG | | | |
| | TTLTVSS | | | |
| | (SEQ ID NO:53) | | | |
| P1G10.2B8 | CAGATCCAGTTGGTGCAGTCTGG | NHGMN | WINTNT | PLYYRN |
| | ACCTGACCTGAAGAAGCCTGGAG | (SEQ ID NO:59) | GEPTYA | GRYFDV |
| | AGACAGTCAAGATCTCCTGCAAG | | DDFKG | (SEQ ID NO:61) |
| | GCTTCTGGGTATACCTTCACAAA | | (SEQ ID NO:60) | |
| | CCATGGAATGAACTGGGTGAAGC | | | |
| | AGGCTCCAGGAAAGGATTTAAAG | | | |
| | TGGATGGGCTGGATAAACACCAA | | | |
| | CACTGGAGAGCCAACATATGCTG | | | |
| | ATGACTTCAAGGGACGGTTTGCC | | | |
| | TTCTCTTTGGAAACCTCTGCCAG | | | |
| | CACTGCCTATTTGCAGATCAACA | | | |
| | ACCTCAAAAATGAGGACACGGCT | | | |
| | ACATATTTCTGTGCAAGTCCCCT | | | |
| | CTACTATAGGAACGGGCGATACT | | | |
| | TCGATGTCTGGGGCGCAGGGACC | | | |
| | ACGGTCACCGTCTCC | | | |
| | (SEQ ID NO:57) | | | |
| | QIQLVQSGPDLKKPGETVKISCK | | | |
| | ASGYTFTNHGMNWVKQAPGKDLK | | | |
| | WMGWINTNTGEPTYADDFKGRFA | | | |
| | FSLETSASTAYLQINNLKNEDTA | | | |
| | TYFCASPLYYRNGRYFDVWGAGT | | | |
| | TVTVSS | | | |
| | (SEQ ID NO:58) | | | |
| P1E2.3B12 | CAGGTCCAACTGCAGCAGCCTGG | SYWMH | EINPTYG | LVRLRY |
| | GGCTGAACTGGTGAAGCCTGGGG | (SEQ ID NO:64) | RSNYNEK | FDV |
| | CTTCAGTGAAGCTGTCCTGTAAG | | FKS | (SEQ ID NO:66) |
| | GCTTCTGGCTACACCTTCACCAG | | (SEQ ID NO:65) | |
| | CTACTGGATGCACTGGGTGAAGC | | | |
| | AGAGGCCTGGACAAGGCCTTGAG | | | |
| | TGGATTGGAGAGATTAATCCTAC | | | |
| | CTACGGTCGTAGTAATTACAATG | | | |
| | AGAAGTTCAAGAGTAAGGCCACA | | | |
| | CTGACTGTAGACAAATCCTCCAG | | | |
| | CACAGCCTACATGCAACTCAGCA | | | |
| | GCCTGACATCTGAGGACTCTGCG | | | |
| | GTCTATTACTGTGCAAGATTAGT | | | |
| | ACGCCTACGGTACTTCGATGTCT | | | |
| | GGGGCGCAGGGACCACGGTCACC | | | |
| | GTCTCCTCA | | | |
| | (SEQ ID NO:62) | | | |
| | QVQLQQPGAELVKPGASVKLSCK | | | |
| | ASGYTFTSYWMHWVKQRPGQGLE | | | |
| | WIGEINPTYGRSNYNEKFKSKAT | | | |
| | LTVDKSSSTAYMQLSSLTSEDSA | | | |
| | VYYCARLVRLRYFDVWGAGTTVT | | | |
| | VSS | | | |
| | (SEQ ID NO:63) | | | |

*Determined by the Kabat system (see supra).
N = nucleotide sequence, P = polypeptide sequence.

As known in the art, “sequence identity” between two polypeptides or two polynucleotides is determined by comparing the amino acid or nucleic acid sequence of one polypeptide or polynucleotide to the sequence of a second polypeptide or polynucleotide. When discussed herein, whether any particular polypeptide is at least about 40%, 45%, 50%, 55%, 60%, 65%, 70%, 75%, 80%, 85%, 90%, 95%, or 100% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

In certain embodiments, a binding molecule or binding moiety comprising the VH encoded by the polynucleotide specifically or preferentially binds to IGF-1R. In certain embodiments the nucleotide sequence encoding the VH polypeptide is altered without altering the amino acid sequence encoded thereby. For instance, the sequence may be altered for improved codon usage in a given species, to remove splice sites, or the remove restriction enzyme sites. Sequence optimizations such as these are described in the examples and are well known and routinely carried out by those of ordinary skill in the art.

In another embodiment, the present invention provides an isolated polynucleotide comprising, consisting essentially of, or consisting of a nucleic acid encoding an immunoglobulin heavy chain variable region (VH) in which the VH-CDR1, VH-CDR2, and VH-CDR3 regions have polypeptide sequences which are identical to the VH-CDR1, VH-CDR2, and VH-CDR3 groups shown in Table 3. Accordingly, the binding moiety (or binding molecule) of the invention may comprise a VH encoded by said polynucleotide. In certain embodiments, a binding molecule or binding moiety comprising the VH encoded by the polynucleotide specifically or preferentially binds to IGF-1R.

In certain embodiments, the invention pertains to a binding moiety or binding molecule comprising, consisting essentially of, or consisting of a VH encoded by one or more of the polynucleotides described above specifically or preferentially binds to the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8, or will competitively inhibit such a monoclonal antibody or fragment from binding to IGF-1R.

In certain embodiments, the invention pertains to a binding moiety or binding molecule comprising, consisting essentially of, or consisting of a VH encoded by one or more of the polynucleotides described above specifically or preferentially binds to an IGF-1R polypeptide or fragment thereof, or a IGF-1R variant polypeptide, with an affinity characterized by a dissociation constant (KD) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In another embodiment, the present invention provides an isolated polynucleotide comprising, consisting essentially of, or consisting of a nucleic acid encoding an immunoglobulin light chain variable region (VL), where at least one of the VL-CDRs of the light chain variable region or at least two of the VL-CDRs of the light chain variable region are at least 80%, 85%, 90%, 95%, or 100% identical to reference light chain VL-CDR1, VL-CDR2, or VL-CDR3 amino acid sequences from monoclonal IGF-1R antibodies disclosed herein. Alternatively, the VL-CDR1, VL-CDR2, and VL-CDR3 regions of the VL are at least 80%, 85%, 90%, 95%, or 100% identical to reference light chain VL-CDR1, VL-CDR2, and VL-CDR3 amino acid sequences from monoclonal IGF-1R antibodies disclosed herein. Thus, according to this embodiment a light chain variable region (e.g., of a binding moiety or binding molecule of the invention) has VL-CDR1, VL-CDR2, or VL-CDR3 polypeptide sequences related to the polypeptide sequences shown in Table 4:

TABLE 4
Reference VL-CDR1, VL-CDR2, and VL-CDR3 amino acid sequences*

| | VL SEQUENCE PN/PP | | | |
|---|---|---|---|---|
| | (VL-CDR1, VL-CDR2, and | | | |
| Antibody | sequences underlined) | VL DR1 | VL CDR2 | VL CDR3 |
| M12-E01 | CAGTACGAATTGACTCAGCCGC | SGSSSNIG | YDDLLP | AAWDD |
| | CCTCGGTGTCTGAGGCCCCCCG | NNAIN | S | NLNGVI |
| | GCAGAGGGTCACCATCTCCTGT | (SEQ ID NO:69) | (SEQ ID NO:70) | (SEQ ID NO:71) |
| | TCTGGAAGCAGCTCCAACATCG | | | |
| | GAAATAATGCTATAAACTGGTA | | | |
| | CCAGCAACTCCCAGGAAAGCCT | | | |
| | CCCAAACTCCTCATCTATTATG | | | |
| | ATGATCTGTTGCCCTCAGGGGT | | | |
| | CTCTGACCGATTCTCTGGCTCC | | | |
| | AAGTCTGGCACCTCAGGCTCCC | | | |
| | TGGCCATCAGTGGGCTGCAGTC | | | |
| | TGAGGATGAGGCTGATTATTAC | | | |
| | TGTGCAGCATGGGATGACAACC | | | |
| | TGAATGGTGTGATTTTCGGCGG | | | |
| | AGGGACCAAGCTGACCGTCCTA | | | |
| | (SEQ ID NO:67) | | | |
| | QYELTQPPSVSEAPRQRVTISC | | | |
| | SGSSSNIGNNAINWYQQLPGKP | | | |
| | PKLLIYYDDLLPSGVSDRFSGS | | | |
| | KSGTSGSLAISGLQSEDEADYY | | | |
| | CAAWDDNLNGVIFGGGTKLTVL | | | |
| | (SEQ ID NO:68) | | | |
| M12-G04 | GACATCCAGATGACCCAGTCTC | RASQSIN | ATSSLQS | QQSYSTP |
| | CACTCTCCCTGTCTGCATCTGT | GYLN | (SEQ ID NO:75) | PYT |
| | AGGAGACAGAGTCACCATCACT | (SEQ ID NO:74) | | (SEQ ID NO:76) |
| | TGCCGGGCAAGTCAGAGCATTA | | | |
| | ACGGCTACTTAAATTGGTATCA | | | |
| | GCAGAAACCAGGGAAAGCCCCT | | | |
| | AACCTCCTGATCTACGCTACAT | | | |
| | CCAGTTTGCAAAGTGGGGTCCC | | | |
| | ATCAAGGTTCAGTGGCAGTGGA | | | |
| | TCTGGGACAGATTTCACTCTCA | | | |
| | CCATCAGCAGTCTGCAACCTGA | | | |
| | AGATTTTGCAACTTACTACTGT | | | |
| | CCAACAGAGTTACAGTACCCCC | | | |
| | CCGTACACTTTTGGCCAGGGGA | | | |
| | CCAAGCTGGAGATCAAA | | | |
| | (SEQ ID NO:72) | | | |
| | DIQMTQSPLSLSASVGDRVTIT | | | |
| | CRASSINGYLNWYQQKPGKAPN | | | |
| | LLIYATSSLQSGVPSRFSGSGS | | | |
| | GTDFTLTISSLQPEDFATYYCQ | | | |
| | QSYSTPPYTFGQGTKLEIK | | | |
| | (SEQ ID NO:73) | | | |
| M13-C06 | GACATCCAGATGACCCAGTCTC | QASRDIR | DASSLQ | QQFDSLP |
| | CACTCTCCCTGTCTGCATCTGT | NYLN | T | HT |
| | AGGAGACAGAGTCACCATCACT | (SEQ ID NO:79) | (SEQ ID NO:80) | (SEQ ID NO:81) |
| | TGCCAGGCGAGTCGGGACATTA | | | |
| | GAAACTATTTAAATTGGTATCA | | | |
| | ACAAAAACCAGGGAAAGCCCCG | | | |
| | AAGCTCCTGATCTACGATGCAT | | | |
| | CCAGTTTGCAAACAGGGGTCCC | | | |
| | ATCAAGGTTCGGTGGCAGTGGA | | | |
| | TCTGGGACAGACTTTAGTTTCA | | | |
| | CCATCGGCAGCCTGCAGCCTGA | | | |
| | AGATATTGCAACATATTACTGT | | | |
| | CAACAGTTTGATAGTCTCCCTC | | | |
| | ACACTTTTGGCCAGGGGACCAA | | | |
| | ACTGGAGATCAAA | | | |
| | (SEQ ID NO:77) | | | |
| | DIQMTQSPLSLSASVGDRVTIT | | | |
| | CQASRDIRNYLNWYQQKPGKAP | | | |
| | KLLIYDASSLQTGVPSRFGGSG | | | |
| | SGTDFSFTIGSLQPEDIATYYC | | | |
| | QQFDSLPHTFGQGTKLEIK | | | |
| | (SEQ ID NO:78) | | | |
| M14-B01 | GACATCCAGATGACCCAGTTTC | RASQSVM | GASKRA | HQRSTW |
| | CAGCCACCCTGTCTGTGTCTCC | RNLA | T | PLGT |
| | AGGGGAAAGAGCCACCCTCTCC | (SEQ ID NO:84) | (SEQ ID NO:85) | (SEQ ID NO:86) |
| | TGCAGGGCCAGTCAGAGTGTTA | | | |
| | TGAGGAACTTAGCCTGGTACCA | | | |
| | GCAGAAACCTGGCCAGCCTCCC | | | |
| | AGGCTCCTCATCTATGGTGCAT | | | |
| | CCAAAAGGGCCACTGGCATCCC | | | |
| | AGCCAGGTTCAGTGGCAGTGGG | | | |
| | TCTGGGACAGCCTTCACTCTCA | | | |
| | CCATCAGCAACCTAGAGCCTGA | | | |
| | AGATTTTGCAGTTTATTACTGT | | | |
| | CACCAACGTAGCACCTGGCCTC | | | |
| | TGGGGACTTTCGGCCCTGGGAC | | | |
| | CAAACTGGAGGCCAAA | | | |
| | (SEQ ID NO:82) | | | |
| | DIQMTQFPATLSVSPGERATLS | | | |
| | CRASQSVMRNLAWYQQKPGQPP | | | |
| | RLLIYGASKRATGIPARFSGSG | | | |
| | SGTAFTLTISNLEPEDFAVYYC | | | |
| | HQRSTWPLGTFGPGTKLEAK | | | |
| | (SEQ ID NO:83) | | | |
| M14-C03 | GACATCCAGATGACCCAGTCTC | RASQSVS | DASNRA | QQRSNW |
| | CAGCCACCCTGTCTTTGTCTCC | SYLA | T | PPEVT |
| | AGGGGAAAGAGCCACCCTCTCC | (SEQ ID NO:89) | (SEQ ID NO:90) | (SEQ ID NO:91) |
| | TGCAGGGCCAGTCAGAGTGTTA | | | |
| | GCAGCTACTTAGCCTGGTACCA | | | |
| | ACAGAAACCTGGCCAGGCTCCC | | | |
| | AGGCTCCTCATCTATGATGCAT | | | |
| | CCAACAGGGCCACTGGCATCCC | | | |
| | AGCCAGGTTCAGTGGCAGTGGG | | | |
| | TCTGGGACAGACTTCACTCTCA | | | |
| | CCATCAGCAGCCTAGAGCCTGA | | | |
| | AGATTTTGCAGTTTATTACTGT | | | |
| | CAGCAGCGTAGCAACTGGCCTC | | | |
| | CGGAGGTCACTTTCGGCCCTGG | | | |
| | GACCAAAGTGGATATCAAA | | | |
| | (SEQ ID NO:87) | | | |
| | DIQMTQSPATLSLSPGERATLS | | | |
| | CRASSVSSYLAWYQQKPGQAPR | | | |
| | LLIYDASNRATGIPARFSGSGS | | | |
| | GTDFTLTISSLEPEDFAVYYCQ | | | |
| | QRSNWPPEVTFGPGTKVDIK | | | |
| | (SEQ ID NO:88) | | | |
| M14-G11 | GACATCCAGATGACCCAGTCTC | KSSQSVL | LASTRES | QQYYST |
| | CAGACTCCCTGGCTGTGTCTCT | YSSNNKN | (SEQ ID NO:95) | WT |
| | GGGCGAGAGGGCCACCATCAAC | YLA | | (SEQ ID NO:96) |
| | TGCAAGTCCAGCCAGAGTGTTT | (SEQ ID NO:94) | | |
| | TATACAGCTCCAACAATAAGAA | | | |
| | CTACTTAGCTTGGTACCAGCAG | | | |
| | AAACCAGGACAGCCTCCTAAGC | | | |
| | TGCTCATTTACTTGGCATCTAC | | | |
| | CCGGGAATCCGGGGTCCCTGAC | | | |
| | CGATTCAGTGGCAGCGGGTCTG | | | |
| | GGACAGATTTCACTCTCACCAT | | | |
| | CAGCAGCCTGCAGGCTGAAGAT | | | |
| | GTGGCAGTTTATTACTGTCAGC | | | |
| | AATATTATAGTACTTGGACGTT | | | |
| | CGGCCAAGGGACCAAGGTGGAA | | | |
| | ATCAAA | | | |
| | (SEQ ID NO:92) | | | |
| | DIQMTQSPDSLAVSLGERATIN | | | |
| | CKSSQSVLYSSNNKNYLAWYQQ | | | |
| | KPGQPPKLLIYLASTRESGVPD | | | |
| | RFSGSGSGTDFTLTISSLQAED | | | |
| | VAVYYCQQYYSTWTFGQGTKVE | | | |
| | IK | | | |
| | (SEQ ID NO:93) | | | |
| P2A7.3E11 | GAAGTTGTGCTCACCCAGTCTC | SASSTLSS | RTSNLAS | QQGSSIP |
| | CAACCGCCATGGCTGCATCTCC | NYLH | (SEQ ID NO:100) | LT |
| | CGGGGAGAAGATCACTATCACC | (SEQ ID NO:99) | | (SE ID NO:101) |
| | TGCAGTGCCAGCTCAACTTTAA | | | |
| | GTTCCAATTACTTGCATTGGTA | | | |
| | TCAGCAGAAGCCAGGATTCTCC | | | |
| | CCTAAACTCTTGATTTATAGGA | | | |
| | CATCCAATCTGGCCTCTGGAGT | | | |
| | CCCAGGTCGCTTCAGTGGCAGT | | | |
| | GGGTCTGGGACCTCTTACTCTC | | | |
| | TCACAATTGGCACCATGGAGGC | | | |
| | TGAAGATGTTGCCACTTACTAC | | | |
| | TGCCAGCAGGGTAGTAGTATAC | | | |
| | CGCTCACGTTCGGTGCTGGGAC | | | |
| | CAAGCTGGAGCTGAAG | | | |
| | (SEQ ID NO:97) | | | |
| | EVVLTQSPTAMAASPGEKITIT | | | |
| | CSASSTLSSNYLHWYQQKPGFS | | | |
| | PKLLIYRTSNLASGVPGRFSGS | | | |
| | GSGTSYSLTIGTMEAEDVATYY | | | |
| | CQQGSSIPLTFGAGTKLELK | | | |
| | (SEQ ID NO:98) | | | |
| 20C8.3B8 | GACATTGTGCTGACACAGTCTC | RASKSVS | LASNLES | QHSRELP |
| | CTGCTTCCTTAGCTGTATCTCT | TSAYSYM | (SEQ ID NO:105) | YT |
| | GGGGCAGAGGGCCACCATCTCA | H | | (SEQ ID NO:106) |
| | TGCAGGGCCAGCAAAAGTGTCA | (SEQ ID NO:104) | | |
| | GTACATCTGCCTATAGTTATAT | | | |
| | GCACTGGTACCAACAGAAACCA | | | |
| | GGACAGCCACCCAAACTCCTCA | | | |
| | TCTATCTTGCATCCAACCTAGA | | | |
| | ATCTGGGGTCCCTGCCAGGTTC | | | |
| | AGTGGCAGTGGGTCTGGGACAG | | | |
| | ACTTCACCCTCAACATCCATCC | | | |
| | TGTGGAGGAGGAGGATGCTGCA | | | |
| | ACCTATTACTGTCAGCACAGTA | | | |
| | GGGAGCTTCCGTATACGTTCGG | | | |
| | AGGGGGGACCAAGCTGGAAATC | | | |
| | (SEQ ID NO:102) | | | |
| | DIVLTQSPASLAVSLGQRATIS | | | |
| | CRASKSVSTSAYSYMHWYQQKP | | | |
| | GQPPKLLIYLASNLESGVPARF | | | |
| | SGSGSGTDFTLNIHPVEEEDAA | | | |
| | TYYCQHSRELPYTFGGGTKLEI | | | |
| | K | | | |
| | (SEQ ID NO:103) | | | |
| P1A2.2B11 | GATATCCAGATGACACAGACTA | RASQDIS | TSRLHS | QQGKTL |
| | CATCCTCCCTATCTGCCTCTCT | NYLN | (SEQ ID NO:110) | PWT |
| | GGGAGACAGAGTCACCATCAGT | (SEQ ID NO:109) | | (SEQ ID NO:111) |
| | TGCAGGGCAAGTCAGGACATTA | | | |
| | GCAATTATTTAAACTGGTATCA | | | |
| | GCAGAAACCAGATGGAACTATT | | | |
| | AAACTCCTGATCTACTACACAT | | | |
| | CAAGATTACACTCAGGAGTCCC | | | |
| | ATCAAGGTTCAGTGGCAGTGGG | | | |
| | TCTGGAACAGATTATTCTCTCA | | | |
| | CCATTAGCAACCTGGAACAAGA | | | |
| | AGATTTTGCCACTTACTTTTGC | | | |
| | CAACAGGGTAAAACGCTTCCGT | | | |
| | GGACGTTCGGTGGAGGCACCAA | | | |
| | GCTGGAAATCAAA | | | |
| | (SEQ ID NO:107) | | | |
| | DIQMTQTTSSLSASLGDRVTIS | | | |
| | CRASQDISNYLNWYQQKPDGTI | | | |
| | KLLIYYTSRLHSGVPSRFSGSG | | | |
| | SGTDYSLTISNLEQEDFATYFC | | | |
| | QQGKTLPWTFGGGTKLEIK | | | |
| | (SEQ ID NO:108) | | | |
| 20D8.24B11 | SAME AS 20C8 | | | |
| P1G10.2B8 | GATATCCAGATGACACAGACTA | RASQDIS | TSRLH | QQGKTL |
| | CATCCTCCCTGTCTGCCTCTCT | NYLN | (SEQ ID NO:115) | PWT |
| | GGGAGACAGAGTCACCATCAGT | (SEQ ID NO:114) | | (SEQ ID NO:116) |
| | TGCAGGGCAAGTCAGGACATTA | | | |
| | GTAATTATTTAAATTGGTATCA | | | |
| | GCAGAAACCAGATGGATCTGTT | | | |
| | AAACTCCTGATCTACTACACAT | | | |
| | CAAGATTACACTCAGGAGTCCC | | | |
| | ATCAAGGTTCAGTGGCAGTGGG | | | |
| | TCTGGAACAGATTATTCTCTCA | | | |
| | CCATTAGCAACCTGGAACAAGA | | | |
| | AGATATTGCCACTTACTTTTGC | | | |
| | CAACAGGGAAAGACGCTTCCGT | | | |
| | GGACGTTCGGTGGAGGCACCAA | | | |
| | GCTGGAAATCAAA | | | |
| | (SEQ ID NO:112) | | | |
| | DIQMTQTTSSLSASLGDRVTIS | | | |
| | CRASQDISNYLNWYQQKPDGSV | | | |
| | KLLIYYTSRLHSGVPSRFSGSG | | | |
| | SGTDYSLTISNLEQEDIATYFC | | | |
| | QQGKTLPTFGGGTKLEIK | | | |
| | (SEQ ID NO:113) | | | |
| P1E2.3B12 | GATATTGTGATGACGCAGGCTG | RSSKSLL | QMSNLA | AQNLEL |
| | CATTCTCCAATCCAGTCACTCT | HSNGITY | S | PYT |
| | TGGAACATCAGCTTCCATCTCC | LY | (SEQ ID NO:120) | (SEQ ID NO:121) |
| | TGCAGGTCTAGTAAGAGTCTCC | (SEQ ID NO:119) | | |
| | TACATAGTAATGGCATCACTTA | | | |
| | TTTGTATTGGTATCTGCAGAAG | | | |
| | CCAGGCCAGTCTCCTCAGCTCC | | | |
| | TGATTTATCAGATGTCCAACCT | | | |
| | TGCCTCAGGAGTCCCAGACAGG | | | |
| | TTCAGTAGCAGTGGGTCAGGAA | | | |
| | CTGATTTCACACTGAGAATCAG | | | |
| | CAGAGTGGAGGCTGAGGATGTG | | | |
| | GGTGTTTATTACTGTGCTCAAA | | | |
| | ATCTAGAACTTCCGTACACGTT | | | |
| | CGGAGGGGGGACCAAGCTGGAA | | | |
| | ATCAAA | | | |
| | (SEQ ID NO:117) | | | |
| | DIVMTQAAFSNPVTLGTSASIS | | | |
| | CRSSKSLLHSNGITYLYWYLQK | | | |
| | PGQSPQLLIYQMSNLASGVPDR | | | |
| | FSSSGSGTDFTLRISRVEAEDV | | | |
| | GVYYCAQNLELPYTFGGGTKLE | | | |
| | IK | | | |
| | (SEQ ID NO:118) | | | |

*Determined by the Kabat system (see supra).
PN = nucleotide sequence, PP = polypeptide sequence.

In another embodiment, the present invention provides an isolated polynucleotide comprising, consisting essentially of, or consisting of a nucleic acid encoding an immunoglobulin light chain variable region (VL) in which the VL-CDR1, VL-CDR2, and VL-CDR3 regions have polypeptide sequences which are identical to the VL-CDR1, VL-CDR2, and VL-CDR3 groups shown in Table 4. Thus, a binding moiety (or binding molecule) of the invention may comprise the VL encoded by said polynucleotide. In certain embodiments, a binding moiety (or binding molecule) comprising the VL encoded by the polynucleotide specifically or preferentially binds to IGF-1R.

In a further aspect, the present invention provides an isolated polynucleotide comprising, consisting essentially of, or consisting of a nucleic acid encoding an immunoglobulin light chain variable region (VL) in which the VL-CDR1, VL-CDR2, and VL-CDR3 regions are encoded by nucleotide sequences which are identical to the nucleotide sequences which encode the VL-CDR1, VL-CDR2, and VL-CDR3 groups shown in Table 4. According, a binding moiety (or binding molecule) of the invention may comprise the VL encoded by said polynucleotide. In certain embodiments, the binding moiety or binding molecule comprising the VL encoded by the polynucleotide specifically or preferentially binds to IGF-1R.

In certain embodiments, the invention pertains to a binding moiety or binding molecule comprising, consisting essentially of, or consisting of a VL encoded by one or more of the polynucleotides described above specifically or preferentially binds to the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B 11,20D8.24B11, P1E2.3B12, and P1G10.2B8, or will competitively inhibit such a monoclonal antibody or fragment from binding to IGF-1R.

In certain embodiments, the invention pertains to a binding moiety (or binding molecule) comprising, consisting essentially of, or consisting of a VL encoded by one or more of the polynucleotides described above specifically or preferentially binds to an IGF-1R polypeptide or fragment thereof, or a IGF-1R variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In a further embodiment, the present invention includes an isolated polynucleotide comprising, consisting essentially of, or consisting of a nucleic acid encoding a VH at least 80%, 85%, 90% 95% or 100% identical to a reference VH polypeptide sequence selected from the group consisting of SEQ ID NOs: 4, 9, 14, 20, 26, 32, 38, 43, 48, 53, 58, and 63. Accordingly a binding moiety (or binding molecule) of the invention may comprise the VH encoded by said polynucleotide. In certain embodiments, the binding moiety or binding molecule comprising the VH encoded by the polynucleotide specifically or preferentially binds to IGF-1R.

In another aspect, the present invention includes an isolated polynucleotide comprising, consisting essentially of, or consisting of a nucleic acid sequence encoding a VH having a polypeptide sequence selected from the group consisting of SEQ ID NOs: 4, 9, 14, 20, 26, 32, 38, 43, 48, 53, 58, and 63. Accordingly a binding moiety (or binding molecule) of the invention may comprise the VH encoded by said polynucleotide. In certain embodiments, the binding moiety or binding molecule comprising the VH encoded by the polynucleotide specifically or preferentially binds to IGF-1R.

In a further embodiment, the present invention includes an isolated polynucleotide comprising, consisting essentially of, or consisting of a VH-encoding nucleic acid at least 80%, 85%, 90% 95% or 100% identical to a reference nucleic acid sequence selected from the group consisting of SEQ ID NOs: 3, 8, 13, 18, 19, 24, 25, 30, 31, 36, 37, 42, 47, 52, 57, and 62. Accordingly a binding moiety (or binding molecule) of the invention may comprise the VH encoded by said polynucleotide. In certain embodiments, the binding moiety or binding molecule comprising the VH encoded by such polynucleotides specifically or preferentially binds to IGF-1R.

In another aspect, the present invention includes an isolated polynucleotide comprising, consisting essentially of, or consisting of a nucleic acid sequence encoding a VH of the invention, where the amino acid sequence of the VH is selected from the group consisting of SEQ ID NOs: 4, 9, 14, 20, 26, 32, 38, 43, 48, 53, 58, and 63. The present invention further includes an isolated polynucleotide comprising, consisting essentially of, or consisting of a nucleic acid sequence encoding a VH of the invention, where the sequence of the nucleic acid is selected from the group consisting of SEQ ID NOs: 3, 8, 13, 18, 19, 24, 25, 30, 31, 36, 37, 42, 47, 52, 57, and 62. Accordingly a binding moiety (or binding molecule) of the invention may comprise the VH encoded by said polynucleotide. In certain embodiments, the binding moiety or binding molecule comprising the VH encoded by such polynucleotides specifically or preferentially binds to IGF-1R.

In certain embodiments, the invention pertains to a binding moiety or binding molecule comprising, consisting essentially of, or consisting of a VH encoded by one or more of the polynucleotides described above specifically or preferentially binds to the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B 11, 20D8.24B 11, P1E2.3B12, and P1G10.2B8, or will competitively inhibit such a monoclonal antibody or fragment from binding to IGF-1R, or will competitively inhibit such a monoclonal antibody from binding to IGF-1R.

In certain embodiments, the invention pertains to a binding moiety binding molecule comprising, consisting essentially of, or consisting of a VH encoded by one or more of the polynucleotides described above specifically or preferentially binds to an IGF-1R polypeptide or fragment thereof, or a IGF-1R variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In a further embodiment, the present invention includes an isolated polynucleotide comprising, consisting essentially of, or consisting of a nucleic acid encoding a VL at least 80%, 85%, 90% 95% or 100% identical to a reference VL polypeptide sequence having an amino acid sequence selected from the group consisting of SEQ ID NOs: 68, 73, 78, 83, 88, 93, 98, 103, 108, 113, and 118. In a further embodiment, the present invention includes an isolated polynucleotide comprising, consisting essentially of, or consisting of a VL-encoding nucleic acid at least 80%, 85%, 90% 95% or 100% identical to a reference nucleic acid sequence selected from the group consisting of SEQ ID NOs: 67, 72, 77, 82, 87, 92, 97, 102, 107, 112, and 117. Accordingly a binding moiety (or binding molecule) of the invention may comprise the VL encoded by said polynucleotide. In certain embodiments, the binding moiety or binding molecule comprising the VL encoded by such polynucleotides specifically or preferentially binds to IGF-1R.

In another aspect, the present invention includes an isolated polynucleotide comprising, consisting essentially of, or consisting of a nucleic acid sequence encoding a VL having a polypeptide sequence selected from the group consisting of SEQ ID NOs: 68, 73, 78, 83, 88, 93, 98, 103, 108, 113, and 118. The present invention further includes an isolated polynucleotide comprising, consisting essentially of, or consisting of a nucleic acid sequence encoding a VL of the invention, where the sequence of the nucleic acid is selected from the group consisting of SEQ ID NOs: 67, 72, 77, 82, 87, 92, 97, 102, 107, 112, and 117. Accordingly a binding moiety (or binding molecule) of the invention may comprise the VL encoded by said polynucleotide. In certain embodiments, the binding molecule or binding moiety comprising the VL encoded by such polynucleotides specifically or preferentially binds to IGF-1R.

In certain embodiments, the invention pertains to a binding moiety or binding molecule comprising, consisting essentially of, or consisting of a VL encoded by one or more of the polynucleotides described above specifically or preferentially binds to the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B 11, 20D8.24B 11, P1E2.3B12, and P1G10.2B8, or will competitively inhibit such a monoclonal antibody or fragment from binding to IGF-1R.

In certain embodiments, the invention pertains to a binding moiety or binding molecule comprising, consisting essentially of, or consisting of a VL encoded by one or more of the polynucleotides described above specifically or preferentially binds to an IGF-1R polypeptide or fragment thereof, or a IGF-1R variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Any of the polynucleotides described above may further include additional nucleic acids, encoding, e.g., a signal peptide to direct secretion of the encoded polypeptide, antibody constant regions as described herein, or other heterologous polypeptides as described herein.

Also, as described in more detail elsewhere herein, the present invention includes compositions comprising one or more of the polynucleotides described above. In one embodiment, the invention includes compositions comprising a first polynucleotide and second polynucleotide wherein said first polynucleotide encodes a VH polypeptide as described herein and wherein said second polynucleotide encodes a VL polypeptide as described herein. Specifically a composition which comprises, consists essentially of, or consists of a VH polynucleotide, and a VL polynucleotide, wherein the VH polynucleotide and the VL polynucleotide encode polypeptides, respectively at least 80%, 85%, 90% 95% or 100% identical to reference VL and VL polypeptide amino acid sequences selected from the group consisting of SEQ ID NOs: 4 and 68, 8 and 73, 14 and 78, 20 and 83, 26 and 88, 32 and 93, 38 and 98, 43 and 103, 48 and 108, 53 and 103, 58 and 113, and 63 and 118. Or alternatively, a composition which comprises, consists essentially of, or consists of a VH polynucleotide, and a VL polynucleotide at least 80%, 85%, 90% 95% or 100% identical, respectively, to reference VL and VL nucleic acid sequences selected from the group consisting of SEQ ID NOs: 3 and 67, 8 and 72, 13 and 77, 18 and 77, 19 and 82, 24 and 82, 25 and 87, 30 and 87, 31 and 92, 36 and 92, 37 and 97, 42 and 102, 47 and 107, 58 and 102, 57 and 112, and 62 and 117. In certain embodiments, an antibody or antigen-binding fragment comprising the VH and VL encoded by the polynucleotides in such compositions specifically or preferentially binds to IGF-1R.

The polynucleotides may be produced or manufactured by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an IGF-1R antibody, or antigen-binding fragment, variant, or derivative thereof may be generated from nucleic acid from a suitable source. If a clone containing a nucleic acid encoding a particular antibody is not available, but the sequence of the antibody molecule is known, a nucleic acid encoding the antibody may be chemically synthesized or obtained from a suitable source (e.g., an antibody cDNA library, or a cDNA library generated from, or nucleic acid, preferably poly A+RNA, isolated from, any tissue or cells expressing the antibody or other IGF-1R antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody or other IGF-1R antibody. Amplified nucleic acids generated by PCR may then be cloned into replicable cloning vectors using any method well known in the art.

Once the nucleotide sequence and corresponding amino acid sequence of the IGF-1R antibody, or antigen-binding fragment, variant, or derivative thereof is determined, its nucleotide sequence may be manipulated using methods well known in the art for the manipulation of nucleotide sequences, e.g., recombinant DNA techniques, site directed mutagenesis, PCR, etc. (see, for example, the techniques described in Sambrook et al., Molecular Cloning, A Laboratory Manual, 2d Ed., Cold Spring Harbor Laboratory, Cold Spring Harbor, N.Y. (1990) and Ausubel et al., eds., Current Protocols in Molecular Biology, John Wiley & Sons, NY (1998), which are both incorporated by reference herein in their entireties), to generate antibodies having a different amino acid sequence, for example to create amino acid substitutions, deletions, and/or insertions.

A polynucleotide encoding an IGF-binding molecule can be composed of a polyribonucleotide or polydeoxyribonucleotide, which may be unmodified RNA or DNA or modified RNA or DNA. For example, a polynucleotide encoding an IGF-1R binding molecule can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, a polynucleotide encoding an IGF-1R binding molecule can be composed of triple-stranded regions comprising RNA or DNA or both RNA and DNA. A polynucleotide encoding an IGF-1R binding molecule may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

An isolated polynucleotide encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis. Preferably, conservative amino acid substitutions are made at one or more non-essential amino acid residues.

The present invention is further directed to isolated polypeptides which make up IGF-1R antibodies, and polynucleotides encoding such polypeptides. IGF-1R binding molecules of the present invention comprise polypeptides, e.g., amino acid sequences encoding IGF-1R-specific antigen binding regions derived from immunoglobulin molecules. A polypeptide or amino acid sequence “derived from” a designated protein refers to the origin of the polypeptide having a certain amino acid sequence. In certain cases, the polypeptide or amino acid sequence which is derived from a particular starting polypeptide or amino acid sequence has an amino acid sequence that is essentially identical to that of the starting sequence, or a portion thereof, wherein the portion consists of at least 10-20 amino acids, at least 20-30 amino acids, at least 30-50 amino acids, or which is otherwise identifiable to one of ordinary skill in the art as having its origin in the starting sequence.

In one embodiment, the present invention provides an isolated polypeptide comprising, consisting essentially of, or consisting of an immunoglobulin heavy chain variable region (VH), where at least one of VH-CDRs of the heavy chain variable region or at least two of the VH-CDRs of the heavy chain variable region are at least 80%, 85%, 90%, 95%, or 100% identical to reference heavy chain VH-CDR1, VH-CDR2 or VH-CDR3 amino acid sequences from monoclonal IGF-1R antibodies disclosed herein. Alternatively, the VH-CDR1, VH-CDR2 and VH-CDR3 regions of the VH are at least 80%, 85%, 90%, 95%, or 100% identical to reference heavy chain VH-CDR1, VH-CDR2 and VH-CDR3 amino acid sequences from monoclonal IGF-1R antibodies disclosed herein. Thus, according to this embodiment a heavy chain variable region of the invention has VH-CDR1, VH-CDR2 and VH-CDR3 polypeptide sequences related to the groups shown in Table 3, supra. While Table 3 shows VH-CDRs defined by the Kabat system, other CDR definitions, e.g., VH-CDRs defined by the Chothia system, are also included in the present invention. In certain embodiments, an antibody or antigen-binding fragment comprising the VH specifically or preferentially binds to IGF-1R.

In another embodiment, the present invention provides an isolated polypeptide comprising, consisting essentially of, or consisting of an immunoglobulin heavy chain variable region (VH) in which the VH-CDR1, VH-CDR2 and VH-CDR3 regions have polypeptide sequences which are identical to the VH-CDR1, VH-CDR2 and VH-CDR3 groups shown in Table 3. In certain embodiments, an antibody or antigen-binding fragment comprising the VH specifically or preferentially binds to IGF-1R.

In another embodiment, the present invention provides an isolated polypeptide comprising, consisting essentially of, or consisting of an immunoglobulin heavy chain variable region (VH) in which the VH-CDR1, VH-CDR2 and VH-CDR3 regions have polypeptide sequences which are identical to the VH-CDR1, VH-CDR2 and VH-CDR3 groups shown in Table 3, except for one, two, three, four, five, or six amino acid substitutions in any one VH-CDR. In larger CDRs, e.g., VH-CDR-3, additional substitutions may be made in the CDR, as long as the VH comprising the VH-CDR specifically or preferentially binds to IGF-1R. In certain embodiments the amino acid substitutions are conservative. In certain embodiments, an antibody or antigen-binding fragment comprising the VH specifically or preferentially binds to IGF-1R.

In a further embodiment, the present invention includes an isolated polypeptide comprising, consisting essentially of, or consisting of a VH polypeptide at least 80%, 85%, 90% 95% or 100% identical to a reference VH polypeptide amino acid sequence selected from the group consisting of SEQ ID NOs: SEQ ID NOs: 4, 9, 14, 20, 26, 32, 38, 43, 48, 53, 58, and 63. In certain embodiments, an antibody or antigen-binding fragment comprising the VH polypeptide specifically or preferentially binds to IGF-1R.

In another aspect, the present invention includes an isolated polypeptide comprising, consisting essentially of, or consisting of a VH polypeptide selected from the group consisting of SEQ ID NOs: 4, 9, 14, 20, 26, 32, 38, 43, 48, 53, 58, and 63. In certain embodiments, an antibody or antigen-binding fragment comprising the VH polypeptide specifically or preferentially binds to IGF-1R.

In certain embodiments, the invention pertains to a binding moiety or binding molecule comprising, consisting essentially of, or consisting of a one or more of the VH polypeptides described above specifically or preferentially binds to the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B 11, 20D8.24B 11, P1E2.3B12, and P1G10.2B8, or will competitively inhibit such a monoclonal antibody or fragment from binding to IGF-1R

In certain embodiments, the invention pertains to a binding moiety or binding molecule comprising, consisting essentially of, or consisting of one or more of the VH polypeptides described above specifically or preferentially binds to an IGF-1R polypeptide or fragment thereof, or a IGF-1R variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In another embodiment, the present invention provides an isolated polypeptide comprising, consisting essentially of, or consisting of an immunoglobulin light chain variable region (VL), where at least one of the VL-CDRs of the light chain variable region or at least two of the VL-CDRs of the light chain variable region are at least 80%, 85%, 90%, 95%, or 100% identical to reference light chain VL-CDR1, VL-CDR2 or VL-CDR3 amino acid sequences from monoclonal IGF-1R antibodies disclosed herein. Alternatively, the VL-CDR1, VL-CDR2 and VL-CDR3 regions of the VL are at least 80%, 85%, 90%, 95%, or 100% identical to reference light chain VL-CDR1, VL-CDR2 and VL-CDR3 amino acid sequences from monoclonal IGF-1R antibodies disclosed herein. Thus, according to this embodiment a light chain variable region of the invention has VL-CDR1, VL-CDR2 and VL-CDR3 polypeptide sequences related to the polypeptides shown in Table 4, supra. While Table 4 shows VL-CDRs defined by the Kabat system, other CDR definitions, e.g., VL-CDRs defined by the Chothia system, are also included in the present invention. In certain embodiments, an antibody or antigen-binding fragment comprising the VL polypeptide specifically or preferentially binds to IGF-1R.

In another embodiment, the present invention provides an isolated polypeptide comprising, consisting essentially of, or consisting of an immunoglobulin light chain variable region (VL) in which the VL-CDR1, VL-CDR2 and VL-CDR3 regions have polypeptide sequences which are identical to the VL-CDR1, VL-CDR2 and VL-CDR3 groups shown in Table 4. In certain embodiments, an antibody or antigen-binding fragment comprising the VL polypeptide specifically or preferentially binds to IGF-1R.

In another embodiment, the present invention provides an isolated polypeptide comprising, consisting essentially of, or consisting of an immunoglobulin heavy chain variable region (VL) in which the VL-CDR1, VL-CDR2 and VL-CDR3 regions have polypeptide sequences which are identical to the VL-CDR1, VL-CDR2 and VL-CDR3 groups shown in Table 4, except for one, two, three, four, five, or six amino acid substitutions in any one VL-CDR. In larger CDRs, additional substitutions may be made in the VL-CDR, as long as the VL comprising the VL-CDR specifically or preferentially binds to IGF-1R. In certain embodiments the amino acid substitutions are conservative. In certain embodiments, an antibody or antigen-binding fragment comprising the VL specifically or preferentially binds to IGF-1R.

In a further embodiment, the present invention includes an isolated polypeptide comprising, consisting essentially of, or consisting of a VL polypeptide at least 80%, 85%, 90% 95% or 100% identical to a reference VL polypeptide sequence selected from the group consisting of SEQ ID NOs: 68, 73, 78, 83, 88, 93, 98, 103, 108, 113, and 118. In certain embodiments, an antibody or antigen-binding fragment comprising the VL polypeptide specifically or preferentially binds to IGF-1R.

In another aspect, the present invention includes an isolated polypeptide comprising, consisting essentially of, or consisting of a VL polypeptide selected from the group consisting of SEQ ID NOs: 68, 73, 78, 83, 88, 93, 98, 103, 108, 113, and 118. In certain embodiments, an antibody or antigen-binding fragment comprising the VL polypeptide specifically or preferentially binds to IGF-1R.

In certain embodiments, the invention pertains to a binding moiety or binding molecule comprising, consisting essentially of, one or more of the VL polypeptides described above specifically or preferentially binds to the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8, or will competitively inhibit such a monoclonal antibody or fragment from binding to IGF-1R.

In certain embodiments, the invention pertains to a binding moiety or binding molecule comprising, consisting essentially of, or consisting of a one or more of the VL polypeptides described above specifically or preferentially binds to an IGF-1R polypeptide or fragment thereof, or a IGF-1R variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In other embodiments, the invention pertains to a binding moiety or binding molecule which comprises, consists essentially of or consists of a VH polypeptide, and a VL polypeptide, where the VH polypeptide and the VL polypeptide, respectively are at least 80%, 85%, 90% 95% or 100% identical to reference VL and VL polypeptide amino acid sequences selected from the group consisting of SEQ ID NOs: 4 and 68, 8 and 73, 14 and 78, 20 and 83, 26 and 88, 32 and 93, 38 and 98, 43 and 103, 48 and 108, 53 and 103, 58 and 113, and 63 and 118. In certain embodiments, an antibody or antigen-binding fragment comprising these VH and VL polypeptides specifically or preferentially binds to IGF-1R.

The polypeptides described above may further include additional polypeptides, e.g., a signal peptide to direct secretion of the encoded polypeptide, antibody constant regions as described herein, or other heterologous polypeptides.

Also, as described in more detail elsewhere herein, the present invention includes binding moiety or binding molecules comprising the polypeptides described above.

It will also be understood by one of ordinary skill in the art that IGF-1R antibody polypeptides as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, or 100% identical to the starting sequence.

Furthermore, nucleotide or amino acid substitutions, deletions, or insertions leading to conservative substitutions or changes at “non-essential” amino acid regions may be made. For example, a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. In other embodiments, a polypeptide or amino acid sequence derived from a designated protein may be identical to the starting sequence except for two or fewer, three or fewer, four or fewer, five or fewer, six or fewer, seven or fewer, eight or fewer, nine or fewer, ten or fewer, fifteen or fewer, or twenty or fewer individual amino acid substitutions, insertions, or deletions. In certain embodiments, a polypeptide or amino acid sequence derived from a designated protein has one to five, one to ten, one to fifteen, or one to twenty individual amino acid substitutions, insertions, or deletions relative to the starting sequence.

Certain IGF-1R binding moiety or binding molecules of the present invention comprise, consist essentially of, or consist of an amino acid sequence derived from a human polypeptide comprising a human amino acid sequence. However, certain IGF-1R antibody polypeptides comprise one or more contiguous amino acids derived from another mammalian species. For example, an IGF-1R antibody of the present invention may include a primate heavy chain portion, hinge portion, or antigen binding region. In another example, one or more murine-derived amino acids may be present in a non-murine antibody polypeptide, e.g., in an antigen binding site of an IGF-1R antibody. In another example, the antigen binding site of an IGF-1R antibody is fully murine. In certain therapeutic applications, IGF-1R-specific antibodies, or antigen-binding fragments, variants, or analogs thereof are designed so as to not be immunogenic in the animal to which the antibody is administered.

In certain embodiments, an IGF-1R binding moiety or binding molecule comprises an amino acid sequence or one or more moieties not normally associated with an antibody. Exemplary modifications are described in more detail below. For example, a single-chain Fv antibody fragment of the invention may comprise a flexible linker sequence, and/or may be modified to add a functional moiety (e.g., PEG, a drug, a toxin, or a label).

An IGF-1R binding moiety or binding molecule of the invention may comprise, consist essentially of, or consist of a fusion protein. Fusion proteins are chimeric molecules which comprise, for example, an immunoglobulin antigen-binding domain with at least one target binding site, and at least one heterologous portion, i.e., a portion with which it is not naturally linked in nature. The amino acid sequences may normally exist in separate proteins that are brought together in the fusion polypeptide or they may normally exist in the same protein but are placed in a new arrangement in the fusion polypeptide. Fusion proteins may be created, for example, by chemical synthesis, or by creating and translating a polynucleotide in which the peptide regions are encoded in the desired relationship.

The term “heterologous” as applied to a polynucleotide or a polypeptide, means that the polynucleotide or polypeptide is derived from a distinct entity from that of the rest of the entity to which it is being compared. For instance, as used herein, a “heterologous polypeptide” to be fused to an IGF-1R binding moiety may be derived from a non-immunoglobulin polypeptide of the same species, or an immunoglobulin or non-immunoglobulin polypeptide of a different species.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Alternatively, in another embodiment, mutations may be introduced randomly along all or part of the immunoglobulin coding sequence, such as by saturation mutagenesis, and the resultant mutants can be incorporated into IGF-1R antibodies for use in the diagnostic and treatment methods disclosed herein and screened for their ability to bind to the desired antigen, e.g., IGF-1R.

In yet other embodiments, binding moieties or binding molecules within the scope of the invention include nucleic acids, peptides, peptidomimetics, dendrimers, and other molecules with binding specificity for an IGF-1R epitope described herein. In one embodiment, binding molecules comprise a binding site which is a nucleic acid, peptide, peptidomimetic, dendrimer, or other molecule with binding specificity for an IGF-1R epitope described herein. For example, binding molecules or binding moieties of the invention include nucleic acid molecules (e.g., small RNAs or aptamers) that are capable of binding with high affinity to an IGF-1R epitope described herein. Methods for selecting or screening nucleic molecules of a desired specificity are well known in the art (see e.g., Ellington and Szostak, Nature 346:818 (1990), Tuerk and Gold, Science 249:505 (1990), U.S. Pat. No. 5,582,981, PCT Publication No. WO 00/20040, U.S. Pat. No. 5,270,163, Lorsch and Szostak, Biochemistry, 33:973 (1994), Mannironi et al., Biochemistry 36:9726 (1997), Blind, Proc. Nat'l. Acad. Sci. USA 96:3606-3610 (1999), Huizenga and Szostak, Biochemistry, 34:656-665 (1995), PCT Publication Nos. WO 99/54506, WO 99/27133, WO 97/42317 and U.S. Pat. No. 5,756,291). An exemplary screening method well known in the art is the SELEX method (Systematic Evolution of Ligands by Exponential Enrichment, see, for example, U.S. Pat. Nos. 5,270,163 and 5,567,588; herein incorporated by reference).

In another embodiment, a binding molecule or binding moiety of the invention is a mimetic, e.g. a peptidomimetic. A number of peptidomimetics of various structures are known in the art. For example, WO 00/68185 discloses peptidomimetics that mimic helical portions of certain proteins. In another embodiment, the present invention is directed to compounds or molecules which mimic the 3-dimensional structure of a binding site (e.g. a CDR, antigen binding site, or paratope) of a binding polypeptide (e.g. an antibody) described herein. As used herein, the term “mimic” means the 3-dimensional placement of atoms of the mimetic such that similar ionic forces, covalent forces, van der Waal's or other forces, and a similar charge complementarity, or electrostatic complementarity, exist between the atoms of the mimetic and the atoms of an antigen binding site or epitope, and/or such that the mimetic has a similar binding affinity for an antigenic epitope (e.g. an IGF-1R epitope) as a binding polypeptide described herein, and/or such that the mimetic has a similar effect on the function of the antigen in vitro or in vivo. Methods isolating or screening for compounds or mimetics which mimic a binding site are well known in the art. For example, it is possible to use an anti-idiotypic antibody which recognizes unique idiotypic determinants located on a IGF-1R binding polypeptide described herein. These determinants are located in the binding site of a binding polypeptide (e.g., the hypervariable region of an antibody) which binds a particular IGF-1R epitope. An anti-idiotypic antibody can be prepared by immunizing an animal with the binding polypeptide of interest such that an antibody which recognizes the idiotypic determinants of the binding site is produced. An anti-idiotypic monoclonal antibody made to a first binding site will have a binding site which is the image of the epitope bound by the first binding site. By using the anti-idiotypic antibodies of the immunized animal, it is possible to identity other antibodies with the same idiotype as the antibody used for immunization. Idiotypic identity between two antibodies demonstrates that the two antibodies are the same with respect to their recognition of the same epitope. Thus, by using anti-idiotypic antibodies, it is possible to identity other antibodies having the same epitopic binding specificity. Since the anti-idiotypic antibody is the image of the epitope bound by the first binding polypeptide, and since the anti-idiotypic antibody effective acts as antigen, it may be used to isolate mimetic from combinatorial libraries of small chemical molecules, peptides, or other molecules, such as peptide phage display libraries (see, e.g., Scott et al, Science, 249: 386-390 (1990); Scott et al., Curr. Opin. Biotechnol., 5: 40-48 (1992); Bonnycastle et al., J. Mol. Biol., 258: 747-762 (1996), which are incorporated herein by reference). For example, peptides or constrained peptide mimics, including those with lipid, carbohydrate, or other moieties, may be cloned (see Harris et al., PNAS, 94: 2454-2459 (1997)).

By techniques well known in the art, compounds or mimetics may also be designed in light of the nucleic acid and amino acid sequences of binding molecules disclosed herein and the three dimensional array or conformations of the amino acids of the binding molecules, as determined X-ray crystallography or NMR of the binding molecules (see e.g., U.S. Pat. No. 5,648,379; Colman et al., Protein Science, 3: 1687-1696 (1994); Malby et al., Structure et al., 2: 733-746 (1994); McCoy et al., J. Mol. Biol., 268: 570-584 (1997); Pallaghy et al., Biochemistry, 34: 3782-3794 (1995), each of which is incorporated herein by reference). Thus, a mimetic or molecule which mimics the 3-dimensional structure of a binding site or moiety described herein (e.g., a paratope) may be designed from the analysis of the interaction of the binding site and an IGF-1R epitope in crystals of the two molecules, or in solutions containing the two molecules. Purely synthetic binding molecules may be designed by the 3-dimensional placement of atoms, such that similar ionic forces, covalent forces, van der Waals, or other forces, and similar charge complementarity, exist between the atoms of the mimetic and the atoms of the binding or moiety. These mimetics may then be screened for high affinity binding to the antigenic epitope and inhibition of B-cell function in vitro and in vivo.

### IV. IGF-1R Epitopes

A. Epitopes Resulting in Competitive Inhibition of Binding

In certain embodiments, an IGF-1R binding moiety may bind to a competitive epitope of IGF-1R such that it competitively blocks binding of a ligand (e.g. IGF1 and/or IGF2) to IGF-1R. Such binding specificities are referred to herein as “competitive binding moieties.” In one embodiment, the competitive binding moiety competitively blocks binding of IGF-1 (but not IGF-2) to IGF-1R. In another embodiment, the competitive binding moiety competitively blocks binding of IGF-2 (but not IGF-1) to IGF-1R. In yet another embodiment, the competitive binding moiety competitively blocks binding of both IGF-1 and IGF-2 to IGF-1R.

A binding molecule is said to “competitively inhibit” or “competitively block” binding of the ligand if it specifically or preferentially binds to the epitope to the extent that binding of the ligand (e.g. IGF) to IGF-1R is inhibited or blocked (e.g. sterically blocked) in a manner that is dependent on the concentration of the ligand. For example, when measured biochemically, competitive inhibition at a given concentration of binding molecule can be overcome by increasing the concentration of ligand in which case the ligand will outcompete the binding molecule for binding to the target molecule (e.g., IGF-1R). Without being bound to any particular theory, competition is thought to occur when the epitope to which the binding molecule binds is located at or near the binding site of the ligand, thereby preventing binding of the ligand. Competitive inhibition may be determined by methods well known in the art and/or described in the Examples, including, for example, competition ELISA assays. In one embodiment, a binding molecule of the invention competitively inhibits binding of the ligand to a given epitope by at least 90%, at least 80%, at least 70%, at least 60%, or at least 50%.

An exemplary competitive epitope is situated within a region encompassing the mid and C-terminal regions of the CRR domain at residues 248-303 of IGF-1R. This epitope of IGF-1R is adjacent (in 3-dimensional space) to the IGF-1/IGF-2 ligand binding site of the L1 domain. An exemplary antibody which competitively binds to this epitope is the human antibody designated M14-G11. The M14-G11 antibody has been shown to competitively block binding of both IGF-1 and IGF-2 to IGF-1R. Chinese Hamster Ovary cell lines which express the Fab antibody fragment of M14-G11 were deposited with the American Type Culture Collection (“ATCC”) on Aug. 29, 2006, and were given ATCC Deposit Number PTA-7855.

Accordingly, in certain embodiments, a binding moiety employed in the compositions of the invention may bind to the same competitive epitope as the M14-G11 antibody. For example, a binding moiety may be derived from an antibody which cross-blocks (i.e., competes for binding with) an M14-G11 antibody or otherwise interferes with the binding of the M14-G11 antibody. In other embodiments, the binding moiety may comprise the M14-G11 antibody itself, or a fragment, variant, or derivative thereof. In other embodiments, a binding moiety may comprise an antigen binding domain, variable region (VL or VH), or CDR therefrom. For example, a competitive binding moiety may comprise all six CDRs (i.e., CDRs 1-6) of a M14-G11 antibody or it may comprise fewer than all six CDRs (e.g., one, two, three, four, or five CDRs) from the M14-G11 antibody. In one exemplary embodiment, the competitive binding specificity comprises CDR-H3 from the M14-G11 antibody.

Other antibodies which bind to a competitive epitope of IGF-1R may be identified using art-recognized methods. For example, once antibodies to various fragments of, or to the full-length IGF-1R without the signal sequence, have been produced, determining which amino acids, or epitope, of IGF-1R to which the antibody or antigen binding fragment binds can be determined by epitope mapping protocols as described herein as well as methods known in the art (e.g. double antibody-sandwich ELISA as described in “Chapter 11—Immunology,” Current Protocols in Molecular Biology, Ed. Ausubel et al., v.2, John Wiley & Sons, Inc. (1996)). Additional epitope mapping protocols may be found in Morris, G. Epitope Mapping Protocols, New Jersey Humana Press (1996), which are both incorporated herein by reference in their entireties. Epitope mapping can also be performed by commercially available means (i.e. ProtoPROBE, Inc. (Milwaukee, Wis.)). Additionally, antibodies produced which bind to a competitive epitope of IGF-1R can then be screened for their ability to competitively inhibit binding of insulin growth factor, e.g., IGF-1, IGF-2, or both IGF-1 and IGF-2 to IGF-1R. Antibodies can be screened for these and other properties according to methods described in detail in the Examples.

In other embodiments, a competitive IGF-1R binding moiety specifically or preferentially binds to a competitive epitope which comprises, consists essentially of, or consists of at least about four to five amino acids of the sequence spanning residues 248-303 of IGF-1R, inclusive. For example, in one embodiment, a competitive IGF-1R binding moiety comprises, at least seven, at least nine, or between at least about 15 to about 30 amino acids of the sequence spanning residues 248-303 of IGF-1R. The amino acids of a given epitope may be, but need not be contiguous or linear. In certain embodiments, the competitive epitope comprises, consists essentially of, or consists of a non-linear epitope formed by the CRR and L2 domain interface of IGF-1R as expressed on the surface of a cell or as a soluble fragment, e.g., fused to an IgG Fc region. Thus, in certain embodiments a competitive epitope of IGF-1R comprises, consists essentially of, or consists of at least 3, at least 4, at least 5, at least 6, at least 7, at least 8, at least 9, at least 10, at least 15, at least 20, at least 25, between about 15 to about 30, or at least 10, 15, 20, 25, 30, 35, 40, or 45 contiguous or non-contiguous amino acids of the sequence spanning residues 248-303 of IGF-1R. In the case of non-contiguous amino acids, the amino acids form an epitope through protein folding.

In other embodiments, the competitive epitope to which the binding moiety binds comprises, consists essentially of, or consists of at least 3, at least 4, at least 5, at least 6, at least 7, at least 8, at least 9, at least 10, at least 15, at least 20, at least 25, between about 15 to about 30, contiguous or non-contiguous amino acids of IGF-1R and at least one of the amino acids of the epitope is selected from the group consisting of amino acid number 248, 250, 254, 257, 259, 260, 263, 265, 301, and 303 of IGF-1R.

In other embodiments, the amino acids bound by a binding moiety of the invention are present in the epitope spanning amino acids 248-303 of IGF-1R. In one embodiment, the epitope bound by a binding moiety of the invention includes at least one amino acid that, when mutated, leads to ablation or large decreases in antibody affinity (e.g., >100-fold decrease in affinity), e.g. IGF-1R residues 248 and/or 250. In another embodiment, the epitope may comprise one or more amino acids of IGF-1R which, when mutated, leads to a moderate decrease in antibody affinity towards the receptor (10≧K_{D}≧100-fold above that of wild-type IGF-1R). In yet other embodiments, the epitope may comprise an amino acid of IGF-1R which, when mutated, leads to small decreases in antibody affinity (e.g., 2.5≧K_{D}≧10 nM) compared to wild-type human IGF-1R, e.g. one or more of residues 254, 257, 259, 260, 263, 265, 301, or 303 of IGF-1R. In a preferred embodiment, the epitope bound by a binding moiety of the invention comprises any one, any two, or all three of IGF-1R residues 248, 250, and/or 254. In a particularly preferred embodiment, a competitive binding moiety binds to an epitope comprising all three amino acids 248, 250, and 254 and simultaneously recognizes these amino acid residues.

B. Epitopes Resulting in Allosteric Inhibition of Binding

In certain embodiments, a binding moiety may bind to an allosteric epitope such that it allosterically blocks binding of an IGF ligand to IGF-1R. Such binding specificities are referred to herein as “allosteric binding moieties”. In one embodiment, the allosteric binding moiety allosterically blocks binding of IGF-1 (but not IGF-2) to IGF-1R. In another embodiment, the allosteric binding moiety allosterically blocks binding of IGF-2 (but not IGF-1) to IGF-1R. In yet another embodiment, an allosteric binding moiety allosterically blocks binding of both IGF-1 and IGF-2 to IGF-1R.

A binding molecule is said to “allosterically inhibit” or “allosterically block” binding of the ligand if it specifically or preferentially binds to the epitope to the extent that binding of the ligand (e.g. IGF1 and/or IGF2) to IGF-1R is inhibited or blocked in a manner that is independent of the concentration of the binding molecule. For example, increases in the concentration of ligand will not effect the potency of inhibition (e.g., IC₅₀ or concentration at which the binding molecule leads to a 50% reduction in its maximal ligand inhibition). Without being bound to any particular theory, allosteric inhibition is thought to occur when there is a conformational or dynamic change in the target molecule (e.g. IGF-1R) that is induced by binding of the binding molecule to the allosteric epitope, such that the affinity of the ligand for the target is reduced. Allosteric inhibition may be determined by methods well known in the art or described in the Examples, including, for example, competition ELISA assays. In one embodiment, a binding molecule may allosterically inhibit binding of the ligand to a given epitope by at least 90%, at least 80%, at least 70%, at least 60%, or at least 50%.

(i) Epitopes Resulting in Allosteric Blocking of IGF-1 and IGF-2

In certain exemplary embodiments, a binding molecule of the invention comprises a binding moiety which binds an allosteric epitope located within a region spanning the entire FnIII-1 domain of IGF-1R and comprising residues 440-586 of IGF-1R. Exemplary antibodies which allosterically bind to an epitope within this region are the human antibodies designated M13-C06 and M14-C03. Both the M13-C06 antibody and the M14-C03 antibody have been shown in the Examples to allosterically block binding of both IGF-1 and IGF-2 to IGF-1R. Chinese Hamster Ovary cell lines which express full-length antibody of M13-C06 and M14-C03 were deposited with the American Type Culture Collection (“ATCC”) on Mar. 28, 2006, and were given ATCC Deposit Numbers PTA-7444 and PTA-7445, respectively. Accordingly, in certain embodiments, a binding moiety employed in the compositions of the invention may bind to the same allosteric epitope as the M13-C06 antibody or the M14-C03 antibody. For example, a binding specificity may be derived from an antibody which cross-blocks (competes with) the M13-C06 antibody or the M14-C03 antibody or otherwise interferes with the binding of the M13-C06 antibody or the M14-C03 antibody. In other embodiments, the binding moiety may comprise either of the M13-C06 or the M14-C03 antibodies themselves, or a fragment, variant, or derivative thereof. In other embodiments, a binding moiety may comprise an antigen binding domain, variable region (VL and/or VH), or CDR therefrom. For example, an allosteric binding moiety may comprise all six CDRs of the M13-C06 antibody or the M14-C03 antibody or it may comprise fewer than all six CDRs (e.g., one, two, three, four, or five CDRs) from the M13-C06 antibody or the M14-C03 antibody. In one exemplary embodiment, the allosteric binding specificity comprises CDR-H3 from the M13-C06 antibody or the M14-C03 antibody.

In certain embodiments, an allosteric IGF-1R binding moiety specifically or preferentially binds to an allosteric epitope which comprises, consists essentially of, or consists of at least about four to five amino acids of the sequence spanning residues 440-586 of IGF-1R, at least seven, at least nine, or between at least about 15 to about 30 amino acids of the sequence spanning residues 440-586 of IGF-1R. The amino acids of a given epitope may be, but need not be, contiguous or linear.

In certain embodiments, the allosteric epitope comprises, consists essentially of, or consists of a non-linear epitope located in L2 and/or FnIII-1 domain of IGF-1R as expressed on the surface of a cell or as a soluble fragment, e.g., fused to an IgG Fc region. Thus, in certain embodiments the allosteric epitope comprises, consists essentially of, or consists of at least 3, at least 4, at least 5, at least 6, at least 7, at least 8, at least 9, at least 10, at least 15, at least 20, at least 25, between about 15 to about 30, or at least 10, 15, 20, 25, 30, or more contiguous or non-contiguous amino acids of the sequence spanning amino acid positions 440-586 of IGF-1R, where the non-contiguous amino acids form an epitope through protein folding.

In another embodiment, the allosteric epitope to which the binding moiety binds comprises, consists essentially of, or consists of at least 3, at least 4, at least 5, at least 6, at least 7, at least 8, at least 9, at least 10, at least 15, at least 20, at least 25, between about 15 to about 30, contiguous or non-contiguous amino acids of IGF-1R and at least one of the amino acids of the epitope is selected from the group consisting of amino acid number 437, 438, 459, 460, 461, 462, 464, 466, 467, 469, 470, 471, 472, 474, 476, 477, 478, 479, 480, 482, 483, 488, 490, 492, 493, 495, 496, 509, 513, 514, 515, 533, 544, 545, 546, 547, 548, 551, 564, 565, 568, 570, 571, 572, 573, 577, 578, 579, 582, 584, 585, 586, and 587 of IGF-1R.

In other embodiments, the epitope bound by a binding moiety of the invention comprises at least one amino acid of IGF-1R selected from residues on the surface of the FnIII-1 domain of IGF-1R within a 14 Å radius of residues 462-464, for example, residues S437, E438, E469, N470, E471, L472, K474, S476, Y477, I478, R479, R488, E490, Y492, W493, P495, D496, E509, Q513, N514, V515, K544, S545, Q546, N547, H548, W551, R577, T578, Y579, K582, D584, I585, I586, and Y587. In other embodiments, a binding moiety of the invention binds to at least one amino acid selected from residues within positions 440-586 of IGF-1R which, when mutated, leads to ablation or large decreases in antibody affinity (e.g., >100-fold decrease in affinity), e.g. IGF-1R residues 459, 460, 461, 462, 464, 480, 482, 483, 490, 533, 570, or 571. In yet other embodiments, the epitope may comprise an amino acid of IGF-1R which, when mutated, leads to small decreases in antibody affinity (e.g., 2.5≧K_{D}≧10 nM) compared to wild-type human IGF-1R, e.g. at residues 466, 467, 478, 533, 564, 565, or 568 of IGF-1R. In a particular preferred embodiment, the epitope bound by a binding moiety of the invention comprises any one, any two, or all three of IGF-1R residues 461, 462, and 464.

(ii) Epitopes Resulting in Allosteric Blocking of IGF-1 and not IGF-2

Another exemplary allosteric epitope is located on the surface of the CRR domain of IGF-1R on a face of the receptor rotated slightly away from the IGF-1/IGF-2 binding pocket. The epitope may span large regions of both the CRR and L2 domains. In one embodiment, the allosteric epitope is located within a region that comprises residues 241-379 of IGF-1R. In certain embodiments, the allosteric epitope is located within a region that includes residues 241-266 of the CRR domain IGF-1R or residues 301-308 and 327-379 of the L2 domain of IGF-1R. Exemplary antibodies which allosterically bind to this epitope include the antibodies designated P1E2 and αIR3. Both the P1E2 antibody and the αIR3 antibody have been shown in the Examples to allosterically block binding of IGF-1 (but not IGF-2) to IGF-1R. In one embodiment, a P1E2 antibody is a chimeric antibody that contains the mouse VH and VL derived from the mouse antibody expressed by the P1E2.3B12 mouse hybridoma) and fused to a human IgG4 Palgy/kappa constant domains (e.g., IgG4 constant domains comprising substitutions S228P and T299A (EU numbering convention)). A hybridoma cell line which expresses a full-length mouse antibody P1E2.3B12 was deposited with the ATCC on Jul. 11, 2006 and given the ATCC Deposit Number PTA-7730.

Accordingly, in certain embodiments, a binding moiety employed in the compositions of the invention may bind to the same allosteric epitope as the P1E2 antibody or the αIR3 antibody. For example, a binding specificity may be derived from an antibody which cross-blocks (competes with) the P1E2 antibody or the αIR3 antibody or otherwise interferes with the binding of the P1E2 antibody or the αIR3 antibody. In other embodiments, the binding specificity may comprise either of the P1E2 or αIR3 antibodies themselves, or a fragment, variant, or derivative thereof. In other embodiments, a binding moiety may comprise an antigen binding domain, variable region (VL and/or VH), or CDR therefrom. For example, an allosteric binding moiety may comprise all six CDRs of the P1E2 antibody or the αIR3 antibody or it may comprise fewer than all six CDRs (e.g., one, two, three, four, or five CDRs) from the P1E2 antibody or the αIR3 antibody. In one exemplary embodiment, the allosteric binding specificity comprises CDR-H3 from the P1E2 antibody or the αIR3 antibody.

Other antibodies which bind to an allosteric epitope of IGF-1R may be identified using art-recognized methods such as those described above. Additionally, antibodies produced which bind to an allosteric epitope of IGF-1R can then be screened for their ability to allosterically block binding of an insulin growth factor, e.g., IGF-1, IGF-2, or both IGF-1 and IGF-2 to IGF-1R. Antibodies can be screened for these and other properties according to methods described in detail in the Examples.

In yet other embodiments, an allosteric IGF-1R binding moiety specifically or preferentially binds to an allosteric epitope which comprises, consists essentially of, or consists of at least about four to five amino acids of the sequence spanning residues 241-266 of IGF-1R, at least seven, at least nine, or between at least about 15 to about 25 amino acids of the sequence spanning amino acid residues 241-266 of IGF-1R. The amino acids of the epitope may be, but need not be contiguous or linear. In certain embodiments, the allosteric epitope comprises, consists essentially of, or consists of a non-linear epitope present on the extracellular surface of the CRR domain of IGF-1R as expressed on the surface of a cell or as a soluble fragment, e.g., fused to an IgG Fc region. Thus, in certain embodiments the allosteric epitope comprises, consists essentially of, or consists of at least 3, at least 4, at least 5, at least 6, at least 7, at least 8, at least 9, at least 10, at least 15, at least 20, at least 25, between about 15 to about 25, or at least 10, 11, 12, 13, 14, 15, 16, 17, 18, 19, 20, 21, 22, 23, 24, or 25 contiguous or non-contiguous amino acids of the sequence spanning amino acid residues about 241 to about 379 (e.g. residues 241-266 or 301-308 or 327-379) of IGF-1R, where the non-contiguous amino acids form an epitope through protein folding.

In other embodiments, the allosteric epitope to which the binding moiety binds comprises, consists essentially of, or consists of at least 3, at least 4, at least 5, at least 6, at least 7, at least 8, at least 9, at least 10, at least 15, at least 20, at least 25, between about 15 to about 30, contiguous or non-contiguous amino acids wherein at least one of the amino acids of the epitope (preferably all of the amino acids of the epitope) is selected from the group consisting of 241, 248, 250, 251, 254, 257, 263, 265, 266, 301, 303, 308, 327, and 379.

In other embodiments, the epitope recognized by a binding moiety of the invention comprises one or more of amino acids 241-266 of IGF-1R which, when mutated, lead to ablation or large decreases in antibody affinity (e.g., >100-fold decrease in affinity), e.g. at least one or all of IGF-1R residues 248, 254, or 265. In another embodiment, the epitope may comprise at least one amino acid which, when mutated, causes a moderate reduction in binding affinity (e.g. 10≧K_{D}≧100-fold above that of wild-type IGF-1R), for example, IGF-1R residues 254 and/or 257. In yet other embodiments, the epitope may comprise an amino acid of IGF-1R which, when mutated, leads to small decreases in antibody affinity (e.g., 2.5≧K_{D}≧10 nM) compared to wild-type human IGF-1R, e.g. at one or more of IGF-1R residues 248, 263, 301, 303, 308, 327, or 379. In a particular preferred embodiment, the epitope comprises any one, any two, any three, any four, any five, or all six of IGF-1R residues 241, 242, 251, 257, 265, and 266.

C. Other IGF-1R Epitopes

In certain embodiments, an IGF-1R binding moiety may bind to the same epitope as an antibody selected from the group consisting of P2A7.3E11, 20C8.3B8, P1A2.2B11, 20D8.24B11, and P1G10.2B8. In one exemplary embodiment of the invention, an IGF-1R binding moiety of a binding molecule of the invention is derived from a parental murine antibody selected from the group consisting of P2A7.3E11, 20C8.3B8, P1A2.2B11, 20D8.24B11, and P1G10.2B8. Hybridoma cell lines which express antibodies P2A7.3E11, 20C8.3B8, and P1A2.2B11 were deposited with the ATCC on Mar. 28, 2006, Jun. 13, 2006, and Mar. 28, 2006, respectively, and were given the ATCC Deposit Numbers PTA-7458, PTA-7732, and, PTA-7457, respectively. Hybridoma cell lines which express full-length antibodies 20D8.24B11 and P1G10.2B8 were deposited with the ATCC on Mar. 28, 2006, and Jul. 11, 2006, respectively, and were given the ATCC Deposit Numbers PTA-7456 and PTA-7731, respectively.

In yet other embodiments, a binding moiety employed in the compositions of the invention may be derived from an antibody which cross-blocks (competes with) with an antibody selected from the group consisting of any antibody selected from the group consisting of P2A7.3E11, 20C8.3B8, P1A2.2B11, 20D8.24B11, and P1G10.2B8 or otherwise interferes with the binding of selected from the group consisting of P2A7.3E11, 20C8.3B8, P1A2.2B11, 20D8.24B11, and P1G10.2B8. In other embodiments, the binding moiety may comprise an antibody selected from the group consisting of P2A7.3E11, 20C8.3B8, P1A2.2B11, 20D8.24B11, and P1G10.2B8, or a fragment, variant, or derivative thereof. In other embodiments, a binding moiety may comprise an antigen binding domain, variable region (VL and/or VH), or CDR therefrom. For example, a binding moiety may comprise all six CDRs of an antibody selected from the group consisting of P2A7.3E11, 20C8.3B8, P1A2.2B11, 20D8.24B1, and P1G10.2B8 or it may comprise fewer than all six CDRs (e.g., one, two, three, four, or five CDRs) from an antibody selected from the group consisting of P2A7.3E11, 20C8.3B8, P1A2.2B11, 20D8.24B11, and P1G10.2B8. In one exemplary embodiment, the binding specificity comprises CDR-H3 from an antibody selected from the group consisting of P2A7.3E11, 20C8.3B8, P1A2.2B11, 20D8.24B11, and P1G10.2B8.

### V. Compositions Comprising Binding Molecules that Bind to Different Epitopes of IGF-1R

The instant invention provides compositions comprising binding molecules that bind to different epitopes of IGF-1R. In certain embodiments, the compositions of the invention comprise two IGF-1R binding moieties or binding molecules having different IGF-1R binding specificities. In other embodiments, the binding compositions of the invention comprise one IGF-1R binding molecule with multiple IGF-1R binding specificities (i.e., a multispecific IGF-1R binding molecule). In preferred embodiments, binding of the binding compositions of the invention to IGF-1R result in reduced IGF-1R signaling as compared to the use of one binding molecule having a single specificity for IGF-1R. For example, in certain embodiments, the compositions of the invention can lead to a synergistic reduction in IGF-1/IGF-2-mediated signaling and/or a synergistic reduction in tumor cell proliferation. Such compositions can lead to complete IGF ligand blockage with greater potency and can also expand the target cell population which may be effectively inhibited by blockade of IGF-1R signaling.

In certain embodiments, the binding compositions or binding molecules of the invention comprise first and second binding molecules or binding moieties independently selected from any one of the binding molecule or binding moieties disclosed supra. In certain embodiments, binding of the first and second binding moiety to IGF-1R blocks IGF-1R-mediated signaling to a greater extent than the binding of the first or second binding moiety alone. As used herein, the term “block IGF-1R-mediated signaling to a greater extent” with respect to the binding of a binding molecule to IGF-1R, refers to a situation where the binding of a first binding moiety that binds to a first epitope of IGF-1R (that blocks the binding of at least one of IGF-1 and IGF-2 to IGF-1R) and a second binding moiety that binds to a second, different epitope of IGF-1R (that blocks the binding of at least one of IGF-1 and IGF-2 to IGF-1R to IGF-1R) blocks IGF-1R-mediated signaling more than the binding of the first or second moiety alone. Inhibition of IGF-1R-mediated signaling can be measured in a number of different ways, e.g., downmodulation of tumor growth (e.g. tumor growth delay), reduction in tumor size or metastasis, the amelioration or minimization of the clinical impairment or symptoms of cancer, an extension of the survival of the subject beyond that which would otherwise be expected in the absence of such treatment, and the prevention of tumor growth in an animal lacking any tumor formation prior to administration, i.e., prophylactic administration. As used herein, the terms “downmodulate”, “downmodulating” or “downmodulation” refer to decreasing the rate at which a particular process occurs, inhibiting a particular process, reversing a particular process, and/or preventing the initiation of a particular process. Accordingly, if the particular process is tumor growth or metastasis, the term “downmodulation” includes, without limitation, decreasing the rate at which tumor growth and/or metastasis occurs; inhibiting tumor growth and/or metastasis; reversing tumor growth and/or metastasis (including tumor shrinkage and/or eradication) and/or preventing tumor growth and/or metastasis.

In one embodiment, when IGF-1R-mediated signaling is blocked to a greater extent, an additive effect is observed. The term “additive effect”, as used herein refers to the scenario wherein sum effect of the binding of a first and second binding moiety in combination is approximately equal to the effect observed when the first or second binding moieties bind alone. An additive effect is typically measured under conditions where the molar ratio of the first or second binding moiety (alone) to IGF-1R is approximately the same as the molar ratio of the first and second binding-moiety (together) to IGF-1R.

In one embodiment, when IGF-1R-mediated signaling is blocked to a greater extent, a synergistic effect is observed. The term “synergistic effect”, as used herein, refers to a greater-than-additive effect which is produced upon binding of the first and second binding moieties, and which exceeds that which would otherwise result from individual administration of either the first or second binding moieties alone. A synergistic effect is typically measured under conditions where the molar ratio of the first or second binding moiety (alone) to IGF-1R is approximately the same as the molar ratio of the first and second binding moiety (together) to IGF-1R. Embodiments of the invention include methods of producing a synergistic effect in downmodulating IGF-1R-mediated signaling via use of said first and second IGF-1R binding moieties, wherein said effect is at least 5%, 10%, 15%, 20%, 25%, 30%, 40%, 50%, 60%, 70%, 80%, 90% or 100% greater than the corresponding additive effect.

In one embodiment, a synergistic effect is measured using the combination index (CI) method of Chou and Talalay (see Chang et al., Cancer Res. 45: 2434-2439, (1985)) which is based on the median-effect principle. This method calculates the degree of synergy, additivity, or antagonism between two drugs at various levels of cytotoxicity. Where the CI value is less than 1, there is synergy between the two drugs. Where the CI value is 1, there is an additive effect, but no synergistic effect. CI values greater than 1 indicate antagonism. The smaller the CI value, the greater the synergistic effect. In another embodiment, a synergistic effect is determined by using the fractional inhibitory concentration (FIC). This fractional value is determined by expressing the IC₅₀ of a drug acting in combination, as a function of the IC₅₀ of the drug acting alone. For two interacting drugs, the sum of the FIC value for each drug represents the measure of synergistic interaction. Where the FIC is less than 1, there is synergy between the two drugs. An FIC value of 1 indicates an additive effect. The smaller the FIC value, the greater the synergistic interaction.

In certain alternative embodiments, a synergistic effect is observed when greater modulation occurs upon combination of two separate compounds (e.g. separate binding moieties) than what is possible when using saturating concentrations or doses of each of the compounds. This form of synergy may occur where the single binding moieties themselves are not capable of leading to a complete effect (e.g., 100% downmodulation is not reached regardless of how high the concentration of the drug is used). In this situation, synergistic effects are not adequately captured by analysis of EC₅₀ or IC₅₀ values. If the combination of two compounds (e.g. binding moieties) leads to a greater downmodulation than what is possible for the single compounds, this is recognized as a powerful synergistic effect.

In certain embodiments the binding compositions of the invention may target two or more different epitopes (e.g., two or more non-overlapping epitopes) on the extracellular region of IGF-1R. In one embodiment, a binding composition of the invention may comprise a first binding molecule which binds a first IGF-1R epitope and a second binding molecule which binds a second IGF-1R epitope. One skilled in the art will appreciate that the first and second IGF-1R epitopes may be located in the same IGF-1R molecule or on different IGF-1R molecules.

In one embodiment, the binding compositions of the invention bind two or more different epitopes wherein the epitopes are independently selected from the group consisting of: an epitope located in the L1 domain, an epitope located in the CRR domain, an epitope located in the L2 domain, an epitope located in the Fn-1 domain, an epitope located in the Fn-2 domain, an epitope located in the Fn-3 domain. For example, a binding composition of the invention may bind a first epitope located in the L2 domain and a second epitope located in the CRR domain. In another embodiment, a binding composition of the invention may bind two or more epitopes located in the same domain. In yet another embodiment, a binding composition of the invention binds two or more epitopes wherein at least one of the epitopes is formed by two or more domains (e.g., an epitope within the binding interface of a L2 domain and a CRR domain).

In one embodiment, a composition of the invention comprises one or more binding molecules which targets two different epitopes of IGF-1R, where each of the epitopes, when bound by a binding moiety, inhibits IGF-1R signaling via a different mechanism. In one embodiment, a binding composition of the invention may target an allosteric epitope and a competitive epitope.

Binding compositions of the invention may bind to competitive or allosteric epitopes within IGF-1R. As used herein, the term “competitive epitope” refers to an epitope which, when bound by a binding molecule, leads to competitive inhibition of ligand binding to its receptor (e.g., binding of IGF-1 and/or IGF-2 to IGF-1R). Competitive epitopes are generally located in the ligand binding site of a receptor. An exemplary competitive epitope of IGF-1R is located on the inside face of the CRR domain in the proximity of the IGF-1 and IGF-2 binding site (see FIG. 1). Binding to this epitope leads to competitive inhibition of IGF-1 and IGF-2 binding. As used herein, the term “allosteric epitope” refers to an epitope which, when bound by a binding molecule, leads to allosteric inhibition of a ligand binding to its receptor. Allosteric epitopes are generally located at a site within a receptor that is distal to the ligand binding site. An exemplary allosteric epitope of IGF-1R is located on the exposed face of the CRR/L2 region (see FIG. 1). Binding to this epitope leads to allosteric IGF-1 blockage, but has little effect on IGF-2 binding. Another exemplary allosteric epitope is located on the outer surface of the FnIII-1 domain (see FIG. 1). Binding to this epitope leads to allosteric blockade of both IGF-1 and IGF-2.

In one embodiment, a composition of the invention comprises one or more binding molecules which target two different epitopes of IGF-1R, wherein a binding moiety that binds to the first epitope does not cross-block (i.e., compete with) the second binding moiety that binds to the second epitope.

A. Combinations of Multiple Binding Molecules

In certain aspects of the invention, there are provided compositions comprising combinations of inhibitory anti-IGF-1R binding molecules (e.g., two or more anti-IGF-1R antibodies, antibody fragments, antibody variants, aptamers, or derivatives thereof) with different IGF-1R binding specificities. For example, the compositions of the invention may comprise a first anti-IGF-1R binding molecule having a first IGF-1R binding specificity and a second anti-IGF-1R binding molecule having a second IGF-1R binding specificity. In preferred embodiments, the first and second binding specificities bind non-overlapping epitopes within the extracellular domain of IGF-1R. The binding molecules of the composition can be administered separately or in combination to a subject. Said compositions can lead to complete ligand blockade with greater potency (e.g. lower concentrations) than conventional compositions. In other embodiments, the compositions can lead to a synergistic reduction in tumor cell proliferation.

It will be recognized by those of skill in the art that the combination of binding molecules in a composition of the invention may comprise any combination of the binding molecules disclosed herein. For example, the composition of the invention may comprise a combination of at a least a first and second binding molecule, wherein said binding molecules independently selected from any one of the binding molecules disclosed herein. Preferably, the combination of binding molecules comprises a first binding molecule comprising an allosteric binding moiety and a second binding molecule comprising a competitive binding moiety. In one embodiment, the combination comprises a first antibody or scFv molecule (e.g., any one of the stabilized scFv molecules disclosed herein) comprising an allosteric binding moiety and a second antibody or scFv molecule comprising a competitive binding moiety.

Binding molecules of the invention may be monovalent, i.e., comprise one target binding site (e.g., as in the case of an scFv molecule) or more than one target binding site. In one embodiment, the binding molecules comprise at least two binding sites. In one embodiment, the binding molecules comprise three binding sites. In another embodiment, the binding molecules comprise four binding sites. In another embodiment, the binding molecules comprise greater than four binding sites.

In one embodiment, the binding molecules of the invention are monomers. In another embodiment, the binding molecules of the invention are multimers. For example, in one embodiment, the binding molecules of the invention are dimers. In one embodiment, the dimers of the invention are homodimers, comprising two identical monomeric subunits. In another embodiment, the dimers of the invention are heterodimers, comprising two non-identical monomeric subunits. The subunits of the dimer may comprise one or more polypeptide chains. For example, in one embodiment, the dimers comprise at least two polypeptide chains. In one embodiment, the dimers comprise two polypeptide chains. In another embodiment, the dimers comprise four polypeptide chains (e.g., as in the case of antibody molecules).

B. Multispecific Binding Molecules

In other aspects, the invention provides compositions comprising multispecific IGF-1R binding molecules (e.g., multispecific anti-IGF-1R antibodies, antibody variants, antibody fragments, or aptamers with two or more IGF-1R binding specificities). The multispecific IGF-1R binding molecules of the invention have two or more different IGF-1R binding specificities. For example, a multispecific IGF-1R binding molecule may comprise a first IGF-1R binding specificity and a second IGF-1R binding specificity. In preferred embodiments, the binding specificities recognize non-overlapping epitopes within the extracellular domain of IGF-1R. In one embodiment, the multi-specific IGF-1R binding molecule of the invention may bind non-overlapping epitopes within the same IGF-1R molecule. In other embodiments, the multi-specific IGF-1R may bind non-overlapping epitopes in separate IGF-1R molecules. In certain embodiments, a multispecific binding molecule of the invention comprises binding specificities from at least one of the antibodies (preferably two) employed in one of the combinations discussed supra. In other embodiments, a multispecific binding molecule of the invention comprises any of the above-identified binding molecules linked or fused to a second binding moiety having a different specificity.

In certain aspects, the multispecific binding molecules of the invention specifically bind to an IGF-1R polypeptide or fragment thereof, or an IGF-1R variant polypeptide, with greater avidity than that of a given reference monospecific antibody. Apparent avidity of the binding molecule and reference antibody may be measured using any method known in the art or described in the Examples (e.g., BIAcore analysis). In specific embodiments, a multispecific binding molecule of the invention binds IGF-1R polypeptides or fragments or variants thereof with a lower k (off) rate than the k (off) rate of the reference antibody (e.g., 2-fold, 5-fold, 10-fold, 50-fold or 100-fold less). In other embodiments, a multispecific binding molecule of the invention binds IGF-1R polypeptides or fragments or variants thereof with an on rate (k(on)) which is greater than that of the reference antibody (e.g., 2-fold, 5-fold, 10-fold, 50-fold or 100-fold more).

In one embodiment, a binding molecule of the invention is multispecific, i.e., has at least one binding specificity that binds to a first target IGF-1R molecule or epitope of the target molecule and at least one second binding specificity that binds to a second, different target IGF-1R molecule or to a second, different epitope of the first target IGF-1R molecule. In certain embodiments, multispecific binding molecules of the invention (e.g. bispecific binding molecules) comprise at least two binding specificities that are independently selected from among the binding specificities described supra. The binding molecule of the invention may bind to an IGF-1R that is present on the surface of a cell or that is soluble.

In one embodiment, the multispecific binding molecules of the invention include those with at least one binding moiety directed against a cell-surface IGF-1R, and at least one binding moiety directed against a soluble IGF-1R molecule. In preferred embodiments, a multispecific binding molecule of the invention has two binding sites that bind to cell surface IGF-1R molecules.

It will be recognized by those of skill in the art that the multispecific binding molecules of the invention may comprise any combination of binding moieties disclosed herein. For example, in certain embodiments, a mutlispecific binding molecule of the invention may comprise at least first and second binding moieties wherein said first and second binding moieties are independently selected from binding moieties derived from the deposited antibodies disclosed herein. In other embodiments, one or more of said binding moieties are scFv molecules independently selected from any one of the scFv molecules (e.g., any one of the stabilized scFv molecules) disclosed herein. In other embodiments, one or more of said binding moieties are antibodies independently selected from among the antibodies disclosed herein. Said antibodies may be of any IgG isotype (e.g., IgG1 or IgG4) or glycosylation state (e.g., glycosylated or aglycosylated).

In one embodiment, a binding molecule of the invention comprises at least one inhibitory IGF-1R binding specificity or binding moiety and at least one allosteric IGF-1R binding specificity or binding moiety. For example, in a preferred embodiment, a binding molecule of the invention comprises at least one competitive binding specificity or binding moiety which competitively blocks binding of IGF-1 and/or IGF-2 to IGF-1R and at least one allosteric binding specificity or binding moiety which allosterically blocks binding of IGF-1 and/or IGF-2 to IGF-1R.

In another embodiment, a binding molecule of the invention comprises at least one allosteric binding specificity or binding moiety which allosterically blocks binding of IGF-1 and IGF-2 to IGF-1R and at least one allosteric binding specificity or binding moiety which allosterically blocks binding of IGF-1 (but not IGF-2) to IGF-1R.

In one embodiment, an IGF-1R binding molecule of the invention is a bispecific IGF-1R binding molecule, e.g., a bispecific antibody, minibody, domain deleted antibody, or fusion protein having binding specificity for more than one epitope, e.g., more than one antigen or more than one epitope on the same antigen. Bispecific IGF-1R binding molecules can bind to two different target sites, e.g., on the same IGF-1R molecule or on different IGF-1R molecules. For example, bispecific molecules of the invention can bind to two different epitopes, e.g., on the same IGF-1R antigen or on two different IGF-1R antigens.

In one embodiment, a bispecific IGF-1R antibody has at least one binding domain specific for at least one epitope on a target polypeptide disclosed herein, i.e., IGF-1R. In one embodiment, a bispecific IGF-1R antibody has at least one binding specificity or binding moiety for a competitive epitope on IGF-1R, and at least one binding specificity for an allosteric epitope on IGF-1R. A bispecific IGF-1R antibody may be a tetravalent antibody that has two binding specificities or binding moieties specific for a first epitope of a target IGF-1R polypeptide disclosed herein and two target binding domains specific for a second epitope of a target IGF-1R target polypeptide. Thus, a tetravalent bispecific IGF-1R antibody may be bivalent for each specificity.

The multi specific binding molecules of the invention may be monovalent for each specificity or multivalent for each specificity. In one embodiment, a bispecific binding molecule of the invention may comprise one binding site that reacts with a first IGF-1R molecule and one binding site that reacts with a second target IGF-1R molecule (e.g. a bispecific antibody molecule, fusion protein, or minibody). In another embodiment, a bispecific binding molecule of the invention may comprise two binding sites that react with a first IGF-1R target molecule and two binding sites that react with a second IGF-1R target molecule (e.g. a bispecific scFv2 tetravalent antibody, tetravalent minibody, or diabody).

In another embodiment, the first and second IGF-1R molecules to which a bispecific binding molecule is capable of binding are located on the same cell or cell type. By crosslinking the first and second receptors on the same cell, the bispecific binding molecules of the invention may inhibit an activity (e.g. signal transduction activity) associated with one or both of the first and second receptors or lead to enhanced receptor downregulation or internalization.

In one embodiment, a multispecific IGF-1R binding molecule of the invention may comprise a binding moiety for an antigen other than IGF-1R. For example, a multispecific binding molecule of the invention may have a binding moiety that is specific for a drug or toxin. In another exemplary embodiment, a multi specific binding molecule of the invention may comprise a binding moiety for IGF-2R or the insulin receptor.

Methods of producing multi specific molecules are well known in the art. For example, recombinant technology can be used to produce multispecific molecules, e.g., diabodies, single-chain diabodies, tandem scFvs, etc. Exemplary techniques for producing multispecific molecules are known in the art (e.g., Kontermann et al. Methods in Molecular Biology Vol. 248: Antibody Engineering: Methods and Protocols. Pp 227-242 US 2003/0207346 A1 and the references cited therein). In one embodiment, a multimeric multi specific molecules are prepared using methods such as those described e.g., in US 2003/0207346 A1 or U.S. Pat. No. 5,821,333, or US2004/0058400.

### VI. Exemplary Forms of Binding Molecules

A Monospecific Binding Molecules

i) IGF-1R Antibodies

In certain embodiments, IGF-1R binding molecules of the invention are antibodies or comprise antibodies as one or more binding moieties within the binding molecule. Antibodies of the present invention can be produced by any method known in the art for the synthesis of antibodies, in particular, by chemical synthesis or preferably, by recombinant expression techniques as described herein. For example, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

Yet other embodiments of the present invention comprise the generation of human or substantially human antibodies in transgenic animals (e.g., mice) that are incapable of endogenous immunoglobulin production (see e.g., U.S. Pat. Nos. 6,075,181, 5,939,598, 5,591,669 and 5,589,369 each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human antibodies upon antigen challenge. Another preferred means of generating human antibodies using SCID mice is disclosed in U.S. Pat. No. 5,811,524 which is incorporated herein by reference. It will be appreciated that the genetic material associated with these human antibodies may also be isolated and manipulated as described herein.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the VH and VL genes can be amplified using, e.g., RT-PCR. The VH and VL genes can be cloned into an antibody expression vector and transfected into cells (e.g., eukaryotic or prokaryotic cells) for expression.

In certain embodiments both the variable and constant regions of IGF-1R antibodies, or antigen-binding fragments, variants, or derivatives thereof are fully human. Fully human antibodies can be made using techniques that are known in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140. Other techniques are known in the art. Fully human antibodies can likewise be produced by various display technologies, e.g., phage display or other viral display systems, as described in more detail elsewhere herein.

Polyclonal antibodies to an epitope of interest can be produced by various procedures well known in the art. For example, an antigen comprising the epitope of interest can be administered to various host animals including, but not limited to, rabbits, mice, rats, chickens, hamsters, goats, donkeys, etc., to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal IGF-1R antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Thus, the term “monoclonal antibody” is not limited to antibodies produced through hybridoma technology. Monoclonal antibodies can be prepared using IGF-1R knockout mice to increase the regions of epitope recognition. Monoclonal antibodies can be prepared using a wide variety of techniques known in the art including the use of hybridoma and recombinant and phage display technology as described elsewhere herein.

Using art recognized protocols, in one example, antibodies are raised in mammals by multiple subcutaneous or intraperitoneal injections of the relevant antigen (e.g., purified IGF-1R or cells or cellular extracts comprising IGF-1R) and an adjuvant. This immunization typically elicits an immune response that comprises production of antigen-reactive antibodies from activated splenocytes or lymphocytes. While the resulting antibodies may be harvested from the serum of the animal to provide polyclonal preparations, it is often desirable to isolate individual lymphocytes from the spleen, lymph nodes or peripheral blood to provide homogenous preparations of monoclonal antibodies (MAbs). Preferably, the lymphocytes are obtained from the spleen. In this well known process (Kohler et al., Nature 256:495 (1975)) the relatively short-lived, or mortal, lymphocytes from a mammal which has been injected with antigen are fused with an immortal tumor cell line (e.g. a myeloma cell line), thus, producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They produce antibodies which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by in vitro assays such as immunoprecipitation, radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, Academic Press, pp 59-103 (1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

Those skilled in the art will also appreciate that DNA encoding antibodies or antibody fragments (e.g., antigen binding sites) may also be derived from antibody libraries, such as phage display libraries. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv OE DAB (individual Fv region from light or heavy chains) or disulfide stabilized Fv antibody domains recombinantly fused to either the phage gene III or gene VIII protein. Exemplary methods are set forth, for example, in EP 368 684 B1; U.S. Pat. No. 5,969,108, Hoogenboom, H. R. and Chames, Immunol. Today 21:371 (2000); Nagy et al. Nat. Med. 8:801 (2002); Huie et al., Proc. Natl. Acad. Sci. USA 98:2682 (2001); Lui et al., J. Mol. Biol. 315:1063 (2002) each of which is incorporated herein by reference. Several publications (e.g., Marks et al., Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al., Nat. Biotechnol. 18:1287 (2000); Wilson et al., Proc. Natl. Acad. Sci. USA 98:3750 (2001); or Irving et al., J. Immunol. Methods 248:31 (2001)). In yet another embodiment, cell surface libraries can be screened for antibodies (Boder et al., Proc. Natl. Acad. Sci. USA 97:10701 (2000); Daugherty et al., J. Immunol. Methods 243:211 (2000)). Yet another exemplary embodiment, high affinity human Fab libraries are designed by combining immunoglobulin sequences derived from human donors with synthetic diversity in selected complementarity determining regions such as CDR H1 and CDR H2 (see, e.g., Hoet et al., Nature Biotechnol., 23:344-348 (2005), which is incorporated herein by reference). Such procedures provide alternatives to traditional hybridoma techniques for the isolation and subsequent cloning of monoclonal antibodies.

In phage display methods, functional antibody domains are displayed on the surface of phage particles which carry the polynucleotide sequences encoding them. For example, DNA sequences encoding VH and VL regions are amplified or otherwise isolated from animal cDNA libraries (e.g., human or murine cDNA libraries of lymphoid tissues) or synthetic cDNA libraries. In certain embodiments, the DNA encoding the VH and VL regions are joined together by an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH or VL regions are usually recombinantly fused to either the phage gene III or gene VIII. Phage expressing an antigen binding domain that binds to an antigen of interest (i.e., an IGF-1R polypeptide or a fragment thereof) can be selected or identified with antigen, e.g., using labeled antigen or antigen bound or captured to a solid surface or bead.

Additional examples of phage display methods that can be used to make antibodies include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Advances in Immunology 57:191-280 (1994); PCT Application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria. For example, techniques to recombinantly produce Fab, Fab′ and F(ab′)₂ fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

For some uses, including in vivo use of antibodies in humans and in vitro detection assays, it may be preferable to use chimeric, humanized, or human antibodies.

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a desired target polypeptide. Monoclonal antibodies directed against the antigen can be obtained from the immunized, transgenic mice using conventional hybridoma technology. The human immunoglobulin transgenes harbored by the transgenic mice rearrange during B-cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar Int. Rev. Immunol. 13:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Abgenix, Inc. (Freemont, Calif.) and GenPharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/Technology 12:899-903 (1988). See also, U.S. Pat. No. 5,565,332.)

Further, antibodies to target polypeptides of the invention can, in turn, be utilized to generate anti-idiotype antibodies that “mimic” target polypeptides using techniques well known to those skilled in the art. (See, e.g., Greenspan & Bona, FASEB J. 7(5):437-444 (1989) and Nissinoff, J. Immunol. 147(8):2429-2438 (1991)). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a desired target polypeptide and/or to bind its ligands/receptors, and thereby block its biological activity.

ii Single Chain Binding Molecules

In other embodiments, a binding molecule of the invention may be a single chain binding molecule (e.g., a singe chain variable region or scFv) or may comprise said single chain binding molecule as a binding moiety. Preferably, the single chain binding molecule specifically or preferentially binds IGF-1R. Techniques described for the production of single chain antibodies (U.S. Pat. No. 4,694,778; Bird, Science 242:423-442 (1988); Huston et al., Proc. Natl. Acad. Sci. USA 85:5879-5883 (1988); and Ward et al., Nature 334:544-554 (1989)) can be adapted to produce single chain binding molecules. Single chain antibodies are formed by linking the heavy and light chain fragments of the Fv region via an amino acid bridge, resulting in a single chain antibody. Techniques for the assembly of functional Fv fragments in E coli may also be used (Skerra et al., Science 242:1038-1041 (1988)).

In certain embodiments, binding molecules of the invention are scFv molecules (e.g., a VH and a VL domain joined by an scFv linker) or comprise such molecules. scFv molecules may be conventional scFv molecules or stabilized scFv molecules. Stabilized scFvs comprising stabilizing mutations, disulfide bonds, or optimized linkers which confer improved stability (e.g., improved thermal stability) to the scFv or to a binding molecule comprising the scFv are described in detail in U.S. patent application Ser. No. 11/725,970, which is incorporated by reference herein in its entirety. In other embodiments, binding molecules of the invention are polypeptides comprising scFv molecules. When a stabilized scFv molecule of the invention is fused to a second molecule, the second molecule may also impart a binding specificity to the fusion protein. Stabilized scFv molecules have improved thermal stability (e.g., melting temperature (Tm) values greater than 54° C. (e.g. 55, 56, 57, 58, 59, 60° C. or greater) or T50 values greater than 39° C. (e.g. greater than 40, 41, 42, 43, 44, 45, 46, 47, 48, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80° C.). In one preferred embodiment, the stabilized scFv molecule has a T50 of greater than 50° C. In another preferred embodiment, the stabilized scFv molecule has a T50 of greater than 60° C.

The stability of scFv molecules of the invention can be determined using methods known in the art, such as those described in U.S. patent application Ser. No. 11/725,970 (US Publication No. 2008/0050370), the contents of which are incorporated by reference herein.

The stability of scFv molecules of the invention or fusion proteins comprising them can be evaluated in reference to the biophysical properties (e.g., thermal stability) of a conventional (non-stabilized) scFv molecule or a binding molecule comprising a conventional scFv molecule. In one embodiment, the binding molecules of the invention have a thermal stability that is greater than about 0.1, about 0.25, about 0.5, about 0.75, about 1, about 1.25, about 1.5, about 1.75, about 2, about 3, about 4, about 5, about 6, about 7, about 8, about 9, or about 10 degrees Celsius than a control binding molecule (eg. a conventional scFv molecule).

In one embodiment, the scFv linker consists of the amino acid sequence (Gly₄Ser)₄ (SEQ ID NO: 135) or comprises a (Gly₄Ser)₄ (SEQ ID NO: 135) sequence. Other exemplary linkers comprise or consist of (Gly₄Ser)₃ (SEQ ID NO: 185) and (Gly₄Ser)₅ (SEQ ID NO: 184) sequences. scFv linkers of the invention can be of varying lengths. In one embodiment, an scFv linker of the invention is from about 5 to about 50 amino acids in length. In another embodiment, an scFv linker of the invention is from about 10 to about 40 amino acids in length. In another embodiment, an scFv linker of the invention is from about 15 to about 30 amino acids in length. In another embodiment, an scFv linker of the invention is from about 17 to about 28 amino acids in length. In another embodiment, an scFv linker of the invention is from about 19 to about 26 amino acids in length. In another embodiment, an scFv linker of the invention is from about 21 to about 24 amino acids in length.

In certain embodiments, the stabilized scFv molecules of the invention comprise at least one disulfide bond which links an amino acid in the VL domain with an amino acid in the VH domain. Cysteine residues are necessary to provide disulfide bonds. Disulfide bonds can be included in an scFv molecule of the invention, e.g., to connect FR4 of VL and FR2 of VH or to connect FR2 of VL and FR4 of VH. Exemplary positions for disulfide bonding include: 43, 44, 45, 46, 47, 103, 104, 105, and 106 of VH and 42, 43, 44, 45, 46, 98, 99, 100, and 101 of VL, Kabat numbering. Exemplary combinations of amino acid positions which are mutated to cysteine residues include: VH44-VL100, VH105-VL43, VH105-VL42, VH44-VL101, VH106-VL43, VH104-VL43, VH44-VL99, VH45-VL98, VH46-VL98, VH103-VL43, VH103-VL44, and VH103-VL45. In one embodiment, a disulfide bond links VH amino acid 44 and VL amino acid 100.

In one embodiment, a stabilized scFv molecule of the invention comprises an scFv linker having the amino acid sequence (Gly₄ Ser)₄ (SEQ ID NO: 135) interposed between a V_{H} domain and a V_{L} domain, wherein the V_{H} and V_{L} domains are linked by a disulfide bond between an amino acid in the V_{H} at amino acid position 44 and an amino acid in the V_{L} at amino acid position 100.

In other embodiments the stabilized scFv molecules of the invention comprise one or more (e.g. 2, 3, 4, 5, or more) stabilizing mutations within a variable domain (VH or VL) of the scFv. In certain embodiments, the stabilizing mutations are introduced into any of the VH or VL variable domains disclosed herein (e.g., a VL domain from a M13-CO6 antibody (SEQ ID NO:78) or M14-G11 antibody (SEQ ID NO:93) or a VH domain from a M13-CO6 antibody (SEQ ID NO:14) or M14-G11 antibody (SEQ ID NO:32)).

In one embodiment, the stabilizing mutation is selected from the group consisting of: a) substitution of an amino acid (e.g., glutamine) at Kabat position 3 of VL, e.g., with an alanine, a serine, a valine, an aspartic acid, or a glycine; (b) substitution of an amino acid (e.g., serine) at Kabat position 46 of VL, e.g., with leucine; (c) substitution of an amino acid (e.g., serine) at Kabat position 49 of VL, e.g., with tyrosine or serine; (d) substitution of an amino acid (e.g., serine or valine) at Kabat position 50 of VL, e.g., with serine, threonine, and arginine, aspartic acid, glycine, or lysine; (e) substitution of amino acids (e.g., serine) at Kabat position 49 and (e.g., serine) at Kabat position 50 of VL, respectively with tyrosine and serine; tyrosine and threonine; tyrosine and arginine; tyrosine and glycine; serine and arginine; or serine and lysine; (f) substitution of an amino acid (e.g., valine) at Kabat position 75 of VL, e.g., with isoleucine; (g) substitution of an amino acid (e.g., proline) at Kabat position 80 of VL, e.g., with serine or glycine; (h) substitution of an amino acid (e.g., phenylalanine) at Kabat position 83 of VL, e.g., with serine, alanine, glycine, or threonine; (i) substitution of an amino acid (e.g., glutamic acid) at Kabat position 6 of VH, e.g., with glutamine; (j) substitution of an amino acid (e.g., lysine) at Kabat position 13 of VH, e.g., with glutamate; (k) substitution of an amino acid (e.g., serine) at Kabat position 16 of VH, e.g., with glutamate or glutamine; (l) substitution of an amino acid (e.g., valine) at Kabat position 20 of VH, e.g., with an isoleucine; (m) substitution of an amino acid (e.g., asparagine) at Kabat position 32 of VH, e.g., with serine; (n) substitution of an amino acid (e.g., glutamine) at Kabat position 43 of VH, e.g, with lysine or arginine; (o) substitution of an amino acid (e.g., methionine) at Kabat position 48 of VH, e.g., with an isoleucine or a glycine; (p) substitution of an amino acid (e.g., serine) at Kabat position 49 of VH, e.g, with glycine or alanine; (q) substitution of an amino acid (e.g., valine) at Kabat position 55 of VH, e.g., with a glycine; (r) substitution of an amino acid (e.g., valine) at Kabat position 67 of VH, e.g., with an isoleucine or a leucine; (s) substitution of an amino acid (e.g., glutamic acid) at Kabat position 72 of VH, e.g., with aspartate or asparagine; (t) substitution of an amino acid (e.g., phenylalanine) at Kabat position 79 of VH, e.g., with serine, valine, or tyrosine; and (u) substitution of an amino acid (e.g., proline) at Kabat position 101 of VH, e.g., with an aspartic acid.

In another embodiment, the stabilizing mutation is selected from the group consisting of: a) substitution of an amino acid (e.g., methionine) at Kabat position 4 of VL, e.g., with leucine; (b) substitution of an amino acid at Kabat position 11 of VL, e.g., with glycine; (c) substitution of an amino acid (e.g., valine) at Kabat position 15 of VL, e.g., with alanine, aspartic acid, glutamic acid, glycine, isoleucine, asparagines, proline, arginine, or serine; (d) substitution of an amino acid at Kabat position 20 of VL, e.g., with arginine; (e) substitution of an amino acid at Kabat position 24 of VL, e.g., with lysine; (f) substitution of an amino acid (e.g., arginine) at Kabat position 30 of VL, e.g., with asparagine, threonine, or tyrosine; (g) substitution of an amino acid (e.g., threnonine) at Kabat position 47 of VL, e.g., with serine; (h) substitution of an amino acid at Kabat position 50 of VL, e.g., with glycine, methionine, or asparagine; (i) substitution of an amino acid (e.g., alanine) at Kabat position 51 of VL, e.g., with glycine; (j) substitution of an amino acid at Kabat position 63 of VL, e.g., with serine; (k) substitution of an amino acid at Kabat position 70 of VL, e.g., with glutamic acid; (l) substitution of an amino acid (e.g., serine) at Kabat position 72 of VL, e.g., with asparagine or tyrosine; (m) substitution of an amino acid (e.g., aspartic acid) at Kabat position 74 of VL, e.g., with serine; (n) substitution of an amino acid at Kabat position 77 of VL, e.g., with glycine; (o) substitution of an amino acid (e.g., isoleucine) at Kabat position 83 of VL, e.g., with aspartic acid, glutamic aci, glycine, methionine, arginine, serine, or valine; (p) substitution of an amino acid at Kabat position 6 of VH, e.g., with glutamine; (q) substitution of an amino acid at Kabat position 21 of VH, e.g., with glutamic acid; (r) substitution of an amino acid (e.g., tryptophan) at Kabat position 47 of VH, e.g., with phenylalanine; (s) substitution of an amino acid at Kabat position 49 of VH, e.g., with alanine; (t) substitution of an amino acid (e.g., arginine) at Kabat position 83 of VH, e.g., with lysine or threonine; and (u) substitution of an amino acid (e.g., threonine) at Kabat position 110 of VH, e.g., with valine.

In yet another embodiment, the scFv molecule comprises stabilizing mutations as compared to a conventional scFv molecule, wherein said mutations are present at: (i) VL amino acid position 50, (ii) VL amino acid position 83; (iii) VH amino acid position 6 and (iv) VH amino acid position 49 (Kabat numbering convention). In another embodiment, said stabilizing mutations are selected from the group consisting of: 6Q, 21E, 47F, 49A, 49G, 83K, 83T and 110V.

In one embodiment, the invention provided a stabilized scFv molecule comprising a sequence encoded by a polynucleotide that is at least 80%, 85%, 90% 95% or 100% identical to a reference polynucleotide sequence selected from the group consisting of SEQ ID NOs: 123, 125 or 127. In other exemplary embodiments, the stabilized scFv molecule comprises an amino acid sequence that is at least 80%, 85%, 90% 95% or 100% identical to an amino acid sequence selected from the group consisting of SEQ ID NOs: 124, 126 or 128. In certain embodiments, the stabilized scFv specifically or preferentially binds to IGF-1R.

It will be appreciated by those skilled in the art that any of the stabilizing mutations discussed above can be introduced into the appropriate variable region (VL or VH) of other, non-scFv, binding molecules (e.g., any of the IGF-1R binding molecules disclosed herein) in order to achieve similar increases in protein stability. For example, one or more of the stabilizing mutations disclosed supra can be introduced into the equivalent amino acid position (according to Kabat numbering) of a VL or VH domain of a Fab molecule or full-length IgG anibody molecule to increase the stability of the molecule.

iii Single Domain Binding Molecules

In certain embodiments, the binding molecule is or comprises a single domain binding molecule (e.g. a single domain antibody), also known as nanobodies. Exemplary single domain molecules include an isolated heavy chain variable domain (V_{H}) of an antibody, i.e., a heavy chain variable domain, without a light chain variable domain, and an isolated light chain variable domain (V_{L}) of an antibody, i.e., a light chain variable domain, without a heavy chain variable domain. Exemplary single-domain antibodies employed in the binding molecules of the invention include, for example, the Camelid heavy chain variable domain (about 118 to 136 amino acid residues) as described in Hamers-Casterman, et al., Nature 363:446-448 (1993), and Dumoulin, et al., Protein Science 11:500-515 (2002). Multimers of single-domain antibodies are also within the scope of the invention. Other single domain antibodies include shark antibodies (e.g., shark Ig-NARs). Shark Ig-NARs comprise a homodimer of one variable domain (V-NAR) and five C-like constant domains (C-NAR), wherein diversity is concentrated in an elongated CDR3 region varying from 5 to 23 residues in length In camelid species (e.g., llamas), the heavy chain variable region, referred to as VHH, forms the entire antigen-binding domain. The main differences between camelid VHH variable regions and those derived from conventional antibodies (VH) include (a) more hydrophobic amino acids in the light chain contact surface of VH as compared to the corresponding region in VHH, (b) a longer CDR3 in VHH, and (c) the frequent occurrence of a disulfide bond between CDR1 and CDR3 in VHH. Methods for making single domain binding molecules are described in U.S. Pat. Nos. 6,005,079 and 6,765,087, both of which are incorporated herein by reference.

iv Minibodies

In certain embodiments, the binding molecules of the invention are minibodies or comprise minibodies. Minibodies can be made using methods described in the art (see e.g., U.S. Pat. No. 5,837,821 or WO 94/09817A1). In certain embodiments, a minibody is a binding molecule that comprises only 2 complementarity determining regions (CDRs) of a naturally or non-naturally (e.g., mutagenized) occurring heavy chain variable domain or light chain variable domain, or combination thereof. An example of such a minibody is described by Pessi et al., Nature 362:367-369 (1993). Another exemplary minibody comprises a scFv molecule that is linked or fused to a CH3 domain or a complete Fc region. Multimers of minibodies are also within the scope of the invention.

v. Non-Immunoglobulin Binding Molecules

In certain embodiments, the binding molecules of the invention are non-immunoglobulin binding molecules or comprise one or more binding moieties derived from a non-immunoglobulin binding molecule. As used herein, the term “non-immunoglobulin binding molecules” are binding molecules whose binding sites comprise a portion (e.g., a scaffold or framework) which are derived from a polypeptide other than an immunoglobulin, but which may be engineered (e.g., mutagenized) to confer a desired binding specificity.

Non-immunoglobulin binding molecules can comprise binding site portions that are derived from a member of the immunoglobulin superfamily that is not an immunoglobulin (e.g. a T-cell receptor or a cell-adhesion protein (e.g., CTLA-4, N-CAM, telokin)). Such binding molecules comprise a binding site portion which retains the conformation of an immunoglobulin fold and is capable of specifically binding an IGF1-R eptitope. In other embodiments, non-immunoglobulin binding molecules of the invention also comprise a binding site with a protein topology that is not based on the immunoglobulin fold (e.g. such as ankyrin repeat proteins or fibronectins) but which nonetheless are capable of specifically binding to a target (e.g. an IGF-1R epitope).

Non-immunoglobulin binding molecules may be identified by selection or isolation of a target-binding variant from a library of binding molecules having artificially diversified binding sites. Diversified libraries can be generated using completely random approaches (e.g., error-prone PCR, exon shuffling, or directed evolution) or aided by art-recognized design strategies. For example, amino acid positions that are usually involved when the binding site interacts with its cognate target molecule can be randomized by insertion of degenerate codons, trinucleotides, random peptides, or entire loops at corresponding positions within the nucleic acid which encodes the binding site (see e.g., U.S. Pub. No. 20040132028). The location of the amino acid positions can be identified by investigation of the crystal structure of the binding site in complex with the target molecule. Candidate positions for randomization include loops, flat surfaces, helices, and binding cavities of the binding site. In certain embodiments, amino acids within the binding site that are likely candidates for diversification can be identified by their homology with the immunoglobulin fold. For example, residues within the CDR-like loops of fibronectin may be randomized to generate a library of fibronectin binding molecules (see, e.g., Koide et al., J. Mol. Biol., 284: 1141-1151 (1998)). Other portions of the binding site which may be randomized include flat surfaces. Following randomization, the diversified library may then be subjected to a selection or screening procedure to obtain binding molecules with the desired binding characteristics, e.g. specific binding to an IGF-1R epitope described supra. For example, selection can be achieved by art-recognized methods such as phage display, yeast display, or ribosome display.

In one embodiment, a binding molecule of the invention comprises a binding site from a fibronectin binding molecule. Fibronectin binding molecules (e.g., molecules comprising the Fibronectin type I, II, or III domains) display CDR-like loops which, in contrast to immunoglobulins, do not rely on intra-chain disulfide bonds. Methods for making fibronectin binding polypeptides are described, for example, in WO 01/64942 and in U.S. Pat. Nos. 6,673,901, 6,703,199, 7,078,490, and 7,119,171, which are incorporated herein by reference.

In another embodiment, a binding molecule of the invention comprises a binding site from an affibody. Affibodies are derived from the immunoglobulin binding domains of staphylococcal Protein A (SPA) (see e.g., Nord et al., Nat. Biotechnol., 15: 772-777 (1997)). Affibody binding sites employed in the invention may be synthesized by mutagenizing an SPA-related protein (e.g., Protein Z) derived from a domain of SPA (e.g., domain B) and selecting for mutant SPA-related polypeptides having binding affinity for an IGF-1R epitope. Other methods for making affibody binding sites are described in U.S. Pat. Nos. 6,740,734 and 6,602,977 and in WO 00/63243, each of which is incorporated herein by reference.

In another embodiment, a binding molecule of the invention comprises a binding site from an anticalin. Anticalins (also known as lipocalins) are members of a diverse β-barrel protein family whose function is to bind target molecules in their barrel/loop region. Lipocalin binding sites may be engineered to bind an IGF-1R epitope by randomizing loop sequences connecting the strands of the barrel (see e.g., Schlehuber et al., Drug Discov. Today, 10: 23-33 (2005); Beste et al., PNAS, 96: 1898-1903 (1999). Anticalin binding sites employed in the binding molecules of the invention may be obtainable starting from polypeptides of the lipocalin family which are mutated in four segments that correspond to the sequence positions of the linear polypeptide sequence comprising amino acid positions 28 to 45, 58 to 69, 86 to 99 and 114 to 129 of the Bilin-binding protein (BBP) of Pieris brassica. Other methods for making anticalin binding sites are described in WO99/16873 and WO 05/019254, each of which is incorporated herein by reference.

In another embodiment, a binding molecule of the invention comprises a binding site from a cysteine-rich polypeptide. Cysteine-rich domains employed in the practice of the present invention typically do not form an α-helix, a β sheet, or a β-barrel structure. Typically, the disulfide bonds promote folding of the domain into a three-dimensional structure. Usually, cysteine-rich domains have at least two disulfide bonds, more typically at least three disulfide bonds. An exemplary cysteine-rich polypeptide is an A domain protein. A-domains (sometimes called “complement-type repeats”) contain about 30-50 or 30-65 amino acids. In some embodiments, the domains comprise about 35-45 amino acids and in some cases about 40 amino acids. Within the 30-50 amino acids, there are about 6 cysteine residues. Of the six cysteines, disulfide bonds typically are found between the following cysteines: C1 and C3, C2 and C5, C4 and C6. The A domain constitutes a ligand binding moiety. The cysteine residues of the domain are disulfide linked to form a compact, stable, functionally independent moiety. Clusters of these repeats make up a ligand binding domain, and differential clustering can impart specificity with respect to the ligand binding. Exemplary proteins containing A-domains include, e.g., complement components (e.g., C6, C7, C8, C9, and Factor I), serine proteases (e.g., enteropeptidase, matriptase, and corin), transmembrane proteins (e.g., ST7, LRP3, LRP5 and LRP6) and endocytic receptors (e.g., Sortilin-related receptor, LDL-receptor, VLDLR, LRP1, LRP2, and ApoER2). Methods for making A domain proteins of a desired binding specificity are disclosed, for example, in WO 02/088171 and WO 04/044011, each of which is incorporated herein by reference.

In other embodiments, a binding molecule of the invention comprises a binding site from a repeat protein. Repeat proteins are proteins that contain consecutive copies of small (e.g., about 20 to about 40 amino acid residues) structural units or repeats that stack together to form contiguous domains. Repeat proteins can be modified to suit a particular target binding site by adjusting the number of repeats in the protein. Exemplary repeat proteins include designed ankyrin repeat proteins (i.e., a DARPins) (see e.g., Binz et al., Nat. Biotechnol., 22: 575-582 (2004)) or leucine-rich repeat proteins (i.e., LRRPs) (see e.g., Pancer et al., Nature, 430: 174-180 (2004)). All so far determined tertiary structures of ankyrin repeat units share a characteristic composed of a β-hairpin followed by two antiparallel α-helices and ending with a loop connecting the repeat unit with the next one. Domains built of ankyrin repeat units are formed by stacking the repeat units to an extended and curved structure. LRRP binding sites from part of the adaptive immune system of sea lampreys and other jawless fishes and resemble antibodies in that they are formed by recombination of a suite of leucine-rich repeat genes during lymphocyte maturation. Methods for making DARpin or LRRP binding sites are described in WO 02/20565 and WO 06/083275, each of which is incorporated herein by reference.

Other non-immunoglobulin binding sites which may be employed in binding molecules of the invention include binding sites derived from Src homology domains (e.g. SH2 or SH3 domains), PDZ domains, beta-lactamase, high affinity protease inhibitors, or small disulfide binding protein scaffolds such as scorpion toxins. Methods for making binding sites derived from these molecules have been disclosed in the art, see e.g., Panni et al, J. Biol. Chem., 277: 21666-21674 (2002), Schneider et al., Nat. Biotechnol., 17: 170-175 (1999); Legendre et al., Protein Sci., 11:1506-1518 (2002); Stoop et al., Nat. Biotechnol., 21: 1063-1068 (2003); and Vita et al., PNAS, 92: 6404-6408 (1995). Yet other binding sites may be derived from a binding domain selected from the group consisting of an EGF-like domain, a Kringle-domain, a PAN domain, a Gla domain, a SRCR domain, a Kunitz/Bovine pancreatic trypsin Inhibitor domain, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an Immunoglobulin-like domain, a C-type lectin domain, a MAM domain, a von Willebrand factor type A domain, a Somatomedin B domain, a WAP-type four disulfide core domain, a F5/8 type C domain, a Hemopexin domain, a Laminin-type EGF-like domain, a C2 domain, and other such domains known to those of ordinary skill in the art, as well as derivatives and/or variants thereof.

vi. Binding Molecule Fragments

Unless it is specifically noted, as used herein a “fragment” in reference to a binding molecule refers to an antigen-binding fragment, i.e., a portion of the binding which specifically binds to the antigen. In one embodiment, a binding molecule of the invention is an antibody fragment or comprises such a fragment. Antibody fragments that recognize specific epitopes may be generated by known techniques. For example, Fab and F(ab′)₂ fragments may be produced recombinantly or by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

B. Multispecific Binding Molecules

Multispecific binding molecules of the invention may comprise at least two binding sites or binding moieties, wherein at least one of the binding sites or binding moieties is derived from or comprises one of the monospecific binding molecules described supra or a binding moiety thereof. In certain embodiments, at least one binding site of a multispecific binding molecule of the invention is an antigen binding region of an antibody or an antigen binding fragment thereof (e.g. an antibody or antigen binding fragment described supra).

(i) Bispecific Antibodies

In certain embodiments, a multispecific binding molecule of the invention is bispecific. Bispecific binding molecules may be bivalent or of a higher valency (e.g., trivalent, tetravalent, hexavalent, and the like). Bispecific bivalent antibodies, and methods of making them, are described, for instance in U.S. Pat. Nos. 5,731,168; 5,807,706; 5,821,333; and U.S. Appl. Publ. Nos. 2003/020734 and 2002/0155537, the disclosures of all of which are incorporated by reference herein. Bispecific tetravalent antibodies and methods of making them are described, for instance, in WO 02/096948 and WO 00/44788, the disclosures of both of which are incorporated by reference herein. See generally, PCT publications WO 93/17715; WO 92/08802; WO 91/00360; WO 92/05793; Tutt et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Bispecific antibodies of the invention may comprise any one of the monospecific binding molecules (e.g., any one of the antibodies described supra) or any one of the binding moieties disclosed supra. For example, in certain embodiments, a bispecific antibody may comprise any one of the deposited antibodies disclosed herein as a first binding moiety and any one of the scFv molecules disclosed herein as a second binding moiety, provided that said first and second binding moieties have different binding specificities. In one exemplary embodiment, a bispecific antibody of the invention may comprise an M14.G11 IgG antibody that is linked or fused to one or more scFv molecules (e.g., one or more stabilized scFv molecules) derived from the variable regions of an M13.C06 IgG antibody. In another exemplary embodiment, a bispecific antibody may comprise an M13.C06 IgG antibody that is linked or fused to scFv molecules (e.g., stabilized scFv molecules) derived from the variable regions of an M14.G11 antibody. The M14.G11 IgG antibody or M13.C06 IgG antibody of said bispecific antibody may comprise the heavy chain constant regions of any isotype (e.g., an IgG1, IgG2, IgG3 or IgG4 isotype). In certain embodiments, the heavy chain constant regions are fully glycosylated. In other embodiments, the heavy chain constant regions lack glycosylation (e.g., the IgG antibody is an “agly” antibody, e.g., an agly IgG1 or agly IgG4 antibody). In one embodiment, scFvs are linked or fused to the mature N-terminus of a heavy chain of the M14.G11 or M13.C06 IgG antibody. In other embodiments, the scFvs are linked or fused to a mature C-terminus of the IgG antibody heavy chain. In yet other embodiments, the scFvs are linked or fused to the mature N-terminus of a light chain of the M14.G11 or M13.C06 IgG antibody. In certain embodiments, a gly/ser connecting peptide (e.g., a (Gly₄Ser)₅ (SEQ ID NO: 184) linker) may be used to connect the scFvs to the IgG antibody of said bispecific antibody.

In a further exemplary embodiment, the present invention provides a bispecific binding molecule comprising a heavy chain encoded by a polynucleotide that is at least 80%, 85%, 90% 95% or 100% identical to a reference polynucleotide sequence selected from the group consisting of SEQ ID NOs: 132, 136, 141 or 143. In other exemplary embodiments, the bispecific molecule comprises a heavy chain that is at least 80%, 85%, 90% 95% or 100% identical to an amino acid sequence selected from the group consisting of SEQ ID NOs: 133, 137, 142, or 144. In certain embodiments, the bispecific amino acid further comprises a light chain encoded by a polynucleotide that is at least 80%, 85%, 90% 95% or 100% identical to a reference polynucleotide sequence of SEQ ID NO: 129 or SEQ ID NO: 139. In other exemplary embodiments, the bispecific molecule further comprises a light chain that is at least 80%, 85%, 90% 95% or 100% identical to SEQ ID NO:130 or SEQ ID NO:140. In certain embodiments, an bispecific antibody specifically or preferentially binds to IGF-1R.

(ii) scFv-Containing Multispecific Binding Molecules

In one embodiment, the multispecific binding molecules of the invention are multispecific binding molecules comprising at least one scFv molecule, e.g. any one of the scFv molecules described herein. In other embodiments, the multispecific binding molecules of the invention comprise two scFv molecules, e.g. a bispecific scFv (Bis-scFv). Said scFv molecules may be the same or different. In certain embodiments, the scFv molecule is a conventional scFv molecule. In other embodiments, the scFv molecule is a stabilized scFv molecule described supra. In certain embodiments, a multispecific binding molecule may be created by linking a scFv molecule (e.g., a stabilized scFv molecule) having with any a parent binding molecule selected from any of the binding molecules described supra, wherein the scFv molecule and the parent binding molecule have different IGF-1R binding moieties (e.g., a competitive binding moiety and an allosteric binding moiety). For example, a binding molecule of the invention may comprise a scFv molecule with a first binding specificity linked to a second scFv molecule or a non-scFv binding molecule (e.g., an IGF-1R antibody), that imparts second IGF-1R binding specificity. In one embodiment, a binding molecule of the invention is a naturally occurring antibody to which a stabilized scFv molecule has been fused.

When a stabilized scFv is linked to a parent binding molecule, linkage of the stabilized scFv molecule preferably improves the thermal stability of the binding molecule by at least about 2° C. or 3° C. In one embodiment, the scFv-containing binding molecule of the invention has a 1° C. improved thermal stability as compared to a conventional binding molecule. In another embodiment, a binding molecule of the invention has a 2° C. improved thermal stability as compared to a conventional binding molecule. In another embodiment, a binding molecule of the invention has a 4, 5, 6° C. improved thermal stability as compared to a conventional binding molecule.

In one embodiment, the binding molecules of the invention are stabilized “antibody” or “immunoglobulin” molecules, e.g., naturally occurring antibody or immunoglobulin molecules (or an antigen binding fragment thereof) or genetically engineered antibody molecules that bind antigen in a manner similar to antibody molecules and that comprise an scFv molecule of the invention. As used herein, the term “immunoglobulin” includes a polypeptide having a combination of two heavy and two light chains whether or not it possesses any relevant specific immunoreactivity.

In one embodiment, the multispecific binding molecules of the invention comprise at least one scFv (e.g. 2, 3, or 4 scFvs, e.g., stabilized scFvs) linked to the C-terminus of an antibody heavy chain, wherein the scFv and antibody have different binding specificities. In another embodiment, the multispecific binding molecules of the invention comprise at least one scFv (e.g. 2, 3, or 4 scFvs, e.g., stabilized scFvs) linked to the N-terminus of an antibody heavy chain, wherein the scFv and antibody have different binding specificities. In another embodiment, the multispecific binding molecules of the invention comprise at least one scFv (e.g. 2, 3, or 4 scFvs or stabilized scFvs) linked to the N-terminus of an antibody light chain, wherein the scFv and antibody have different binding specificities. In another embodiment, the multispecific binding molecules of the invention comprise at least one scFv (e.g., 2, 3, or 4 scFvs or stabilized scFvs) linked to the N-terminus of the antibody heavy chain or light chain and at least one scFv (e.g., 2, 3, or 4 scFvs or stabilized scFvs) linked to the C-terminus of the heavy chain, wherein the scFvs have different binding specificity.

(iii) Multivalent Minibodies

In one embodiment, the multispecific binding molecules of the invention are multivalent minibodies having at least one scFv fragment with a first binding specificity and at least one scFv with a second binding specificity. In preferred embodiments, at least one of the scFv molecules is stabilized. An exemplary bispecific bivalent minibody construct comprises a CH3 domain fused at its N-terminus to a connecting peptide which is fused at its N-terminus to a VH domain which is fused via its N-terminus to a (Gly4Ser)n (SEQ ID NO: 182) flexible linker which is fused at its N-terminus to a VL domain. In certain embodiments, multivalent minibodies may be biavalent, trivalent (e.g., triabodies), bispecific (e.g., diabodies), or tetravalent (e.g., tetrabodies).

In another embodiment, the binding molecules of the invention are scFv tetravalent minibodies, with each heavy chain portion of the scFv tetravalent minibody containing first and second scFv fragments having different binding specificities. In preferred embodiments at least one of the scFv molecules is stabilized. Said second scFv fragment may be linked to the N-terminus of the first scFv fragment (e.g. bispecific N_{H} scFv tetravalent minibodies or bispecific N_{L} scFv tetravalent minibodies). Alternatively, the second scFv fragment may be linked to the C-terminus of said heavy chain portion containing said first scFv fragment (e.g. bispecific C-scFv tetravalent minibodies). Where the first and second scFv fragments of a first heavy chain portion of a bispecific tetravalent minibody bind the same target IGF-1R molecule, at least one of the first and second scFv fragments of the second heavy chain portion of the bispecific tetravalent minibody may bind the same or different IGF-1R target molecule.

(iv) Multispecific Diabodies

In other embodiments, the binding molecules of the invention are multispecific diabodies. In one embodiment, the multispecific binding molecules of the invention are bispecific diabodies, with each arm of the diabody comprising tandem scFv fragments. In preferred embodiments, at least one of the scFv fragments is stabilized. In one embodiment, a bispecific diabody may comprise a first arm with a first binding specificity and a second arm with a second binding specificity. In another embodiment, each arm of the diabody may comprise a first scFv fragment with a first binding specificity and a second scFv fragment with a second binding specificity. In certain embodiments, a multispecific diabody can be directly fused to complete Fc region or an Fc portion (e.g. a CH3 domain).

(v) scFv2 Tetravalent Antibodies

In other embodiments, the multi specific binding molecules of the invention are scFv2 tetravalent antibodies with each heavy chain portion of the scFv2 tetravalent antibody containing an scFv molecule. Said scFv molecules may be independently selected from any one of the scFv molecules disclosed herein. In preferred embodiments, at least one of the scFv molecules are stabilized. The scFv fragments may be linked to the N-termini of a variable region of the heavy chain portions (e.g. bispecific N_{H} scFv2 tetravalent antibodies or bispecific N_{L} scFv2 tetravalent antibodies). Alternatively, the scFv fragments may be linked to the C-termini of the heavy chain portions of the scFv2 tetravalent antibody. Each heavy chain portion of the scFv2 tetravalent antibody may have variable regions and scFv fragments that bind the same or different target IGF-1R molecule or epitope. Where the scFv fragment and variable region of a first heavy chain portion of a bispecific scFc2 tetravalent antibody bind the same target molecule or epitope, at least one of the first and second scFv fragments of the second heavy chain portion of the bispecific tetravalent minibody binds a different target molecule or epitope.

(vi) Multispecific Binding Molecule Fragments

In certain embodiments, binding molecule fragments of the invention may be made to be multispecific. Multispecific binding molecules of the invention include bispecific Fab2 or multispecific (e.g. trispecific) Fab3 molecules. For example, a multispecific binding molecule fragment may comprise chemically conjugated multimers (e.g. dimers, trimers, or tetramers) of Fab or scFv molecules having different specificities.

(vii) Tandem Variable Domain Binding Molecules

In other embodiments, the multispecific binding molecule of the invention may comprise a binding molecule comprising tandem antigen binding sites. For example, a variable domain may comprise an antibody heavy chain that is engineered to include at least two (e.g., two, three, four, or more) variable heavy domains (VH domains) that are directly fused or linked in series, and an antibody light chain that is engineered to include at least two (e.g., two, three, four, or more) variable light domains (VL domains) that are direct fused or linked in series. The VH domains interact with corresponding VL domains to forms a series of antigen binding sites wherein at least two of the binding sites bind different epitopes of IGF-1R. For example, one of the binding sites may cross-react with a competitive epitope described supra, while another antigen binding site cross-reacts with an allosteric eptitope described supra. Tandem variable domain binding molecules may comprise two or more of heavy or light chains and are of higher order valency (e.g., bivalent or tetravalent). Methods for making tandem variable domain binding molecules are known in the art, see e.g. WO 2007/024715.

(viii) Dual Specificity Binding Molecules

In other embodiments, the multispecific binding molecule of the invention may comprise a single binding site having dual binding specificity. For example, a dual specificity binding molecule of the invention may comprise a binding site which cross-reacts with a competitive epitope described supra and an allosteric eptiope described supra. In another embodiment, a dual specificity binding molecule of the invention may comprise a binding site which cross-reacts with any two of the allosteric epitopes described supra (e.g., an allosteric eptiope which allosterically blocks IGF-1 and IGF-2 and an allosteric epitope which allosterically blocks IGF-1, but not IGF-2). Art-recognized methods for producing dual specificity binding molecules are known in the art. For example, dual specificity binding molecules can be isolated by screening for binding molecules which bind both a first epitope and counter-screening the isolated binding molecules for the ability to bind to a second epitope.

(ix) Multispecific Fusion Proteins

In another embodiment, a multispecific binding molecule of the invention is a multispecific fusion protein. As used herein the phrase “multispecific fusion protein” designates fusion proteins (as hereinabove defined) having at least two binding specificities described supra. Multispecific fusion proteins can be assembled, e.g., as heterodimers, heterotrimers or heterotetramers, essentially as disclosed in WO 89/02922 (published Apr. 6, 1989), in EP 314, 317 (published May 3, 1989), and in U.S. Pat. No. 5,116,964 issued May 2, 1992. Preferred multispecific fusion proteins are bispecific. In certain embodiments, at least of the binding specificities of the multispecific fusion protein comprises an scFv, e.g., a stabilized scFv.

A variety of other multivalent antibody constructs may be developed by one of skill in the art using routine recombinant DNA techniques, for example as described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; Beidler et al. (1988) J. Immunol. 141:4053-4060; and Winter and Milstein, Nature, 349, pp. 293-99 (1991)). Preferably non-human antibodies are “humanized” by linking the non-human antigen binding domain with a human constant domain (e.g. Cabilly et al., U.S. Pat. No. 4,816,567; Morrison et al., Proc. Natl. Acad. Sci. U.S.A., 81, pp. 6851-55 (1984)).

Other methods which may be used to prepare multivalent antibody constructs are described in the following publications: Ghetie, Maria-Ana et al. (2001) Blood 97:1392-1398; Wolff, Edith A. et al. (1993) Cancer Research 53:2560-2565; Ghetie, Maria-Ana et al. (1997) Proc. Natl. Acad. Sci. 94:7509-7514; Kim, J. C. et al. (2002) Int. J. Cancer 97(4):542-547; Todorovska, Aneta et al. (2001) Journal of Immunological Methods 248:47-66; Coloma M. J. et al. (1997) Nature Biotechnology 15:159-163; Zuo, Zhuang et al. (2000) Protein Engineering (Suppl.) 13(5):361-367; Santos A. D., et al. (1999) Clinical Cancer Research 5:3118s-3123s; Presta, Leonard G. (2002) Current Pharmaceutical Biotechnology 3:237-256; van Spriel, Annemiek et al., (2000) Review Immunology Today 21(8) 391-397.

C. Modified Binding Molecules

In certain embodiments, at least one of the binding molecules of the invention (e.g., a multispecific binding molecule of the invention or a monospecific binding molecule employed in a combination of the invention) may comprise one or more modifications. Modified forms of IGF-1R binding molecules of the invention can be made from whole precursor or parent antibodies using techniques known in the art.

In certain embodiments, modified IGF-1R binding molecules of the present invention are polypeptides which have been altered so as to exhibit additional features not found on the native polypeptide. In one embodiment, one or more residues of the binding molecule may chemically derivatized by reaction of a functional side group. In one embodiment, a binding molecule may be modified to include one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

In one embodiment, an IGF-1R binding molecule of the invention comprises a synthetic constant region wherein one or more domains are partially or entirely deleted (“domain-deleted binding molecules”). In certain embodiments compatible modified binding molecules will comprise domain deleted constructs or variants wherein the entire CH2 domain has been removed (ΔCH2 constructs). For other embodiments a short connecting peptide may be substituted for the deleted domain to provide flexibility and freedom of movement for the variable region. Those skilled in the art will appreciate that such constructs are particularly preferred due to the regulatory properties of the CH2 domain on the catabolic rate of the antibody. Domain deleted constructs can be derived using a vector encoding an IgG₁ human constant domain (see, e.g., WO 02/060955A2 and WO02/096948A2). This vector is engineered to delete the CH2 domain and provide a synthetic vector expressing a domain deleted IgG₁ constant region.

In one embodiment, an IGF-1R binding molecule of the invention comprises an immunoglobulin heavy chain having deletion or substitution of a few or even a single amino acid as long as it permits association between the monomeric subunits. For example, the mutation of a single amino acid in selected areas of the CH2 domain may be enough to substantially reduce Fc binding and thereby increase tumor localization. Similarly, it may be desirable to simply delete that part of one or more constant region domains that control the effector function (e.g. complement binding) to be modulated. Such partial deletions of the constant regions may improve selected characteristics of the antibody (serum half-life) while leaving other desirable functions associated with the subject constant region domain intact. Moreover, as alluded to above, the constant regions of the binding molecule may be synthetic through the mutation or substitution of one or more amino acids that enhances the profile of the resulting construct. In this respect it may be possible to disrupt the activity provided by a conserved binding site (e.g. Fc binding) while substantially maintaining the configuration and immunogenic profile of the modified binding molecule. Yet other embodiments comprise the addition of one or more amino acids to the constant region to enhance desirable characteristics such as effector function or provide for more cytotoxin or carbohydrate attachment. In such embodiments it may be desirable to insert or replicate specific sequences derived from selected constant region domains.

The present invention also provides binding molecule that comprise, consist essentially of, or consist of, variants (including derivatives) of binding moieties (e.g., the VH regions and/or VL regions of an antibody molecule) described herein, which binding moieties or fragments thereof immunospecifically bind to an IGF-1R polypeptide or fragment or variant thereof. Standard techniques known to those of skill in the art can be used to introduce mutations in the nucleotide sequence encoding an IGF-1R binding molecule, including, but not limited to, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH region, VH-CDR1, VH-CDR2, VH-CDR3, VL region, VL-CDR1, VL-CDR2, or VL-CDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind an IGF-1R polypeptide).

For example, it is possible to introduce mutations only in framework regions or only in CDR regions of a binding molecule of the invention (e.g., an antibody molecule). Introduced mutations may be silent or neutral missense mutations, i.e., have no, or little, effect on the ability to bind antigen, indeed some such mutations do not alter the amino acid sequence whatsoever. These types of mutations may be useful to optimize codon usage, or improve a hybridoma's antibody production. Codon-optimized coding regions encoding IGF-1R binding molecules of the present invention are disclosed elsewhere herein. Alternatively, non-neutral missense mutations may alter a binding molecule's ability to bind antigen. For example, in an antibody the location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein may routinely be expressed and the functional and/or biological activity of the encoded protein, (e.g., ability to immunospecifically bind at least one epitope of an IGF-1R polypeptide) can be determined using techniques described herein or by routinely modifying techniques known in the art.

(i) Covalent Attachment

IGF-1R binding molecules of the invention may be modified, e.g., by the covalent attachment of a molecule to the binding molecule such that covalent attachment does not prevent the binding molecule from specifically binding to its cognate epitope. For example, but not by way of limitation, the binding molecules of the invention may be modified by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

As discussed in more detail elsewhere herein, binding molecules of the invention may further be recombinantly fused to a heterologous polypeptide at the N- or C-terminus or chemically conjugated (including covalent and non-covalent conjugations) to polypeptides or other compositions. For example, IGF-1R-specific IGF-1R binding molecules may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

An IGF-1R binding molecule of the invention can be composed of amino acids joined to each other by peptide bonds or modified peptide bonds, i.e., peptide isosteres, and may contain amino acids other than the 20 gene-encoded amino acids. IGF-1R-specific binding molecules may be modified by natural processes, such as posttranslational processing, or by chemical modification techniques which are well known in the art. Such modifications are well described in basic texts and in more detailed monographs, as well as in a voluminous research literature. Modifications can occur anywhere in the IGF-1R-specific binding molecule, including the peptide backbone, the amino acid side-chains and the amino or carboxyl termini, or on moieties such as carbohydrates. It will be appreciated that the same type of modification may be present in the same or varying degrees at several sites in a given IGF-1R-specific binding molecule. Also, a given IGF-1R-specific binding molecule may contain many types of modifications. IGF-1R-specific binding molecule may be branched, for example, as a result of ubiquitination, and they may be cyclic, with or without branching. Cyclic, branched, and branched cyclic IGF-1R-specific binding molecule may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, T. E. Creighton, W.H. Freeman and Company, New York 2nd Ed., (1993); Posttranslational Covalent Modification Of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)).

The present invention also provides for fusion proteins comprising an IGF-1R binding molecule, and a heterologous polypeptide. The heterologous polypeptide to which the antibody is fused may be useful for function or is useful to target the IGF-1R polypeptide expressing cells. In one embodiment, a fusion protein of the invention comprises, consists essentially of, or consists of, a polypeptide having the amino acid sequence of any one or more of the binding sites of a binding molecule of the invention and a heterologous polypeptide sequence. In another embodiment, a fusion protein for use in the diagnostic and treatment methods disclosed herein comprises, consists essentially of, or consists of a polypeptide having the amino acid sequence of any one, two, three of the VH-CDRs of an IGF-1R-specific binding molecule, or the amino acid sequence of any one, two, three of the VL-CDRs of an IGF-1R-specific binding molecule, and a heterologous polypeptide sequence. In one embodiment, the fusion protein comprises a polypeptide having the amino acid sequence of a VH-CDR3 of an IGF-1R-specific binding molecule of the present invention, and a heterologous polypeptide sequence, which fusion protein specifically binds to at least one epitope of IGF-1R. In another embodiment, a fusion protein comprises a polypeptide having the amino acid sequence of at least one VH region of an IGF-1R-specific binding molecule of the invention and the amino acid sequence of at least one VL region of an IGF-1R-specific binding molecule of the invention or fragments, derivatives or variants thereof, and a heterologous polypeptide sequence. Preferably, the VH and VL regions of the fusion protein correspond to a single source binding molecule which specifically binds at least one epitope of IGF-1R. In yet another embodiment, a fusion protein for use in the diagnostic and treatment methods disclosed herein comprises a polypeptide having the amino acid sequence of any one, two, three or more of the VH CDRs of an IGF-1R-specific binding molecule and the amino acid sequence of any one, two, three or more of the VL CDRs of an IGF-1R-specific binding molecule, and a heterologous polypeptide sequence. Preferably, two, three, four, five, six, or more of the VH-CDR(s) or VL-CDR(s) correspond to single source binding molecule of the invention. Nucleic acid molecules encoding these fusion proteins are also encompassed by the invention.

Exemplary fusion proteins reported in the literature include fusions of the T cell receptor (Gascoigne et al., Proc. Natl. Acad. Sci. USA 84:2936-2940 (1987)); CD4 (Capon et al., Nature 337:525-531 (1989); Traunecker et al., Nature 339:68-70 (1989); Zettmeissl et al., DNA Cell Biol. USA 9:347-353 (1990); and Byrn et al., Nature 344:667-670 (1990)); L-selectin (homing receptor) (Watson et al., J. Cell. Biol. 110:2221-2229 (1990); and Watson et al., Nature 349:164-167 (1991)); CD44 (Aruffo et al., Cell 61:1303-1313 (1990)); CD28 and B7 (Linsley et al., J. Exp. Med. 173:721-730 (1991)); CTLA-4 (Lisley et al., J. Exp. Med. 174:561-569 (1991)); CD22 (Stamenkovic et al., Cell 66:1133-1144 (1991)); TNF receptor (Ashkenazi et al., Proc. Natl. Acad. Sci. USA 88:10535-10539 (1991); Lesslauer et al., Eur. J. Immunol. 27:2883-2886 (1991); and Peppel et al., J. Exp. Med. 174:1483-1489 (1991)); and IgE receptor a (Ridgway and Gorman, J. Cell. Biol. Vol. 115, Abstract No. 1448 (1991)).

As discussed elsewhere herein, IGF-1R antibodies, or antigen-binding fragments, variants, or derivatives thereof of the invention may be fused to heterologous polypeptides to increase the in vivo half life of the polypeptides or for use in immunoassays using methods known in the art. For example, in one embodiment, PEG can be conjugated to the IGF-1R binding molecules of the invention to increase their half-life in vivo. Leong, S. R., et al., Cytokine 16:106 (2001); Adv. in Drug Deliv. Rev. 54:531 (2002); or Weir et al., Biochem. Soc. Transactions 30:512 (2002).

Moreover, IGF-1R binding molecules of the invention can be fused to marker sequences, such as a peptide to facilitate their purification or detection. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)), the “flag” tag and the “myc” tag.

Fusion proteins can be prepared using methods that are well known in the art (see for example U.S. Pat. Nos. 5,116,964 and 5,225,538). The precise site at which the fusion is made may be selected empirically to optimize the secretion or binding characteristics of the fusion protein. DNA encoding the fusion protein is then transfected into a host cell for expression.

A binding molecule of the invention may be administered alone or in conjunction with additional therapeutics (e.g., biologics or chemotherapeutic agents) to reduce or inhibit an IGF-1R-mediated effected on a cell (e.g., to inhibit tumor cell proliferation or to treat or slow the progression of a hyperproliferative disorder).

In one embodiment, IGF-1R binding molecules of the present invention may be used in non-conjugated form or may be conjugated to at least one of a variety of molecules, e.g., to improve the therapeutic properties of the molecule, to facilitate target detection, or for imaging or therapy of the patient. IGF-1R binding molecules of the invention can be labeled or conjugated either before or after purification, when purification is performed.

In particular, IGF-1R binding molecules of the invention may be conjugated to therapeutic agents, prodrugs, peptides, proteins, enzymes, viruses, lipids, biological response modifiers, pharmaceutical agents, or PEG.

Those skilled in the art will appreciate that conjugates may also be assembled using a variety of techniques depending on the selected agent to be conjugated. For example, conjugates with biotin are prepared e.g. by reacting a binding polypeptide with an activated ester of biotin such as the biotin N-hydroxysuccinimide ester. Similarly, conjugates with a fluorescent marker may be prepared in the presence of a coupling agent, e.g. those listed herein, or by reaction with an isothiocyanate, preferably fluorescein-isothiocyanate. Conjugates of the IGF-1R binding molecules of the invention are prepared in an analogous manner.

The present invention further encompasses IGF-1R binding molecules of the invention conjugated to a diagnostic or therapeutic agent. The IGF-1R binding molecules can be used diagnostically to, for example, monitor the development or progression of a neurological disease as part of a clinical testing procedure to, e.g., determine the efficacy of a given treatment and/or prevention regimen. Detection can be facilitated by coupling the IGF-1R binding molecule to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to antibodies for use as diagnostics according to the present invention. Examples of suitable enzymes include horseradish peroxidase, alkaline phosphatase, β-galactosidase,or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

An IGF-1R binding molecule also can be detectably labeled by coupling it to a chemiluminescent compound. The presence of the chemiluminescent-tagged IGF-1R binding molecules is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

One of the ways in which an IGF-1R binding molecule can be detectably labeled is by linking the same to an enzyme and using the linked product in an enzyme immunoassay (EIA) (Voller, A., “The Enzyme Linked Immunosorbent Assay (ELISA)” Microbiological Associates Quarterly Publication, Walkersville, Md., Diagnostic Horizons 2:1-7 (1978)); Voller et al., J. Clin. Pathol. 31:507-520 (1978); Butler, J. E., Meth. Enzymol. 73:482-523 (1981); Maggio, E. (ed.), Enzyme Immunoassay, CRC Press, Boca Raton, Fla., (1980); Ishikawa, E. et al., (eds.), Enzyme Immunoassay, Kgaku Shoin, Tokyo (1981). The enzyme, which is bound to the IGF-1R binding molecule will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the IGF-1R binding molecule, it is possible to detect the binding molecule through the use of a radioimmunoassay (RIA) (see, for example, Weintraub, B., Principles of Radioimmunoassays, Seventh Training Course on Radioligand Assay Techniques, The Endocrine Society, (March, 1986)), which is incorporated by reference herein). The radioactive isotope can be detected by means including, but not limited to, a gamma counter, a scintillation counter, or autoradiography.

An IGF-1R binding molecule can also be detectably labeled using fluorescence emitting metals such as 152Eu, or others of the lanthanide series. These metals can be attached to the binding molecules using such metal chelating groups as diethylenetriaminepentacetic acid (DTPA) or ethylenediaminetetraacetic acid (EDTA).

Techniques for conjugating various moieties to binding molecules are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivey (2nd Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. 62:119-58 (1982).

In particular, binding molecules for use in the diagnostic and treatment methods disclosed herein may be conjugated to cytotoxins (such as radioisotopes, cytotoxic drugs, or toxins) therapeutic agents, cytostatic agents, biological toxins, prodrugs, peptides, proteins, enzymes, viruses, lipids, biological response modifiers, pharmaceutical agents, immunologically active ligands (e.g., lymphokines or other antibodies wherein the resulting molecule binds to both the neoplastic cell and an effector cell such as a T cell), or PEG. In another embodiment, a binding molecule for use in the diagnostic and treatment methods disclosed herein can be conjugated to a molecule that decreases vascularization of tumors. In other embodiments, the disclosed compositions may comprise binding molecules coupled to drugs or prodrugs. Still other embodiments of the present invention comprise the use of binding molecules conjugated to specific biotoxins or their cytotoxic fragments such as ricin, gelonin, Pseudomonas exotoxin or diphtheria toxin. The selection of which conjugated or unconjugated binding molecule to use will depend on the type and stage of cancer, use of adjunct treatment (e.g., chemotherapy or external radiation) and patient condition. It will be appreciated that one skilled in the art could readily make such a selection in view of the teachings herein.

It will be appreciated that, in previous studies, anti-tumor antibodies labeled with isotopes have been used successfully to destroy cells in solid tumors as well as lymphomas/leukemias in animal models, and in some cases in humans. Exemplary radioisotopes include: ⁹⁰Y, ¹²⁵I, ¹³¹I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. The radionuclides act by producing ionizing radiation which causes multiple strand breaks in nuclear DNA, leading to cell death. The isotopes used to produce therapeutic conjugates typically produce high energy α- or β-particles which have a short path length. Such radionuclides kill cells to which they are in close proximity, for example neoplastic cells to which the conjugate has attached or has entered. They have little or no effect on non-localized cells. Radionuclides are essentially non-immunogenic.

With respect to the use of radiolabeled conjugates in conjunction with the present invention, binding molecules may be directly labeled (such as through iodination) or may be labeled indirectly through the use of a chelating agent. As used herein, the phrases “indirect labeling” and “indirect labeling approach” both mean that a chelating agent is covalently attached to a binding molecule and at least one radionuclide is associated with the chelating agent. Such chelating agents are typically referred to as bifunctional chelating agents as they bind both the polypeptide and the radioisotope. Particularly preferred chelating agents comprise 1-isothiocycmatobenzyl-3-methyldiothelene triaminepentaacetic acid (“MX-DTPA”) and cyclohexyl diethylenetriamine pentaacetic acid (“CHX-DTPA”) derivatives. Other chelating agents comprise P-DOTA and EDTA derivatives. Particularly preferred radionuclides for indirect labeling include ¹¹¹In and ⁹⁰Y.

As used herein, the phrases “direct labeling” and “direct labeling approach” both mean that a radionuclide is covalently attached directly to a polypeptide (typically via an amino acid residue). More specifically, these linking technologies include random labeling and site-directed labeling. In the latter case, the labeling is directed at specific sites on the polypeptide, such as the N-linked sugar residues present only on the Fc portion of the conjugates. Further, various direct labeling techniques and protocols are compatible with the instant invention. For example, Technetium-99 labeled polypeptides may be prepared by ligand exchange processes, by reducing pertechnate (TcO₄⁻) with stannous ion solution, chelating the reduced technetium onto a Sephadex column and applying the binding polypeptides to this column, or by batch labeling techniques, e.g. by incubating pertechnate, a reducing agent such as SnCl₂, a buffer solution such as a sodium-potassium phthalate-solution, and the binding molecules. In any event, preferred radionuclides for directly labeling polypeptides are well known in the art and a particularly preferred radionuclide for direct labeling is ¹³¹I covalently attached via tyrosine residues. Binding molecules for use in the methods disclosed herein may be derived, for example, with radioactive sodium or potassium iodide and a chemical oxidizing agent, such as sodium hypochlorite, chloramine T or the like, or an enzymatic oxidizing agent, such as lactoperoxidase, glucose oxidase and glucose.

Patents relating to chelators and chelator conjugates are known in the art. For instance, U.S. Pat. No. 4,831,175 of Gansow is directed to polysubstituted diethylenetriaminepentaacetic acid chelates and protein conjugates containing the same, and methods for their preparation. U.S. Pat. Nos. 5,099,069, 5,246,692, 5,286,850, 5,434,287 and 5,124,471 of Gansow also relate to polysubstituted DTPA chelates. These patents are incorporated herein by reference in their entireties. Other examples of compatible metal chelators are ethylenediaminetetraacetic acid (EDTA), diethylenetriaminepentaacetic acid (DPTA), 1,4,8,11-tetraazatetradecane, 1,4,8,11-tetraazatetradecane-1,4,8,11-tetraacetic acid, 1-oxa-4,7,12,15-tetraazaheptadecane-4,7,12,15-tetraacetic acid, or the like. Cyclohexyl-DTPA or CHX-DTPA is particularly preferred and is exemplified extensively below. Still other compatible chelators, including those yet to be discovered, may easily be discerned by a skilled artisan and are clearly within the scope of the present invention.

Compatible chelators, including the specific bifunctional chelator used to facilitate chelation U.S. Pat. Nos. 6,682,134, 6,399,061, and 5,843,439, incorporated herein by reference in their entireties, are preferably selected to provide high affinity for trivalent metals, exhibit increased tumor-to-non-tumor ratios and decreased bone uptake as well as greater in vivo retention of radionuclide at target sites, i.e., B-cell lymphoma tumor sites. However, other bifunctional chelators that may or may not possess all of these characteristics are known in the art and may also be beneficial in tumor therapy.

It will also be appreciated that, in accordance with the teachings herein, binding molecules may be conjugated to different radiolabels for diagnostic and therapeutic purposes. To this end the aforementioned U.S. Pat. Nos. 6,682,134, 6,399,061, and 5,843,439 disclose radiolabeled therapeutic conjugates for diagnostic “imaging” of tumors before administration of therapeutic antibody. “In2B8” conjugate comprises a murine monoclonal antibody, 2B8, specific to human CD20 antigen, that is attached to ¹¹¹In via a bifunctional chelator, i.e., MX-DTPA (diethylenetriaminepentaacetic acid), which comprises a 1:1 mixture of 1-isothiocyanatobenzyl-3-methyl-DTPA and 1-methyl-3-isothiocyanatobenzyl-DTPA. ¹¹¹In is particularly preferred as a diagnostic radionuclide because between about 1 to about 10 mCi can be safely administered without detectable toxicity; and the imaging data is generally predictive of subsequent ⁹⁰Y-labeled antibody distribution. Most imaging studies utilize 5 mCi ¹¹¹In-labeled antibody, because this dose is both safe and has increased imaging efficiency compared with lower doses, with optimal imaging occurring at three to six days after antibody administration. See, for example, Murray, J. Nuc. Med. 26: 3328 (1985) and Carraguillo et al., J. Nuc. Med. 26: 67 (1985).

As indicated above, a variety of radionuclides are applicable to the present invention and those skilled in the can readily determine which radionuclide is most appropriate under various circumstances. For example, ¹³¹I is a well known radionuclide used for targeted immunotherapy. However, the clinical usefulness of ¹³¹I can be limited by several factors including: eight-day physical half-life; dehalogenation of iodinated antibody both in the blood and at tumor sites; and emission characteristics (e.g., large gamma component) which can be suboptimal for localized dose deposition in tumor. With the advent of superior chelating agents, the opportunity for attaching metal chelating groups to proteins has increased the opportunities to utilize other radionuclides such as ¹¹¹In and ⁹⁰Y. ⁹⁰Y provides several benefits for utilization in radioimmunotherapeutic applications: the 64 hour half-life of ⁹⁰Y is long enough to allow antibody accumulation by tumor and, unlike e.g., ¹³¹I, ⁹⁰Y is a pure beta emitter of high energy with no accompanying gamma irradiation in its decay, with a range in tissue of 100 to 1,000 cell diameters. Furthermore, the minimal amount of penetrating radiation allows for outpatient administration of ⁹⁰Y-labeled antibodies. Additionally, internalization of labeled antibody is not required for cell killing, and the local emission of ionizing radiation should be lethal for adjacent tumor cells lacking the target molecule.

Additional preferred agents for conjugation to binding molecules, e.g., binding polypeptides are cytotoxic drugs, particularly those which are used for cancer therapy. As used herein, “a cytotoxin or cytotoxic agent” means any agent that is detrimental to the growth and proliferation of cells and may act to reduce, inhibit or destroy a cell or malignancy. Exemplary cytotoxins include, but are not limited to, radionuclides, biotoxins, enzymatically active toxins, cytostatic or cytotoxic therapeutic agents, prodrugs, immunologically active ligands and biological response modifiers such as cytokines. Any cytotoxin that acts to retard or slow the growth of immunoreactive cells or malignant cells is within the scope of the present invention.

Exemplary cytotoxins include, in general, cytostatic agents, alkylating agents, anti-metabolites, anti-proliferative agents, tubulin binding agents, hormones and hormone antagonists, and the like. Exemplary cytostatics that are compatible with the present invention include alkylating substances, such as mechlorethamine, triethylenephosphoramide, cyclophosphamide, ifosfamide, chlorambucil, busulfan, melphalan or triaziquone, also nitrosourea compounds, such as carmustine, lomustine, or semustine. Other preferred classes of cytotoxic agents include, for example, the maytansinoid family of drugs. Other preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, adriamycin, caminomycin, daunorubicin (daunomycin), doxorubicin, aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, or podophyllotoxin derivatives such as etoposide or etoposide phosphate, melphalan, vinblastine, vincristine, leurosidine, vindesine, leurosine and the like. Still other cytotoxins that are compatible with the teachings herein include taxol, taxane, cytochalasin B, gramicidin D, ethidium bromide, emetine, tenoposide, colchicin, dihydroxy anthracin dione, mitoxantrone, procaine, tetracaine, lidocaine, propranolol, and puromycin and analogs or homologs thereof. Hormones and hormone antagonists, such as corticosteroids, e.g. prednisone, progestins, e.g. hydroxyprogesterone or medroprogesterone, estrogens, e.g. diethylstilbestrol, antiestrogens, e.g. tamoxifen, androgens, e.g. testosterone, and aromatase inhibitors, e.g. aminogluthetimide are also compatible with the teachings herein. One skilled in the art may make chemical modifications to the desired compound in order to make reactions of that compound more convenient for purposes of preparing conjugates of the invention.

Examples of particularly preferred cytotoxins comprise members or derivatives of the enediyne family of anti-tumor antibiotics, including calicheamicin, esperamicins or dynemicins. These toxins are extremely potent and act by cleaving nuclear DNA, leading to cell death. Unlike protein toxins which can be cleaved in vivo to give many inactive but immunogenic polypeptide fragments, toxins such as calicheamicin, esperamicins and other enediynes are small molecules which are essentially non-immunogenic. These non-peptide toxins are chemically-linked to the dimers or tetramers by techniques which have been previously used to label monoclonal antibodies and other molecules. These linking technologies include site-specific linkage via the N-linked sugar residues present only on the Fc portion of the constructs. Such site-directed linking methods have the advantage of reducing the possible effects of linkage on the binding properties of the constructs.

As previously alluded to, compatible cytotoxins for preparation of conjugates may comprise a prodrug. As used herein, the term “prodrug” refers to a precursor or derivative form of a pharmaceutically active substance that is less cytotoxic to tumor cells compared to the parent drug and is capable of being enzymatically activated or converted into the more active parent form. Prodrugs compatible with the invention include, but are not limited to, phosphate-containing prodrugs, thiophosphate-containing prodrugs, sulfate containing prodrugs, peptide containing prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs that can be converted to the more active cytotoxic free drug. Further examples of cytotoxic drugs that can be derivatized into a prodrug form for use in the present invention comprise those chemotherapeutic agents described above. Among other cytotoxins, it will be appreciated that binding molecules disclosed herein can also be associated with or conjugated to a biotoxin such as ricin subunit A, abrin, diptheria toxin, botulinum, cyanginosins, saxitoxin, shigatoxin, tetanus, tetrodotoxin, trichothecene, verrucologen or a toxic enzyme. Preferably, such constructs will be made using genetic engineering techniques that allow for direct expression of the antibody-toxin construct. Other biological response modifiers that may be associated with the binding molecules disclosed herein comprise cytokines such as lymphokines and interferons. In view of the instant disclosure it is submitted that one skilled in the art could readily form such constructs using conventional techniques.

Another class of compatible cytotoxins that may be used in association with or conjugated to the disclosed binding molecules are radiosensitizing drugs that may be effectively directed to tumor or immunoreactive cells. Such drugs enhance the sensitivity to ionizing radiation, thereby increasing the efficacy of radiotherapy. A binding molecule conjugate internalized by the tumor cell would deliver the radiosensitizer nearer the nucleus where radiosensitization would be maximal. The unbound radiosensitizer linked binding molecules of the invention would be cleared quickly from the blood, localizing the remaining radiosensitization agent in the target tumor and providing minimal uptake in normal tissues. After rapid clearance from the blood, adjunct radiotherapy would be administered in one of three ways: 1.) external beam radiation directed specifically to the tumor, 2.) radioactivity directly implanted in the tumor or 3.) systemic radioimmunotherapy with the same targeting antibody. A potentially attractive variation of this approach would be the attachment of a therapeutic radioisotope to the radiosensitized immunoconjugate, thereby providing the convenience of administering to the patient a single drug.

In certain embodiments, a moiety that enhances the stability or efficacy of a binding molecule, e.g., a binding polypeptide, e.g., a IGF-1R-specific antibody or immunospecific fragment thereof can be conjugated. For example, in one embodiment, PEG can be conjugated to the binding molecules of the invention to increase their half-life in vivo. Leong, S. R., et al., Cytokine 16:106 (2001); Adv. in Drug Deliv. Rev. 54:531 (2002); or Weir et al., Biochem. Soc. Transactions 30:512 (2002).

The present invention further encompasses the use of binding molecules conjugated to a diagnostic or therapeutic agent. The binding molecules can be used diagnostically to, for example, monitor the development or progression of a tumor as part of a clinical testing procedure to, e.g., determine the efficacy of a given treatment and/or prevention regimen. Detection can be facilitated by coupling the binding molecule, to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions that can be conjugated to antibodies for use as diagnostics according to the present invention. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

A binding molecule can be detectably labeled by coupling it to a chemiluminescent compound. The presence of the chemiluminescent-tagged binding molecule is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. One of the ways in which a binding molecule can be detectably labeled is by linking the same to an enzyme and using the linked product in an enzyme immunoassay (EIA) (Voller, A., “The Enzyme Linked Immunosorbent Assay (ELISA)” Microbiological Associates Quarterly Publication, Walkersville, Md., Diagnostic Horizons 2:1-7 (1978)); Voller et al., J. Clin. Pathol. 31:507-520 (1978); Butler, J. E., Meth. Enzymol. 73:482-523 (1981); Maggio, E. (ed.), Enzyme Immunoassay, CRC Press, Boca Raton, Fla., (1980); Ishikawa, E. et al., (eds.), Enzyme Immunoassay, Kgaku Shoin, Tokyo (1981). The enzyme, which is bound to the binding molecule, will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the binding molecule, it is possible to detect cancer antigens through the use of a radioimmunoassay (RIA) (see, for example, Weintraub, B., Principles of Radioimmunoassays, Seventh Training Course on Radioligand Assay Techniques, The Endocrine Society, (March, 1986)), which is incorporated by reference herein). The radioactive isotope can be detected by means including, but not limited to, a gamma counter, a scintillation counter, or autoradiography.

A binding molecule can also be detectably labeled using fluorescence emitting metals such as 152Eu, or others of the lanthanide series. These metals can be attached to the binding molecule using such metal chelating groups as diethylenetriaminepentacetic acid (DTPA) or ethylenediaminetetraacetic acid (EDTA).

Techniques for conjugating various moieties to a binding molecule are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivey (2nd Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies'84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. 62:119-58 (1982).

(ii) Reducing Immunogenicity

In certain embodiments, IGF-1R binding molecules of the invention or portions thereof are modified to reduce their immunogenicity using art-recognized techniques. For example, binding molecules or portions thereof can be humanized, primatized, or deimmunized. In one embodiment, chimeric binding molecules can be made or binding molecules may comprise at least a portion of a chimeric antibody molecule. In such case a non-human IGF-1R binding molecule, typically a murine or primate binding molecule, that retains or substantially retains the antigen-binding properties of the parent binding molecule, but which is less immunogenic in humans is constructed. This may be achieved by various methods, including (a) grafting the entire non-human variable domains onto human constant regions to generate chimeric binding molecule; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81:6851-6855 (1984); Morrison et al., Adv. Immunol. 44:65-92 (1988); Verhoeyen et al., Science 239:1534-1536 (1988); Padlan, Molec. Immun. 28:489-498 (1991); Padlan, Molec. Immun. 31:169-217 (1994), and U.S. Pat. Nos. 5,585,089, 5,693,761, 5,693,762, and 6,190,370, all of which are hereby incorporated by reference in their entirety.

In one embodiment, a binding molecule (e.g., an antibody) of the invention or portion thereof may be chimeric. A chimeric binding molecule is a binding molecule in which different portions of the binding molecule are derived from different animal species, such as antibodies having a variable region derived from a murine monoclonal antibody and a human immunoglobulin constant region. Methods for producing chimeric binding moleculs are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397, which are incorporated herein by reference in their entireties. Techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) may be employed for the synthesis of said molecules. For example, a genetic sequence encoding a binding specificity of a mouse IGF-1R antibody molecule may be fused together with a sequence from a human antibody molecule of appropriate biological activity can be used. As used herein, a chimeric binding molecule is a molecule in which different portions are derived from different animal species, such as those having a variable region derived from a murine monoclonal antibody and a human immunoglobulin constant region, e.g., humanized antibodies.

In another embodiment, a binding molecule of the invention or portion thereof is primatized. Methods for primatizing antibodies are disclosed by Newman, Biotechnology 10: 1455-1460 (1992). Specifically, this technique results in the generation of antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

In another embodiment, a binding molecule (e.g., an antibody) of the invention or portion thereof is humanized. Humanized binding molecules are binding molecules having a binding specificity from non-human species antibody that binds the desired antigen having one or more complementarity determining regions (CDRs) from the non-human species antibody and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

De-immunization can also be used to decrease the immunogenicity of a binding molecule. As used herein, the term “de-immunization” includes alteration of an binding molecule to modify T cell epitopes (see, e.g., WO9852976A1, WO0034317A2). For example, VH and VL sequences from the starting antibody may analyzed and a human T cell epitope “map” from each V region showing the location of epitopes in relation to complementarity-determining regions (CDRs) and other key residues within the sequence. Individual T cell epitopes from the T cell epitope map are analyzed in order to identify alternative amino acid substitutions with a low risk of altering activity of the final antibody. A range of alternative VH and VL sequences are designed comprising combinations of amino acid substitutions and these sequences are subsequently incorporated into a range of binding polypeptides, e.g., IGF-1R-specific antibodies or immunospecific fragments thereof for use in the diagnostic and treatment methods disclosed herein, which are then tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

(iii) Effector Functions and Fc Modifications

IGF-1R binding molecules of the invention may comprise a constant region which mediates one or more effector functions. For example, binding of the C1 component of complement to an antibody constant region may activate the complement system. Activation of complement is important in the opsonisation and lysis of cell pathogens. The activation of complement also stimulates the inflammatory response and may also be involved in autoimmune hypersensitivity. Further, antibodies bind to receptors on various cells via the Fc region, with a Fc receptor binding site on the antibody Fc region binding to a Fc receptor (FcR) on a cell. There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

Certain embodiments of the invention include IGF-1R binding molecules in which at least one amino acid in one or more of the constant region domains has been deleted or otherwise altered so as to provide desired biochemical characteristics such as reduced effector functions, the ability to non-covalently dimerize, increased ability to localize at the site of a tumor, reduced serum half-life, or increased serum half-life when compared with a whole, unaltered antibody of approximately the same immunogenicity. For example, certain binding molecules for use in the diagnostic and treatment methods described herein are domain deleted antibodies which comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the CH2 domain will be deleted.

In certain IGF-1R binding molecules, the Fc portion may be mutated to decrease effector function using techniques known in the art. For example, the deletion or inactivation (through point mutations or other means) of a constant region domain may reduce Fc receptor binding of the circulating modified binding molecule thereby increasing tumor localization. In other cases it may be that constant region modifications consistent with the instant invention moderate complement binding and thus reduce the serum half life and nonspecific association of a conjugated cytotoxin. Yet other modifications of the constant region may be used to modify disulfide linkages or oligosaccharide moieties that allow for enhanced localization due to increased antigen specificity or flexibility. The resulting physiological profile, bioavailability and other biochemical effects of the modifications, such as tumor localization, biodistribution and serum half-life, may easily be measured and quantified using well know immunological techniques without undue experimentation.

In certain embodiments, an Fc domain employed in a binding polypeptide of the invention is an Fc variant. As used herein, the term “Fc variant” refers to an Fc domain having at least one amino acid substitution relative to the wild-type Fc domain from which said Fc domain is derived. For example, wherein the Fc domain is derived from a human IgG1 antibody, the Fc variant of said human IgG1 Fc domain comprises at least one amino acid substitution relative to said Fc domain.

The amino acid substitution(s) of an Fc variant may be located at any position (ie., any EU convention amino acid position) within the Fc domain. In one embodiment, the Fc variant comprises a substitution at an amino acid position located in a hinge domain or portion thereof. In another embodiment, the Fc variant comprises a substitution at an amino acid position located in a CH2 domain or portion thereof. In another embodiment, the Fc variant comprises a substitution at an amino acid position located in a CH3 domain or portion thereof. In another embodiment, the Fc variant comprises a substitution at an amino acid position located in a CH4 domain or portion thereof.

The binding polypeptides of the invention may employ any art-recognized Fc variant which is known to impart an improvement (e.g., reduction or enhancement) in effector function and/or FcR binding. Said Fc variants may include, for example, any one of the amino acid substitutions disclosed in International PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351A2, WO04/074455A2, WO04/099249A2, WO05/040217A2, WO05/070963A1, WO05/077981A2, WO05/092925A2, WO05/123780A2, WO06/019447A1, WO06/047350A2, and WO06/085967A2 or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; and 7,083,784, each of which is incorporated by reference herein.

In preferred embodiments, binding polypeptide may comprise an Fc variant comprising an amino acid substitution an EU amino acid position that is within the “15 Angstrom Contact Zone” of the Fc domain. The 15 Angstrom Zone includes residues located at EU positions 243 to 261, 275 to 280, 282-293, 302 to 319, 336 to 348, 367, 369, 372 to 389, 391, 393, 408, and 424-440 of the Fc region.

The certain embodiments, a binding polypeptide of the invention comprising an Fc variant comprising an amino acid substitution which alters the antigen-independent effector functions of the antibody, in particular the circulating half-life of the antibody. Such binding polypeptides exhibit either increased or decreased binding to FcRn when compared to binding polypeptides lacking these substitutions, therefore, have an increased or decreased half-life in serum, respectively. Fc variants with improved affinity for FcRn are anticipated to have longer serum half-lives, and such molecules have useful applications in methods of treating mammals where long half-life of the administered polypeptide is desired, e.g., to treat a chronic disease or disorder. In contrast, Fc variants with decreased FcRn binding affinity are expected to have shorter half-lives, and such molecules are also useful, for example, for administration to a mammal where a shortened circulation time may be advantageous, e.g. for in vivo diagnostic imaging or in situations where the starting polypeptide has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity may be desired include those applications in which localization the brain, kidney, and/or liver is desired. In one exemplary embodiment, the altered polypeptides of the invention exhibit reduced transport across the epithelium of kidney glomeruli from the vasculature. In another embodiment, the altered polypeptides of the invention exhibit reduced transport across the blood brain barrier (BBB) from the brain, into the vascular space. In one embodiment, a binding polypeptide with altered FcRn binding comprises an Fc domain having one or more amino acid substitutions within the “FcRn binding loop” of an Fc domain. The FcRn binding loop is comprised of amino acid residues 280-299 (according to EU numbering). In other embodiment, a binding polypeptide of the invention having altered FcRn binding affinity comprises an Fc domain having one or more amino acid substitutions within the 15 \acute{Å} FcRn “contact zone.” As used herein, the term 15 \acute{Å} FcRn “contact zone” includes residues at the following positions 243-261, 275-280, 282-293, 302-319, 336-348, 367, 369, 372-389, 391, 393, 408, 424, 425-440 (EU numbering). In preferred embodiments, a binding polypeptide of the invention having altered FcRn binding affinity comprises an Fc domain having one or more amino acid substitutions at any one of the following positions: 256, 277-281, 283-288, 303-309, 313, 338, 342, 376, 381, 384, 385, 387, 434, and 438. Exemplary amino acid substitutions which altered FcRn binding activity are disclosed in International PCT Publication No. WO05/047327 which is incorporated by reference herein.

In other embodiments, certain binding molecules for use in the diagnostic and treatment methods described herein have s constant region, e.g., an IgG4 heavy chain constant region, which is altered to reduce or eliminate glycosylation. For example, a binding polypeptide of the invention may also comprise an Fc variant comprising an amino acid substitution which alters the glycosylation of the binding polypeptide. For example, said Fc variant may have reduced glycosylation (e.g., N- or O-linked glycosylation) or may comprise an altered glycoform of the wild-type Fc domain (e.g., a low fucose or fucose-free glycan). In exemplary embodiments, the Fc variant comprises reduced glycosylation of the N-linked glycan normally found at amino acid position 297 (EU numbering). In another exemplary embodiment, the Fc variant comprises a low fucose or fucose free glycan at amino acid position 297 (EU numbering). In another embodiment, the binding polypeptide has an amino acid substitution near or within a glycosylation motif, for example, an N-linked glycosylation motif that contains the amino acid sequence NXT or NXS. In a particular embodiment, the binding polypeptide comprises an Fc variant with an amino acid substitution at amino acid position 228 or 299 (EU numbering). In more particular embodiments, the binding molecule comprises an IgG4 constant region comprising an S228P and a T299A mutation (EU numbering).

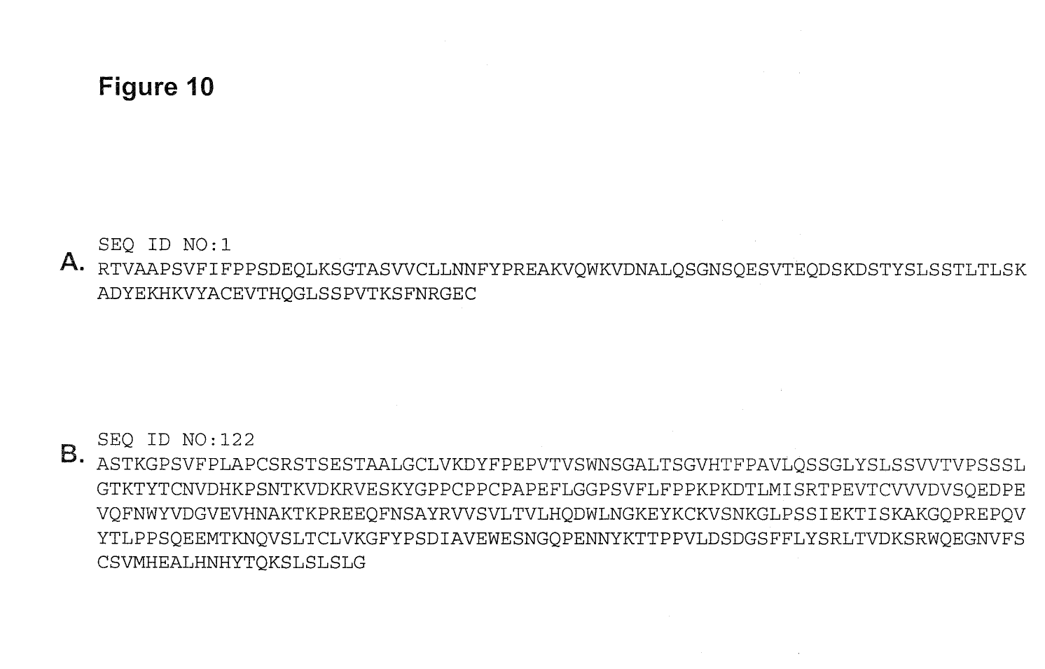

Exemplary amino acid substitutions which confer reduce or altered glycosylation are disclosed in International PCT Publication No. WO05/018572, which is incorporated by reference herein. In preferred embodiments, the binding molecules of the invention are modified to eliminate glycosylation. Such binding molecules may be referred to as “agly” binding molecules (e.g. “agly” antibodies). While not being bound by theory, it is believed that “agly” binding molecules may have an improved safety and stability profile in vivo. Exemplary agly binding molecules comprise an aglycosylated Fc region of an IgG4 antibody (“IgG4.P”) which is devoid of Fc-effector function thereby eliminating the potential for Fc mediated toxicity to the normal vital organs that express IGF-1R. In particular embodiments, agly binding molecules of the invention may comprise the IgG4.P constant region set foth as SEQ ID NO:132 (see FIG. 10(b)).

### VII. Methods of Making Binding Molecules

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligo dT cellulose. Suitable techniques are familiar in the art.

In one embodiment, cDNAs that encode separate chains of a binding molecule of the invention, e.g., the light and the heavy chains of an antibody, may be made, either simultaneously or separately, using reverse transcriptase and DNA polymerase in accordance with well known methods. For example, PCR may be initiated by consensus constant region primers or by more specific primers based on the published DNA and amino acid sequences. As discussed above, PCR also may be used to isolate DNA clones encoding separate binding molecule chains. In this case the libraries may be screened by consensus primers or larger homologous probes, such as mouse constant region probes. DNA, typically plasmid DNA, may be isolated from the cells using techniques known in the art, restriction mapped and sequenced in accordance with standard, well known techniques set forth in detail, e.g., in the foregoing references relating to recombinant DNA techniques. Of course, the DNA may be synthetic according to the present invention at any point during the isolation process or subsequent analysis. Following manipulation of the isolated genetic material to provide binding molecules of the invention, the polynucleotides encoding the IGF-1R binding molecules are typically inserted in an expression vector for introduction into host cells that may be used to produce the desired quantity of IGF-1R binding molecule.

Recombinant expression of a binding molecule, e.g., a heavy or light chain of an antibody which binds to a target molecule described herein, e.g., IGF-1R, requires construction of an expression vector containing a polynucleotide that encodes the binding molecule. Once a polynucleotide encoding a binding molecule (or a chain or portion thereof) of the invention has been obtained, the vector for the production of the binding molecule may be produced by recombinant DNA technology using techniques well known in the art. Thus, methods for preparing a protein by expressing a polynucleotide containing a binding molecule encoding nucleotide sequence are described herein. Methods which are well known to those skilled in the art can be used to construct expression vectors containing binding molecule coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding a binding molecule of the invention, or a chain or domain thereof, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the nucleotide encoding the binding molecule (or chain or domain thereof) may be cloned into such a vector for expression of the entire binding molecule.

Where the binding molecule of the invention is a dimer, the host cell may be co-transfected with two expression vectors of the invention, the first vector encoding a first polypeptide monomer and the second vector encoding a second polypeptide monomer. The two vectors may contain identical selectable markers which enable equal expression of the monomers. Alternatively, a single vector may be used which encodes both monomers. In embodiments the monomers are antibody light and heavy chains, the light chain is advantageously placed before the heavy chain to avoid an excess of toxic free heavy chain (Proudfoot, Nature 322:52 (1986); Kohler, Proc. Natl. Acad. Sci. USA 77:2197 (1980)). The coding sequences for the monomers of a binding molecule may comprise cDNA or genomic DNA. The term “vector” or “expression vector” is used herein to mean vectors used in accordance with the present invention as a vehicle for introducing into and expressing a desired gene in a host cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include signal sequences, splice signals, as well as transcriptional promoters, enhancers, and termination signals. In particularly preferred embodiments the cloned variable region genes are inserted into an expression vector along with the heavy and light chain constant region genes (preferably human) synthetic as discussed above. In one embodiment, this is effected using a proprietary expression vector of Biogen IDEC, Inc., referred to as NEOSPLA (disclosed in U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression of antibodies upon incorporation of variable and constant region genes, transfection in CHO cells, followed by selection in G418 containing medium and methotrexate amplification. Of course, any expression vector which is capable of eliciting expression in eukaryotic cells may be used in the present invention. Examples of suitable vectors include, but are not limited to plasmids pcDNA3, pHCMV/Zeo, pCR3.1, pEF1/His, pIND/GS, pRc/HCMV2, pSV40/Zeo2, pTRACER-HCMV, pUB6/V5-His, pVAX1, and pZeoSV2 (available from Invitrogen, San Diego, Calif.), and plasmid pCI (available from Promega, Madison, Wis.). In general, screening large numbers of transformed cells for those which express suitably high levels if immunoglobulin heavy and light chains is routine experimentation which can be carried out, for example, by robotic systems. Vector systems are also taught in U.S. Pat. Nos. 5,736,137 and 5,658,570, each of which is incorporated by reference in its entirety herein. This system provides for high expression levels, e.g., >30 pg/cell/day. Other exemplary vector systems are disclosed e.g., in U.S. Pat. No. 6,413,777.

In other preferred embodiments the binding molecules of the invention may be expressed using polycistronic constructs such as those disclosed in United States Patent Application Publication No. 2003-0157641 A1, filed Nov. 18, 2002 and incorporated herein in its entirety. In these novel expression systems, multiple gene products of interest such as heavy and light chains of antibodies may be produced from a single polycistronic construct. These systems advantageously use an internal ribosome entry site (IRES) to provide relatively high levels of IGF-1R binding molecules thereof in eukaryotic host cells. Compatible IRES sequences are disclosed in U.S. Pat. No. 6,193,980 which is also incorporated herein. Those skilled in the art will appreciate that such expression systems may be used to effectively produce the full range of IGF-1R binding molecules disclosed in the instant application.

More generally, once the vector or DNA sequence encoding a monomeric subunit of the IGF-1R binding molecule has been prepared, the expression vector may be introduced into an appropriate host cell. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Vectors, Rodriguez and Denhardt, Eds., Butterworths, Boston, Mass., Chapter 24.2, pp. 470-472 (1988). Typically, plasmid introduction into the host is via electroporation. The host cells harboring the expression construct are grown under conditions appropriate to the production of the binding molecule, and assayed for binding molecule synthesis. Exemplary assay techniques include enzyme-linked immunosorbent as say (ELISA), radioimmunoas say (RIA), or fluorescence-activated cell sorter analysis (FACS), immunohistochemistry and the like.

The expression vector is transferred to a host cell by conventional techniques and the transfected cells are then cultured by conventional techniques to produce a binding moleucle for use in the methods described herein. Thus, the invention includes host cells containing a polynucleotide encoding a binding molecule of the invention, or a monomer or chain thereof, operably linked to a heterologous promoter. In preferred embodiments for the expression of double-chained or dimeric binding molecules, vectors which separately encode binding molecule chains may be co-expressed in the host cell for expression of the entire binding molecule, as detailed below.

As used herein, “host cells” refers to cells which harbor vectors constructed using recombinant DNA techniques and encoding at least one heterologous gene. In descriptions of processes for isolation of binding molecules from recombinant hosts, the terms “cell” and “cell culture” are used interchangeably to denote the source of binding molecule unless it is clearly specified otherwise. In other words, recovery of polypeptide from the “cells” may mean either from spun down whole cells, or from the cell culture containing both the medium and the suspended cells.

A variety of host-expression vector systems may be utilized to express binding molecules for use in the methods described herein. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing binding molecule coding sequences; yeast (e.g., Saccharomyces, Pichia) transformed with recombinant yeast expression vectors containing binding molecule coding sequences; insect cell systems infected with recombinant virus expression vectors (e.g., baculovirus) containing binding molecule coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing binding molecule coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant binding moleculea, are used for the expression of a recombinant binding molecule. For example, mammalian cells such as Chinese hamster ovary cells (CHO) in conjunction with a vector such as the major intermediate early gene promoter element from human cytomegalovirus is an effective expression system for antibodies and other binding molecules (Foecking et al., Gene 45:101 (1986); Cockett et al., Bio/Technology 8:2 (1990)).

The host cell line used for protein expression is often of mammalian origin; those skilled in the art are credited with ability to preferentially determine particular host cell lines which are best suited for the desired gene product to be expressed therein. Exemplary host cell lines include, but are not limited to, CHO (Chinese Hamster Ovary), DG44 and DUXB11 (Chinese Hamster Ovary lines, DHFR minus), HELA (human cervical carcinoma), CVI (monkey kidney line), COS (a derivative of CVI with SV40 T antigen), VERY, BHK (baby hamster kidney), MDCK, 293, W138, R1610 (Chinese hamster fibroblast) BALBC/3T3 (mouse fibroblast), HAK (hamster kidney line), SP2/O (mouse myeloma), P3×63-Ag3.653 (mouse myeloma), BFA-1c1BPT (bovine endothelial cells), RAJI (human lymphocyte) and 293 (human kidney). CHO cells are particularly preferred. Host cell lines are typically available from commercial services, the American Tissue Culture Collection or from published literature. In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used.

For long-term, high-yield production of recombinant proteins, stable expression is preferred. For example, cell lines which stably express the binding molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which stably express the binding molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 1980) genes can be employed in tk-, hgprt- or aprt-cells, respectively. Also, anti-metabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); TIB TECH 11(5):155-215 (May, 1993); and hygro, which confers resistance to hygromycin (Santerre et al., Gene 30:147 (1984). Methods commonly known in the art of recombinant DNA technology which can be used are described in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of a binding molecule can be increased by vector amplification (for a review, see Bebbington and Hentschel, The use of vectors based on gene amplification for the expression of cloned genes in mammalian cells in DNA cloning, Academic Press, New York, Vol. 3. (1987)). When a marker in the vector system expressing the binding molecule is amplifiable, increase in the level of inhibitor present in culture of host cell will increase the number of copies of the marker gene. Since the amplified region is associated with the binding molecule, production of the binding molecule will also increase (Crouse et al., Mol. Cell. Biol. 3:257 (1983)).

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography In certain embodiments, the binding molecules of the invention are produced at high yield when produced commercial scale (e.g., in cell cultures or bioreactors of 25 L, 50 L, 100 L, 1000 L size or greater). For example, the binding molecules of the invention may be produced by a host cell such that at least 5 mg (e.g., at least 10 mg, 20 mg, 30 mg, 40 mg, 50 mg, 75 mg, 100 mg, 200 mg, 500 mg, 750 mg, 1 g, 1.5 g, 2 g, 2.5 g, or 5 g) of binding molecule is produced for every liter of the host cell culture medium.

Preferably, the binding molecules of the invention do not have the propensity to form aggregates. Aggregation can be measured by a number of non-limiting biochemical or biophysical techniques. For example, the aggregation of a composition of the invention may be evaluated using chromatography, e.g. Size-Exclusion Chromatograpy (SEC). SEC separates molecules on the basis of size. A column is filled with semi-solid beads of a polymeric gel that will admit ions and small molecules into their interior but not large ones. When a protein composition is applied to the top of the column, the compact folded proteins (ie. non-aggregated proteins) are distributed through a larger volume of solvent than is available to the large protein aggregates. Consequently, the large aggregates move more rapidly through the column, and in this way the mixture can be separated or fractionated into its components. Each fraction can be separately quantified (e.g. by light scattering) as it elutes from the gel. Accordingly, the % aggregation of a composition of the invention can be determined by comparing the concentration of a fraction with the total concentration of protein applied to the gel. Stable compositions elute from the column as essentially a single fraction and appear as essentially a single peak in the elution profile or chromatogram.

In preferred embodiments, SEC is used in conjunction with in-line light scattering (e.g. classical or dynamic light scattering) to determine the % aggregation of a composition. In certain preferred embodiments, static light scattering is employed to measure the mass of each fraction or peak, independent of the molecular shape or elution position. In other preferred embodiments, dynamic light scattering is employed to measure the hydrodynamic size of a composition. Other exemplary methods for evaluating protein stability include High-Speed SEC (see e.g. Corbett et al., Biochemistry. 23(8):1888-94, 1984).

Genes encoding IGF-1R binding molecules of the invention can also be expressed non-mammalian cells such as bacteria or insect or yeast or plant cells. Bacteria which readily take up nucleic acids include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the heterologous polypeptides typically become part of inclusion bodies. The heterologous polypeptides must be isolated, purified and then assembled into functional molecules. Where tetravalent forms of binding molecules are desired, the subunits will then self-assemble into tetravalent binding molecules (e.g. tetravalent antibodies (WO02/096948A2)).

In bacterial systems, a number of expression vectors may be advantageously selected depending upon the use intended for the binding molecule being expressed. For example, when a large quantity of such a protein is to be produced, for the generation of pharmaceutical compositions of a binding molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the binding molecule coding sequence may be ligated individually into the vector in frame with the lacZ coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In addition to prokaryotes, eukaryotic microbes may also be used. Saccharomyces cerevisiae, or common baker's yeast, is the most commonly used among eukaryotic microorganisms although a number of other strains are commonly available, e.g., Pichia pastoris.

For expression in Saccharomyces, the plasmid YRp7, for example, (Stinchcomb et al., Nature 282:39 (1979); Kingsman et al., Gene 7:141 (1979); Tschemper et al., Gene 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics 85:12 (1977)). The presence of the trp1 lesion as a characteristic of the yeast host cell genome then provides an effective environment for detecting transformation by growth in the absence of tryptophan.

In an insect system, Autographa californica nuclear polyhedrosis virus (AcNPV) is typically used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

Once a binding molecule of the invention has been recombinantly expressed, it may be purified by any method known in the art for purification of a binding molecule, for example, by chromatography (e.g., ion exchange, affinity, particularly by affinity for the specific antigen after Protein A, and sizing column chromatography), centrifugation, differential solubility, or by any other standard technique for the purification of proteins. Alternatively, a preferred method for increasing the affinity of binding molecules (e.g. antibodies) of the invention is disclosed in US 2002 0123057 A1.

### VIII. Methods Using Compositions Comprising Binding Molecules Which Bind to Different Epitopes of IGF-1R

The binding molecules of the instant invention reduce or inhibit IGF-1R-mediated effects on cells, such as proliferation in cells, e.g., tumor cells expressing IGF-1R. In another embodiment, the binding molecules of the invention inhibit IGF-1R-mediated signaling in cells, e.g., tumor cells expressing IGF-1R. The inhibition of IGF-1R-mediated signaling can be measured by determining at the activation of one or more signaling pathways or by determining a more downstream measure of activation such as cell proliferation. Such measurements can be made using standard methods known in the art or described herein, e.g., in the Examples.

For example, in one embodiment, a binding molecule of the invention reduces or inhibits IGF-1 or IGF-2-mediated IGF-1R phosphorylation, AKT or MAPK phosphorylation, AKT mediated survival signalling. In another embodiment, the binding molecules of the invention inhibit tumor cell growth, e.g., in vitro or in vivo. In yet another embodiment, a binding molecule of the invention induces IGF-1R internalization. In one embodiment, a binding molecule of the invention inhibits a parameter of IGF-1R-mediated cellular activation to a greater extent than one of the individual binding moieties present in the molecule (e.g., than a monoclonal antibody comprising that binding specificity) or than a combination comprising (i) a first monospecific binding molecule comprising said first binding moiety and (ii) a second monospecific binding molecule comprising said second moiety.

One embodiment of the present invention provides methods for treating (e.g., slowing the progression of, ameliorating at least one symptom of, reducing the spread of) a hyperproliferative disease or disorder, e.g., cancer, a malignancy, a tumor, or a metastasis thereof, in an animal suffering from such disease or predisposed to contract such disease, the method comprising, consisting essentially of, or consisting of administering to the animal an effective amount of a binding molecule or composition of the invention described herein.

A binding molecule of the present invention which specifically binds to IGF-1R or a variant thereof, to be used in treatment methods disclosed herein can be prepared and used as a therapeutic agent that stops, reduces, prevents, or inhibits cellular activities involved in cellular hyperproliferation, e.g., cellular activities that induce the altered or abnormal pattern of vascularization that is often associated with hyperproliferative diseases or disorders. Binding molecules according to the invention can be used in unlabeled or unconjugated form, or can be coupled or linked to cytotoxic moieties such as radiolabels and biochemical cytotoxins to produce agents that exert therapeutic effects.

The present invention provides methods for treating various hyperproliferative disorders, e.g., by inhibiting tumor growth, in a mammal, comprising, consisting essentially of, or consisting of administering to the mammal an effective amount of a binding molecule which specifically or preferentially binds to IGF-1R, e.g., human IGF-1R.

The present invention is more specifically directed to a method of treating a hyperproliferative disease, e.g., inhibiting or preventing tumor formation, tumor growth, tumor invasiveness, and/or metastasis formation, in an animal, e.g., a mammal, e.g., a human, comprising, consisting essentially of, or consisting of administering to an animal in need thereof an effective amount of binding molecule of the invention.

In other embodiments, the present invention includes a method for treating a hyperproliferative disease, e.g., inhibiting or reducing tumor formation, tumor growth (e.g., cell proliferation), tumor invasiveness, and/or metastasis formation in an animal, e.g., a human patient, where the method comprises administering to an animal in need of such treatment an effective amount of a composition comprising, consisting essentially of, or consisting of, in addition to a pharmaceutically acceptable carrier, a binding molecule of the invention (e.g. a multi specific binding molecule of the invention) or a combination of binding molecules (e.g. two or more monospecific binding molecules which bind different IGF-1R epitopes.

In other embodiments, the present invention includes a method for treating a hyperproliferative disease, e.g., inhibiting tumor formation, tumor growth, tumor invasiveness, and/or metastasis formation in an animal, e.g., a human patient, where the method comprises administering to an animal in need of such treatment an effective amount of a composition comprising, consisting essentially of, or consisting of, in addition to a pharmaceutically acceptable carrier, a binding molecule of the invention (e.g. a multi specific binding molecule of the invention) or a combination of binding molecules (e.g. two or more monospecific binding molecules which bind different IGF-1R epitopes, and an additional moiety which modifies a binding molecule, e.g., a carbohydrate moiety may be included such that the binding molecule binds with higher affinity to modified target protein than it does to an unmodified version of the protein. Alternatively, the binding molecule does not bind the unmodified version of the target protein at all.

More specifically, the present invention provides a method of treating cancer in a human, comprising administering to a human in need of treatment a composition comprising an effective amount of an IGF-1R-specific binding molecule of the invention (e.g. a multispecific binding molecule of the invention) or a combination of binding molecules (e.g. two or more monospecific binding molecules which bind different IGF-1R epitopes) and a pharmaceutically acceptable carrier. Types of cancer to be treated include, but are not limited to, stomach cancer, renal cancer, brain cancer, bladder cancer, colon cancer, lung cancer, breast cancer, pancreatic cancer, ovarian cancer, and prostate cancer. IX. Diagnostic or Prognostic Methods Using IGF-1R-Specific Binding Molecules and Nucleic Acid Amplification Assays

IGF-1R-specific binding molecules or compositions of the invention, can be used for diagnostic purposes to detect, diagnose, or monitor diseases, disorders, and/or conditions associated with the aberrant expression and/or activity of IGF-1R. IGF-1R expression is increased in tumor tissue and other neoplastic conditions.

IGF-1R-specific binding molecules are useful for diagnosis, treatment, prevention and/or prognosis of hyperproliferative disorders in mammals, preferably humans. Such disorders include, but are not limited to, cancer, neoplasms, tumors and/or as described under elsewhere herein, especially IGF-1R-associated cancers such as stomach cancer, renal cancer, brain cancer, bladder cancer, colon cancer, lung cancer, breast cancer, pancreatic cancer, ovarian cancer, and prostate cancer.

For example, as disclosed herein, IGF-1R expression is associated with at least stomach, renal, brain, bladder, colon, lung, breast, pancreatic, ovarian, and prostate tumor tissues. Accordingly, binding molecules of the invention may be used to detect particular tissues expressing increased levels of IGF-1R. These diagnostic assays may be performed in vivo or in vitro, such as, for example, on blood samples, biopsy tissue or autopsy tissue.

Thus, the invention provides a diagnostic method useful during diagnosis of a cancers and other hyperproliferative disorders, which involves measuring the expression level of IGF-1R protein or transcript in tissue or other cells or body fluid from an individual and comparing the measured expression level with a standard IGF-1R expression levels in normal tissue or body fluid, whereby an increase in the expression level compared to the standard is indicative of a disorder.

One embodiment provides a method of detecting the presence of abnormal hyperproliferative cells, e.g., precancerous or cancerous cells, in a fluid or tissue sample, comprising assaying for the expression of IGF-1R in tissue or body fluid samples of an individual and comparing the presence or level of IGF-1R expression in the sample with the presence or level of IGF-1R expression in a panel of standard tissue or body fluid samples, where detection of IGF-1R expression or an increase in IGF-1R expression over the standards is indicative of aberrant hyperproliferative cell growth.

More specifically, the present invention provides a method of detecting the presence of abnormal hyperproliferative cells in a body fluid or tissue sample, comprising (a) assaying for the expression of IGF-1R in tissue or body fluid samples of an individual using IGF-1R-specific binding molecules of the present invention, and (b) comparing the presence or level of IGF-1R expression in the sample with a the presence or level of IGF-1R expression in a panel of standard tissue or body fluid samples, whereby detection of IGF-1R expression or an increase in IGF-1R expression over the standards is indicative of aberrant hyperproliferative cell growth.

With respect to cancer, the presence of a relatively high amount of IGF-1R protein in biopsied tissue from an individual may indicate the presence of a tumor or other malignant growth, may indicate a predisposition for the development of such malignancies or tumors, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

IGF-1R-specific binding molecules of the present invention can be used to assay protein levels in a biological sample using classical immunohistological methods known to those of skill in the art (e.g., see Jalkanen, et al., J. Cell. Biol. 101:976-985 (1985); Jalkanen, et al., J. Cell Biol. 105:3087-3096 (1987)). Other antibody-based methods useful for detecting protein expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹¹²In), and technetium (⁹⁹Tc); luminescent labels, such as luminol; and fluorescent labels, such as fluorescein and rhodamine, and biotin. Suitable assays are described in more detail elsewhere herein.

One aspect of the invention is a method for the in vivo detection or diagnosis of a hyperproliferative disease or disorder associated with aberrant expression of IGF-1R in an animal, preferably a mammal and most preferably a human. In one embodiment, diagnosis comprises: a) administering (for example, parenterally, subcutaneously, or intraperitoneally) to a subject an effective amount of a labeled binding molecule of the present invention, which specifically binds to IGF-1R; b) waiting for a time interval following the administering for permitting the labeled binding molecule to preferentially concentrate at sites in the subject where IGF-1R is expressed (and for unbound labeled molecule to be cleared to background level); c) determining background level; and d) detecting the labeled molecule in the subject, such that detection of labeled molecule above the background level indicates that the subject has a particular disease or disorder associated with aberrant expression of IGF-1R. Background level can be determined by various methods including comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of, e.g., ⁹⁹Tc. The labeled binding molecule, e.g., antibody or antibody fragment, will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 7 to 10 days.

Presence of the labeled binding molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the binding molecule is labeled with a radioisotope and is detected in the patient using a radiation responsive surgical instrument (Thurston et al., U.S. Pat. No. 5,441,050). In another embodiment, the binding molecule is labeled with a fluorescent compound and is detected in the patient using a fluorescence responsive scanning instrument. In another embodiment, the binding molecule is labeled with a positron emitting metal and is detected in the patent using positron emission-tomography. In yet another embodiment, the binding molecule is labeled with a paramagnetic label and is detected in a patient using magnetic resonance imaging (MRI).

Antibody labels or markers for in vivo imaging of IGF-1R expression include those detectable by X-radiography, nuclear magnetic resonance imaging (NMR), MRI, CAT-scans or electron spin resonance imaging (ESR). For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma. Where in vivo imaging is used to detect enhanced levels of IGF-1R expression for diagnosis in humans, it may be preferable to use human antibodies or “humanized” chimeric monoclonal antibodies as described elsewhere herein.

In a related embodiment to those described above, monitoring of an already diagnosed disease or disorder is carried out by repeating any one of the methods for diagnosing the disease or disorder, for example, one month after initial diagnosis, six months after initial diagnosis, one year after initial diagnosis, etc.

Where a diagnosis of a disorder, including diagnosis of a tumor, has already been made according to conventional methods, detection methods as disclosed herein are useful as a prognostic indicator, whereby patients continuing to exhibiting enhanced IGF-1R expression will experience a worse clinical outcome relative to patients whose expression level decreases nearer the standard level.

By “assaying the expression level of the tumor associated IGF-1R polypeptide” is intended qualitatively or quantitatively measuring or estimating the level of IGF-1R polypeptide in a first biological sample either directly (e.g., by determining or estimating absolute protein level) or relatively (e.g., by comparing to the cancer associated polypeptide level in a second biological sample). Preferably, IGF-1R polypeptide expression level in the first biological sample is measured or estimated and compared to a standard IGF-1R polypeptide level, the standard being taken from a second biological sample obtained from an individual not having the disorder or being determined by averaging levels from a population of individuals not having the disorder. As will be appreciated in the art, once the “standard” IGF-1R polypeptide level is known, it can be used repeatedly as a standard for comparison.

By “biological sample” is intended any biological sample obtained from an individual, cell line, tissue culture, or other source of cells potentially expressing IGF-1R. As indicated, biological samples include body fluids (such as sera, plasma, urine, synovial fluid and spinal fluid), and other tissue sources which contain cells potentially expressing IGF-1R. Methods for obtaining tissue biopsies and body fluids from mammals are well known in the art.

In an additional embodiment, binding molecule of the invention may be used to quantitatively or qualitatively detect the presence of IGF-1R gene products or conserved variants or peptide fragments thereof. This can be accomplished, for example, by immunofluoresence techniques employing a fluorescently labeled binding molecule coupled with light microscopic, flow cytometric, or fluorimetric detection.

Cancers that may be diagnosed, and/or prognosed using the methods described above include but are not limited to, stomach cancer, renal cancer, brain cancer, bladder cancer, colon cancer, lung cancer, breast cancer, pancreatic cancer, ovarian cancer, and prostate cancer.

### X. Immunoassays

IGF-1R-specific binding molecule disclosed herein may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, Current Protocols in Molecular Biology, John Wiley & Sons, Inc., New York, Vol. 1 (1994), which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the binding molecule of interest to the cell lysate, incubating for a period of time (e.g., 1-4 hours) at 4.degree. C., adding protein A and/or protein G sepharose beads to the cell lysate, incubating for about an hour or more at 4.degree. C., washing the beads in lysis buffer and resuspending the beads in SDS/sample buffer. The ability of the binding molecule of interest to immunoprecipitate a particular antigen can be assessed by, e.g., western blot analysis. One of skill in the art would be knowledgeable as to the parameters that can be modified to increase the binding of the binding molecule to an antigen and decrease the background (e.g., pre-clearing the cell lysate with sepharose beads). For further discussion regarding immunoprecipitation protocols see, e.g., Ausubel et al., eds, Current Protocols in Molecular Biology, John Wiley & Sons, Inc., New York, Vol. 1 (1994) at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary binding molecule (the binding molecule of interest) diluted in blocking buffer, washing the membrane in washing buffer, blocking the membrane with a secondary binding molecule (which recognizes the primary antibody, e.g., an anti-human antibody) conjugated to an enzymatic substrate (e.g., horseradish peroxidase or alkaline phosphatase) or radioactive molecule (e.g., 32P or 1251) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, Current Protocols in Molecular Biology, John Wiley & Sons, Inc., New York Vol. 1 (1994) at 10.8.1.

ELISAs comprise preparing antigen, coating the well of a 96 well microtiter plate with the antigen, adding the binding molecule of interest conjugated to a detectable compound such as an enzymatic substrate (e.g., horseradish peroxidase or alkaline phosphatase) to the well and incubating for a period of time, and detecting the presence of the antigen. In ELISAs the binding molecule of interest does not have to be conjugated to a detectable compound; instead, a second binding molecule (which recognizes the binding molecule of interest) conjugated to a detectable compound may be added to the well. Further, instead of coating the well with the antigen, the binding molecule may be coated to the well. In this case, a second binding molecule conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds, Current Protocols in Molecular Biology, John Wiley & Sons, Inc., New York, Vol. 1 (1994) at 11.2.1.

The binding affinity of binding molecule to an antigen and the off-rate of a binding molecule-antigen interaction can be determined by competitive binding assays. One example of a competitive binding assay is a radioimmunoassay comprising the incubation of labeled antigen (e.g., ³H or ¹²⁵I) with the binding molecule of interest in the presence of increasing amounts of unlabeled antigen, and the detection of the binding molecule bound to the labeled antigen. The affinity of the binding molecule of interest for a particular antigen and the binding off-rates can be determined from the data by Scatchard plot analysis. Competition with a second binding molecule can also be determined using radioimmunoassays. In this case, the antigen is incubated with antibody of interest is conjugated to a labeled compound (e.g., ³H or ¹²⁵I) in the presence of increasing amounts of an unlabeled second binding molecule.

IGF-1R-specific binding molecules may, additionally, be employed histologically, as in immunofluorescence, immunoelectron microscopy or non-immunological assays, for in situ detection of cancer antigen gene products or conserved variants or peptide fragments thereof. In situ detection may be accomplished by removing a histological specimen from a patient, and applying thereto a labeled IGF-1R-specific binding molecule, preferably applied by overlaying the labeled antibody (or fragment) onto a biological sample. Through the use of such a procedure, it is possible to determine not only the presence of IGF-1R protein, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays and non-immunoassays for IGF-1R gene products or conserved variants or peptide fragments thereof will typically comprise incubating a sample, such as a biological fluid, a tissue extract, freshly harvested cells, or lysates of cells which have been incubated in cell culture, in the presence of a detectably labeled binding molecule capable of binding to IGF-1R or conserved variants or peptide fragments thereof, and detecting the bound binding molecule by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled IGF-1R-specific binding molecule. The solid phase support may then be washed with the buffer a second time to remove unbound antibody. Optionally the binding molecule is subsequently labeled. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding a binding molecule or antigen, or will be able to ascertain the same by use of routine experimentation.

The binding activity of a given lot of IGF-1R-specific binding molecule may be determined according to well known methods. Those skilled in the art will be able to determine operative and optimal assay conditions for each determination by employing routine experimentation.

There are a variety of methods available for measuring the affinity of a binding molecule-antigen interaction, but relatively few for determining rate constants. Most of the methods rely on either labeling a binding molecule or antigen, which inevitably complicates routine measurements and introduces uncertainties in the measured quantities.

Surface plasmon resonance (SPR) as performed on BIAcore offers a number of advantages over conventional methods of measuring the affinity of antibody-antigen interactions: (i) no requirement to label either antibody or antigen; (ii) antibodies do not need to be purified in advance, cell culture supernatant can be used directly; (iii) real-time measurements, allowing rapid semi-quantitative comparison of different monoclonal antibody interactions, are enabled and are sufficient for many evaluation purposes; (iv) biospecific surface can be regenerated so that a series of different monoclonal antibodies can easily be compared under identical conditions; (v) analytical procedures are fully automated, and extensive series of measurements can be performed without user intervention. BIAapplications Handbook, version AB (reprinted 1998), BIACORE code No. BR-1001-86; BIAtechnology Handbook, version AB (reprinted 1998), BIACORE code No. BR-1001-84.

SPR based binding studies require that one member of a binding pair be immobilized on a sensor surface. The binding partner immobilized is referred to as the ligand. The binding partner in solution is referred to as the analyte. In some cases, the ligand is attached indirectly to the surface through binding to another immobilized molecule, which is referred as the capturing molecule. SPR response reflects a change in mass concentration at the detector surface as analytes bind or dissociate.

Based on SPR, real-time BIAcore measurements monitor interactions directly as they happen. The technique is well suited to determination of kinetic parameters. Comparative affinity ranking is extremely simple to perform, and both kinetic and affinity constants can be derived from the sensorgram data.

When analyte is injected in a discrete pulse across a ligand surface, the resulting sensorgram can be divided into three essential phases: (i) Association of analyte with ligand during sample injection; (ii) Equilibrium or steady state during sample injection, where the rate of analyte binding is balanced by dissociation from the complex; (iii) Dissociation of analyte from the surface during buffer flow.

The association and dissociation phases provide information on the kinetics of analyte-ligand interaction (kₐ and k_{d}, the rates of complex formation and dissociation, k_{d}/kₐ=K_{D}). The equilibrium phase provides information on the affinity of the analyte-ligand interaction (K_{D}).

BIAevaluation software provides comprehensive facilities for curve fitting using both numerical integration and global fitting algorithms. With suitable analysis of the data, separate rate and affinity constants for interaction can be obtained from simple BIAcore investigations. The range of affinities measurable by this technique is very broad ranging from mM to pM.

Epitope specificity is an important characteristic of a binding molecule. Epitope mapping with BIAcore, in contrast to conventional techniques using radioimmunoassay, ELISA or other surface adsorption methods, does not require labeling or purified binding molecules, and allows multi-site specificity tests using a sequence of several binding molecules. Additionally, large numbers of analyses can be processed automatically.

Pair-wise binding experiments test the ability of two binding molecules to bind simultaneously to the same antigen. Binding molecules directed against separate epitopes will bind independently, whereas MAbs directed against identical or closely related epitopes will interfere with each other's binding. These binding experiments with BIAcore are straightforward to carry out.

For example, one can use a capture molecule to bind the first binding molecule, followed by addition of antigen and second binding molecule sequentially. The sensorgrams will reveal: 1. how much of the antigen binds to first binding molecule, 2. to what extent the second binding molecule binds to the surface-attached antigen, 3. if the second binding molecule does not bind, whether reversing the order of the pair-wise test alters the results.

Peptide inhibition is another technique used for epitope mapping. This method can complement pair-wise antibody binding studies, and can relate functional epitopes to structural features when the primary sequence of the antigen is known. Peptides or antigen fragments are tested for inhibition of binding of different binding molecules to immobilized antigen. Peptides which interfere with binding of a given binding molecule are assumed to be structurally related to the epitope defined by that binding molecule.

### XI. Pharmaceutical Compositions and Administration Methods

Methods of preparing and administering IGF-1R-specific binding molecules to a subject in need thereof are well known to or are readily determined by those skilled in the art. The route of administration of the binding molecule may be, for example, oral, parenteral, by inhalation or topical. The term parenteral as used herein includes, e.g., intravenous, intraarterial, intraperitoneal, intramuscular, subcutaneous, rectal or vaginal administration. While all these forms of administration are clearly contemplated as being within the scope of the invention, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, binding molecules can be delivered directly to the site of the adverse cellular population thereby increasing the exposure of the diseased tissue to the therapeutic agent.

Preparations for parenteral administration includes sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a binding molecule of the invention) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 (US-2002-0102208 A1), which is incorporated herein by reference in its entirety. Such articles of manufacture will preferably have labels or package inserts indicating that the associated compositions are useful for treating a subject suffering from, or predisposed to autoimmune or neoplastic disorders.

Effective doses of the compositions of the present invention, for treatment of hyperproliferative disorders as described herein vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

For treatment of hyperproliferative disorders with an antibody or fragment thereof, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered falls within the ranges indicated.

IGF-1R-specific binding molecules disclosed herein can be administered on multiple occasions. Intervals between single dosages can be weekly, monthly or yearly. Intervals can also be irregular as indicated by measuring blood levels of target polypeptide or target molecule in the patient. In some methods, dosage is adjusted to achieve a plasma polypeptide concentration of 1-1000 μg/ml and in some methods 25-300 μg/ml. Alternatively, binding molecules can be administered as a sustained release formulation, in which case less frequent administration is required. Dosage and frequency vary depending on the half-life of the antibody in the patient. The half-life of a binding molecule can also be prolonged via fusion to a stable polypeptide or moiety, e.g., albumin or PEG. In general, humanized antibodies show the longest half-life, followed by chimeric antibodies and nonhuman antibodies. In one embodiment, the binding molecules of the invention can be administered in unconjugated form, In another embodiment, the binding molecules for use in the methods disclosed herein can be administered multiple times in conjugated form. In still another embodiment, the binding molecules of the invention can be administered in unconjugated form, then in conjugated form, or vise versa.

The dosage and frequency of administration can vary depending on whether the treatment is prophylactic or therapeutic. In prophylactic applications, compositions comprising antibodies or a cocktail thereof are administered to a patient not already in the disease state or in a pre-disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In therapeutic applications, a relatively high dosage (e.g., from about 1 to 400 mg/kg of binding molecule, e.g., antibody per dose, with dosages of from 5 to 25 mg being more commonly used for radioimmunoconjugates and higher doses for cytotoxin-drug conjugated molecules) at relatively short intervals is sometimes required until progression of the disease is reduced or terminated, and preferably until the patient shows partial or complete amelioration of symptoms of disease. Thereafter, the patent can be administered a prophylactic regime.

In one embodiment, a subject can be treated with a nucleic acid molecule encoding an IGF-1R-specific antibody or immunospecific fragment thereof (e.g., in a vector). Doses for nucleic acids encoding polypeptides range from about 10 ng to 1 g, 100 ng to 100 mg, 1 μg to 10 mg, or 30-300 μg DNA per patient. Doses for infectious viral vectors vary from 10-100, or more, virions per dose.

Therapeutic agents can be administered by parenteral, topical, intravenous, oral, subcutaneous, intraarterial, intracranial, intraperitoneal, intranasal or intramuscular means for prophylactic and/or therapeutic treatment. In some methods, agents are injected directly into a particular tissue where IGF-1R-expressing cells have accumulated, for example intracranial injection. Intramuscular injection or intravenous infusion are preferred for administration of antibody. In some methods, particular therapeutic antibodies are injected directly into the cranium. In some methods, antibodies are administered as a sustained release composition or device, such as a Medipad™ device.

IGF-1R binding molecules can optionally be administered in combination with other agents that are effective in treating the disorder or condition in need of treatment (e.g., prophylactic or therapeutic).

Effective single treatment dosages (i.e., therapeutically effective amounts) of ⁹⁰Y-labeled binding polypeptides range from between about 5 and about 75 mCi, more preferably between about 10 and about 40 mCi. Effective single treatment non-marrow ablative dosages of ¹³¹I-labeled antibodies range from between about 5 and about 70 mCi, more preferably between about 5 and about 40 mCi. Effective single treatment ablative dosages (i.e., may require autologous bone marrow transplantation) of ¹³¹I-labeled antibodies range from between about 30 and about 600 mCi, more preferably between about 50 and less than about 500 mCi. In conjunction with a chimeric antibody, owing to the longer circulating half life vis-à-vis murine antibodies, an effective single treatment non-marrow ablative dosages of iodine-131 labeled chimeric antibodies range from between about 5 and about 40 mCi, more preferably less than about 30 mCi. Imaging criteria for, e.g., the ¹¹¹In label, are typically less than about 5 mCi.

While a great deal of clinical experience has been gained with ¹³¹I and ⁹⁰Y, other radiolabels are known in the art and have been used for similar purposes. Still other radioisotopes are used for imaging. For example, additional radioisotopes which are compatible with the scope of the instant invention include, but are not limited to, ¹²³I, ¹²⁵I, ³²P, ⁵⁷Co, ⁶⁴Cu, ⁶⁷Cu, ⁷⁷Br, ⁸¹Rb, ⁸¹Kr, ⁸³Sr, ¹¹³In, ¹²⁷Cs, ¹²⁹Cs, ¹³²I, ⁹⁷Hg, ²⁰³Pb, ²⁰⁶Bi, ¹⁷⁷Lu, ¹⁸⁶Re, ²¹²Pb, ²¹²Bi, 47Sc, ¹⁰⁵Rh, ¹⁰⁹Pd, ¹⁵³Sm, ¹⁸⁸Re, ¹⁹⁹Au, ²²⁵Ac, ²¹¹At, and ²¹³Bi. In this respect alpha, gamma and beta emitters are all compatible with in the instant invention. Further, in view of the instant disclosure it is submitted that one skilled in the art could readily determine which radionuclides are compatible with a selected course of treatment without undue experimentation. To this end, additional radionuclides which have already been used in clinical diagnosis include ¹²⁵I, ¹²³I, ⁹⁹Tc, ⁴³K, ⁵²Fe, ⁶⁷Ga, ⁶⁸Ga, as well as ¹¹¹In. Antibodies have also been labeled with a variety of radionuclides for potential use in targeted immunotherapy (Peirersz et al. Immunol. Cell Biol. 65: 111-125 (1987)). These radionuclides include ¹⁸⁸Re and ¹⁸⁶Re as well as ¹⁹⁹Au and ⁶⁷Cu to a lesser extent. U.S. Pat. No. 5,460,785 provides additional data regarding such radioisotopes and is incorporated herein by reference.

Whether or not IGF-1R-specific binding molecules disclosed herein are used in a conjugated or unconjugated form, it will be appreciated that a major advantage of the present invention is the ability to use these molecules in myelosuppressed patients, especially those who are undergoing, or have undergone, adjunct therapies such as radiotherapy or chemotherapy. That is, the beneficial delivery profile (i.e. relatively short serum dwell time, high binding affinity and enhanced localization) of the molecules makes them particularly useful for treating patients that have reduced red marrow reserves and are sensitive to myelotoxicity. In this regard, the unique delivery profile of the molecules make them very effective for the administration of radiolabeled conjugates to myelosuppressed cancer patients. As such, the IGF-1R-specific binding molecules disclosed herein are useful in a conjugated or unconjugated form in patients that have previously undergone adjunct therapies such as external beam radiation or chemotherapy. In other preferred embodiments, binding molecules of the invention (again in a conjugated or unconjugated form) may be used in a combined therapeutic regimen with chemotherapeutic agents. Those skilled in the art will appreciate that such therapeutic regimens may comprise the sequential, simultaneous, concurrent or coextensive administration of the disclosed antibodies or other binding molecules and one or more chemotherapeutic agents. Particularly preferred embodiments of this aspect of the invention will comprise the administration of a radiolabeled binding polypeptide.

While IGF-1R-specific binding molecules may be administered as described immediately above, it must be emphasized that in other embodiments conjugated and unconjugated binding molecules may be administered to otherwise healthy patients as a first line therapeutic agent. In such embodiments binding molecules may be administered to patients having normal or average red marrow reserves and/or to patients that have not, and are not, undergoing adjunct therapies such as external beam radiation or chemotherapy. However, as discussed above, selected embodiments of the invention comprise the administration of IGF-1R-specific binding molecule to myelosuppressed patients or in combination or conjunction with one or more adjunct therapies such as radiotherapy or chemotherapy (i.e. a combined therapeutic regimen). As used herein, the administration of IGF-1R-specific binding molecule in conjunction or combination with an adjunct therapy means the sequential, simultaneous, coextensive, concurrent, concomitant or contemporaneous administration or application of the therapy and the disclosed binding molecules. Those skilled in the art will appreciate that the administration or application of the various components of the combined therapeutic regimen may be timed to enhance the overall effectiveness of the treatment. For example, chemotherapeutic agents could be administered in standard, well known courses of treatment followed within a few weeks by radioimmunoconjugates described herein. Conversely, cytotoxin-conjugated binding molecules could be administered intravenously followed by tumor localized external beam radiation. In yet other embodiments, binding molecules may be administered concurrently with one or more selected chemotherapeutic agents in a single office visit. A skilled artisan (e.g. an experienced oncologist) would be readily be able to discern effective combined therapeutic regimens without undue experimentation based on the selected adjunct therapy and the teachings of the instant specification.

In this regard it will be appreciated that the combination of a binding molecule (with or without cytotoxin) and the chemotherapeutic agent may be administered in any order and within any time frame that provides a therapeutic benefit to the patient. That is, the chemotherapeutic agent and IGF-1R-specific binding molecule may be administered in any order or concurrently. In selected embodiments IGF-1R-specific binding molecules of the present invention will be administered to patients that have previously undergone chemotherapy. In yet other embodiments, IGF-1R-specific antibodies of the present invention will be administered substantially simultaneously or concurrently with the chemotherapeutic treatment. For example, the patient may be given the binding molecule while undergoing a course of chemotherapy. In preferred embodiments the binding molecule will be administered within 1 year of any chemotherapeutic agent or treatment. In other preferred embodiments the polypeptide will be administered within 10, 8, 6, 4, or 2 months of any chemotherapeutic agent or treatment. In still other preferred embodiments the binding molecule will be administered within 4, 3, 2 or 1 week of any chemotherapeutic agent or treatment. In yet other embodiments the binding molecule will be administered within 5, 4, 3, 2 or 1 days of the selected chemotherapeutic agent or treatment. It will further be appreciated that the two agents or treatments may be administered to the patient within a matter of hours or minutes (i.e. substantially simultaneously).

Moreover, in accordance with the present invention a myelosuppressed patient shall be held to mean any patient exhibiting lowered blood counts. Those skilled in the art will appreciate that there are several blood count parameters conventionally used as clinical indicators of myelosuppression and one can easily measure the extent to which myelosuppression is occurring in a patient. Examples of art accepted myelosuppression measurements are the Absolute Neutrophil Count (ANC) or platelet count. Such myelosuppression or partial myeloablation may be a result of various biochemical disorders or diseases or, more likely, as the result of prior chemotherapy or radiotherapy. In this respect, those skilled in the art will appreciate that patients who have undergone traditional chemotherapy typically exhibit reduced red marrow reserves. As discussed above, such subjects often cannot be treated using optimal levels of cytotoxin (i.e. radionuclides) due to unacceptable side effects such as anemia or immunosuppression that result in increased mortality or morbidity.

More specifically conjugated or unconjugated IGF-1R-specific antibodies binding molecules of the present invention may be used to effectively treat patients having ANCs lower than about 2000/mm³ or platelet counts lower than about 150,000/mm³. More preferably IGF-1R-specific binding molecules of the present invention may be used to treat patients having ANCs of less than about 1500/mm³, less than about 1000/mm³ or even more preferably less than about 500/mm³. Similarly, IGF-1R-specific binding molecules of the present invention may be used to treat patients having a platelet count of less than about 75,000/mm³, less than about 50,000/mm³ or even less than about 10,000/mm³. In a more general sense, those skilled in the art will easily be able to determine when a patient is myelosuppressed using government implemented guidelines and procedures.

As indicated above, many myelosuppressed patients have undergone courses of treatment including chemotherapy, implant radiotherapy or external beam radiotherapy. In the case of the latter, an external radiation source is for local irradiation of a malignancy. For radiotherapy implantation methods, radioactive reagents are surgically located within the malignancy, thereby selectively irradiating the site of the disease. In any event, IGF-1R-specific binding molecules of the present invention may be used to treat disorders in patients exhibiting myelosuppression regardless of the cause.

In this regard it will further be appreciated that IGF-1R-specific binding molecules of the present invention may be used in conjunction or combination with any chemotherapeutic agent or agents (e.g. to provide a combined therapeutic regimen) that eliminates, reduces, inhibits or controls the growth of neoplastic cells in vivo. As discussed, such agents often result in the reduction of red marrow reserves. This reduction may be offset, in whole or in part, by the diminished myelotoxicity of the compounds of the present invention that advantageously allow for the aggressive treatment of neoplasias in such patients. In other embodiments, radiolabeled immunoconjugates disclosed herein may be effectively used with radiosensitizers that increase the susceptibility of the neoplastic cells to radionuclides. For example, radiosensitizing compounds may be administered after the radiolabeled binding molecule has been largely cleared from the bloodstream but still remains at therapeutically effective levels at the site of the tumor or tumors.

With respect to these aspects of the invention, exemplary chemotherapeutic agents that are compatible with the instant invention include alkylating agents, vinca alkaloids (e.g., vincristine and vinblastine), procarbazine, methotrexate and prednisone. The four-drug combination MOPP (mechlethamine (nitrogen mustard), vincristine (Oncovin), procarbazine and prednisone) is very effective in treating various types of lymphoma and comprises a preferred embodiment of the present invention. In MOPP-resistant patients, ABVD (e.g., adriamycin, bleomycin, vinblastine and dacarbazine), ChlVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphomas, in Harrison's Principles of Internal Medicine 1774-1788 (Kurt J. Isselbacher et al., eds., 13^{th} ed. 1994) and V. T. DeVita et al., (1997) and the references cited therein for standard dosing and scheduling. These therapies can be used unchanged, or altered as needed for a particular patient, in combination with one or more IGF-1R-specific antibodies or immunospecific fragments thereof of the present invention.

Additional regimens that are useful in the context of the present invention include use of single alkylating agents such as cyclophosphamide or chlorambucil, or combinations such as CVP (cyclophosphamide, vincristine and prednisone), CHOP (CVP and doxorubicin), C-MOPP (cyclophosphamide, vincristine, prednisone and procarbazine), CAP-BOP(CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). Those skilled in the art will readily be able to determine standard dosages and scheduling for each of these regimens. CHOP has also been combined with bleomycin, methotrexate, procarbazine, nitrogen mustard, cytosine arabinoside and etoposide. Other compatible chemotherapeutic agents include, but are not limited to, 2-chlorodeoxyadenosine (2-CDA), 2′-deoxycoformycin and fludarabine.

For patients with intermediate- and high-grade malignancies, who fail to achieve remission or relapse, salvage therapy is used. Salvage therapies employ drugs such as cytosine arabinoside, cisplatin, carboplatin, etoposide and ifosfamide given alone or in combination. In relapsed or aggressive forms of certain neoplastic disorders the following protocols are often used: IMVP-16 (ifosfamide, methotrexate and etoposide), MIME (methyl-gag, ifosfamide, methotrexate and etoposide), DHAP (dexamethasone, high dose cytarabine and cisplatin), ESHAP (etoposide, methylpredisolone, HD cytarabine, cisplatin), CEPP(B) (cyclophosphamide, etoposide, procarbazine, prednisone and bleomycin) and CAMP (lomustine, mitoxantrone, cytarabine and prednisone) each with well known dosing rates and schedules.

The amount of chemotherapeutic agent to be used in combination with the IGF-1R-specific binding molecules of the present invention may vary by subject or may be administered according to what is known in the art. See for example, Bruce A Chabner et al., Antineoplastic Agents, in Goodman & Gilman's The Pharmacological Basis of Therapeutics 1233-1287 (Joel G. Hardman et al., eds., 9^{th} ed. (1996)).

In another embodiment, an IGF-1R-specific binding molecule of the present invention is administered in conjunction with a biologic. Biologics useful in the treatment of cancers are known in the art and a binding molecule of the invention may be administered, for example, in conjunction with such known biologics.

For example, the FDA has approved the following biologics for the treatment of breast cancer: Herceptin® (trastuzumab, Genentech Inc., South San Francisco, Calif.; a humanized monoclonal antibody that has anti-tumor activity in HER2-positive breast cancer); Faslodex® (fulvestrant, AstraZeneca Pharmaceuticals, LP, Wilmington, Del.; an estrogen-receptor antagonist used to treat breast cancer); Arimidex® (anastrozole, AstraZeneca Pharmaceuticals, LP; a nonsteroidal aromatase inhibitor which blocks aromatase, an enzyme needed to make estrogen); Aromasin® (exemestane, Pfizer Inc., New York, N.Y.; an irreversible, steroidal aromatase inactivator used in the treatment of breast cancer); Femara® (letrozole, Novartis Pharmaceuticals, East Hanover, N.J.; a nonsteroidal aromatase inhibitor approved by the FDA to treat breast cancer); and Nolvadex® (tamoxifen, AstraZeneca Pharmaceuticals, LP; a nonsteroidal antiestrogen approved by the FDA to treat breast cancer). Other biologics with which the binding molecules of the invention may be combined include: Avastin™ (bevacizumab, Genentech Inc.; the first FDA-approved therapy designed to inhibit angiogenesis); and Zevalin® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: Avastin™; Erbitux™ (cetuximab, ImClone Systems Inc., New York, N.Y., and Bristol-Myers Squibb, New York, N.Y.; is a monoclonal antibody directed against the epidermal growth factor receptor (EGFR)); Gleevec® (imatinib mesylate; a protein kinase inhibitor); and Ergamisol® (levamisole hydrochloride, Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For use in treatment of Non-Hodgkin's Lymphomas currently approved therapies include: Bexxar® (tositumomab and iodine I-131 tositumomab, GlaxoSmithKline, Research Triangle Park, N.C.; a multi-step treatment involving a mouse monoclonal antibody (tositumomab) linked to a radioactive molecule (iodine I-131)); Intron® A (interferon alfa-2b, Schering Corporation, Kenilworth, N.J.; a type of interferon approved for the treatment of follicular non-Hodgkin's lymphoma in conjunction with anthracycline-containing combination chemotherapy (e.g., cyclophosphamide, doxorubicin, vincristine, and prednisone [CHOP])); Rituxan® (rituximab, Genentech Inc., South San Francisco, Calif., and Biogen Idec, Cambridge, Mass.; a monoclonal antibody approved for the treatment of non-Hodgkin's lymphoma; Ontak® (denileukin diftitox, Ligand Pharmaceuticals Inc., San Diego, Calif.; a fusion protein consisting of a fragment of diphtheria toxin genetically fused to interleukin-2); and Zevalin® (ibritumomab tiuxetan, Biogen Idec; a radiolabeled monoclonal antibody approved by the FDA for the treatment of B-cell non-Hodgkin's lymphomas).

For treatment of Leukemia, exemplary biologics which may be used in combination with the binding molecules of the invention include Gleevec®; Campath®-1H (alemtuzumab, Berlex Laboratories, Richmond, Calif.; a type of monoclonal antibody used in the treatment of chronic Lymphocytic leukemia). In addition, Genasense (oblimersen, Genta Corporation, Berkley Heights, N.J.; a BCL-2 antisense therapy under development to treat leukemia may be used (e.g., alone or in combination with one or more chemotherapy drugs, such as fludarabine and cyclophosphamide) may be administered with the claimed binding molecules.

For the treatment of lung cancer, exemplary biologics include Tarceva™ (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include Velcade® Velcade (bortezomib, Millennium Pharmaceuticals, Cambridge Mass.; a proteasome inhibitor). Additional biologics include Thalidomid® (thalidomide, Clegene Corporation, Warren, N.J.; an immunomodulatory agent and appears to have multiple actions, including the ability to inhibit the growth and survival of myeloma cells and anti-angiogenesis).

Other exemplary biologics include the MOAB IMC-C225, developed by ImClone Systems, Inc., New York, N.Y.

As previously discussed, IGF-1R-specific binding molecules of the present invention, or recombinants thereof may be administered in a pharmaceutically effective amount for the in vivo treatment of mammalian hyperproliferative disorders. In this regard, it will be appreciated that the disclosed binding molecules will be formulated so as to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of IGF-1R-specific binding molecules of the present invention, or recombinant thereof, conjugated or unconjugated to a therapeutic agent, shall be held to mean an amount sufficient to achieve effective binding to a target and to achieve a benefit, e.g., to ameliorate symptoms of a disease or disorder or to detect a substance or a cell. In the case of tumor cells, the binding molecule will be preferably be capable of interacting with selected immunoreactive antigens on neoplastic or immunoreactive cells, or on non neoplastic cells, e.g., vascular cells associated with neoplastic cells. and provide for an increase in the death of those cells. Of course, the pharmaceutical compositions of the present invention may be administered in single or multiple doses to provide for a pharmaceutically effective amount of the binding molecule.

In keeping with the scope of the present disclosure, IGF-1R-specific binding molecules of the present invention may be administered to a human or other animal in accordance with the aforementioned methods of treatment in an amount sufficient to produce a therapeutic or prophylactic effect. The IGF-1R-specific antibodies binding molecules of the present invention can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of binding molecules according to the present invention may prove to be particularly effective.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

General principles of antibody engineering are set forth in Antibody Engineering, 2nd edition, C. A. K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies and antibody-hapten binding are set forth in: Nisonoff, A., Molecular Immunology, 2nd ed., Sinauer Associates, Sunderland, Mass. (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al. (eds), Basic and Clinical-Immunology (8th ed.), Appleton & Lange, Norwalk, Conn. (1994) and Mishell and Shiigi (eds), Selected Methods in Cellular Immunology, W.H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984), Kuby Immunology 4^{th} ed. Ed. Richard A. Goldsby, Thomas J. Kindt and Barbara A. Osborne, H. Freemand & Co. (2000); Roitt, I., Brostoff, J. and Male D., Immunology 6^{th} ed. London: Mosby (2001); Abbas A., Abul, A. and Lichtman, A., Cellular and Molecular Immunology Ed. 5, Elsevier Health Sciences Division (2005); Kontermann and Dubel, Antibody Engineering, Springer Verlan (2001); Sambrook and Russell, Molecular Cloning: A Laboratory Manual. Cold Spring Harbor Press (2001); Lewin, Genes VIII, Prentice Hall (2003); Harlow and Lane, Antibodies: A Laboratory Manual, Cold Spring Harbor Press (1988); Dieffenbach and Dveksler, PCR Primer Cold Spring Harbor Press (2003).

All of the references cited above, as well as all references cited herein, are incorporated herein by reference in their entireties. In addition, further aspects of the invention are set forth in the sequence listing portion of the description and the drawings.

### EXAMPLES

### Example 1

### The M13.C06 Antibody Recognizes an Epitope that is Distinct from Other Inhibitory Anti-IGF-1R Antibodies

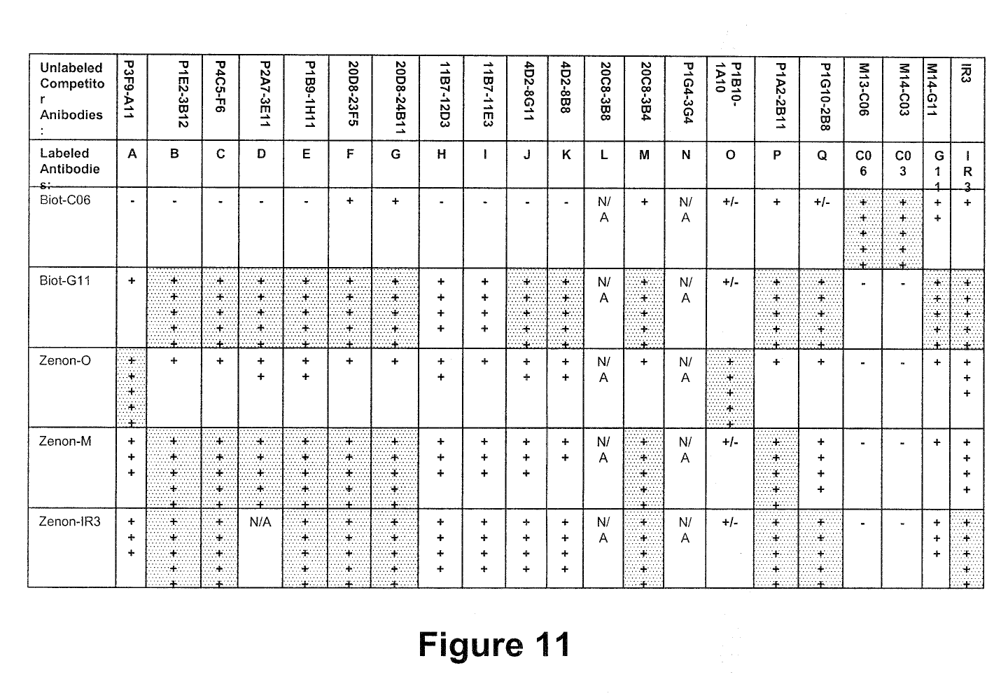

A cross-competition antibody binding study was performed to compare the IGF-1R antibody binding epitopes of M13.C06.G4.P.agly and other IGF-1R antibodies. See, FIG. 11. Unlabeled competitor antibodies were analyzed for their ability to cross-compete with five different labeled antibodies for binding to soluble IGF-1R. The five labeled antibodies used were biotin-labeled M13.C06.G4.P.agly (“Biotin-C06”), biotin labeled M14-G11 (“Biotin-G11”), zenon-labeled P1B10-1A10 (“Zenon-O”), zenon-labeled 20C8-3B4 (“Zenon-M”), or zenon-labeled IR3 antibody (“Zenon-IR3”). See, FIG. 11. Antibodies were labeled with Biotin using a Biotinylation kit from Pierce Chemical (#21335). Zenon labeling was performed using Zenon mouse IgG labeling kit from Molecular Probes (Z25000).

The results of this analysis indicate that M13.C06.G4.P.agly and M14.C03.G4.P.agly antibodies bind to the same or a similar region of IGF-1R, which is distinct from all other antibodies tested. In particular, only biotin-labeled M13.C06.G4.P.agly antibody was effectively competed from IGF-1R binding by unlabeled M13.C06.G4.P.agly or by unlabeled M14.C03.G4.P.agly. It is also notable that M13.C06.G4.P.agly does not cross-compete with the well-studied IR3 antibody. Hence, these two antibodies, in particular, bind to different IGF-1R epitopes.

### Example 2

### The M13.C06 Antibody Binds the N-Terminal Region of the FnIII-1 Domain and Allosterically Decreases the Binding Affinity of IGF-1 and IGF-2 for IGF-1R

a. Methods:

i. IGF-1/IGF-1R Binding Experiments in the Presence and Absence of M13-C06 Antibody

Several constructs were used to investigate antibody/IGF-1 binding to the IGF-1R receptor or insulin receptor: human IGF-1R(1-902)-His₁₀ (denoted hIGF-1R-His₁₀, from R&D systems), human INSR(28-956)-His₁₀ (denoted INSR, from R&D systems), human IGF-1R(1-903)-Fc (denoted hIGF-1R-Fc, generated by Biogen Idec), human IGF-1R(1-462)-Fc (denoted hIGF-1R(1-462)-Fc, generated by Biogen Idec), and murine IGF-1R(1-903)-Fc (denoted mIGF-1R-Fc, generated by Biogen Idec). “His₁₀” denotes a 10-residue histidine tag on the C-terminus of the constructs. “Fc” denotes a C-terminal human IgG1-Fc tag.

Human IGF-1 was purchased from Millipore. The affinity of IGF-1 for hIGF-1R-His₁₀ was determined using surface plasmon resonance (SPR). A biotin-labeled anti-HisTag antibody (biotin-PENTA-His, Qiagen Cat. No. 34440) was immobilized to saturation on a Biacore SA chip (Cat. No. BR-1000-32) surface by injection at 500 nM in HBS-EP buffer. For each sensorgram, hIGF-1R-His₁₀ was captured on the biotin-PENTA-His surface by injecting 20 □L of 40 nM protein at 2 μL/min. Subsequent to hIGF-1R-His₁₀ injection, the flow rate was increased to 20 μL/min. A second, 130 μL injection containing IGF-1 was performed to investigate interaction of the growth hormone with its receptor. IGF-1 was serially diluted from 64 nM to 0.125 nM to obtain concentration dependent kinetic binding curves. Each injection series was regenerated using 3×10 □L injections of 10 mM Acetate, pH 4.0, at 20 μL/min. Each curve was double referenced using (1) data obtained from a streptavidin surface devoid of PENTA-His antibody and (2) data from a primary injection of hIGF-1R-His₁₀ followed by a secondary injection of HBS-EP buffer. The concentration series for IGF-1 was fit to the 1:1 binding model provided within the BiaEvaluation software of the manufacturer. Two sets of data were obtained, one in the absence and another in the presence of 400 nM M13-C06 in the running buffer, hIGF-1R-His₁₀ injection buffer, and IGF-1 injection buffer.

ii. Pull-Down and Western Blot Analysis of IGF-1/IGF-1R/M13-C06 Antibody Ternary Complexes

Resuspended Protein A/G beads (300 μl, Pierce Cat. No. 20422) were washed with 1×PBS and mixed with 1.0 mg M13-C06 in a 1.5 ml Eppendorf tube on a rotary shaker for two hours at room temperature. In a separate tube, 12 μg hIGF-1R-His₁₀ (R&D systems) and 460 ng human IGF-1 (Chemicon International Cat. No. GF006) were mixed (1:1 protein:protein ratio) for one hour at room temperature. Protein A/G with bound M13-C06 was washed with PBS and incubated with the hIGF-1R-His₁₀/IGF-1 mixture for 30 minutes at room temperature. Protein A/G with bound protein was washed with PBS followed by elution of bound protein with 300 μL 100 mM glycine, pH 3.0. For the negative control, the addition of 12 μg human IGF-1R(1-902)-His₁₀ was omitted. Eluted proteins were detected by Western Blot with an anti-human IGF-1 antibody (Rabbit anti-Human IGF-1 Biotin, USBiological Cat. No. I7661-01B) and an anti-human IGF-1R antibody (IGF-1Rα 1H7, Santa Cruz Biotechnology Cat. No. sc-461) as primary antibodies, followed by HRP-labeled streptavidin (Southern Biotech Cat. No. 7100-05) and HRP-labeled goat anti-mouse IgG (USBiological Cat. No. I1904-40J) as secondary antibodies. To demonstrate the ability of IGF-1/IGF-1R/M13-C06 to form a ternary complex, the concentrations of the IGF-1 and IGF-1R used in this experiment were well in excess (>15-fold above) the normal physiological levels of these proteins (particularly IGF-1 in the serum) as well as the measured equilibrium dissociation constant for IGF-1R/IGF-1. See, for example, Hankinson et al., 1997.

iii. Construction of IGF-1R(1-462)-Fc and Comparative Antibody Binding Studies Versus the Full-Length Receptor Ectodomain

Construction of the IGF-1/IGF-2 binding domains, L1-CR-L2 (residues 1-462), of human IGF-1R was published previously (McKern 1997). Utilizing this information, we subcloned human IGF-1R residues 1-462 (along with the N-terminal signal sequence) into the same in-house PV90 vector that was used to produce the full-length human ectodomain (residues 1-903, hIGF-1R-Fc). Expression in CHO was facilitated using methods described previously (Brezinsky 2003). The protein was purified from CHO supernatants by passage over a protein A affinity column as described previously for other Fc-fusion proteins (Demarest 2006). The protein construct is denoted hIGF-1R(1-462)-Fc.

The ability of M13-C06, M14-C03, and M14-G11 antibodies to bind hIGF-1R(1-462)-Fc and the full-length ectodomain construct, hIGF-1R-Fc, was determined by SPR using a Biacore3000. The instrument was set to 25° C. and the running buffer was HBS-EP, pH 7.2 (Biacore, Cat. No. BR-1001-88). The fully human antibodies, M13-C06, M14-C03, and M14-G11, were immobilized to ˜10,000 RU on Biacore CM5 Research Grade Sensor Chip (Cat. No. BR-1000-14) surfaces using the standard NHS/EDC-amine reactive chemistry according to protocols supplied by Biacore. For immobilization, the antibodies were diluted to 40 μg/mL in a 10 mM Acetate pH 4.0 buffer. To investigate the relative kinetics of association and dissociation of hIGF-1R-Fc and hIGF-1R(1-462)-Fc to each of the human antibodies, increasing concentrations of each receptor construct were injected over the sensorchip surfaces. The hIGF-1R-Fc concentration series ranged from 1.0 nM to 100 nM while the hIGF-1R(1-462)-Fc concentration series ranged from 1.0 nM to 2 μM. All antibody surfaces were reliably regenerated with 100 mM Glycine, pH 2.0. Repeated regenerations did not lead to activity losses for any of the antibody surfaces. Flow rates were 20 μl/min.

b. Results

Inhibition of IGF-1 and/or IGF-2 binding to hIGF-1R-Fc by M13-C06 was demonstrated as previously described. Even at saturating conditions, most antibodies do not fully inhibit IGF-1 or IGF-2 binding to hIGF-1R-Fc. Particularly for M13-C06, it was hypothesized that inhibition of ligand binding might be non-competitive or allosteric. To test this hypothesis, the affinity of IGF-1 for hIGF-1R-His₁₀ was determined in the presence and absence of 400 nM M13-C06 antibody (˜4000-fold above the affinity of the antibody for hIGF-1R-His₁₀). Using SPR hIGF-1R-His₁₀ was immobilized to chip surfaces using an anti-Histag antibody followed by injection of increasing concentrations of IGF-1 (up to 64 nM). IGF-1 binding to hIGF-1R-His₁₀ was evident in the absence and presence of 400 nM M13-C06. (Data not shown: Surface plasmon resonance demonstrating binding of IGF-1 to hIGF-1R-His₁₀ in the absence and presence of 400 nM M13-C06. The SPR association phase was between 1400-1800 seconds while the dissociation phase was between 1800-3000 seconds. In the absence of M13-C06, IGF-1 bound to hIGF-1R-His₁₀ with K_{D}=17 nM (kₐ=2.4×10⁻⁵/M*s). In the presence, of 400 nM M13-C06, IGF-1 bound to hIGF-1R-His₁₀ with K_{D}=59 nM (kₐ=7.1×10⁻⁴/M*s).) The kinetic association rate constant of IGF-1 binding to hIGF-1R-His₁₀ was reduced approximately 3-fold in the presence of M13-C06, suggesting that M13-C06 allosterically reduces the affinity of the ligand for the receptor.

Supporting evidence that M13-C06 does not directly compete with IGF-1 for binding to hIGF-1R-His₁₀ was generated by performing a co-immunoprecipitation of hIGF-1R-His₁₀ and IGF-1 using M13-C06 at concentrations well above the apparent affinities of both IGF-1 and M13-C06 for hIGF-1R-His₁₀. Western blot analysis demonstrated that ˜70-100% of the IGF-1 material mixed with hIGF-1R-His₁₀ was pulled down with M13-C06, thereby demonstrating that co-engagement of M13-C06 and IGF-1 with hIGF-1R-His₁₀ to form the ternary complex is possible (data not shown). These results demonstrate the allosteric nature of M13-C06 inhibition of IGF-1 binding at normal IGF-1 serum concentrations and suggest that the binding site of M13-C06 does not overlap with the IGF-1R ligand-binding pocket.

Next, it was determined whether M13-C06 binds the putative ligand binding domains of IGF-1R (L1-CR-L2). A truncated version of the receptor containing the N-terminal three domains (residues 1-462) fused to an IgG1-Fc was generated and its ability to bind M13-C06, M14-C03, and M14-G11 was compared to that of the full-length receptor ectodomain construct, hIGF-1R-Fc, using surface plasmon resonance (SPR). M14-G11 demonstrated equivalent binding to the truncated version of the receptor, while the binding of M13-C06 and M14-C03 was dramatically reduced. (Data not shown: Surface immobilized M13-C06, M14-C03, and M14-G11 antibodies were tested for binding to hIGF-1R(1-903)Fc and truncated hIGF-1R(1-462)-Fc at concentrations ranging from 2 □M, 100 nM, 30 nM, 10 nM, 5 nM and 1 nM. The SPR association phase was between 480-960 seconds while the dissociation phase was between 960-1170 seconds.) Residual binding was apparent for both M13-C06 and M14-C03; however, the data suggests that at least a good portion of the epitopes of these antibodies resides in an IGF-1R region outside the ligand binding domains.

In conclusion, it was demonstrated that M13-C06 antibody does not block IGF-1 and IGF-2 binding to IGF-1R by competitively interacting with the growth factor binding site, but instead binds to FnIII-1 and allosterically inhibits IGF-1/IGF-2 binding and signaling. FnIII-1 is believed to facilitate receptor homodimerization of both IGF-1R and INSR (McKern 2006) and, upon binding ligand, transmit an activating signal through the transmembrane region to the C-terminal tyrosine kinase domains via a quarternary structure change. The data from this example suggests M13-C06 antibody inhibits conformational changes induced by IGF-1/IGF-2 that lead to downstream receptor signaling.

### Example 3

### Preliminary Epitope Mapping of M13.C06 Antibodies

a. Methods

i. Epitope Mapping Mutations

The choice of mutants to probe for the epitope of M13-C06 antibody on IGF-1R were based on the observation that the binding affinity of M13-C06 to mouse IGF-1R was significantly reduced or non-detectable in Biacore and FRET binding experiments. Mouse and human IGF-1R share 95% primary amino acid sequence identity. Human IGF-1R and human INSR share 57% identity (73% similarity). 33 residues that differ between mouse and human IGF-1R in the ectodomain (Table 5). Twenty of these residues were targeted for mutation because the homologous positions within the INSR ectodomain were exposed to solvent based on the recent INSR crystal structure (pdb code 2DTG, McKern 2006). Accessible surface areas were calculated using StrucTools (http://molbio.info.nih.gov/structbio/basic.html) with a 1.4 Å probe radius. Four additional residues not in the structure of INSR were also chosen for mutagenesis as they resided in the unstructured loop region of the FnIII-2 domain that has been demonstrated to be important for IGF-1/IGF-2 binding (Whittaker 2001; Sorensen 2004). The list of the 24 mutations chosen for the epitope mapping study are shown in Table 6.

TABLE 5
Amino acid differences between human and mouse IGF-1R. Solvent
accessibility of each residue position was determined based on the
publicly available structure of the homologous INSR ectodomain.
Residues shown in bold/italics exposed greater than 30% of their
surface area to solvent and were mutagenized to screen for the
IGF-1R epitope of M13-C06.

| | Human | Mouse | Human | IR | % Solvent |
|---|---|---|---|---|---|
| Residue # | IGF1R | IGF1R | INSR | pdb # | Accessibility |
| [symbol] | [symbol] | [symbol] | [symbol] | [symbol] | [symbol] |
| 125 | V | I | I | 131 | 0 |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| 214 | N | D | D | 221 | 25.7 |
| 215 | D | N | P | 222 | 20.4 |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| 257 | L | P | H | 263 | 19.2 |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| 326 | F | L | I | 335 | 25.5 |
| [symbol] | [symbol] | [symbol] | [symbol] | [symbol] | [symbol] |
| 411 | I | V | T | 421 | 0.5 |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| 471 | S | W | S | 481 | 26.4 |
| [symbol] | [symbol] | [symbol] | [symbol] | [symbol] | [symbol] |
| [symbol] | [symbol] | [symbol] | [symbol] | [symbol] | [symbol] |
| 605 | S | T | S | 615 | N/A |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |

The 24 mutant epitope mapping library was constructed by mutagenizing the wild-type hIGF-1R-Fc PV-90 plasmid using the Stratagene site-directed mutagenesis kit following the manufacturer's protocols. Incorporation of each mutant (or double mutant in the case of the SD004, SD011, SD014, SD016, and SD019 library members) into the PV-90 vector was confirmed by our in-house DNA sequencing facility. Plasmids were miniprepped and maxiprepped using the Qiagen Miniprep Kit and Qiagen Endotoxin-Free Maxikits, respectively. 200 □g of each mutant plasmid was transiently transfected into 100 mL HEK293 T cells at 2×10⁶ cells/mL using the PolyFect transfection kit (Qiagen) for soluble protein secretion into the media. Cells were cultured in DMEM (IvrineScientific), 10% FBS (low IgG bovine serum, Invitrogen—further depleted of bovine IgG by passage over a 20 mL protein A column) at 37° C. in a CO₂ incubator. After 7 days, supernatants containing each IGF-1R-Fc mutant were collected by centrifugation at 1200 rpm and filtration through a 0.2 μm filter. Each mutant was affinity purified by passage of the supernatants over a 1.2 mL protein A Sepharose FF column pre-equilibrated with 1×PBS. The mutants were eluted from the column using 0.1 M glycine, pH 3.0, neutralized with 1 M Tris, pH 8.5, 0.1% Tween-80, and concentrated to ˜300 μL using VivaSpin 6 MWCO 30,000 centrifugal concentration devices (Sartorius, Cat. No. VS0621).

ii. Western Blot Analysis of IGF-1R Mutants

hIGF-1R-Fc mutant samples were run on 4-20% Tris-Glycine gels (Invitrogen Cat. No. EC6028) using Xcell SureLock Mini Cell (Invitrogen Cat. No. EI0001) following standard manufacturer protocol. Samples were transferred to nitrocellulose using the iBlot Dry Blotting System (Invitrogen Cat. No. IB1001) and Transfer Stacks (Invitrogen Cat. No. IB3010-01 or 3010-02) following standard manufacturer protocol. Membranes were blocked overnight at 4° C. in 25 ml of PBST; 5 mg/ml non-fat dry milk. After blocking, membranes were washed once with 25 ml PBST for 5 min at room temperature. Membranes were incubated with a primary anti-IGF-1RP antibody (Santa Cruz Biotechnology Cat. No. sc-9038) at 1:100 in 10 ml PBST for 1 hr at room temperature. The membranes were subsequently washed three times in 25 ml PBST for 5 min. For detection, membranes were incubated with a secondary HRP-conjugated Goat anti-Rabbit IgG-Fc antibody (US Biological Cat. No. 11904-40J) at a 1:1000 dilution in 10 ml PBST for 1 hr at room temperature. Membranes were washed three times in 25 ml PBST for 5 min followed by one wash in 25 ml PBST for 20 min. Protein bands were detected using the Amersham ECL Western Blotting Analysis System (GE Healthcare Cat. No. RPN2108) following standard manufacturer protocol.

iii. Biacore Analysis of the IGF-1R-Fc Mutant Library

Both mIGF-1R-Fc and hIGF-1R-Fc bind with high apparent affinity to the M13-C06, M14-C03, and M14-G11 sensorchip surfaces described above due to their highly multivalent nature induced by the incorporation of two separate homodimeric regions (IGF-1R and IgG1-Fc). To distinguish between the actual high affinity binding hIGF-1R-Fc to M13-C06 and the low affinity binding of mIGF-1R-Fc to M13-C06, the receptor-Fc fusions were captured on the M13-C06 sensorchip surface followed by an additional soluble M13-C06 Fab binding event. Receptor-Fc constructs were captured to the M13-C06 chip surface (prepared as described above) by injection of 60 μL of the affinity purified, concentrated material at a 1 μl/min flow rate. After, completion of the receptor-Fc loading step, flow rates were elevated to 5 μl/min. 10 nM, 3 nM, and 1 nM M13-C06 Fab concentrations were injected (50 μL) subsequent to the loading of each receptor-Fc construct. At the end of each sensorgram, the flow rate was elevated to 30 μl/min and the chip surface was regenerated by 2×10 μL injections of 0.1 M glycine, pH 2.

iv. Time-Resolved Fluorescence Resonance Energy Transfer (tr-FRET) Assay for IGF-1R-Fc Mutant Screening

Serial dilutions of mutant receptor, starting at 0.25-0.5 μg (25 μl) were mixed with 0.05 □g IGF1R-His₁₀-Cy5 (12.5 μl) and 0.00375 μg Eu:C06 (12.5 μl) in 384-well microtiter plates (white from Costar). The conjugation levels for IGF1R-His₁₀-Cy5 were 6.8:1 Cy5:IGF1R-His₁₀, and for Eu-C06 were 10.3:1 Eu:C06. The total volume was 50 μl for each sample. Plates were incubated for 1 hr at room temperature on a plate agitator. Fluorescence measurements were carried out on a Wallac Victor² fluorescent plate reader (Perkin Elmer) using the LANCE protocol with the excitation wavelength at 340 nm and emission wavelength at 665 nm. All data were fitted to a one-site binding model from which the corresponding IC₅₀ values were determined.

The fact that murine IGF-1R does not bind M13-C06 antibody was utilized to design a library of mouse mutations within hIGF-1R-Fc to assess the location of the M13-C06 binding site on IGF-1R. The various mutations in hIGF-1R tested are shown in Table 6. Western blot analysis was used to confirm expression of each hIGF-1R-Fc mutant and to develop a standard curve to approximate the relative concentration of each mutant protein; using purified hIGF-1R-Fc as a positive control (data not shown).

TABLE 6
Affect of mutations on IGF-1R binding to M13-C06. SD015 is
bold-faced as it was the only residue to demonstrate little to no
binding to M13-C06 in the two assay formats.

| Mutation | | | IC50 values |
|---|---|---|---|
| Number | Individual Mutants | Biacore Relative RUmax | (μg/ml) |
| SDWT | Wild-type | 1.0 | 1.5 |
| [symbol] | — | [symbol] | [symbol] |
| SD001 | Y28A | 0.6 | 1.0 |
| SD002 | M156A | 1.2 | 0.3 |
| SD003 | T188F | 1.0 | 0.2 |
| SD004 | S210H_A211Q | 0.8 | ND |
| SD005 | A217T | 0.9 | ND |
| SD006 | A227K | 1.7 | 0.2 |
| SD007 | N237G | 1.3 | <0.1 |
| SD008 | S258F | 1.5 | <0.1 |
| SD009 | E264K | 0.6 | 7.7 |
| SD010 | G271D | 0.8 | 0.1 |
| SD011 | G285S_S286T | 1.8 | <0.1 |
| SD012 | E303G | 0.3 | 0.9 |
| SD013 | D405K | 0.7 | <0.1 |
| SD014 | K412A_A413Q | 0.6 | <0.1 |
| [symbol] | [symbol] | [symbol] | [symbol] |
| SD016 | D531Q_V532N | 2.0 | 0.1 |
| SD017 | I650S | 2.0 | 0.2 |
| SD018 | E665A | 1.7 | <0.1 |
| SD019 | A739W_I741F | 1.9 | 0.2 |

ND = not determined.

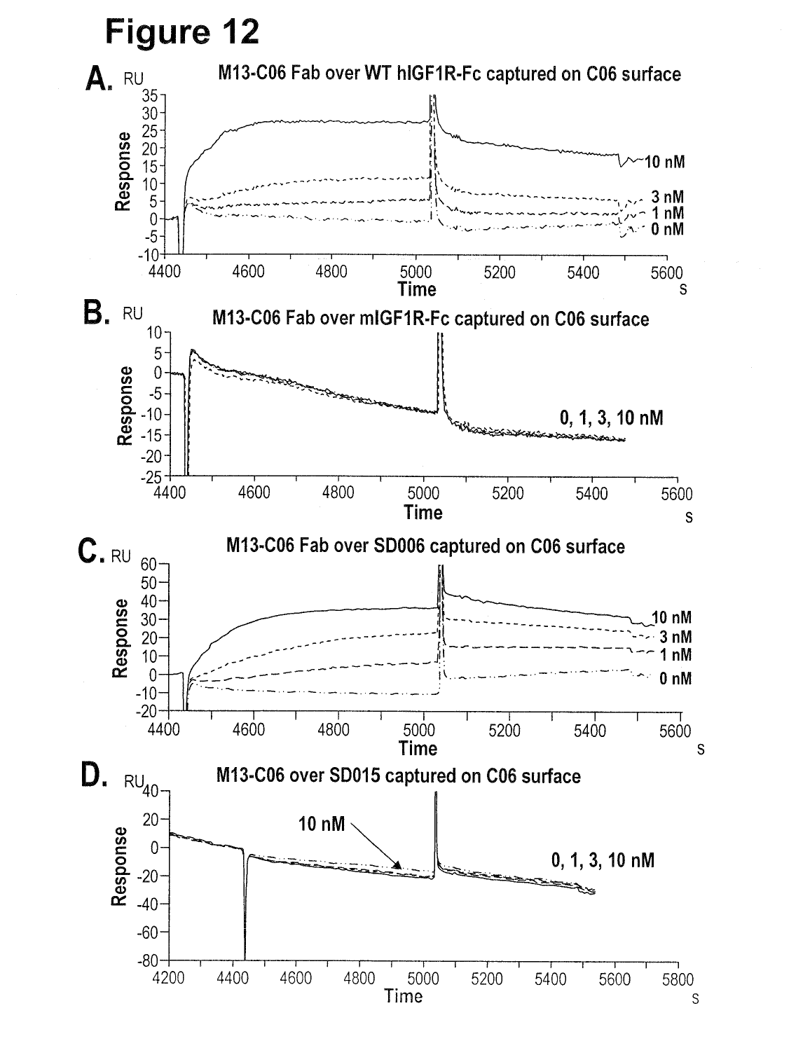

SPR and tr-FRET was used to screen for mutations that inhibit the binding of IGF-1R-Fc to M13-C06. Except for the SD015 mutant, all mutant IGF-1R constructs demonstrated M13-C06 binding activity, or M13-C06 Fab binding activity in the SPR experiments. See: FIG. 12; Table 6; and, data not shown (competitive inhibition analysis was used to establish binding curves for displacement of Eu-M13-C06 bound to Cy5-labeled IGF1R by increasing concentrations of unlabeled hIGF1R-Fc (SDWT), mouse IGF1R-Fc (mIGF1R-Fc) and mutant hIGF1R-Fc constructs).

There was some deviation in the IC₅₀ values determined using tr-FRET and relative binding strengths determined using SPR due to natural variations in expression and quantitation by Western Blot; however, SD015 was the only mutant to demonstrate virtually no binding activity toward M13-C06 in both assays and to parallel the results determined for the mIGF-1R-Fc control. His464 is located 2 amino acids C-terminal in primary amino acid sequence to the C-terminus of the truncated version of hIGF-1R-Fc construct (i.e., hIGF-1R(1-462)-Fc). The residual binding activity of M13-C06 to truncated hIGF-1R(1-462) suggests that the M13-C06 binding epitope minimally encompasses residues Val462-His464. Additional residues are likely involved in the antibody-epitope binding interaction as evidence indicates that M13-C06's epitope is conformationally dependent. Notably, however, residues Val462 and His464 are predicted to reside on the exterior surface of the FnIII-1 domain (FIG. 1).

In an attempt to characterize the extent of the M13-C06 epitope (i.e., what residues periperhal to 462-464 are important for antibody binding and activity), a structural modeling approach was utilized. Human IGF-1R and human INSR share 57% identity (73% similarity) and a similar tertiary structure. Previous analyses of X-ray crystal structure protein antigen:antibody binding surfaces has suggested an average binding surface of 700 Å² (angstroms-squared) with an approximate radius of 14 Å from the center of the binding epitope (Davies 1996). Using the X-ray crystal structure of the homologous ectodomain of INSR (pdb code 2DTG, (McKern 2006)), the residues on the surface of the FnIII-1 domain within a 14 Å radius of residues 462-464 were calculated by mapping the IGF-1R residues V462 through H464 to INSR residues L472 and K474. The distances cut-off was applied for any atom-to-atom distance within 14 Å, while the average distance was calculated from the Cα to Cα distance of L472 and K474 to each residue within the surface patch. The average distance calculated is listed as 14 Å for residues for which the Cα to Cα distance was greater than 14 Å but in which the sidechains are within the 14 Å cut-off. Residues of likely importance for M13-C06 binding and activity are listed in Table 7.

TABLE 7
Residues within IGF-1R predicted to be important for M13-C06
binding and activity. Residues 462 and 464 are italicized as these represent
the predicted center of the IGF-1R binding epitope and experimental
data demonstrates the importance of these residues in M13-C06 binding.

| | | | | Distance | Distance | |
|---|---|---|---|---|---|---|
| | | | | to | to | |
| IR AA | | | | 472 (Å) | 474 (Å) | Average |
| residue # | Surface | IGF1R AA | | (C□ to | (C□ to | distance |
| (2DTG) | accessibility | residue # | | C□) | C□) | (Å) |
| S437 | 0.477792 | S | 427 | 13.785 | 14 | 13.8925 |
| E438 | 0.337716 | E | 428 | 14 | 14 | 14 |
| E469 | 0.320544 | E | 459 | 9.95 | 14 | 11.975 |
| N470 | 0.8196 | S | 460 | 6.29 | 12.42 | 9.355 |
| E471 | 0.349164 | D | 461 | 3.79 | 9.57 | 6.68 |
| [symbol] | [symbol] | [symbol] | [symbol] | | 6.25 | 6.25 |
| [symbol] | [symbol] | [symbol] | [symbol] | 6.25 | 14 | 10.125 |
| S476 | 0.477792 | T | 466 | 12.45 | 6.43 | 9.44 |
| Y477 | 0.524048 | S | 467 | 14 | 9.15 | 11.575 |
| I478 | 0.5405 | T | 468 | 14 | 11.03 | 12.515 |
| R479 | 0.362378 | T | 469 | 14 | 14 | 14 |
| R488 | 0.375476 | T | 478 | 13.98 | 8.75 | 11.365 |
| E490 | 0.37206 | H | 480 | 9.18 | 5.84 | 7.51 |
| Y492 | 0.313493 | Y | 482 | 10.45 | 11.24 | 10.845 |
| W493 | 0.87318 | R | 483 | 11.17 | 13.03 | 12.1 |
| P495 | 0.824499 | P | 485 | 14 | 14 | 14 |
| D496 | 1 | D | 486 | 14 | 14 | 14 |
| E509 | 0.520884 | E | 499 | 14 | 14 | 14 |
| Q513 | 0.515108 | K | 503 | 14 | 14 | 14 |
| N514 | 0.68983 | N | 504 | 14 | 14 | 14 |
| V515 | 0.644094 | V | 505 | 14 | 14 | 14 |
| K544 | 0.865258 | N | 529 | 14 | 14 | 14 |
| S545 | 0.699624 | K | 530 | 14 | 14 | 14 |
| Q546 | 1 | D | 531 | 14 | 14 | 14 |
| N547 | 0.87424 | V | 532 | 14 | 14 | 14 |
| H548 | 0.406778 | E | 533 | 14 | 10.89 | 12.445 |
| W551 | 0.523908 | I | 536 | 14 | 14 | 14 |
| R577 | 0.41477 | H | 563 | 14 | 14 | 14 |
| T578 | 0.43254 | I | 564 | 13.19 | 14 | 13.595 |
| Y579 | 0.603591 | R | 565 | 9.54 | 14 | 11.77 |
| K582 | 0.34027 | K | 568 | 5.54 | 8.98 | 7.26 |
| D584 | 0.602475 | E | 570 | 7.01 | 7.4 | 7.205 |
| I585 | 0.340515 | I | 571 | 10.79 | 10 | 10.395 |
| I586 | 0.308085 | L | 572 | 13.04 | 10.49 | 11.765 |
| Y587 | 0.580196 | Y | 573 | 14 | 13.65 | 13.825 |

Published work has shown that antibodies that recognize residues 440-586 can be both inhibitory and agonistic to IGF-1 binding (Soos 1992; Keyhanfar 2007). 440-586 represents all of L2 and FnIII-1 with many potential non-overlapping surfaces accessible to anti-IGF-1R antibodies. The instant example provides the first instance where the inhibitory epitope of IGF-1R has been mapped to a particular residue(s). A recent structure of INSR was co-crystallized with anti-INSR antibody known to inhibit insulin binding to its receptor (Soos 1986; McKern 2006). The homologous residue to His464 of IGF-1R (K474 of INSR) is part of the binding surface of this antibody with INSR. It is possible that M13-C06 shares a similar inhibitory mechanism for inhibiting IGF-1 binding to IGF-1R as the antagonistic anti-INSR antibody.

### Example 4

### Cross-Blocking Studies with Anti-IGF-1R Antibodies

a. Materials

The anti-IGF-1R antibodies M13-C06, M14-G11, M13-C06, M14-C03, and P1E2 were subcloned, expressed, and purified as described previously (see U.S. application Ser. No. 11/727,887 which is incorporated by reference herein). A commercially available inhibitory IGF-1R antibody (αIR3, (Jacobs 1986)) was purchased from Calbiochem (Cat. No. GR11LSP5). Human IGF-1 and IGF-2 with N-terminal octahistidine tags were produced recombinantly in Pichia and purified using Ni²⁺-NTA agarose. A recombinant soluble human IGF-1R ectodomain construct containing a C-terminal 10-histidine tag, denoted hIGF-1R(1-902)-His₁₀, was purchased from R&D systems (Cat. No. 305-GR-050). Human and mouse IGF-1R(1-903)-IgG1-Fc fusion proteins were constructed and purified using standard protein A chromatography methods.

b. Methods

(i) Antibody:Antibody Cross-Blocking Studies

The ability of various antibodies to block M13-C06 or M14-G11 from binding hIGF-1R was determined using biotinylated versions of the antibodies and hIGF-1R-Fc. Briefly, 50 μL of 2 μg/mL hIGF-1R-Fc in 1×PBS were coated per well of a 96-well clear MaxiSorp plate (Nunc) for 2 hours at room temperature (RT, no shaking). Plates were washed with 1×PBS and blocked overnight at 2-8° C. using a PBS/1% BSA solution. Plates were washed and incubated with a 100 μL mixture of biotinylated M13-C06 or biotinylated M14-G11 (80 ng/mL) and inhibitor antibody for 1 hour at RT. Inhibitor antibodies were serially diluted (5-fold dilutions) from 40 μg/mL to 3 ng/mL. M13-C06 and M14-G11 were biotinylated using EZ-Link Sulfo-NHS-LC-Biotin according to protocol provided by the manufacturer (Pierce Cat. No. 21335). A control was also performed by serial dilution of a non-IGF-1R specific IgG4 isotype control antibody with biotinylated M13-C06 or biotinylated M14-G11. Plates were washed and shaken for 1 hour at RT with 100 μL/well streptavidin-HRP (1:4000 dilution into blocking buffer, Southern Biotech Cat. No. 7100-05). Plates were washed and 100 μL/well SureBlue Reserve TMB Microwell Peroxidase Substrate (KPL, Cat. No. 53-00-01) was added to the wells. Detection of the presence of biotinylated M13-C06 or M14-G11 was performed by reading the absorbance at 650 nm every 5 minutes using a Wallac 1420-041 Multilabel Counter plate reader.

The ability of various antibodies to block murine αIR3 was determined using “Zenon-Fab-HRP” labeled αIR3 and hIGF-1R-Fc. αIR (IgG1) was Zenon®-Fab-HRP labeled as described by the manufacturer (Invitrogen Cat. No. Z25054). Briefly, 50 μL of 2 μg/mL hIGF-1R-Fc in 1×PBS were coated per well of a 96-well clear MaxiSorp plate (Nunc) for 2 hours at RT (no shaking). Plates were washed with 1×PBS and blocked overnight at 2-8° C. using a PBS/1% BSA solution. Plates were washed and incubated with a 100 μL mixture of Zenon-labeled αIR3 (40 ng/mL) and inhibitor antibody for 1 hour at RT. Inhibitor antibodies were serially diluted (5-fold dilutions) from 40 μg/mL to 3 ng/mL. A control inhibition was performed by serial dilution of a non-IGF-1R specific IgG4 isotype control antibody with Zenon-labeled αIR3. Plates were washed and 100 μL/well SureBlue Reserve TMB Microwell Peroxidase Substrate (KPL, Cat. No. 53-00-01) was added to the wells. Detection of Zenon-labeled αIR3 was performed by reading the absorbance at 650 nm every 5 minutes using a Wallac 1420-041 Multilabel Counter plate reader.

(ii) IGF-1/IGF-2 Ligand:Antibody Cross-Blocking Studies

The ability of IGF-1 and IGF-2 to block hIGF-1R-His from binding M13-C06 and M14-G11 was determined by SPR using a Biacore3000. M13-C06 and M14-G11 at 40 μg/mL in 10 mM Acetate pH 4.0 were immobilized to ˜2,000 RU on Biacore CM5 Research Grade Sensor Chip (Cat. No. BR-1000-14) surfaces using the standard NHS/EDC chemistry protocol of the manufacturer (Biacore). To test the ability of IGF-1 or IGF-2 to inhibit hIGF-1R-His binding to immobilized antibody surfaces, 160 μL of 40 nM hIGF-1R-His in the presence of IGF-1 or IGF-2 at concentrations ranging from 500 pM to 4 μM was injected over the sensorchip surfaces at 20 μL/min. Additionally, the anti-IGF-1R antibodies, M13-C06 and M14-G11, and their antibody Fabs were used to investigate their ability to block IGF-1R to the same sensorchip surfaces. Similar antibody serial dilutions (in the presence of hIGF-1R-His) were performed as used for the IGF-1 and IGF-2 blocking experiments. Regeneration was achieved by three 10 μL injections of 0.1 M glycine, pH 2.0. 100% hIGF-1R-His binding to each antibody was determined by the signal above the baseline under mass transport-limiting conditions 60 seconds into the injection. Attenuation of the signal at 60 seconds based on the presence of IGF-1 or IGF-2 in solution was used as a measure of ligand-mediated blockade of antibody binding.

(iii) IGF-1/IGF-2 Ligand Blocking by Single Antibodies and Antibody Combinations

hIGF-1R-Fc was biotinylated using EZ-Link Sulfo-NHS-LC-Biotin according to the protocol provided by the manufacturer (Pierce Cat. No. 21335). Biotinylated human IGF-1R Fc (NB12453-9) at 5 μg/ml was added to the wells of SigmaScreen streptavidin-coated 96-well plates (Sigma, Cat. No. M5432-5EA) at 100 μL/well and incubated overnight at 2-8° C. The plates were then washed four times with 200 μL/well PBST. Human IGF-1 His (NB12111-85) was prepared at 320 nM in PBST, 1.0 mg/ml BSA. Serial dilutions of anti-IGF-1R antibodies M13-C06 (NB11054-82), M14-C03 (NB11055-147), M14-G11 (NB11016-120), P1E2 (DE12 Chimera comprising mouse VH and VL derived from the antibody expressed by the P1E2 hybridoma cell line fused to human IgG4agly/kappa constant domains), and αIR3 (Calbiochem, Cat. No. GR11LSP5) were made up in the 320 nM IGF-1 His solution. Dilutions were made from 1.3 μM to 10 pM for M13-C06 and M14-C03, from 5.2 μM to 10 pM for M14-G11, and from 2.6 μM to 10 pM for both P1E2 and αIR3. Human IGF-2 His (NB12110-10) was prepared at 320 nM in PBST, 1.0 mg/ml BSA. The antibodies were serial diluted (from 1.3 μM to 5 pM for M13-C06 and M14-C03, from 5.2 μM to 5 pM for M14-G11 and αIR3, and from 5.2 μM to 20 pM for P1E2) using a solution of 320 nM IGF-2 His. The dilutions were added to the plates in duplicate at 100 μL/well and the plates were incubated at RT for 1 hour. The plates were then washed four times with 200 μL/well PBST. An HRP-conjugated anti-Histag antibody (Penta-His HRP Conjugate, QIAGEN, Cat. No. 1014992) was diluted 1:1000 in PBST and added to plates at 100 μL/well, and the plates were incubated at RT for one hour. The plates were then washed four times with 200 μL/well PBST. SureBlue Reserve TMB Microwell Peroxidase Substrate (KPL, Cat. No. 53-00-01) was added to plates at 100 μL/well followed by 1% phosphoric acid at 100 μL/well once the desired reaction was observed. The absorbance of each well was determined at 450 nm, and the results were normalized and plotted against the log of antibody concentration.

c. Results

(i) Cross-Blocking Properties of the Anti-IGF-1R Antibodies

The antibodies were all tested for their ability to cross-block one another in an IGF-1R ELISA binding assay (Table 8). M13-C06 and M14-C03 cross-blocked one another in the assay, but had no cross-blocking activity towards P1E2, αIR3 or M14-G11 in the assay. P1E2 and αIR3 were both able to completely cross-block labeled αIR3 and M14-G11 in the assays. M14-G11 demonstrated moderate cross-blocking activity towards αIR3 suggesting that M14-G11's epitope may overlap, but not be identical to the epitope(s) of αIR3 and P1E2.

TABLE 8
Summary results of antibody cross-blocking experiments.

| | M13-C06 cross- | M14-G11 cross- | αIR3 cross- |
|---|---|---|---|
| Antibody Inhibitor | blocking | blocking | blocking |
| M13-C06 | +++++ | − | − |
| M14-C03 | +++++ | − | − |
| M14-G11 | − | +++++ | +++ |
| αIR3 | − | +++++ | +++++ |
| P1E2 | − | +++++ | +++++ |

+++++ = antibody binding competition relative to itself (90-100%)
++++ = 70-90% competition
+++ = 50-70% competition
++ = 30-50% competition
+ = 10-30% competition
+/− = 0-10% competition
N/A = results not available.

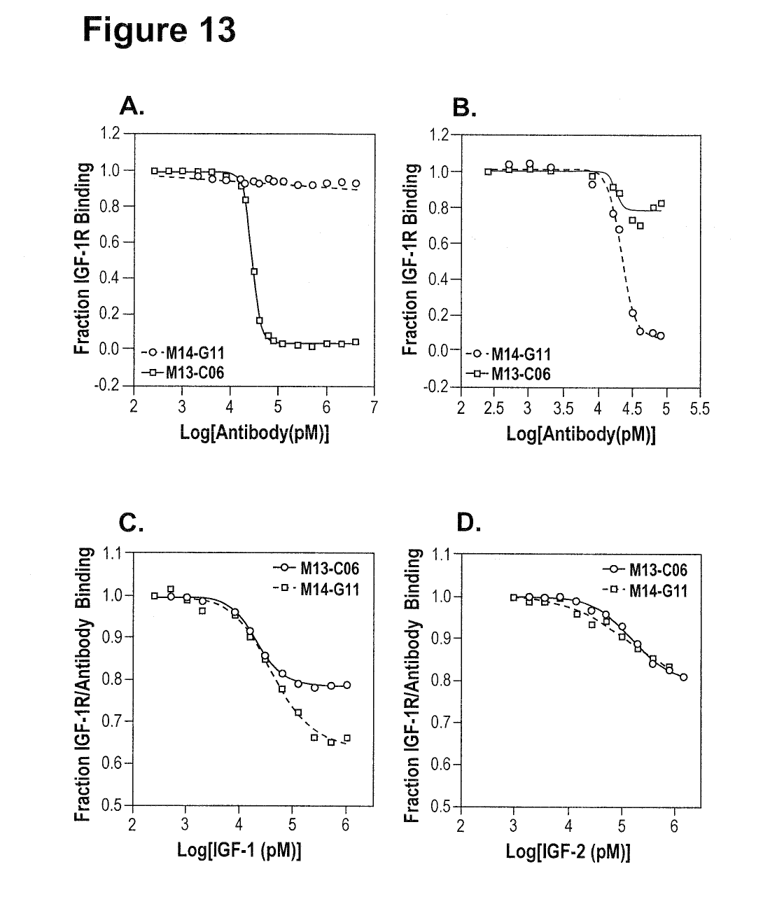

Similar cross-blocking results were obtained using a SPR-based assay (FIG. 13A,B). The cross-blocking studies were performed at 40 nM IGF-1R concentrations (˜400-fold above the affinity of M13-C06 and M14-G11 for IGF-1R). The concentrations at which each antibody was capable of completely cross-blocking itself should therefore be a measure of the antibody/IGF-1R stoichiometry. Both M13-C06 and M14-G11 reached self-cross-blocking saturation at 40 nM and 80 nM antibody and Fab concentrations, respectively (FIG. 13A,B; Fab data not shown). The data suggests that both antibodies recognize two sites on the IGF-1R homodimer. While there are two sites, the epitope for each antibody is likely unique to a particular structural site on the molecule that appears twice due to the homodimeric nature of the receptor. Analytical size exclusion/static light scattering experiments were performed to demonstrate that the hIGF-1R-His ectodomain construct is a homodimer in solution.

(ii) Ligand: Antibody Cross-Blocking Properties

IGF-1 and IGF-2 both reduced the ability of hIGF-1R-His to bind to the M13-C06 and M14-G11 surfaces in the SPR assay (FIGS. 13 C and D). Reduction of IGF-1R binding to the M13-C06 surface was only ˜20-25% even at saturating levels of IGF-1 and IGF-2 suggesting that the ligand is allosterically reducing the affinity of IGF-1R for M13-C06. The IGF-1 and IGF-2 inhibition curves never reached saturation for IGF-1R binding to the M14-G11 surface suggesting possible direct antibody/ligand competition. The stoichiometry of IGF-1 and IGF-2 binding to the receptor is 1:1, even though two possible binding sites exist within the receptor. Ligand binding to one of these sites appears to exclude the ability to recognize the second site on the opposite side of the receptor. The IGF-1 stoichiometry results are consistent with published work (Jannson M. et al., J. Biol. Chem., (1997) 8189-8197).

(iii) IGF-1 and IGF-2 Blocking Properties of the Anti-IGF-1R Antibodies

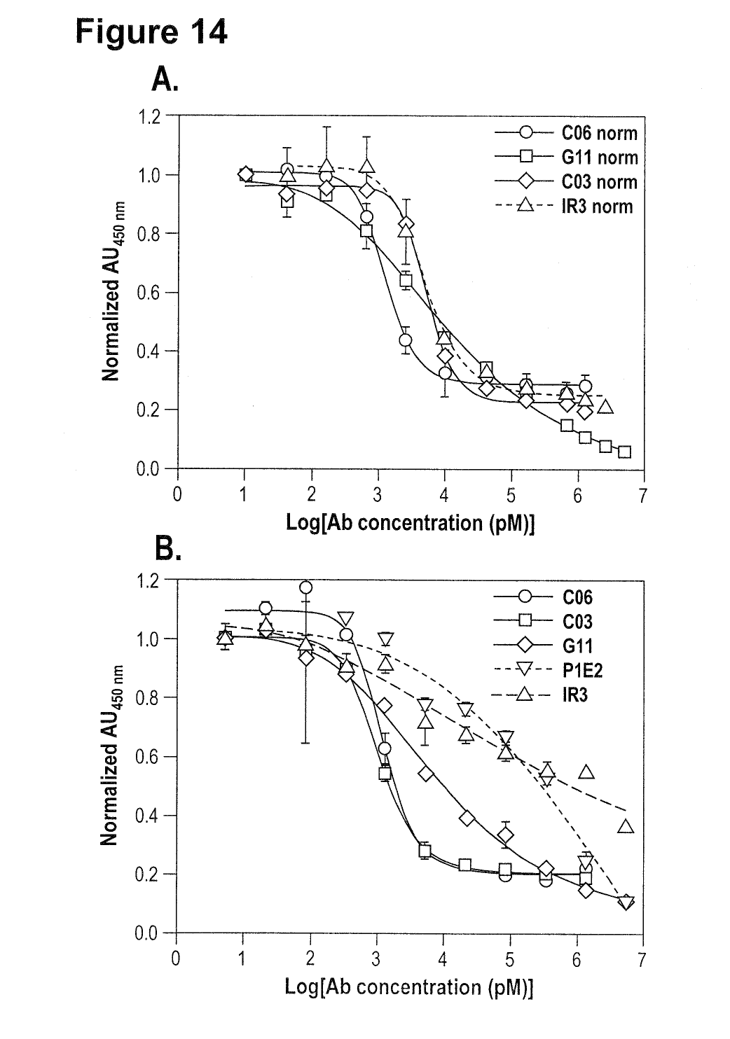

Five antibodies (M13-C06, M14-C03, M14-G11, P1E2, and αIR3) were tested for their ability to block IGF-1 and IGF-2 from binding IGF-1R in an ELISA-based competition assay. M13-C06 and M14-C03 block both IGF-1 and IGF-2 binding to IGF-1R (FIGS. 13A-D). Partial IGF-1 or IGF-2 binding could be restored by increasing the concentration of ligand in the assay even in the presence of saturating levels of M13-C06 or M14-C03. Additionally, the midpoint of the inhibition curves of M13-C06 and M14-C03 (IC₅₀) was independent of the concentration of IGF-1 or IGF-2 in the assay. Both results suggest an allosteric mechanism of ligand blockade. Titrating human IGF-1 His in the assay in the presence and absence of saturating levels of M13-C06 allowed us to measure an apparent affinity loss of the ligand for hIGF-1R-Fc. The data suggests that the presence of the M13-C06 antibody leads to an approximately 50-fold loss in affinity of human IGF-1 His for hIGF-1R-Fc (FIG. 14A). P1E2 and αIR3 also block IGF-1 allosterically, but have little effect on IGF-2 binding to IGF-1R (FIGS. 13A-D). These results for αIR3 are consistent with published results (Jacobs 1986). M14-G11 appeared to block both IGF-1 and IGF-2 in a competitive fashion (FIGS. 13A-D). The IC₅₀ of M14-G11 depended on the IGF-1 concentration used in the assay. Saturating levels of the M14-G11 managed to block 100% of both ligands, albeit at much higher M14-G11 concentrations than the IC₅₀ of the allosteric blockers.

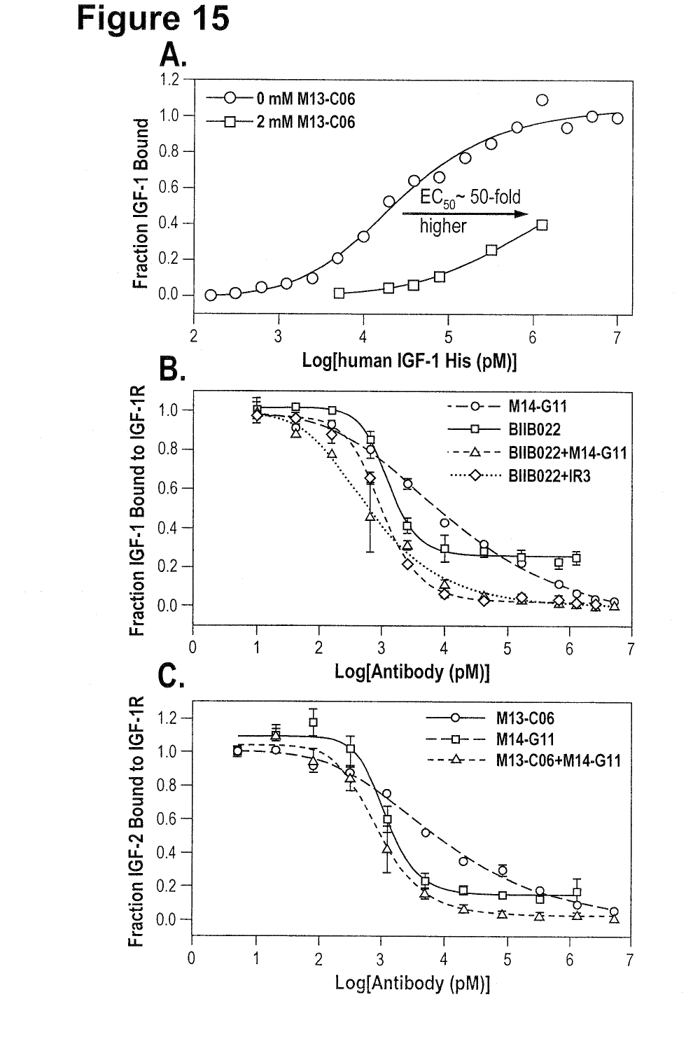

Combination of antibodies with unique and non-overlapping epitopes leads to both an increase in the blocking potency (IC₅₀) of anti-IGF-1R antibodies as well as apparent complete ligand blockade (FIGS. 15B and C). M13-C06 has been shown to bind a large surface on the FnIII-1 domain opposite and distal to the ligand-binding site—consistent with an allosteric blocking mechanism. M14-G11 and αIR3 bind to overlapping surfaces on the CRR and L2 domains. M14-G11's epitope is on a face of the CRR domain that is directly adjacent to the ligand binding site—consistent with its apparent competitive ligand-blocking behavior. αIR3's epitope is on a surface perpendicular to the ligand binding site—consistent with its demonstrated allosteric blocking behavior. Combinations of the allosteric inhibitor M13-C06 with either the competitive inhibitor M14-G11 or the allosteric inhibitor αIR3 led to virtually 100% blocking of IGF-1 and IGF-2 at IC₅₀ values below that of the antibodies in isolation. This data, which indicates synergistic activity of inhibitory anti-IGF-1R antibody combinations, is shown in Tables 9 and 10).

TABLE 9
IGF-1 blocking potency and percent IGF-1inhibition of the
anti-IGF-1R antibodies M13-C06, M14-G11, and αIR3.

| | | % IGF-1 (at 320 nM) bound to |
|---|---|---|
| Antibody | IC50 (IGF-1 | IGF-1R at saturating |
| (Combination) | blocking, nM) | antibody concentrations |
| M13-C06 | 1.3 | 25% |
| M14-G11 | 13.0 | 1% |
| αIR3 | 4.9 | 23% |
| M13-C06 + □IR3 | 1.0 | 1% |
| M13-C06 + M14-G11 | 0.67 | 1% |

TABLE 10
IGF-2 blocking potency and percent IGF-2 inhibition of the
anti-IGF-1R antibodies M13-C06, M14-G11, and □IR3.

| | | % IGF-2 (at 640 nM) bound to |
|---|---|---|
| Antibody | IC50 (IGF- | IGF-1R at saturating |
| (Combination) | 2blocking, nM) | antibody concentrations |
| M13-C06 | 1.9 | 16% |
| M14-G11 | 7.9 | 1% |
| αIR3 | N/A | Did not block |
| M13-C06 + M14-G11 | 0.87 | 1% |

### Example 5

### Residue Specific Epitope Mapping of Allosteric and Competitive Antibody Inhibitors of IGF-1R

a. Methods

i. Epitope Mapping Mutations

The 46 mutant epitope mapping library was constructed by mutagenizing the wild-type hIGF-1R-Fc PV-90 plasmid using the Stratagene site-directed mutagenesis kit following the manufacturer's protocols. Incorporation of each mutant (or double mutant) within the PV-90 vector was confirmed by DNA sequencing. For DNA production, plasmids were transformed into DH5a (Invitrogen, Cat. No. 18258-012), cultured overnight at 37° C., and miniprepped or maxiprepped using the Qiagen Miniprep Kit or Qiagen Endotoxin-Free MaxiPrep Kit, respectively. 200 μg of each mutant plasmid was transiently tranfected into 100 mL HEK293 T cells at 2×10⁶ cells/mL using the PolyFect transfection kit (Qiagen) for soluble protein secretion into the media. Cells were cultured in DMEM (IvrineScientific), 10% FBS (low IgG bovine serum, Invitrogen—further depleted of bovine IgG by passage over a 20 mL protein A column) at 37° C. in a CO₂ incubator. After 7 days, supernatants containing each IGF-1R-Fc mutant were collected by centrifugation at 1200 rpm and filtration through a 0.2 μm filter. Each mutant was affinity purified by passage of its supernatant over a 1.2 mL protein A Sepharose FF column pre-equilibrated with 1×PBS. The mutants were eluted from the column using 0.1 M glycine, pH 3.0, neutralized with 1 M Tris, pH 8.5, 0.1% Tween-80, and concentrated to ˜300 μL using VivaSpin 6 MWCO 30,000 centrifugal concentration devices (Sartorius, Cat. No. VS0621).

ii. Western Blot Analysis of IGF-1R Mutants

hIGF-1R-Fc mutant samples were run on 4-20% Tris-Glycine gels (Invitrogen Cat. No. EC6028) using the Xcell SureLock Mini Cell (Invitrogen, Cat. No. EI0001) following the standard manufacturer protocol. Samples were transferred to nitrocellulose using the iBlot Dry Blotting System (Invitrogen, Cat. No. IB1001) and Transfer Stacks (Invitrogen, Cat. No. IB3010-01 or 3010-02) following the standard manufacturer protocol. Membranes were blocked overnight at 4° C. in 25 ml of PBST; 5 mg/mL non-fat dry milk. After blocking, membranes were washed once with 25 ml PBST for 5 min at room temperature. Membranes were incubated with a primary anti-IGF-1Rβ antibody (Santa Cruz Biotechnology Cat. No. sc-9038) at 1:100 in 10 mL PBST for 1 hr at room temperature. The membranes were subsequently washed three times in 25 ml PBST for 5 min. For detection, membranes were incubated with a secondary HRP-conjugated Goat anti-Rabbit IgG-Fc antibody (US Biological Cat. No. I1904-40J) at a 1:1000 dilution in 10 mL PBST for 1 hr at room temperature. Membranes were washed three times in 25 mL PBST for 5 min followed by one wash in 25 mL PBST for 20 min. Protein bands were detected using the Amersham ECL Western Blotting Analysis System (GE Healthcare, Cat. No. RPN2108) following the standard manufacturer protocol.

iii. Surface Plasmon Resonance Analysis of the IGF-1R-Fc Mutant Library

Surface plasmon resonance (SPR) experiments were performed on a Biacore 3000 instrument set to 25° C. Both mIGF-1R-Fc and hIGF-1R-Fc bind with high apparent affinity to research grade CM5 sensorchip surfaces containing immobilized M13-C06, M14-C03, and M14-G11. The antibody sensorchip surfaces were prepared by injecting each antibody (diluted 100 μg/mL in 10 mM Acetate, pH 4.0) over EDC/NHS-activated sensorchip surfaces according to the standard protocol of the manufacturer. The ability of mIGF-1R-Fc to bind the antibody surfaces was the result of high apparent avidity of the protein. Both hIGF-1R-Fc and mIGF-1R-Fc proteins oligomerize due to the incorporation of two separate homodimeric regions (IGF-1R and IgG1-Fc). To distinguish between actual high affinity antibody binding to hIGF-1R-Fc and low affinity antibody binding to mIGF-1R-Fc, the receptor-Fc fusions were captured on the M13-C06 and M14-G11 sensorchip surfaces followed by an additional injection of antibody (αIR3 and P1E2) or antibody Fab (M13-C06, M14-C03, and M14-G11). Receptor-Fc constructs were captured onto antibody surfaces by injection of 60 μL of the affinity-purified, concentrated material at a 1 μl/min over the sensorchip surfaces. After, completion of the receptor-Fc loading step, flow rates were elevated to 5 μl/min. Solutions containing M13-C06 Fab or αIR3 antibody at 10 nM, 3 nM, or 1 nM or M14-C03 Fab, M14-G11 Fab, or P1E2 antibody at 30 nM, 10 nM, or 3 nM were injected (50 μL) subsequent to the loading of each receptor-Fc construct. Dissociation was measured for 7 minutes after the antibody injections were complete. Finally, the flow rate was elevated to 30 μL/min and the chip surfaces were regenerated by 2×10 μL injections of 0.1 M glycine, pH 2.

b. Results

i. Preliminary Epitope Mapping—Determination of the Epitope Locations

A preliminary set of 19 mutations was constructed to determine the location of the inhibitory anti-IGF-1R antibody epitopes. Based on the observation that M13-C06, M14-C03, and M14-G11 demonstrated little activity towards mouse IGF-1R, we identified a limited set of mutations within human IGF-1R that should enable our ability to locate the epitopes of the inhibitory anti-IGF-1R antibodies. Mouse and human IGF-1R share 95% primary amino acid sequence identity. Thirty-three (33) residues differ between mouse and human IGF-1R in the ectodomain. Twenty (20) of these residues were targeted for mutation because their homologous positions within the homologous INSR ectodomain structure were exposed to solvent (pdb code 2DTG, (McKem 2006)). Accessible surface areas were calculated using StrucTools (hypertext transfer protocol://molbio.info.nih.gov/structbio/basic.html) with a 1.4 Å probe radius. Four pairs of these mutants were identified where the proposed mutations were next to one another in primary sequence. In these cases, each pair was double mutated within a single construct. Therefore, the 20 residues positions led to 16 initial mutant constructs. Four additional mutations were constructed due to mouse/human IGF-1R amino acid differences within the unstructured loop region of the FnIII-2 domain known to be important for IGF-1/IGF-2 binding (Whittaker 2001; Sorensen 2004). Two of these positions were close in primary sequence and could be combined within a single mutant construct. The final list of the 19 preliminary mutations (SD001-SD019) is provided in Table 11. The residue numbering shown in Table 11 assumes that the 30-residue IGF-1R signal sequence has been cleaved. Each of the constructs were expressed by transient transfection in 100 mL HEK293 cells for 1 week and purified using protein A chromatography. Purified mutant IGF-1R constructs were concentrated and assayed for expression/folding by Western Blot analysis. Expression was 10-30 μg for all the mutant constructs.

M13-C06, M14-C03, M14-G11, P1E2 and αIR3 were assayed for their ability to interact with each of the mutant IGF-1R-Fc fusion constructs using surface plasmon resonance (Biacore). To remove the uncertain concentrations of the IGF-1R-Fc fusion constructs as a variable in the assay, each mutant construct was captured on a research grade CM5 chip containing ˜10,000 RU immobilized M13-C03, M14-C03, and M14-G11 antibody. To enhance our ability to visualize attenuations in antibody binding to the captured mutant IGF-1R constructs, we utilized enzymatically derived M13-C06, M14-C03, and M14-G11 antigen binding fragments (Fabs).

Of these preliminary 19 mutant constructs, only SD015 (E464H) affected the ability of the M13-C06 and M14-C03 Fabs to bind IGF-1R. Mutation of residue 464 to histidine led to complete ablation of the binding reaction for both Fabs. All other mutant IGF-1R constructs bound with comparative equilibrium dissociation constants (K_{D}=1 nM and 5 nM for the M13-C06 and M14-C03 Fabs, respectively). These experiments localize the epitope of the M13-C06 and M14-C03 antibodies to the surface of the FnIII-1 domain. The VH CDR regions of the two antibodies are highly similar (26 of 38 residues are identical) while the CDR regions of the VL domain are unrelated suggesting a strong V_{H} bias towards antigen recognition. Not surprisingly, the two antibodies effectively cross-block one another. Soos and coworkers have shown using IR/IGF-1R chimeras that one or more epitopes within the 2^{nd} leucine rich repeat domain (L2) and 1^{st} fibronectin type III domain (FnIII-1) can lead to receptor inhibition (Soos 1992). This spans residues 333-609; a total of 276 residues. In contrast, the detailed epitope mapping studies described here demonstrate that the epitope spans multiple, non-overlapping residues and that a single residue within the FnIII-1 domain, E464, is a particularly important residue for antibody binding.

Of the 19 mutants, only SD008 (S257F) and SD012 (E303G), mutations in cysteine rich repeat (CRR) and L2 domains, respectively, attenuated the ability of the M14-G11 Fab to recognize human IGF-1R (Table 11). In both cases, mutation led to approximately 3-fold losses in affinity based on the measured K_{D}. All other mutant IGF-1R constructs, including SD015, which demonstrated no reactivity towards M13-C06 and M14-C03, bound the M14-G11 Fab with wild-type affinity (K_{D}˜4-6 nM).

αIR3 and P1E2, were also screened against the preliminary mutant library. Both of these antibodies exhibited a similar reduction in their affinity to SD012 compared to wild-type human IGF-1R-Fc; however, only P1E2 exhibited reduced binding to SD008 (Table 11).

ii. Detailed Epitope Mapping—Residue Specific Definition of the M13-C06 and M14-C03 Antibody Epitopes

Based on the results of the preliminary IGF-1R mutant library that localized the M13-C06 and M14-C03 epitope(s) to the FnIII-1 domain of IGF-1R, a second set of mutations were designed to probe the surface of IGF-1R surrounding the original mutation, E464H, that led to ablation of antibody binding. A total of 21 residues were chosen for mutagenesis based on their 3D proximity to E464 (including a different mutation at residue 464 than the original histidine mutation). The 3D structure of the insulin receptor was used to estimate the proximity of residues surrounding 464. 7 pairs of residues were identified for mutation that were adjacent in primary sequence. Mutation of these residue pairs was done simultaneously to yield double mutants. Therefore, the second set of mutations consisted of 14 total constructs listed in Table 11 as SD101-SD114.

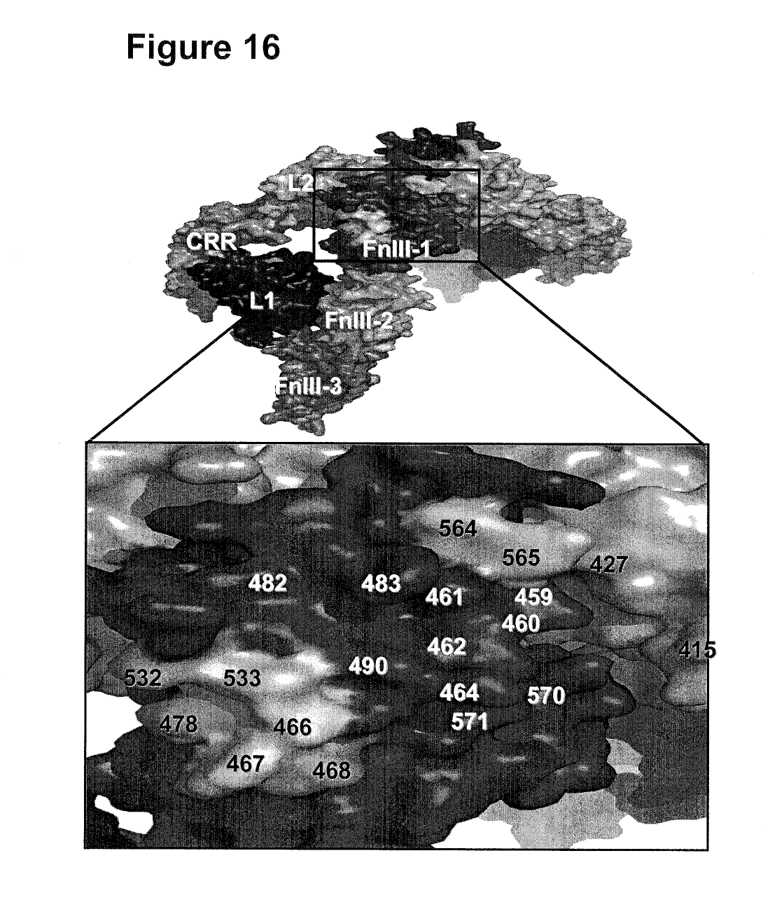

Expression, purification, and quality control of the 14 mutant constructs was performed as described for the first set of preliminary mutations (SD001-SD019). All 14 constructs expressed well and appeared folded based on Western Blot analysis except SD114. This construct expressed poorly and did not react in our Biacore experiment with M13-C06, M14-C03, or M14-G11—which recognizes a completely different epitope. Therefore, the data for this mutant construct was disregarded. The other 13 constructs allowed the precise, residue-specific definition of the M13-C06 and M14-C03 epitope. The residue-specific results are listed in Table 11. In summary, the epitopes of M13-C06 and M14-C03 were nearly identical and entirely contained within the FnIII-1 domain. The most crucial (perhaps central) residues were 461 and 462. SD103, which contains mutations at residues 461 and 462, demonstrated no reactivity towards the M13-C06 and M14-C03 Fabs and no reactivity towards the M13-C06 and M14-C03 surfaces. SD103 binding to the M14-G11 surface was no different than for any other FnIII-2 mutant construct indicating that this complete ablation was epitope specific. Other mutations that led to ablation or large decreases in antibody affinity (>100-fold decrease in affinity) for IGF-1R were found at IGF-1R residues 459, 460, 464, 480, 482, 483, 533, 570, and 571. Mutations that led to small decreases in antibody affinity (2.5≧K_{D}≧10 nM) compared to wild-type human IGF-1R were found at residues 466, 467, 564, 565, and 568. The positions of these residues were mapped to the surface of the homologous 1R structure (FIG. 16, reference McKem).

Based on the position and surface area coverage of the epitope, it is not surprising that both M13-C06 and M14-C03 were shown to allosterically inhibit IGF-1 and IGF-2 from binding IGF-1R. The epitope is on a receptor face opposite to the known ligand binding surface (Whittaker 2001; Sorensen 2004). Published work has shown that antibodies that recognize residues 440-586 can be both inhibitory and agonistic to IGF-1 binding (Soos 1992; Keyhanfar, 2007). Within IGF-1R, amino acid residues 440-586 represent all of L2 and FnIII-1 with many potential non-overlapping surfaces accessible to anti-IGF-1R antibodies. Our study is the first study that we are aware of that localizes the inhibitory epitope to a specific area on the receptor at residue specific resolution. A recent structure of the insulin receptor (IR) was co-crystallized with an anti-1R antibody known to inhibit insulin binding to its receptor (McKern 2006). The homologous residue to His464 of IGF-1R (K474 of IR) is part of the binding surface of this antibody with IR. It is possible that M13-C06 shares a similar inhibitory mechanism for inhibiting IGF-1 binding to IGF-1R as the antagonistic anti-1R antibody. Based on Biacore results, M13-C06 appears to inhibit IGF-1 (and likely IGF-2) by reducing the kinetic association rate. The antibody appears to trap the receptor ectodomain in a conformation that makes it difficult for IGF-1 and IGF-2 to access the receptor-binding site.

iii. Detailed Epitope Mapping—Residue Specific Definition of the M14-G11, P1E2, and αIR3 Antibody Epitopes

Based on the results of the preliminary IGF-1R mutant library that localized the M14-G11, P1E2, and αIR3 epitopes to the CRR and L2 domains of IGF-1R, a third set of mutations were designed that cover the surface of IGF-1R surrounding the original mutations, S257F and E303G, that led to a reduction of antibody affinity towards the receptor. A total of 15 residues were chosen for mutagenesis based on their 3D proximity to S257 and E303 (including a different mutation at residue 257 than the original phenylalanine mutation). The 3D structure of the insulin receptor was used to estimate the proximity of residues surrounding S257 and E303. Two (2) pairs of residues were identified for mutation that were adjacent in primary sequence. Mutation of these residue pairs was done simultaneously to yield double mutants. Therefore, the second set of mutations consisted of 13 total constructs listed in Table 11 as SD201-SD213.

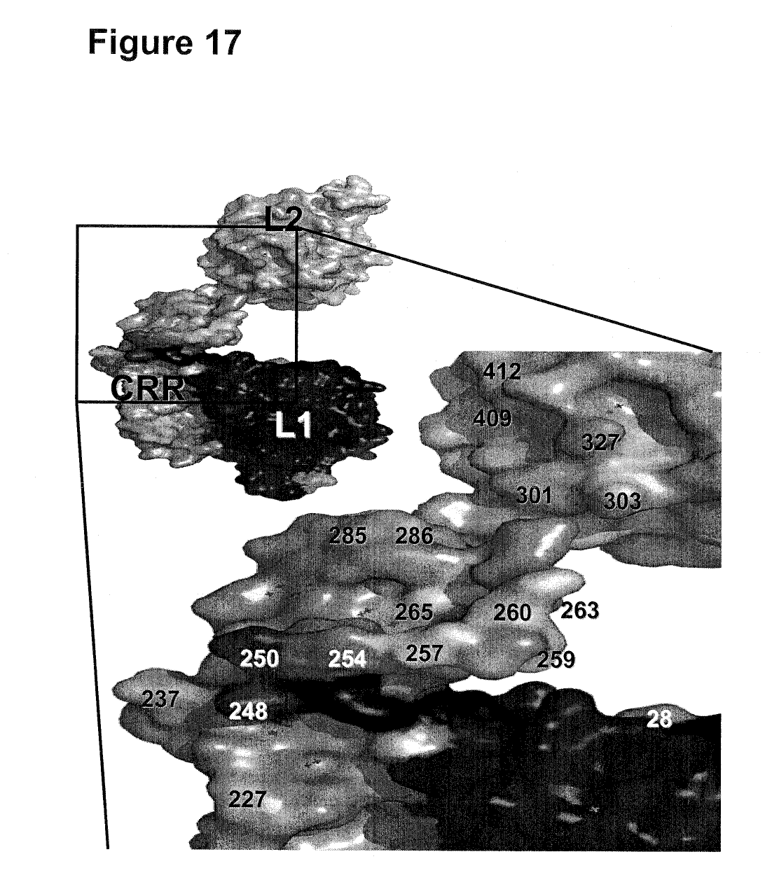

Expression, purification, and quality control of the 13 mutant constructs was performed as described for the first set of preliminary mutations (SD001-SD019). All of these constructs expressed well and appeared folded based on Western Blot analysis except for SD213. Data for SD213 was disregarded due to the ambiguity surrounding the folded state of the receptor. The other 12 mutant constructs led to the precise, residue-specific definition of the M14-G11, P1E2, and αIR3 epitopes. The residue-specific results are listed in Table 11. The epitopes differed between M14-G11, P1E2 and αIR3. This was not surprising, considering M14-G11 was shown to be a competitive inhibitor of both IGF-1 and IGF-2 while P1E2 and αIR3 were shown to allosterically inhibit the binding of IGF-1 only. The epitope of M14-G11 is near the center of the CRR domain on a surface that directly contacts residues that are known to have an effect on ligand binding (Whittaker 2001; Sorensen 2004). Mutations that ablated M14-G11 binding were found at positions 248 and 250. Mutation at residue 254 led to a moderate decrease in antibody affinity towards the receptor (10≧K_{D}≧100-fold above that of wild-type IGF-1R). Many other mutations predominantly in the CRR marginally reduced M14-G111 affinity for the receptor (2.5≧K_{D}≧10-fold above that of wild-type IGF-1R) including residues 257, 259, 260, 263, 265, and 303. The positions of these residues were mapped to the surface of the published structure of the first three ectodomains of IGF-1R (FIG. 17, (Garrett 1998))

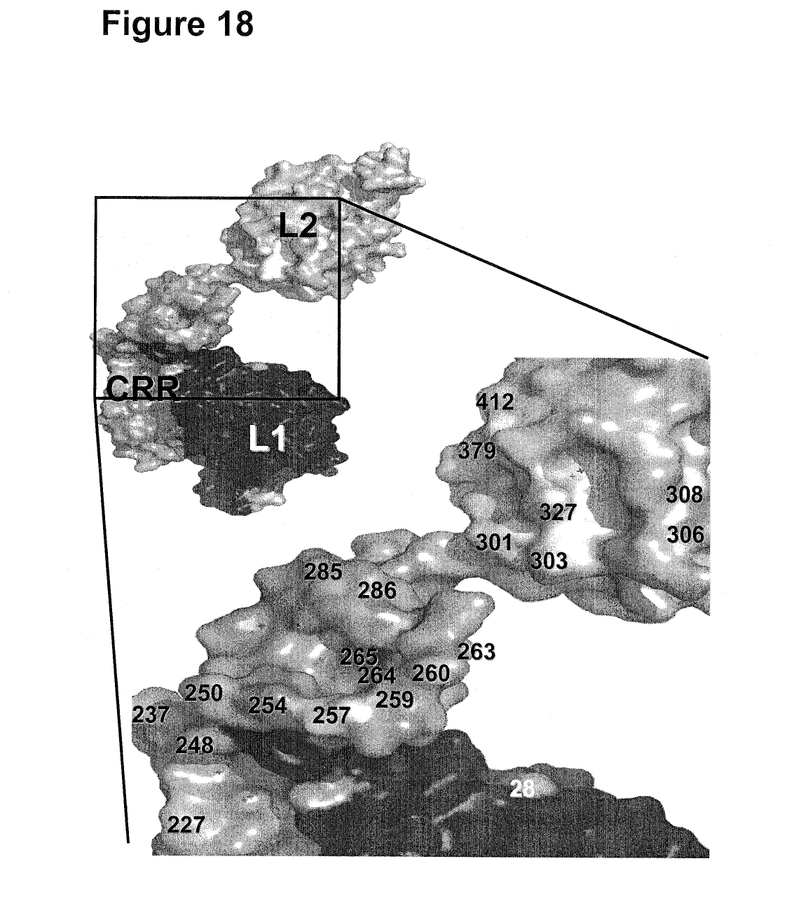

The epitopes of P1E2 and αIR3 were similar to one another, with a few minor differences. The epitopes are primarily within the CRR domain on residues overlapping with those of M14-G11, but residing on a face of the receptor rotated slightly away from the IGF-1/IGF-2 binding pocket. Additionally, residues at the C-terminus of the CRR domain and well into the L2 domain (beyond those that had any effect on M14-G11 binding) were found to marginally reduce the affinity of αIR3 alone, (Table 11). P1E2 binding to IGF-1R was ablated by mutation at residues 254 and 265; moderately reduced (10≧K_{D}≧100-fold above that of wild-type IGF-1R) by mutation at residue 257; and marginally reduced (2.5≧K_{D}≧10-fold above that of wild-type IGF-1R) by mutation at residues 248 and 303. αIR3 binding to IGF-1R was ablated by mutation at residues 248 and 265; moderately reduced (10≧K_{D}≧100-fold above that of wild-type IGF-1R) by mutation at residue 254; and marginally reduced (2.5≧K_{D}≧10-fold above that of wild-type IGF-1R) by mutation at residues 263, 301, 303, 308, 327, and 379. The position of the residues that affect P1E2 and αIR3 binding to IGF-1R (the average affect on the two antibodies) were mapped to the surface of the published structure of the first three ectodomains of IGF-1R (FIG. 18, (Garrett 1998)). αIR3 and P1E2 appear to have the same allosteric/IGF-1 only blocking characteristic of two antibodies described recently in the literature (Keyhanfar 2007). It was shown that residues 241, 242, 251, and 266 affect the ability of these antibodies to bind receptor. Our data is consistent with this report and suggests additional importance for residues 257 and 265.

The major difference between M14-G11 (competitive IGF-1 and IGF-2 blocker) and P1E2/αIR3 epitopes are in the area adjacent to the IGF-1 binding site. The ability to simultaneously recognize residues 248, 250, and 254 may be a defining factor that enables M14-G11 to competitively block both IGF-1 and IGF-2 binding. Both P1E2 and αIR3 are completely unaffected by the D250S mutation, which completely ablates M14-G11 binding to the receptor. The binding of M14-G11 to IGF-1R is also attenuated by mutations on the inner cleft of the CRR domain near the IGF-1 binding site (residues 259 and 260, FIGS. 17 & 18) perhaps explaining how this antibody sterically and competitively blocks ligand from engaging the receptor. Mutations at these positions had no effect on P1E2 or αIR3 binding. P1E2 and αIR3 affinity is attenuated by mutations on a surface slightly outside the IGF-1 binding groove (FIGS. 17 & 18). Therefore, residues that appear to be specifically recognized by M14-G11 that may lead to competitive ligand blockade are D250, E259, and S260.

Residue mutations that attenuate αIR3 and M14-G11 binding to IGF-1R extend from the center of the CRR domain into the L2 domain. It is unlikely that all these residues engage in simultaneous direct interactions with the antibodies based on published results describing average antibody epitope areas (Davies 1996). Recent data has demonstrated that the stability and folding of repeat proteins is different from most globular domains (Kajander 2005). Repeat domains tend to be elongated structures that undergo non-cooperative folding/unfolding reactions similar to helix-coil transitions of isolated α-helices. From a simplistic view, globular domains are generally cooperatively folded and exist in either a single natively folded state or a denatured state. The structures of globular domains are not partially disrupted by single mutations provided the mutation does not lead to the overall unfolding of the domain. In contrast, folded repeat domains may gradually revert to unfolded domains upon mutation. Thus, mutations along the surface of the IGF-1R CRR or L2 domains that affect antibody binding may do so by modifying the overall structure (or order) of the these domain. This mechanism also explains how antibody stabilization of a particular CRR or L2 domain conformation may affect the dynamic binding reaction of the CRR domain with ligand. This would be expected to happen in an allosteric fashion (as observed for P1E2 and αIR3) provided the antibody doesn't also sterically block ligand from binding (as observed for M14-G11).

TABLE 11
Complete list of IGF-1R mutants and their affect on antibody binding.

| Mutation | | | | | | | | |
|---|---|---|---|---|---|---|---|---|
| IGF- | | | | | | | | |
| 1R | | | | | | | | |
| position | | | IGF- | M13- | | | | |
| (w/out | | IR 3D | 1R | C06 | C03 | G11 | P1E2 | αIR3 |
| signal) | SD# | struct # | Domain | binding^{a} | binding | binding | binding | binding |
| Y28A | SD001 | 32 | L1 | NE | nd | NE | nd | nd |
| M156A | SD002 | 163 | L1 | NE | nd | NE | nd | nd |
| T188F | SD003 | 195 | L1 | NE | nd | NE | nd | nd |
| S210H | SD004 | 218 | CRR | NE | NE | NE | nd | nd |
| A211Q | | | | | | | | |
| A217T | SD005 | 224 | CRR | NE | NE | NE | nd | nd |
| A227K | SD006 | 234 | CRR | NE | NE | NE | NE | NE |
| N237G | SD007 | 244 | CRR | NE | NE | NE | NE | NE |
| S257F | SD008 | 264 | CRR | NE | NE | W | W | NE |
| E264K | SD009 | 275 | CRR | NE | NE | NE | NE | NE |
| G271D | SD010 | 282 | CRR | NE | NE | NE | NE | NE |
| G285S | SD011 | 295 | CRR | NE | NE | NE | NE | NE |
| S286T | | 296 | | | | | | |
| E303G | SD012 | 313 | L2 | NE | NE | W | W | W |
| D405K | SD013 | 415 | L2 | NE | NE | NE | nd | Nd |
| K412A | SD014 | 422 | L2 | NE | NE | NE | NE | NE |
| A413Q | | | | | | | | |
| H464E | SD015 | 474 | FnIII-1 | S | S | NE | nd | nd |
| D531Q | SD016 | 547 | FnIII-1 | NE | NE | NE | nd | nd |
| V532N | | | | | | | | |
| I650S | SD017 | * | FnIII- | NE | NE | NE | nd | nd |
| | | | 2 loop | | | | | |
| E665A | SD018 | * | FnIII- | NE | NE | NE | nd | nd |
| | | | 2 loop | | | | | |
| E739W | SD019 | * | FnIII- | NE | NE | NE | nd | nd |
| L741F | | | 2 loop | | | | | |
| S427L | SD101 | 437 | L2 | NE | NE | nd | nd | nd |
| E459A | SD102 | 469 | FnIII-1 | S | S | nd | nd | nd |
| S460A | | 470 | | | | | | |
| D461A | SD103 | 471 | FnIII-1 | S-most | S- | nd | nd | nd |
| V462T | | 472 | | critical | most | | | |
| | | | | | critical | | | |
| H464A | SD104 | 474 | FnIII-1 | S | S | nd | nd | nd |
| T466L | SD105 | 476 | FnIII-1 | W | W | nd | nd | nd |
| S467Y | | 477 | | | | | | |
| T468R | SD106 | 478 | FnIII-1 | NE | NE | nd | nd | nd |
| T478R | SD107 | 488 | FnIII-1 | NE | W | nd | nd | nd |
| H480E | SD108 | 490 | FnIII-1 | S | S | nd | nd | nd |
| Y482A | SD109 | 492 | FnIII-1 | S | S | nd | nd | nd |
| R483W | | 493 | | | | | | |
| E533H | SD110 | 548 | FnIII-1 | W | S | nd | nd | nd |
| I564T | SD111 | 578 | FnIII-1 | W | W | nd | nd | nd |
| R565A | | 579 | | | | | | |
| K568A | SD112 | 582 | FnIII-1 | NE | W | nd | nd | nd |
| E570A | SD113 | 584 | FnIII-1 | S | S | nd | nd | nd |
| I571T | | 585 | | | | | | |
| L572D | SD114 | 586 | FnIII-1 | *Fold | *Fold | nd | nd | nd |
| Y573D | | 587 | | Affected | Affect | | | |
| D248A | SD201 | 255 | CRR | nd | nd | S | W | S |
| D250S | SD202 | 257 | CRR | nd | nd | S | NE | NE |
| N254A | SD203 | 261 | CRR | nd | nd | M | S | M |
| S257K | SD204 | 264 | CRR | nd | nd | W | M | NE |
| E259K | SD205 | 270 | CRR | nd | nd | W | NE | NE |
| S260N | | 271 | | | | | | |
| S263R | SD206 | 274 | CRR | nd | nd | W | NE | W |
| G265Y | SD207 | 276 | CRR | nd | nd | W | S | S |
| V301Y | SD208 | 311 | L2 | nd | nd | NE | NE | W |
| K306E | SD209 | 316 | L2 | nd | nd | NE | NE | NE |
| T308E | SD210 | 318 | L2 | nd | nd | NE | NE | W |
| K327N | SD211 | 337 | L2 | nd | nd | NE | NE | W |
| L379R | SD212 | 389 | L2 | nd | nd | NE | NE | W |
| E381K | SD213 | 391 | L2 | nd | nd | *Fold | *Fold | *Fold |
| E382L | | 392 | | | | Affected | Affected | Affected |

^{a}No effect (NE): measured K_{D} within 2.5-fold of WT hIGF-1R-Fc; Weak (W): measured K_{D} between 2.5-10-fold higher than WT; Medium (M): measured K_{D} between 10-100-fold higher than WT; Strong (S): binding to antibody was ablated by mutation; and nd: not determined.
*“Fold Affect” implies that the mutant receptor expression was attenuated and the protein behaved in an aberrant fashion presumably because the “folding” of the receptor was “affected”.

In conclusion, it has been demonstrated that two separate epitopes on the surface of the IGF-1R ectodomain can lead to inhibition of the receptor. Novel, residue specific epitope mapping information concerning these two epitopes was based on a dataset of 46 individual or double IGF-1R mutations. The first epitope resides in FnIII-1 and leads to allosteric blockade of both IGF-1 and IGF-2 binding. The second epitope is within the CRR domain and near the putative IGF-1/IGF-2 binding site. It was discovered that subtle differences in antibody epitope within this region differentiate the ability to allosterically block the binding of a single ligand, IGF-1, from the ability to block both IGF-1 and IGF-2 competitively. Particular residues that must be targeted to achieve competitive blockade of both ligands have been identified here for the first time.

### Example 6

### Combined Targeting of Distinct IGF-1R Epitopes with Ligand-Blocking Antibodies Results in Enhanced Tumor Cell Growth Inhibition

a. Methods

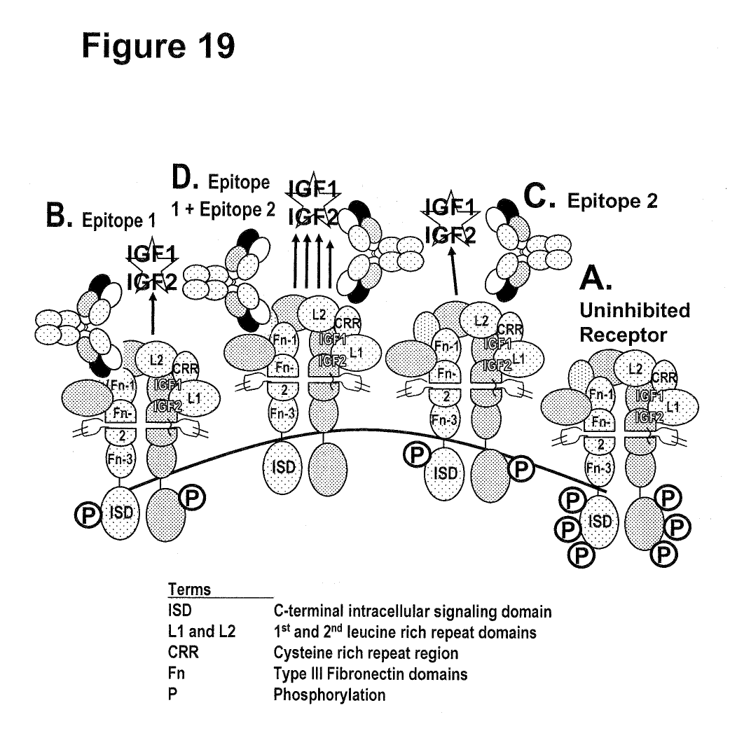

It was reasoned that antibodies which bind different IGF-1R epitopes may exhibit enhanced tumor cell growth inhibition as compared to the individual antibodies themselves (see FIG. 19). Accordingly, the ability of antibodies to block IGF-1 and IGF-2 driven tumor cell growth was tested using a cell viability assay. BxPC3 (human pancreas adenocarcinoma) and H322M (human non-small cell lung tumor)(ATCC) tumor lines were purchased from ATCC. Cell lines were grown in complete growth medium containing RPMI-1640 (ATCC) and 10% fetal bovine serum (Irvine Scientific Inc.). Trypsin-EDTA solution (Sigma) was used for removal of adherent cells from culture vessels. Phosphate buffered saline, pH 7.2, was from MediaTech Inc. The 96-well clear bottom plates for a luminescent assay was purchased from Wallac Inc. Cells grown to 80% monolayers were trypsinized, washed, resuspended, and plated into 96-well plates in 200 μl of 0.5% growth medium at 8×10³ cells/well for both BxPC3 and H322M cells. After 24 hours, the culture medium was replaced with 50 μl or 100 μl of serum free medium (SFM), and 50 μl of serially diluted antibodies at 4× concentration was added. Following another 30 minutes of incubation at 37° C., 50 μl of IGF-1 and IGF-2 at 4× concentration was added to make the final concentration 1×. All treatments were done in triplicates. The cells were incubated for another 72 hours until lysed to determine the amount of ATP present using the CELL TITER-GLO™ Luminescent Cell Viability Assay ((Promega Corporation, 2800 Woods Hollow Rd., Madison, Wis. 53711 USA). A1:1 mixture of lysis reagent and SFM (luminescent substrate) was added at 200 μl/well. Luminescence was detected on a Wallac (Boston, Mass.) plate reader. Inhibition was calculated as [1−(Ab−SFM)/(IGF−SFM)]×100%. An isotype matched antibody, IDEC-151 (human G4), was used as a negative control (“ctr” or “ctrl”).

b. Results

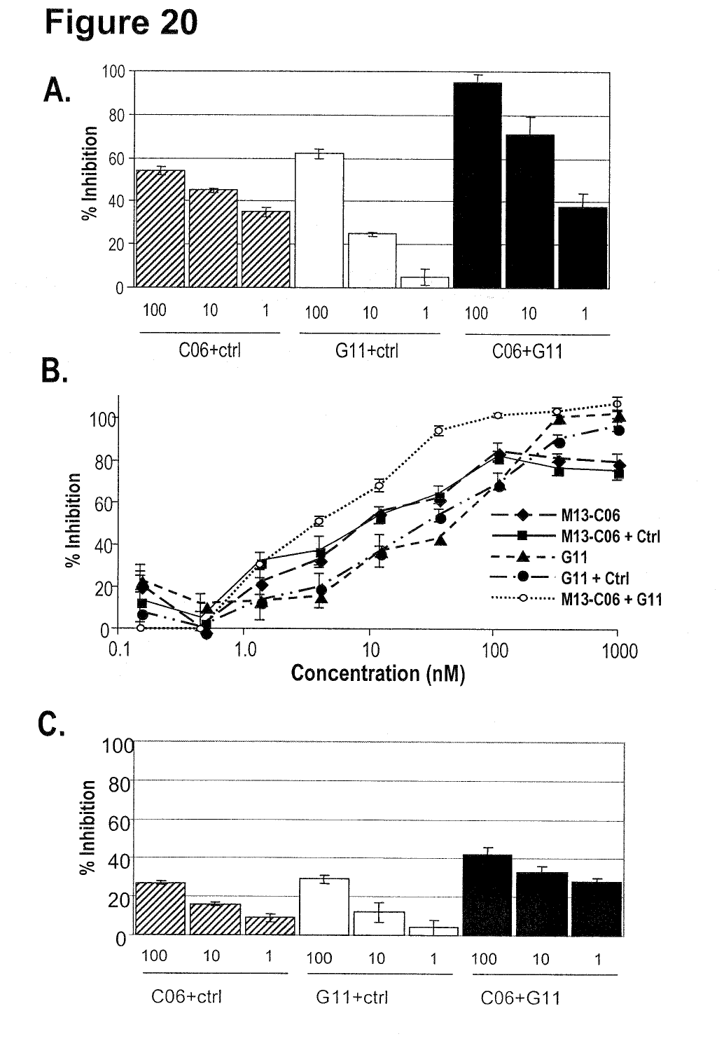

The ability of M13.C06.G4.P.agly (C06) and M14.G11.G4.P.agly (G11) anti-IGF1-R antibodies to inhibit tumor cell growth in vitro was measured indirectly by relative comparisons of cellular ATP as a measure of metabolic activity. Both C06 and G1 inhibited IGF-1 and IGF-2 stimulated growth of BxPC3 pancreatic tumor cell lines under serum-free conditions in a dose dependent manner (FIG. 20A). Importantly the cells exposed to equimolar amounts of C06 and G11 antibodies gave a significantly enhanced inhibition of growth at 10 and 1 nM concentrations compared to that of either antibody alone (FIG. 20A). These results were further confirmed in an experiment where a combination of C06 and G11 was tested at wide range of antibody concentrations (1 uM to 0.15 nM). FIG. 20B, shows that the combination of equimolar amounts of C06 and G11 antibodies resulted in significantly enhanced cell growth inhibition between 500 nM and 5 nM as compared to that observed with either antibody alone at corresponding antibody concentrations.

To demonstrate that the inhibition observed with the pancreatic cancer cell line (BxPC3) is also applicable to other tumor types, the combinations of C06 and G11 was evaluated in H322M cell line of non-small cell lung cancer origin. FIG. 20C shows an example of the effects observed in H322M grown under standard cell culture conditions in the presence of 10% fetal bovine serum, where a significantly greater inhibition of cell growth was observed by C06/G11 antibody combination compared to either antibody alone.

### Example 7

### Combined Targeting of Distinct IGF-1R Epitopes with Tetravalent Bispecific Antibodies

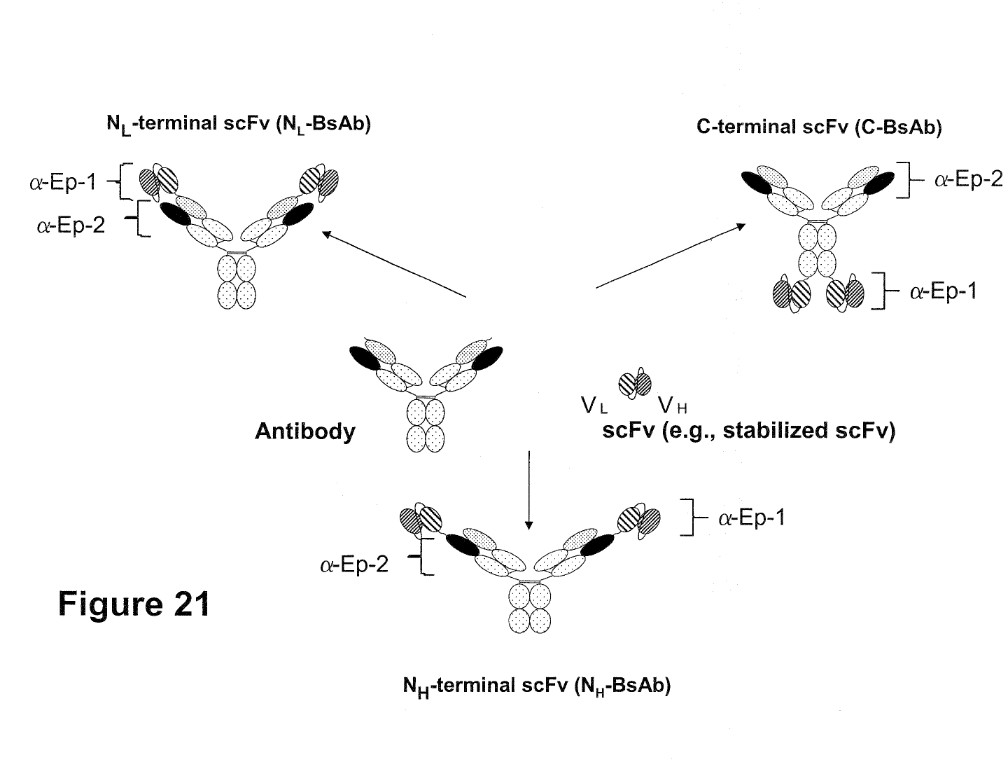

Tetravalent bispecific antibodies can be designed which combine binding sites of two monospecific antibodies of different binding specificity which exhibit enhanced or synergistic anti-IGF-1R properties when combined. An exemplary tetravalent bispecific binding molecule of the invention comprises scFv molecules with a first binding specificity and a bivalent antibody with a second binding specificity (see FIG. 21). scFv molecules may be linked or fused to the C-terminus of the heavy chain or the N-terminus of the light (NL) or heavy chain (NH) of the bivalent antibody to create a bispecific binding molecule. It is also possible using the methods described in this invention to engineer tetravalent bispecific antibodies consisting solely of stabilized scFvs fused directly to hinge regions or to CH2 or CH3 domains of the bivalent antibody. Said antibodies may comprise full-length Fc regions or CH2-domain deleted Fc regions. In other exemplary embodiments, two or more scFv domains may be fused to the same terminus of a heavy or light chain. In preferred embodiments, at least one of the scFv molecules is a stabilized scFv molecules. scFv molecules can be stabilized by introducing, via PCR-directed mutagenesis, a disulfide bond between VH 44 and VL 100 and/or a linker of optimized length (e.g., Gly4Ser4) (SEQ ID NO: 135) between the VH and VL domain of the scFv.

In certain exemplary embodiments, a tetravalent bispecific antibody may comprise M13.C06.G4.P.agly (C06) antibody that is linked or fused to stabilized scFv molecules derived from the variable regions of M14.G11.G4.P.agly (G11). Alternatively, a tetravalent bispecific antibody may comprise an M14.G11.G4.P.agly (G1) antibody that is linked or fused to scFv molecules derived from the variable regions of M13.C06.G4.P.agly (C06). For example, a (Gly₄Ser)₅ (SEQ ID NO: 184) linker may be used to connect (via PCR amplification) the scFvs to the mature N- or C-terminus of the antibody heavy chain or the N-terminus of the antibody light chain.

PCR products encoding scFvs may be purified and ligated into a mammalian vector (e.g. pN5KG1) containing the full-length precursor polypeptide sequence of the heavy or light chain. The mammalian expression vector pN5KG1 contains a translation-impaired, modified (intron-containing) neomycin phosphotransferase gene to select for transcriptionally active integration events, and a murine dihydrofolate reductase gene to permit amplification with methotrexate (Barnett, et al., Antibody Expression and Engineering. (Imanaka, H. Y. W. a. T., ed), pp. 27-40, Oxford University Press, New York, N.Y., (1995)). The ligation mixtures may be used to transform E. coli strain TOP 10 competent cells (Invitrogen Corporation, Carlsbad, Calif.). E. coli colonies transformed to ampicillin drug resistance are screened for presence of inserts. DNA sequence analysis may be used to confirm the correct sequence of the final constructs.

Plasmid DNAs are used to transform CHO DG44 cells for transient production of antibody protein. Each 20 ug of plasmid DNA is combined with 4×10⁶ cells in a volume of 0.4 mls of 1×PBS. The mixture is added to a 0.4 cm cuvette (BioRad) and placed on ice for 15 min. The cells were electroporated at 600 uF and 350 volts with a Gene Pulser electroporator (BioRad). The cells were placed in the CHO-SSFM II media containing 100 uM Hypoxanthine and 16 uM Thymidine into a T-25 flask and incubated at 37° for 4 days.

Supernatants containing tetravent bispecific antibody produced by this transient CHO expression system are collected and evaluated by Western Blot or tested for individual binding activity to recombinantly produced IGF-1R receptors in ELISA assays. The antibody can then be tested using an in vitro cell viability assay or an in vivo tumor growth inhibition assay. For example, tumor cell lines WiDr, (ATCC CCL-218) a human colon carcinoma cell line, MeI 80, (ATCC HTB 33) a human cervical epithelial carcinoma cell line, and MDA231, (Dr. Dajun Yang, University of Michigan) a human breast carcinoma cell line are cultured in MEM-Earles with 10% FCS, 2 mM L-Glutamine, 1× non-essential amino acids, 0.5 mM sodium pyruvate, and Penicillin/Streptomycin. Tumor cell lines were rinsed once with PBS and cells released by digestion with trypsin. Cells are collected by centrifugation, resuspended in complete media, counted and 96-well tissue culture plates seeded at 5000 cells/well for WiDr and Me180 and 1500 cell/well for MDA231.

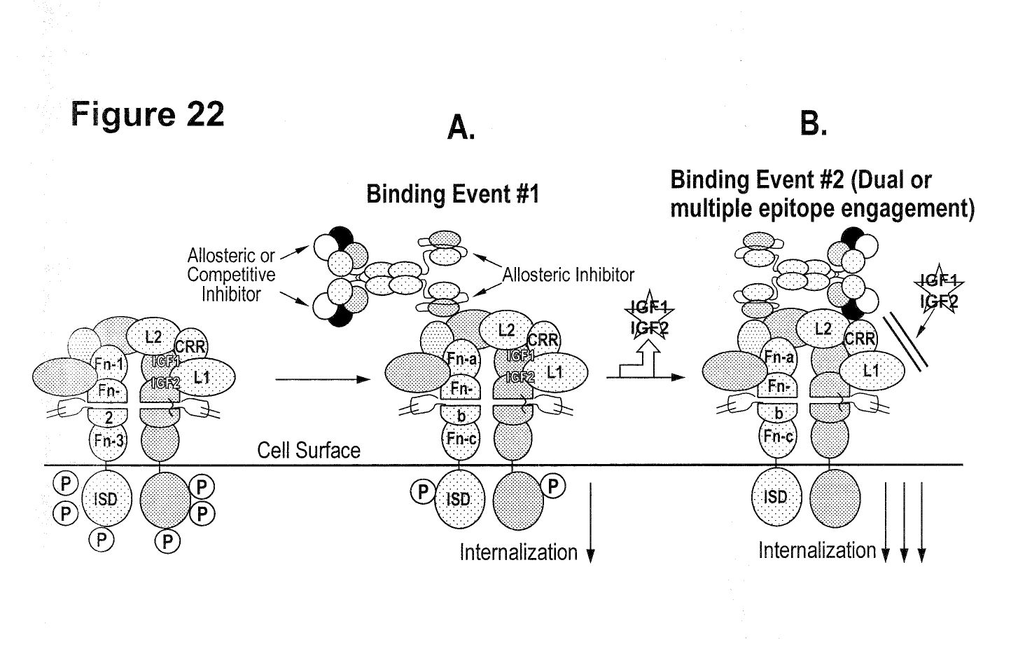

Bispecific tetravalent IGF-1R antibodies may produce enhanced tumor cell killing relative to monospecific IGF-1R antibodies. For example, bispecific tetravalent IGF-1R antibodies can bind to either an allosteric epitope or a competitive epitope in IGF-1R and elicit IGF-1R internalization (see Binding event #1, FIG. 22A). Alternatively, bispecific tetravalent IGF-1R antibodies can bind to two or more epitopes (see Binding event #2, FIG. 22B) and thereby elicit enhanced IGF-1R internalization.

### Example 8

### Stability-Engineering, Molecular Biology, and Protein Expression of Anti-IGF-1R Bispecific Antibodies

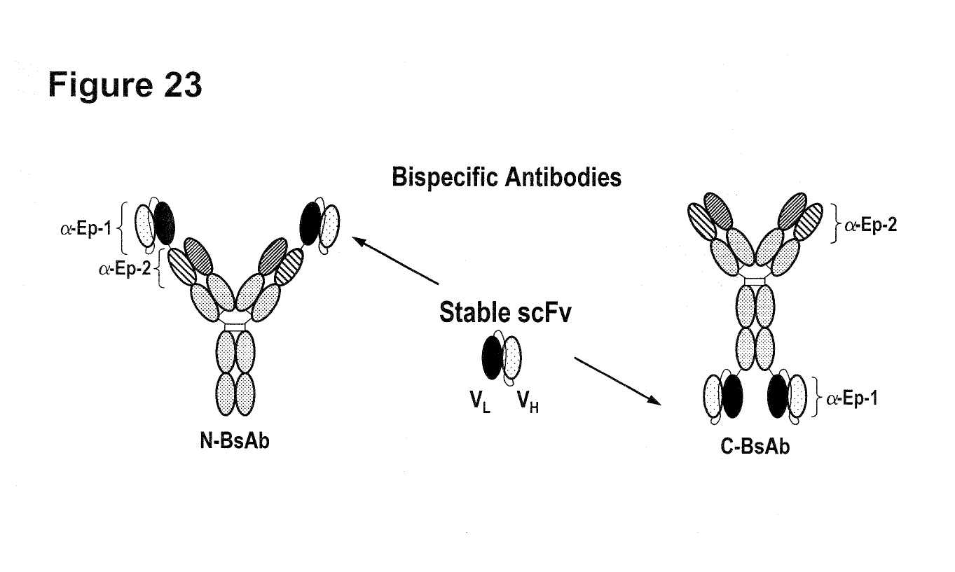

FIG. 23 shows a schematic representation of anti-IGF-1R IgG-like bispecific antibodies (“BsAbs”) of the invention. The designs consisted of a stabilized scFv that binds to an epitope (e.g. Ep-1) present on IGF-1R and is genetically connected, through a flexible linker, to either the amino (N-BsAb) or carboxyl terminus (C-BsAb) of a full-length anti-IGF-1R antibody that specifically binds to a second distinct epitope (e.g. Ep-2). In the example shown in FIG. 23 the scFv is built in the VL→VH orientation but the scFv may also be designed to function in the VH→VL format.

i. Expression Constructs

In general, unless otherwise indicated, the expression constructs for scFvs and antibody heavy chain in the following Examples included a nucleotide sequence encoding an N-terminal signal peptide having the amino acid sequence MGWSLILLFLVAVATRVLS (SEQ ID NO: 134). Expression constructs for antibody light chains included a nucleotide sequence encoding the amino acid sequence MDMRVPAQLLGLLLLWLPGARC (SEQ ID NO: 131). Expression constructs for scFv molecules included C-terminal Myc and His tags comprising the sequence DDDKSFLEQKLISEEDLNSAVDHHHHHH to facilitate purification.

b. Preparation of Anti-IGF-1R scFv and Fab Proteins

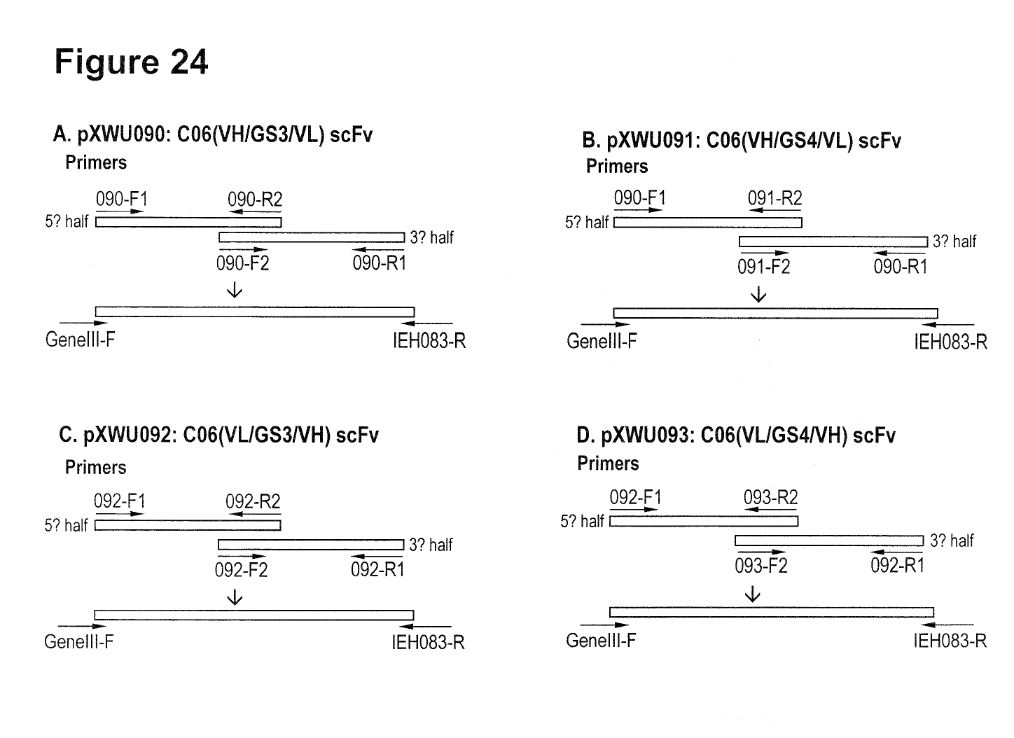

Anti-IGF-1R C06 scFvs were subcloned and assembled using a two-step PCR amplification protocol from plasmids described in U.S. Patent Application 20070243194. The following four C06 scFvs were prepared in the indicated orientations and with the indicated linkers: 1) VH→(Gly₄Ser)₃ linker (SEQ ID NO: 185)→VL (VH/GS3/VL), 2) VH→(Gly₄Ser)₄ linker (SEQ ID NO: 135)→VL (VH/GS4/VL), 3) VL→(Gly₄Ser)₃ linker (SEQ ID NO: 185)→VH (VL/GS3/VH), and 4) VL→(Gly₄Ser)₄ linker (SEQ ID NO: 135)→VH (VL/GS4/VH). FIG. 24 shows a schematic representation of the protocol for the two-step PCR assembly of C06 scFvs. Oligonucleotides used in the construction are shown in Table 12. As an example, C06 (VL/GS3/VH) scFv was constructed by first creating two separate PCR products—the 5′ half of the scFv and the 3′ half. The two halves were then joined through the common (Gly₄Ser)₃ (SEQ ID NO: 185) overlapping region in a second PCR reaction. Briefly, the 5′ half of C06 (VL/GS3/VH) scFv was generated by PCR using the forward 5′ VL primer 092-F1 which consists of 28 bases encoding part of the gpIII leader sequence followed by 22 bases of sequence complementary to the C06 N-terminal variable light domain gene and the reverse 3′ VL primer 092-R2 which consists of 22 bases of sequence complementary to the C06 C-terminal variable light domain gene and nucleotide sequences encoding the (Gly₄Ser)₃ (SEQ ID NO: 185) linker peptide. The 3′ half of C06 (VL/GS3/VH) scFv was generated by PCR using the forward 5′ VH primer 092-F2 which consists of nucleotide sequences encoding the (Gly₄Ser)₃ (SEQ ID NO: 185) linker peptide and 22 bases of sequence complementary to the C06 N-terminal variable heavy domain gene and the reverse 3′ VH primer 092-R1 which consists of 22 bases of sequence complementary to the C06 C-terminal variable heavy domain gene followed by nucleotide sequences encoding a Myc-tag. The C06 (VH/GS3/VL) scFv was then finally assembled in a second PCR reaction using the two previously synthesized 5′ and 3′ half gene fragments, each containing sequences encoding the overlapping (Gly₄Ser) 3 (SEQ ID NO: 185) linker peptide, the 5′ forward primer GeneIII-F which contained a unique Nde I endonuclease site followed by sequence encoding the N-terminal portion of the gpIII leader sequence and the 3′ reverse primer IEH083-R which contained a Sal I site and sequence encoding a Myc-tag.

A PCR product corresponding to the expected size was resolved by agarose gel electrophoresis, excised, and purified using the Millipore Ultrafree-DA extraction kit according to manufacturer's instructions (Millipore; Bedford, Mass.). The purified PCR product was subsequently digested with Nde I and Sal I and cloned into the Nde I/Sal I sites of a modified E. coli expression vector designed to drive recombinant protein expression under the control of an inducible ara C promoter. The expression vector contained a modification encoding a unique Nde I site overlapping the start codon of the C06 scFv and a His-tag at the C-terminus followed by a stop codon. The gel purified PCR product was ligated to the Nde I/Sal I digested expression plasmid and a portion of the ligation mixture was used to transform E. coli strain XL1-Blue. Ampicillin drug resistant colonies were screened and DNA sequence analysis confirmed the correct sequence of the final plasmid pXWU092 encoding the C06 (VL/GS3/VH) scFv. The scFv construct contained a gIII signal peptide and terminated with a c-myc-His₆ detection tag.

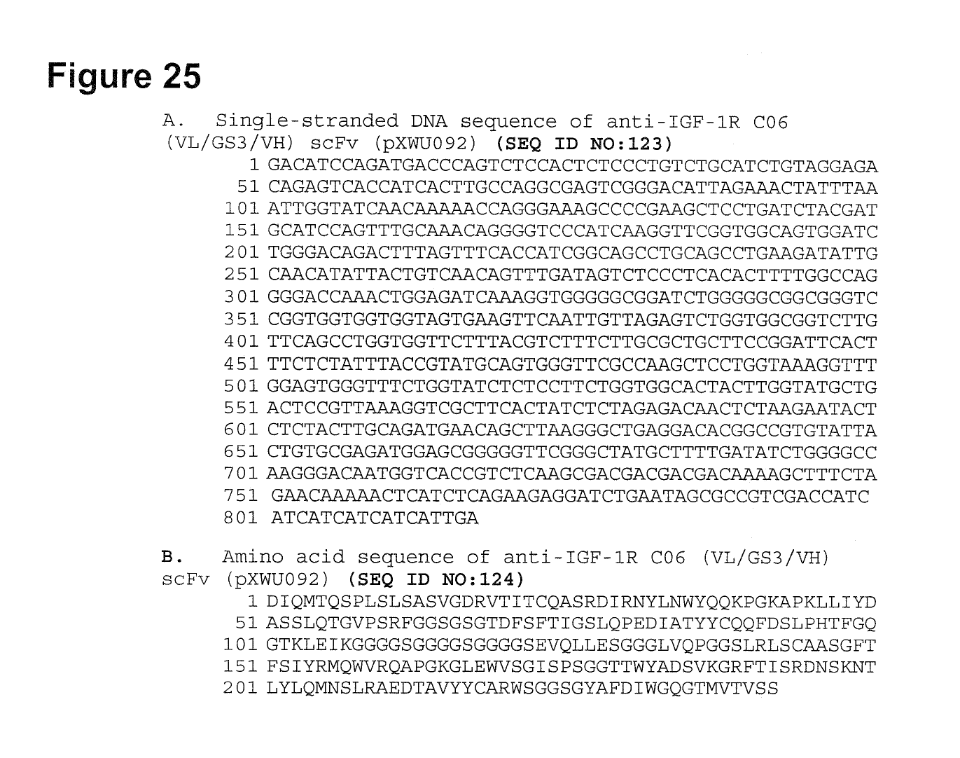

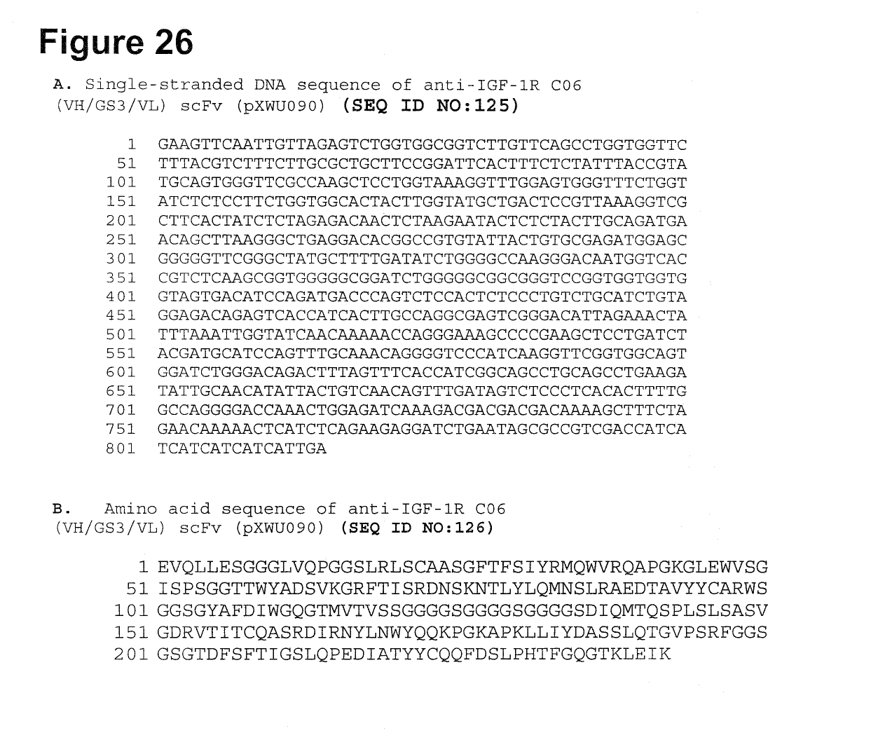

The remaining three anti-IGF-1R C06 scFvs were prepared in a similar fashion as described above using oligonucleotides listed in Table 12 and following the schema shown in FIG. 24. Plasmid pXWU090 encoded C06 (VH/GS3/VL) scFv, pXWU091 encoded C06 (VH/GS4/VL) scFv, and plasmid pXWU093 encoded C06 (VL/GS4/VH) scFv. DNA and amino acid sequences of exemplary scFvs C06 (VL/GS3/VH) scFv (pXWU092) and C06 (VH/GS3/VL) scFv (pXWU090) are shown in FIGS. 25A and 25B, and in FIGS. 26A and 26B, respectively.

TABLE 12

Oligonucleotides for PCR
amplification of conventional C06 scFvs.

| | |
|---|---|
| Primers | Sequence |

| | |
|---|---|
| GeneIII-F | 5′-GAATTACATATGAAAAAACTGCTGTTCGCGA |
| (SEQ ID NO:145) | TTCCGCTGGTGGTGCCGTTCTATAGC-3′ |
| IEH083-R | 5′-ATGATGGTCGACGGCGCTATTCAGATCCTCT |
| (SEQ ID NO:146) | TCTGAGATGAGTTTTTGTTCTAGAAAGC-3′ |
| 090-F1 | 5′-GCTGGTGGTGCCGTTCTATAGCCATAGTGAA |
| (SEQ ID NO:147) | GTTCAATTGTTAGAGTCTGG-3′ |
| 090-F2 | 5′-TCTGGGGGCGGCGGGTCCGGTGGTGGTGGTA |
| (SEQ ID NO:148) | GTGACATCCAGATGACCCAGTCTCC-3′ |
| 090-R1 | 5′-GAGTTTTTGTTCTAGAAAGCTTTTGTCGTCG |
| (SEQ ID NO:149) | TCGTCTTTGATCTCCAGTTTGGTCCCCTG-3′ |
| 090-R2 | 5′-CCGGACCCGCCGCCCCCAGATCCGCCCCCAC |
| (SEQ ID NO:150) | CGCTTGAGACGGTGACCATTGTC-3′ |
| 091-F2 | 5′-GGGCGGATCTGGGGGCGGCGGGTCCGGTGGT |
| (SEQ ID NO:151) | GGTGGTAGTGACATCCAGATGACCCAGTCTC-3′ |
| 091-R2 | 5′-CCCGCCGCCCCCAGATCCGCCCCCACCGGAC |
| (SEQ ID NO:152) | CCTCCACCGCCGCTTGAGACGGTGACCATTGT |
| | C-3′ |
| 092-F1 | 5′-GCTGGTGGTGCCGTTCTATAGCCATAGTGAC |
| (SEQ ID NO:153) | ATCCAGATGACCCAGTCTC-3′ |
| 092-F2 | 5′-ATCTGGGGGCGGCGGGTCCGGTGGTGGTGGT |
| (SEQ ID NO:154) | AGTGAAGTTCAATTGTTAGAGTCTGGTGG-3′ |
| 092-R1 | 5′-GAGTTTTTGTTCTAGAAAGCTTTTGTCGTCG |
| (SEQ ID NO:155) | TCGTCGCTTGAGACGGTGACCATTGTC-3′ |
| 092-R2 | 5′-ACCGGACCCGCCGCCCCCAGATCCGCCCCCA |
| (SEQ ID NO:156) | CCTTTGATCTCCAGTTTGGTCCCCTG-3′ |
| 093-F2 | 5′-GGGCGGATCTGGGGGCGGCGGGTCCGGTGGT |
| (SEQ ID NO;157) | GGTGGTAGTGAAGTTCAATTGTTAGAGTCTG-3′ |
| 093-R2 | 5′-CCGCCGCCCCCAGATCCGCCCCCACCGGACC |
| (SEQ ID NO:158) | CTCCACCGCCTTTGATCTCCAGTTTGGTCCCCT |
| | G-3′ |

For expression of conventional C06 scFvs, freshly isolated colonies of E. coli strain W3110 (ATCC, Manassas, Va. Cat. # 27325) transformed with plasmids pXWU090, pXWU091, pXWU092, and pXWU093 were cultivated and either culture supernatants or periplasm extracts were prepared as described in U.S. patent application Ser. No. 11/725,970, which is incorporated by reference herein in its entirety. C06 FAb was prepared by enzymatic digestion of C06 IgG. Purified FAb was concentrated to ˜2 mg/mL. Fab concentration was determined using an ε₂₈₀ nm=1.5 mL mg⁻¹ cm⁻¹.

c. Thermal Stability of Conventional C06 scFv Antibodies

A thermal challenge assay described in U.S. patent application Ser. No. 11/725,970 was employed as a stability screen to determine the temperature at which 50% of the C06 (VL/GS3/VH) and C06 (VH/GS3/VL) scFvs molecules retain their antigen binding activity following a thermal challenge event.

E. coli strain W3110 (ATCC, Manassas, Va. Cat. # 27325) was transformed with plasmids pXWU090, pXWU091, pXWU092 and pXWU093 encoding the various C06 scFvs under the control of an inducible ara C promoter. Transformants were grown overnight in expression media consisting of SB (Teknova, Half Moon Bay, Ca. Cat. # S0140) supplemented with 0.6% glycine, 0.6% Triton X100, 0.02% arabinose, and 50 μg/ml carbenicillin at 30° C. Bacteria was pelleted by centrifugation and supernatants harvested for further treatment.

After thermal challenge, the aggregated material was removed by centrifugation and soluble scFv samples remaining in the treated, cleared supernatant were assayed for binding to cognate soluble IGF-1R-Fc antigen by DELFIA assay. A 96-well plate (MaxiSorp, Nalge Nunc, Rochester, N.Y., Cat. # 437111) was coated overnight at 4° C. with soluble IGF-1R-Fc antigen at 1 μg/ml in PBS, and then blocked with DELFIA assay buffer (DAB, 10 mM Tris HCl, 150 mM NaCl, 20 μM EDTA, 0.5% BSA, 0.02% Tween 20, 0.01% NaN₃, pH 7.4) for one hour with shaking at room temperature. The plate was washed 3 times with DAB without BSA (Wash buffer), and test samples diluted in DAB were added to the plates in a final volume of 100 □l. The plate was incubated for one hour with shaking at room temperature, and then washed 3 times with Wash buffer to remove unbound and functionally inactivated scFv molecules. Bound scFv was detected by addition of 100 □l per well of DAB containing 250 ng/ml of Eu-labeled anti-His₆ antibody (Perkin Elmer, Boston, Mass., Cat. # AD0109) and incubated at room temperature with shaking for one hour. The plate was washed 3 times with Wash buffer, and 100 □l of DELFIA enhancement solution (Perkin Elmer, Boston, Mass., Cat. # 4001-0010) was added per well. Following incubation for 15 minutes, the plate was read using the Europium method on a Victor 2 (Perkin Elmer, Boston, Mass.). Data was analyzed using Prism 4 software (GraphPad Software, San Diego, Calif.) using a sigmoidal dose response with variable slope as the model. The values obtained for the mid-point of the thermal denaturation curves are referred to as T₅₀ values, and are not construed as being equivalent to biophysically derived Tm values.

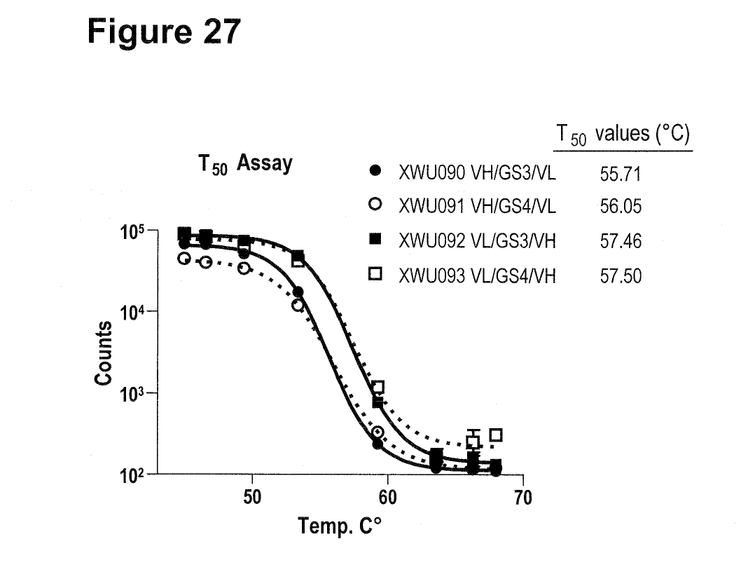

Results from this assay determined the T₅₀ value of C06 (VL/GS3/VH) to be 57.46° C. and C06 (VH/GS3/VL) to be slightly lower at 55.71° C. (FIG. 27). Given the absence of meaningful differences in the T₅₀ values for constructs varying only by linker length, and the observation that of the two orientations C06 (VL→(Gly₄Ser)_{n=3 or 4} (SEQ ID NO: 185 or 135)→VH) scFvs were slightly more thermally stabile than C06 (VH→(Gly₄Ser)_{n=3 or 4} (SEQ ID NO: 185 or 135)→VL) scFvs, C06 (VL/GS3/VH) containing the (Gly₄Ser)₃ linker (SEQ ID NO: 185)(plasmid pXWU092) was selected for further stability engineering.

d. Construction of C06 scFv Molecules with Improved Thermal Stability

Individual variants and libraries were designed to contain desired amino acid replacements in the conventional C06 (VL/GS3/VH) scFv (pXWU092) using oligonucleotides listed in Table 13. In Table 13, each oligonucleotide name gives reference to desired amino acid substitutions at position(s) in VH or VL according to Kabat numbering system.

TABLE 13

Oligonucleotides and rationale for
construction of variant C06 (VL/GS3/VH) scFvs.

| | | | |
|---|---|---|---|
| Oligo name | Rationale | Sequence | SEQ ID NO: |

| | | | |
|---|---|---|---|
| VL_M4L | COVAR | CATAGTGACATCCAGCTGACCCAGTCTCCACTC | 159 |
| VH_W47IF | COMP | GGTAAAGGTTTGGAGWTCGTTTCTGGTATCTCTC | 160 |
| VL_V15PLDSAG | COMP, COVAR | CTCCCTGTCTGCATCTVNCGGAGACAGAGTCACC | 161 |
| VL_Q24AKR | CONS | GTCACCATCACTTGCRVGGCGAGTCGGGACATTAG | 162 |
| VL_T20R | COVAR | GTAGGAGACAGAGTCCGCATCACTTGCCAGGCG | 163 |
| VL_R30TN | COMP | GCGAGTCGGGACATTAMCAACTATTTAAATTGG | 164 |
| VH_S21F | COMP | GGTTCTTTACGTCTTGAATGCGCTGCTTCCGG | 165 |
| VL_A51G | COMP | CTCCTGATCTACGATGGCTCCAGTTTGCAAACAG | 166 |
| VL_S72TY | COVAR | CTGGGACAGACTTTWMCTTCACCATCGGCAGC | 167 |
| VL_I83ETALFS | COVAR, COMP | CTGCAGCCTGAAGATNNKGCAACATATTACTGTC | 168 |
| VH_R83KT | COVAR, COMP | CTGCAGCCTGAAGATNNKGCAACATATTACTGTC | 169 |
| VH_T110V | COVAR | CAAGGGACAATGGTCGTGGTCTCAAGCGACGAC | 170 |

“Rationale” refers to the design method employed. Said methods are described in detail in U.S. Pat. Application No. 11/725,970.
Abbreviations are: “COMP”- Computational Analysis,
“COVAR”-Covariation Analysis,
“CONS”-Consensus Scoring,
“DEGEN”- degenerate codons,
Ambiguous bases are abbreviated as follows: W = A or T, V = A or C or G, Y = C or T, S = C or G, M = A or C, N = A or C or G or T, R = A or G, K = G or T, B = C or G or T (J Biol Chem. 261(1):13-7 (1986)).

Mutagenesis reactions were performed and individual transformed colonies were picked into deep-well 96 well dishes, processed, and screened according to the methods detailed in U.S. patent application Ser. No. 11/725,970. Transformants were grown overnight in expression media consisting of SB (Teknova, Half Moon Bay, Calif. Cat. # S0140) supplemented with 0.6% glycine, 0.6% Triton X100, 0.02% arabinose, and 50 μg/ml carbenicillin at either 30° C. or 32° C.

Each library was screened in duplicate using a thermal challenge assay with supernatant from one replicate subjected to treatment conditions and the second supernatant serving as untreated reference. After thermal challenge, the aggregated material was removed by centrifugation and assayed in the soluble IGF-1R Fc DELFIA as described in Example 3.

Assay data was processed using Spotfire DecisionSite software (Spotfire, Somerville, Mass.) and expressed as the ratio of the DELFIA counts observed at challenge temperature to the reference temperature for each clone. Clones that reproducibly gave ratios greater than or equal to twice what was observed for the parental plasmid were considered hits. Plasmid DNAs from these positive clones were isolated by mini-prep (Wizard Plus, Promega, Madison, Wis.) and retransformed back into E. coli W3110 for confirmation secondary thermal challenge assays as well as for DNA sequence determination.

Primary and confirmatory results from these assays are shown in Table 14. Many of the stabilized scFv molecules of the invention resulted in improvements in binding activity (T₅₀>58° C.) as compared with the conventional C06 scFv (pXWU092). In particular, the T₅₀ values of variant C06 scFvs from library position VL4 (M4L), library position V_{L}15 (V15N, V15S, V15D, V15I, V15R, V15A and V15P), library position V_{L}24 (Q24K), library position V_{L}30 (R30T), library position V_{L}51 (A51G), library position V_{L}72 (S72N), and library position V_{L}83 (I83M, I83V, I83G, I83D, I83Q, I83E and I83S), exhibited increases in thermal stability (□T₅₀ values) ranging from +3° C. to +7° C. relative to the conventional C06 scFv. The T₅₀ values of variant C06 scFvs from library position V_{H}47 (W47F), library position V_{H}83 (R83K and R83T), and library position V_{H}110 (T110V) exhibited increases in thermal stability ranging from +4° C. to +6° C. relative to the conventional C06 scFv. T₅₀ value of variant C06 scFvs from library position V_{L}72 (S72Y and S72N) exhibited increases in thermal stability of +2° C. to +5° C., respectively, relative to the conventional C06 scFv.

Two variant C06 scFvs contained stabilizing mutations, V_{L} D70E and V_{L} T74S, that serendipitously arose from either PCR errors or mutations in the oligonucleotides primers and exhibited increases in thermal stability of +4° C. and +5° C., respectively, relative to the conventional C06 scFv and were included in further analyses.

TABLE 14
C06 VH and VL library positions, library composition,
and screening results.

| Position | Library | Hit Seq. Observed | ΔT₅₀ (° C.) |
|---|---|---|---|
| VL4 | L | M4L | +4 |
| VL15 | N, T, I, S, H, P, R, L, | V15N, V15I, V15S, | +6, +3. +3, |
| | D, A, G, V | V15D, V15R, | +3, +3, |
| | | V15P, V15A, V15G | +3, +3, +3, |
| VL24 | K, T, R, E, A, G | Q24K | +3 |
| VL30 | N, T | R30T | +3 |
| VL47 | * | T47S | +5 |
| VL51 | G | A51G | +7 |
| VL72 | N, T, Y, S | S72N, S72Y | +5, +2 |
| VL74 | * | D70E | +4 |
| VL83 | All amino acids | I83M, I83V, I83D, | +5, +4, +4, |
| | | I83Q, I83E, I83S, | +5, +6, +5, |
| | | I83G | +6 |
| VH47 | F | W47F | +6 |
| VH83 | K, T | R83K, R83T | +5, +4 |
| VH110 | V | T110V | +6 |

* Non-targeted mutations arising from PCR artifacts

Plasmids consisting of various permutations of stabilizing mutations VL V15N, VL V15S, VL T20R, VL R30N, VL R30Y, VL G63S, VL S72N, VL S72Y, VL S77G, VL I83G, VL I83Q, and VH T110V were then constructed to identify combinations that further enhanced thermal stability. Individual transformed colonies were picked into deep-well 96 well dishes and processed for screening as described above. C06 scFv proteins containing individual and combined stabilizing mutations were tested for: 1) thermal stability in the T₅₀ thermal challenge assay, and 2) kinetic off-rates (dissociation constant or k_{d}) to IGF-1R-Fc by biolayer interferometry (Octet QK System, ForteBio, Inc. Menlo Park, Calif.). For off-rate analyses IGF-1R Fc was immobilized onto the surface of Protein A biosensors. Culture supernatants containing variant C06 scFvs from thirty clones were screened against the antigen for binding and subsequent dissociation constant analysis. ScFvs were allowed to bind to immobilized IGF-1R Fc for 300-seconds. Off-rate was subsequently assayed for 600 seconds. Dissociation constants (k_{d}) were calculated using software provided by the manufacturer and off-rates compared to the k_{d} of the conventional C06 scFv. Table 15 summarizes the results of the T₅₀ thermal challenge assay and off-rate analyses. Individual and combined stabilizing mutations were identified that exhibited increases in thermal stability ranging from +8° C. to +10° C. relative to the conventional C06 scFv. The T₅₀ values of combined C06 mutations at positions V_{L}15 and V_{H}110 (V_{L} L15S: V_{H} T110V) and positions V_{L}77 and V_{L}83 (S77G:I83Q) enhanced the thermal stability (T₅₀) of the scFv by +8-10° C. relative to the conventional C06 scFv.

All of the stability-engineered C06 scFvs tested had less than a 2-fold change in off-rate compared to the conventional C06 scFv (pXWU092). Based on stability (high T₅₀ value) and off-rate properties one stability-engineered C06 scFv, MJF045, was selected as an example for the construction and production of stable anti-IGF-1R bispecific antibodies.

TABLE 15
Amino acid substitutions of stabilized C06 scFv proteins, T₅₀
results from thermal challenge assay, and off-rate determination.
Proteins exhibiting T₅₀ ≧ 66° C. are shown in bold.

| Name | Mutation | T₅₀ (° C.) | K_{d} (1/sec) |
|---|---|---|---|
| XWU092 | WT | 60-61 | 1.00 |
| MJF014 | VL M4L | 64-65 | 0.86 |
| MJF015 | VH W47F | >70 | 1.37 |
| MJF016 | VL D70E | 64-65 | 1.03 |
| MJF017 | VL T74S | 65-66 | 1.02 |
| MJF018 | VL V15N | 66-67 | 1.16 |
| MJF020 | VL V15E | 63-64 | 0.76 |
| MJF021 | VL V15I | 63-64 | 0.81 |
| MJF022 | VL V15R | 63-64 | 0.76 |
| MJF023 | VL L11G, V15N | 64-65 | 0.68 |
| MJF024 | VL V15A | 63-64 | 0.89 |
| MJF025 | VL V15P | 63-64 | 1.18 |
| MJF026 | VL V15S; VH T110V | >70 | 0.99 |
| MJF027 | VL Q24K | 63-64 | 1.12 |
| MJF029 | VL T20R, R30Y | 62 | 0.68 |
| MJF031 | VL R30N | 63-64 | 0.59 |
| MJF033 | VL T20R, R30N; VH S21E | 64-65 | 0.78 |
| MJF034 | VL A51G | 67 | 0.81 |
| MJF035 | VL S72N | 65-66 | 0.54 |
| MJF037 | VL S72Y | 62-63 | 0.76 |
| MJF038 | VL G63S, S72N | 63-64 | 0.76 |
| MJF039 | VL T20R, R30N, G63S, S72N | 63-65 | 0.56 |
| MJF040 | VL I83M | 65 | 0.76 |
| MJF042 | VL S77G, I83G | 64-65 | 0.55 |
| MJF043 | VL I83D | 64-65 | 1.12 |
| MJF045 | VL I83E | 69 | 1.47 |
| MJF047 | VL I83G | 66-67 | 1.44 |
| MJF048 | VH R83K | 65-66 | 1.37 |
| MJF049 | VH R83T | 64-65 | 1.41 |
| MJF050 | VH T110V | 66-67 | 1.68 |
| MJF051 | VL S77G, I83Q | 68 | 1.10 |

e. Production of Stabilized Anti-IGF-1R Bispecific Antibodies

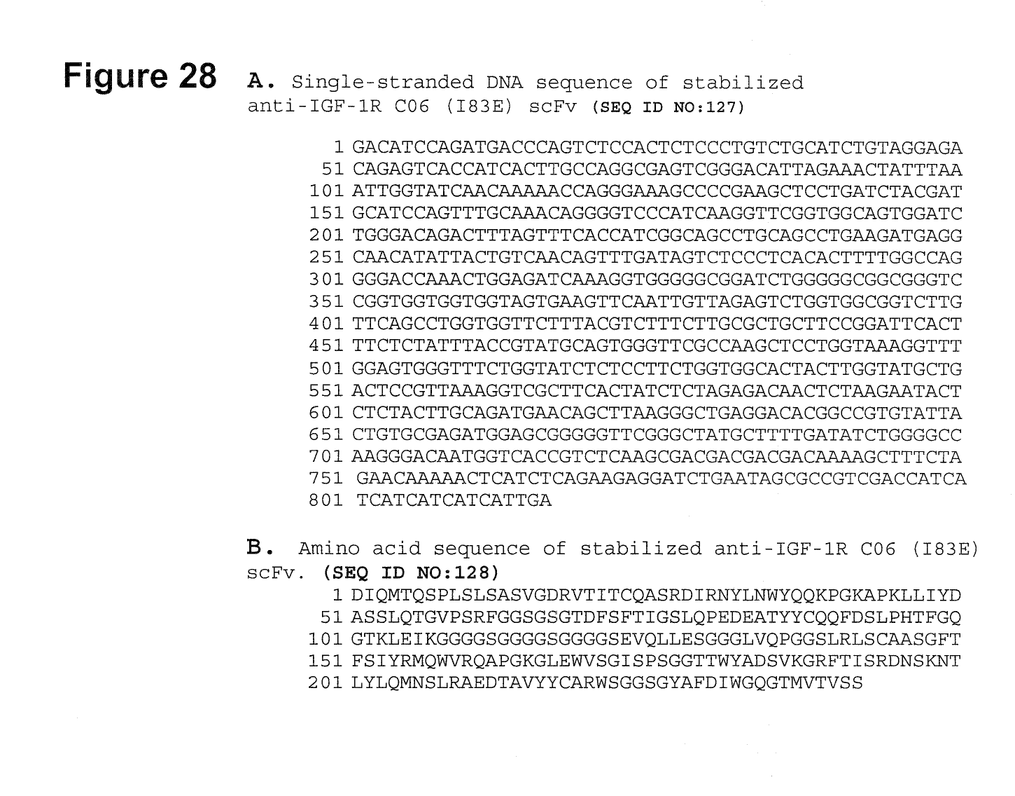

Stabilized C06 V_{L} I83E scFv (MJF045) of the invention was used to construct bispecific antibodies as both N-terminal and C-terminal scFv fusions as shown in FIG. 23. DNA and amino acid sequences of C06 MJF045 scFv are shown in FIGS. 28A and 28B, respectively.

i. Construction of N-Terminal Anti-IGF-1R Bispecific Antibodies

The MJF045 C06 scFv DNA described in Example 4 supra was used to construct an N-terminal anti-IGF-1R bispecific antibody using methods similar to that described in U.S. patent application Ser. No. 11/725,970. A (Gly₄Ser)₅ (SEQ ID NO: 184) linker was used to connect the stabilized C06 scFv to the mature amino terminus of the G11 IgG lys (−) heavy chain. First, an intermediate N-terminal anti-IGF-1r BsAb heavy chain vector was constructed by subcloning a Mlu I+BamH I DNA fragment encoding the G11 IgG lys (−) sequence from the anti-IGF-1R G11 plasmid described in U.S. Patent Application 20070243194. The intermediate vector, pXWU117, contained a synthetic heavy-chain leader sequence, the G11 IgG lys (−) sequence followed by a BamH I site at the carboxyl terminus of the IgG1 CH3 domain. Next, the MJF045 C06 scFv was subcloned and modified using a two-step PCR amplification protocol using the oligonucleotide primers described in Table 16. Briefly, in the first reaction a PCR product was produced using the stabilized C06 scFv (MJF045) as template, the forward 5′C06 scFv VL PCR primer 136-F which included a Mlu I restriction endonuclease site followed by sequences encoding the last three amino acids of the heavy chain signal peptide followed by sequences complementary to the amino terminus of C06 scFv VL and the reverse 3′C06 scFv VH primer, 136-R2. 136-R2 consisted of sequences complementary to the carboxyl terminus of C06 scFv VH followed by nucleotide sequences encoding a portion of (Gly4Ser)₅ (SEQ ID NO: 184) linker. After several cycles of PCR a second reverse primer, 136-R1a, consisting of sequences complementary to the (Gly4Ser)₅ (SEQ ID NO: 184) linker and the N-terminus of G11 VH followed by an internal Nhe I site was added to the PCR mixture and the reaction resumed for an additional 20 cycles.

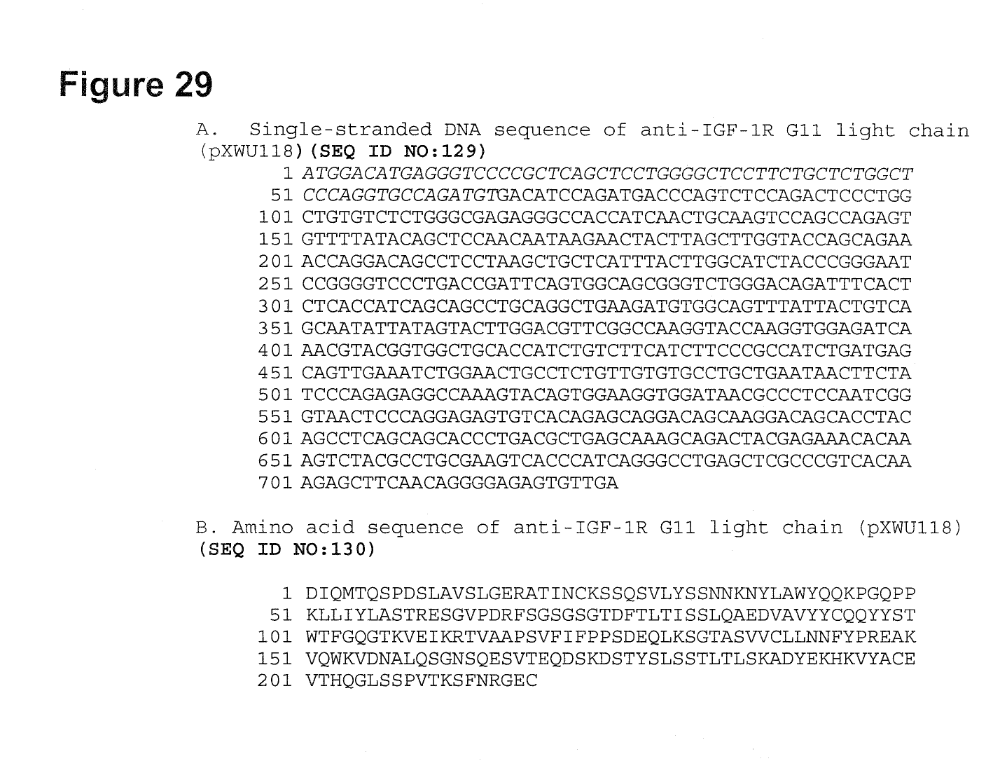

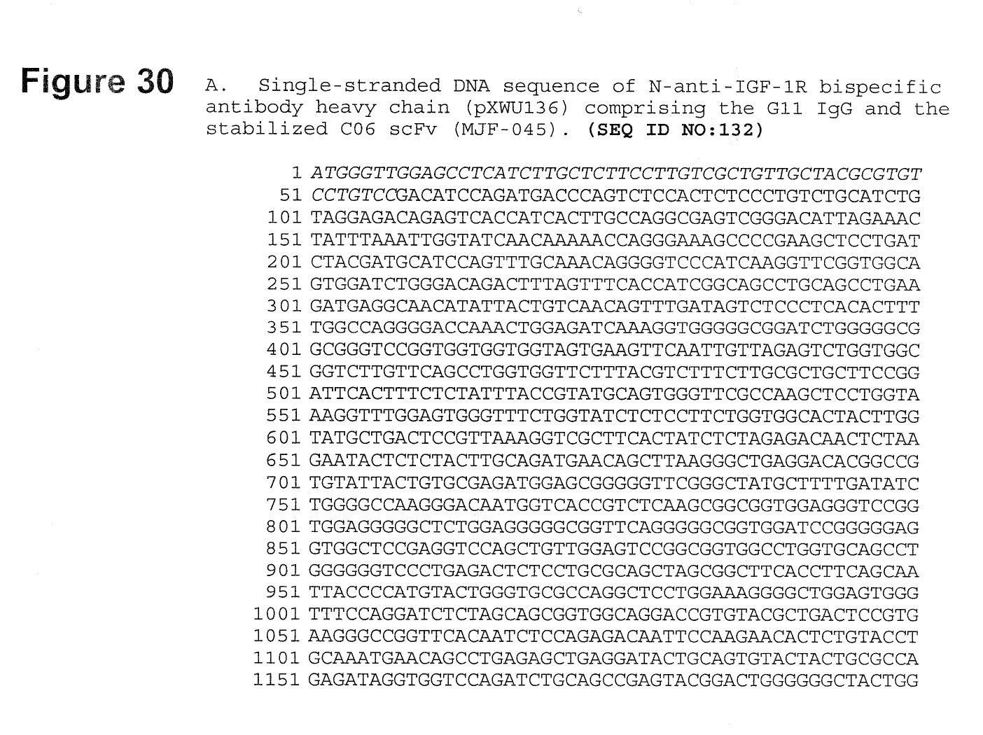

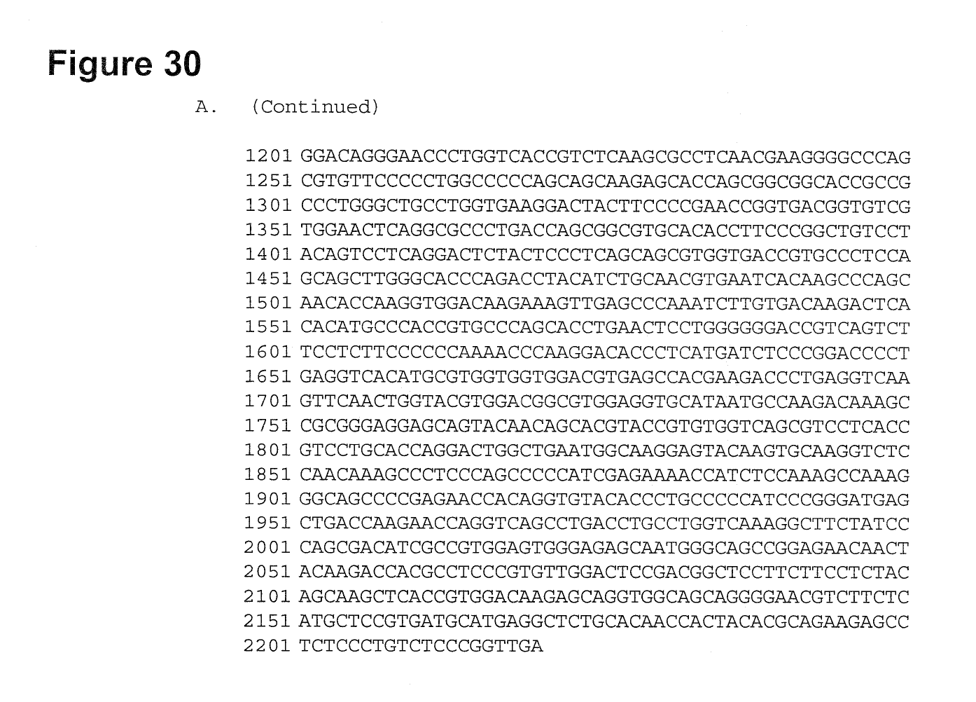

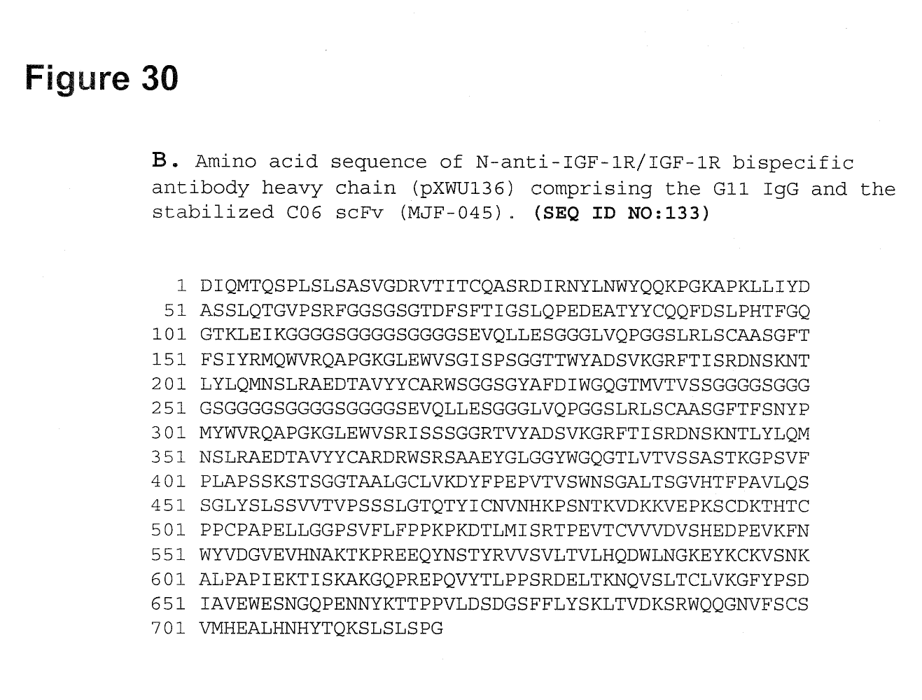

The resulting PCR fragment was digested with Mlu I and Nhe I restriction endonucleases and ligated to the Mlu I/Nhe I digested intermediate vector pXWU117. This resulted in a fusion product of the stabilized C06 scFv to the amino terminus of the G11 antibody VH domain through a 25 amino acid (Gly₄Ser)₅ (SEQ ID NO: 184) linker. The ligation mixture was used to transform E. coli strain TOP 10 competent cells (Invitrogen Corporation, Carlsbad, Calif.). E. coli colonies transformed to ampicillin drug resistance are screened for presence of inserts. DNA sequence analysis was used to confirm the correct sequence of the final construct pXWU136 encoding N-terminal anti-IGF-1R bispecific antibody comprising a stability-engineered C06 scFv and the G11 IgG. The G1 light chain (pXWU118) used is common among the anti-IGF-1R N- and C-bispecific antibodies and DNA and amino acid sequences are shown in FIGS. 29A and 29B, respectively. Heavy chain DNA and amino acid sequences (pXWU136) for N-terminal anti-IGF-1R bispecific antibody comprising the stabilized MJF045 scFv is shown in FIGS. 30A and 30B, respectively.

TABLE 16

Oligonucleotides for construction
of an N-terminal anti-IGF-1R bispecific
antibody with stabilized C06 scFvs.

| | |
|---|---|
| 136-F | 5′-GTTGCTACGCGTGTCCTGTCCGACATCCAGA |
| (SEQ ID NO:171) | TGACCCAGTC 3′ |
| 136-R1a | 5′-GAAGCCGCTAGCTGCGCAGGAGAGTCTCAGG |
| (SEQ ID NO:172) | GACCCCCCAGGCTGCACCAGGCCACCGCCGGACT |
| | CCAACAGCTGGACCTCGGAGCCACCTCCCCCGGA |
| | TCCACC-3′ |
| 136-R2 | 5-′ACCTCCCCCGGATCCACCGCCCCCTGAACCG |
| (SEQ ID NO:173) | CCCCCTCCAGAGCCCCCTCCACCGGACCCTCCAC |
| | CGCCGCTTGAGACGGTGACCATTG-3′ |

Restriction endonuclease sites are shown underlined.

ii. Construction of C-Terminal Anti-IGF-1R Bispecific Antibodies

The MJF045 C06 scFv DNA described in Example 4 supra was used to construct a C-terminal anti-IGF-1R bispecific antibody using methods similar to that described in U.S. patent application Ser. No. 11/725,970. A Ser(Gly₄Ser)₃ (SEQ ID NO: 138) linker was used to connect the stabilized C06 scFvs to the carboxyl terminus of G11 IgG heavy chain. First, an intermediate C-terminal anti-IGF-1R BsAb heavy chain vector was constructed by subcloning a PCR-generated DNA fragment encoding the G11 IgG sequence from the anti-IGF-1R G1 plasmid described in U.S. Patent Publication No. 20070243194. The PCR reaction consisted of the forward 5′-primer MB-04F (Table 17) consisting of sequences complementary to the IgG CH1 constant domain and included an Age I restriction endonuclease site for cloning, the reverse 3′-primer 106mR2 consisting of sequences complementary to the carboxyl terminus of the IgG heavy chain followed by sequences encoding a portion of the in-frame Ser(Gly₄Ser)₃ (SEQ ID NO: 138) linker containing an internal BamH I site followed by a Dra III site and a terminal Bgl II site (Table 17), and the G11 heavy chain as template. The resulting PCR product removed an existing stop codon at the 3′ end of the IgG heavy chain gene and added nucleotides coding for a portion of the Ser(Gly₄Ser)₃ (SEQ ID NO: 138) linker immediately followed by a BamH I restriction endonuclease site, a Dra III site, and terminated with a Bgl II site. The PCR fragment was digested with Age I and BglI restriction endonucleases and ligated into the Age I/BamH I digested modified pV90 vector. This resulted in an intermediate vector, pXWU106m2, that contained the synthetic heavy-chain leader sequence, the G11 IgG sequence, and sequences encoding a portion of the Ser(Gly₄Ser)₃ (SEQ ID NO: 138) linker containing an internal BamH I site followed by a Dra III site.

Next, DNA sequences from stabilized C06 scFv (MJF045) were amplified by PCR using the forward 5′ VL PCR primer 135-F (includes a BamHI restriction endonuclease site followed by sequences encoding the remaining portion of the Ser(Gly₄Ser)₃ (SEQ ID NO: 138) linker peptide and the amino terminus of C06 scFv VL) and the reverse 3′ VH PCR primer 135-R (includes the carboxyl terminus of C06 scFv VH and a stop codon followed by a DraIII site). The PCR product was gel isolated, digested with BamHI and DraIII restriction endonucleases and ligated to the BamHI/DraIII digested intermediate vector pXWU106m2. This resulted in a fusion product of the stabilized C06 scFvs to the carboxyl terminus of the G11 antibody CH3 domain through a 16 amino acid Ser(Gly₄Ser)₃ (SEQ ID NO: 138) linker. The ligation mixture was used to transform E. coli strain TOP 10 competent cells (Invitrogen Corporation, Carlsbad, Calif.). E. coli colonies transformed to ampicillin drug resistance are screened for presence of inserts. DNA sequence analysis was used to confirm the correct sequence of the final constructs pXWU135 encoding C-terminal anti-IGF-1R bispecific antibody comprising a stability-engineered C06 scFv and the G11 IgG.

TABLE 17

Oligonucleotides for construction of a
C-terminal anti-IGF-1R bispecific antibody
with stabilized C06 scFvs.

| | |
|---|---|
| 135-F | 5′-GGGGGTGGATCCGGTGGAGGGGGCTCCGGCG |
| (SEQ ID NO:174) | GTGGCGGGTCCGACATCCAGATGACCCAGTC3′ |
| 135-R | 5′-GGATCTCACTGGGTGTCAGCTTGAGACGGTG |
| (SEQ ID NO:175) | ACCATTG-3′ |
| MB-04F | 5′-CTTCCCCGAACCGGTGACGGTG 3′ |
| (SEQ ID NO:176) | |
| 106mR2 | 5′-GGGCAGAGATCTCACTGGGTGTCAGCCGGGA |
| (SEQ ID NO:177) | TCCACCCCCGCCGGATTTACCCGGAGACAGGGAG |
| | AGG-3′ |

Restriction endonuclease sites are shown underlined.

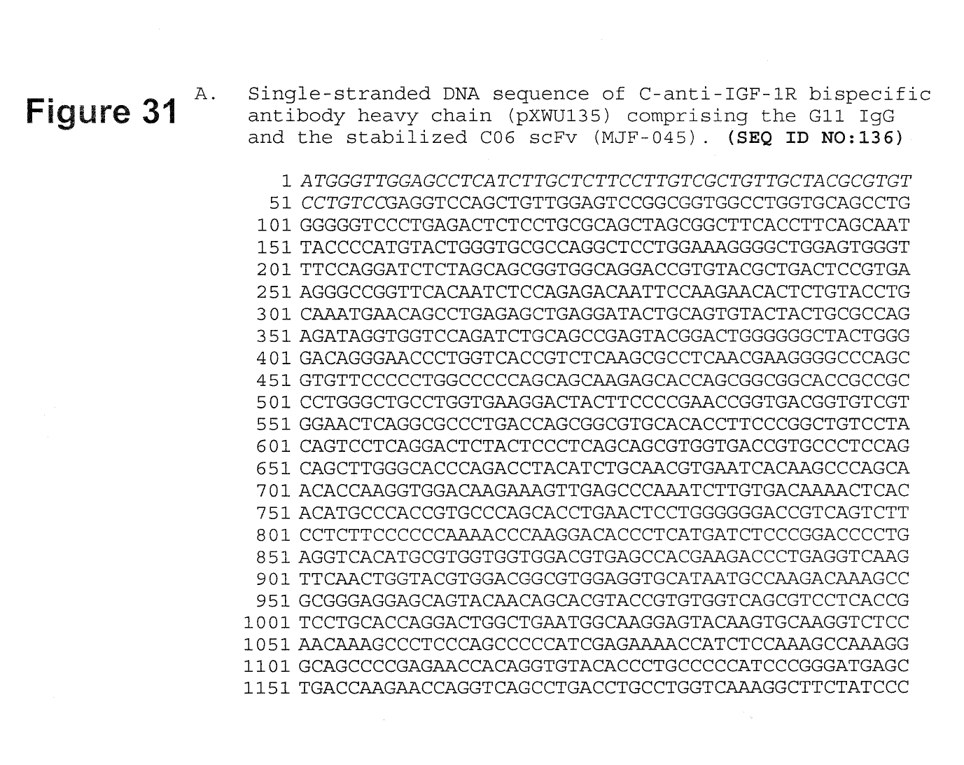

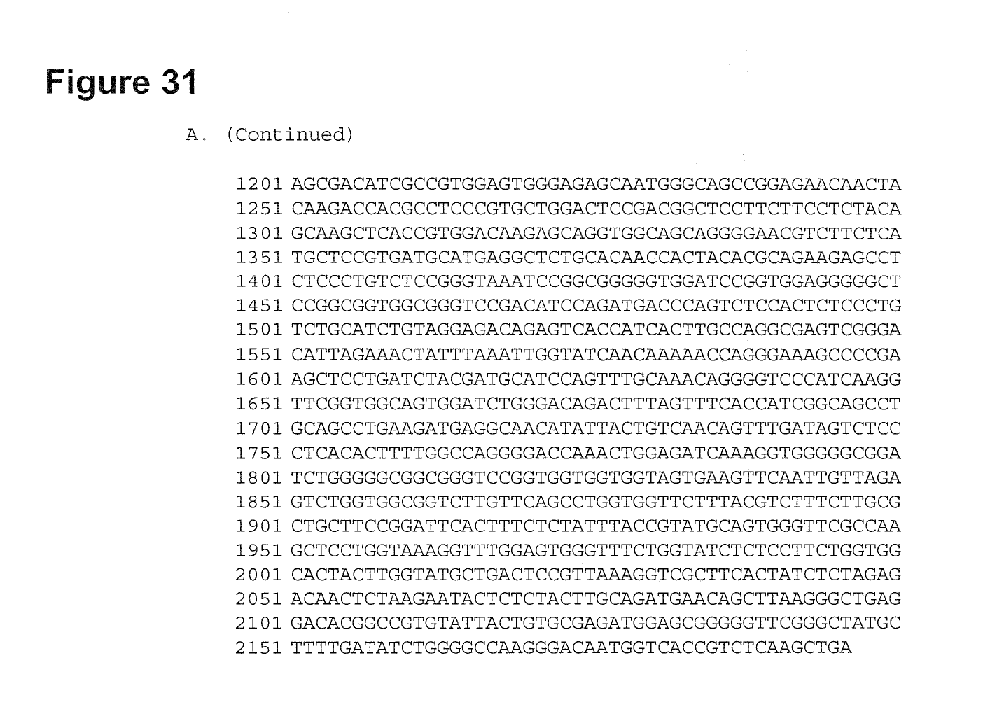

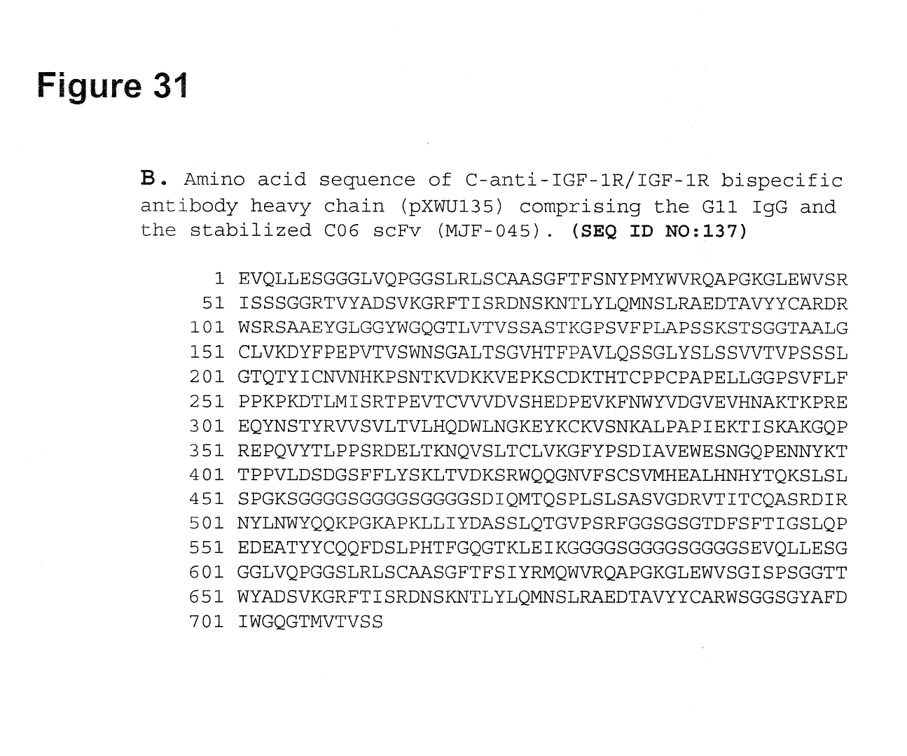

The G11 light chain (pXWU118) used is common among the anti-IGF-1R N- and C-bispecific antibodies and DNA and amino acid sequences are shown in FIGS. 29A and 29B, respectively. Heavy chain DNA and amino acid sequences (pXWU135) for C-terminal anti-IGF-1R bispecific antibody comprising the stabilized MJF045 scFv are shown in FIGS. 31A and 31B, respectively.

f. Construction of Alternative Anti-IGF-1R Bispecific Antibodies

Plasmids encoding stability-engineered C06 scFv molecules as listed in Table 15 can also be used for constructing stability-engineered N- and C-terminal anti-IGF-1R bispecific antibodies using methods similar to those described above. Table 18 lists plasmid names and the positions of stabilizing VH and VL mutations in the C06 scFv according to the Kabat numbering system. Positions of VH and VL mutations are shown within full-length heavy chain sequences for both C-anti-IGF-1R bispecific antibodies and N-anti-IGF-1R bispecific antibodies according to sequential numbering using FIGS. 30B (N-anti-IGF-1R bispecific antibody) and 31B (C-anti-IGF-1R bispecific antibody) as reference sequences.

TABLE 18
Stabilizing amino acid residues in the anti-IGF-1R C06 scFv used for constructing anti-
IGF-1R bispecific antibodies. Positions of stabilizing VH and VL mutations are shown according to
the Kabat numbering system (Kabat:). Positions of VH and VL mutations are also shown within full-
length heavy chain sequences for both C-anti-IGF-1R bispecific antibodies (C:) and N-anti-IGF-1R
bispecific antibodies (N:) according to sequential numbering using FIGS. 30B (N-anti-IGF-1R
bispecific antibody) and 31B (C-anti-IGF-1R bispecific antibody) as reference sequences,
respectively.

| | VL | | | | | | | | | | | | | VH | | | |
|---|---|---|---|---|---|---|---|---|---|---|---|---|---|---|---|---|---|
| | Kabat: | | | | | | | | | | | | | | | | |
| | 4 | 11 | 15 | 20 | 24 | 30 | 51 | 63 | 70 | 72 | 74 | 77 | 83 | 21 | 47 | 83 | 110 |
| | C: | | | | | | | | | | | | | | | | |
| | 493 | 500 | 504 | 509 | 513 | 519 | 540 | 552 | 559 | 561 | 563 | 566 | 572 | 632 | 658 | 698 | 728 |
| | N: | | | | | | | | | | | | | | | | |
| Plasmid | 23 | 30 | 34 | 39 | 43 | 49 | 70 | 82 | 89 | 91 | 93 | 96 | 102 | 162 | 188 | 228 | 258 |
| wt | M | L | V | T | Q | R | A | G | D | S | T | S | I | S | W | R | T |
| MJF-014 | L | | | | | | | | | | | | | | | | |
| MJF-015 | | | | | | | | | | | | | | | F | | |
| MJF-016 | | | | | | | | | E | | | | | | | | |
| MJF-017 | | | | | | | | | | | S | | | | | | |
| MJF-018 | | | N | | | | | | | | | | | | | | |
| MJF-019 | | | S | | | | | | | | | | | | | | |
| MJF-020 | | | E | | | | | | | | | | | | | | |
| MJF-021 | | | I | | | | | | | | | | | | | | |
| MJF-022 | | | R | | | | | | | | | | | | | | |
| MJF-023 | | G | N | | | | | | | | | | | | | | |
| MJF-024 | | | A | | | | | | | | | | | | | | |
| MJF-025 | | | P | | | | | | | | | | | | | | |
| MJF-026 | | | S | | | | | | | | | | | | | | V |
| MJF-027 | | | | | K | | | | | | | | | | | | |
| MJF-029 | | | | R | | Y | | | | | | | | | | | |
| MJF-030 | | | | | | T | | | | | | | | | | | |
| MJF-031 | | | | | | N | | | | | | | | | | | |
| MJF-032 | | | | R | | N | | | | | | | | | | | |
| MJF-033 | | | | R | | N | | | | | | | | E | | | |
| MJF-034 | | | | | | | G | | | | | | | | | | |
| MJF-035 | | | | | | | | | | N | | | | | | | |
| MJF-036 | | | | | | | | S | | Y | | | | | | | |
| MJF-037 | | | | | | | | | | Y | | | | | | | |
| MJF-038 | | | | | | | | S | | N | | | | | | | |
| MJF-039 | | | | R | | N | | S | | N | | | | | | | |
| MJF-040 | | | | | | | | | | | | | M | | | | |
| MJF-041 | | | | | | | | | | | | | V | | | | |
| MJF-042 | | | | | | | | | | | | G | G | | | | |
| MJF-043 | | | | | | | | | | | | | D | | | | |
| MJF-044 | | | | | | | | | | | | | Q | | | | |
| MJF-045 | | | | | | | | | | | | | E | | | | |
| MJF-046 | | | | | | | | | | | | | S | | | | |
| MJF-047 | | | | | | | | | | | | | G | | | | |
| MJF-048 | | | | | | | | | | | | | | | | K | |
| MJF-049 | | | | | | | | | | | | | | | | T | |
| MJF-050 | | | | | | | | | | | | | | | | | V |
| MJF-051 | | | | | | | | | | | | G | Q | | | | |

wt = wild-type sequence.

g. Construction of G11 scFv Molecules with Improved Thermal Stability

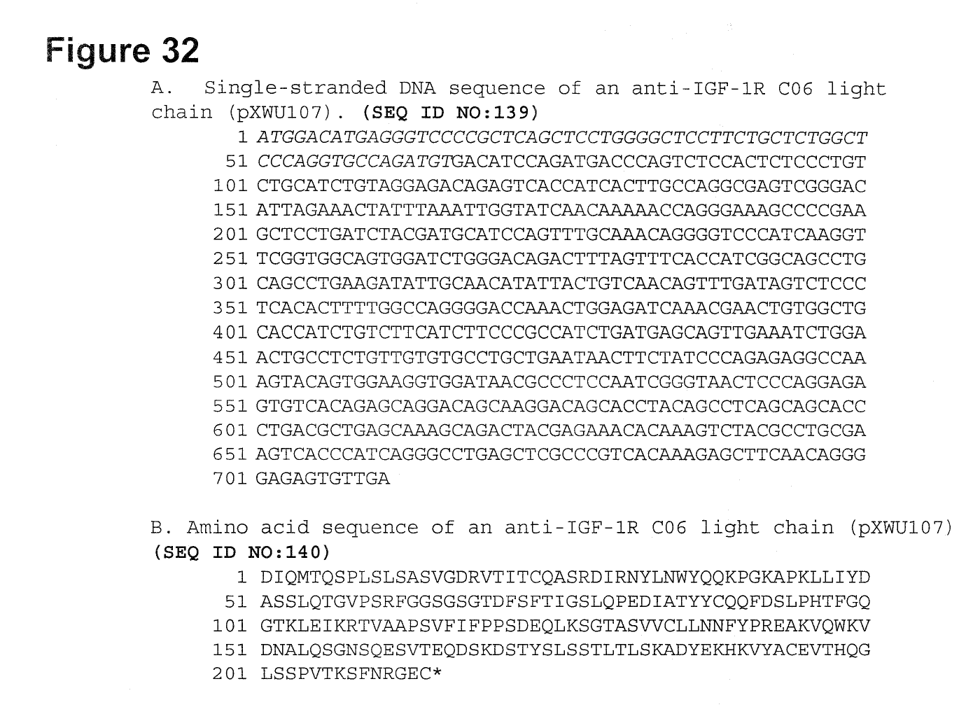

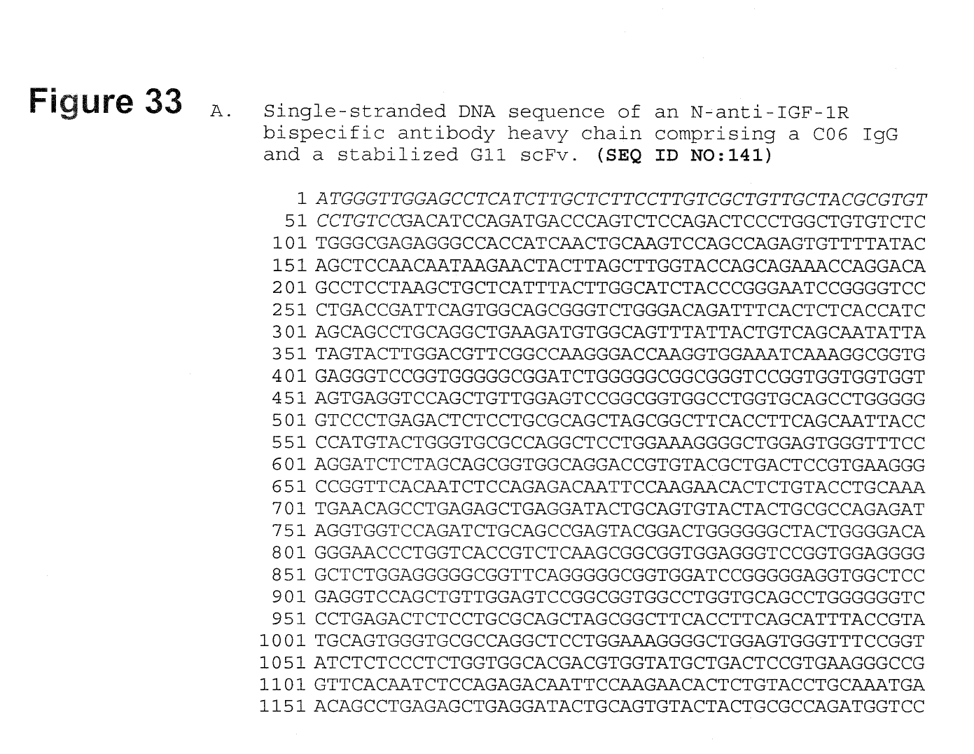

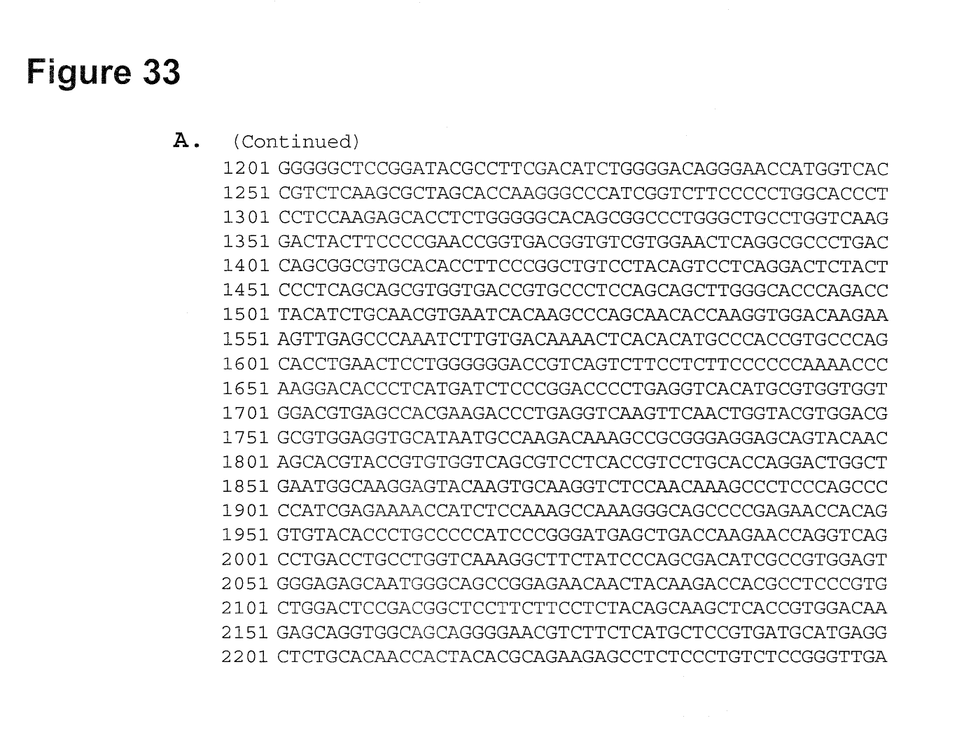

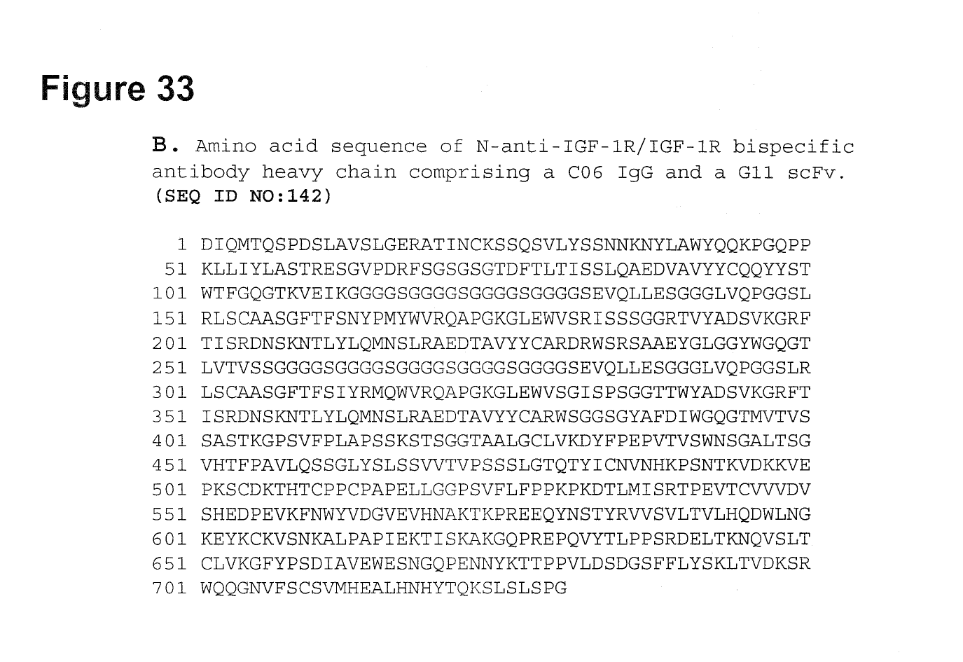

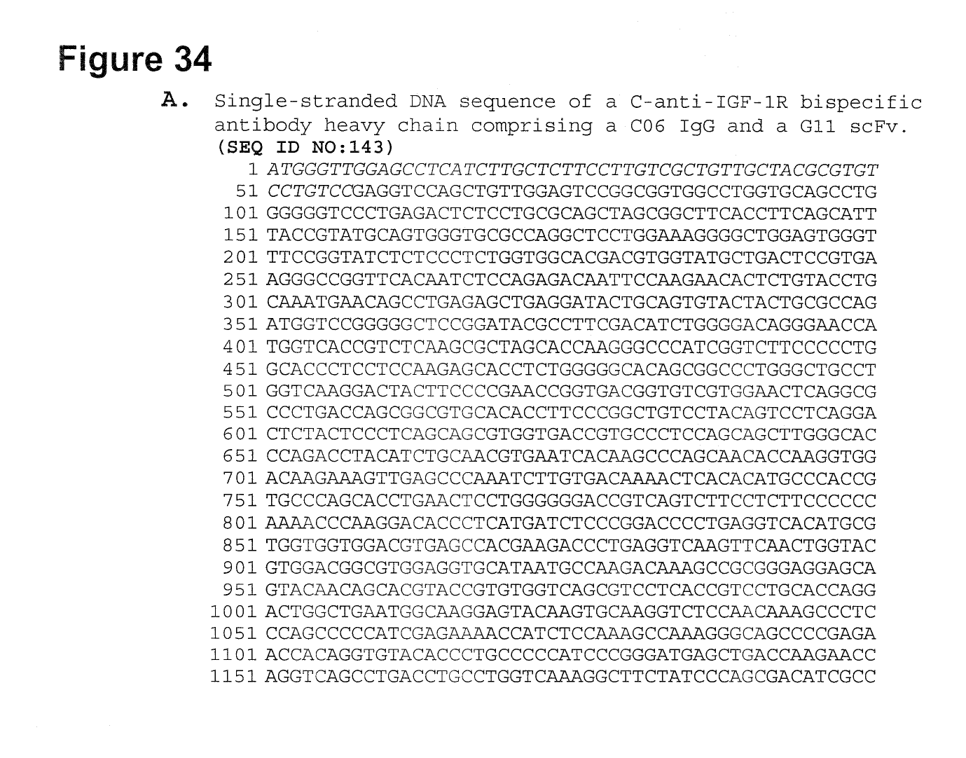

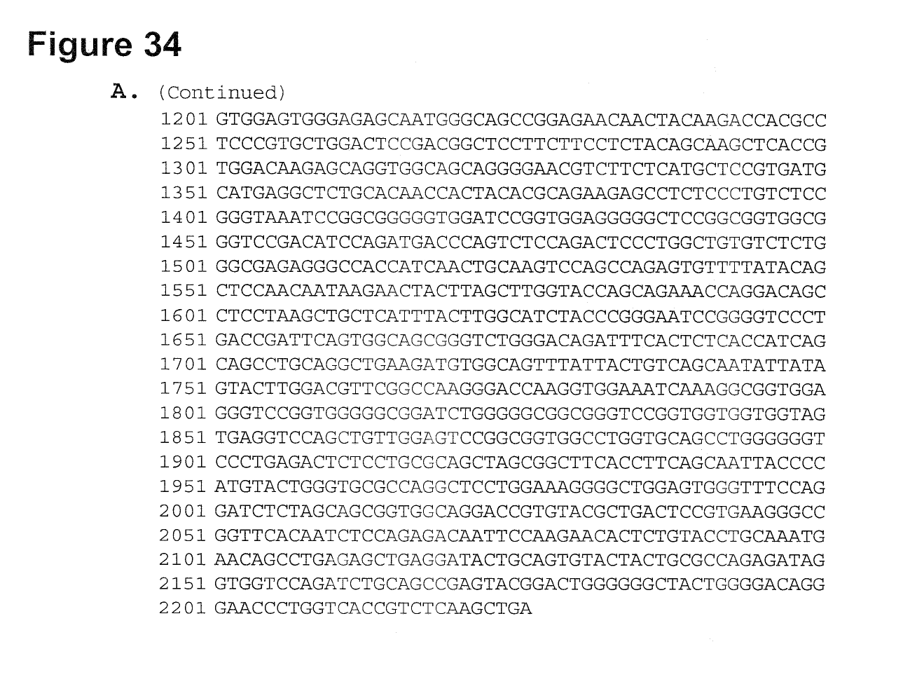

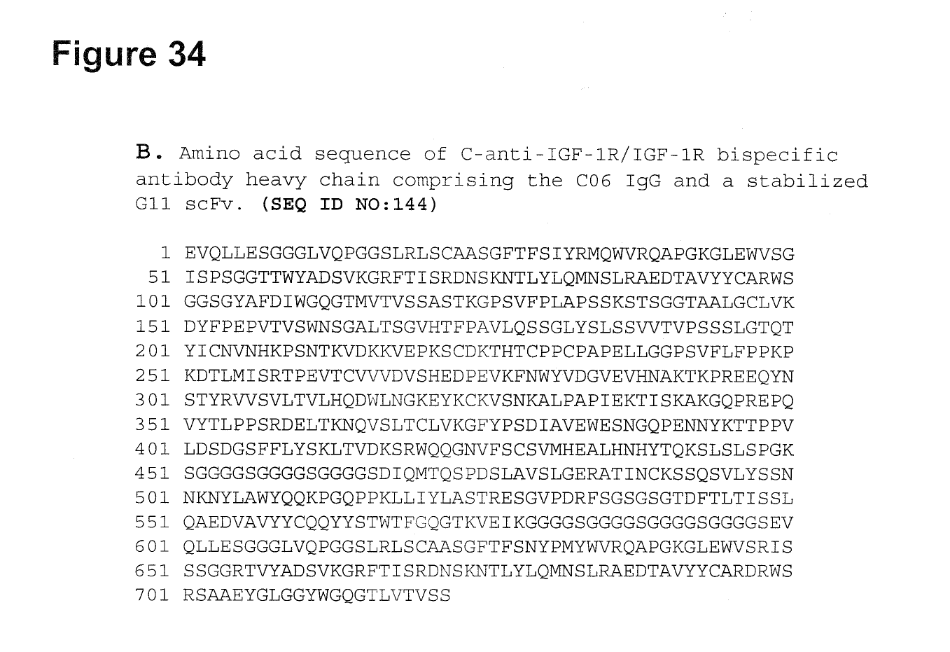

Anti-IGF-1R bispecific antibodies can be designed by stability-engineering a scFv prepared from the G11 IgG using methods similar to those described above and as described in U.S. patent application Ser. No. 11/725,970. A stability-engineered G11 scFv can be used to construct bispecific antibodies as both N-terminal and C-terminal scFv fusions as shown in FIG. 23 by fusing the scFv to either the amino of carboxyl terminus of a full-length C06 IgG heavy chain. The C06 light chain would be common among anti-IGF-1R N- and C-bispecific antibodies and DNA and amino acid sequences are shown in FIGS. 32A and 32B, respectively. Examples of heavy chain DNA and amino acid sequences coding for an anti-IGF-1R N-bispecific antibody constructed from G11 scFv and C06 IgG are shown in FIGS. 33A and 33B. Examples of heavy chain DNA and amino acid sequences coding for an anti-IGF-1R C-bispecific antibody constructed from G11 scFv and C06 IgG are shown in FIGS. 34A and 34B.

Individual variants and libraries were designed to contain desired amino acid replacements in the conventional G11 (VL/GS4/VH) scFv (pMJF060) using oligonucleotides listed in Table 19. In Table 19, each oligonucleotide name gives reference to desired amino acid substitutions at position(s) in VH or VL according to Kabat numbering system.

TABLE 19

Oligonucleotides and rationale
for construction of variant
G11 (VL/GS4/VH) scFvs.

| Oligo | | | SEQ ID |
|---|---|---|---|
| name | Rationale | Sequence | NO: |
| VH_E6Q | COMP | GAGGTCCAGCTGTTGCAGTCCGGC | 178 |
| | | GGTGGCCTG | |
| VH_S49AG | COVAR | GGGCTGGAGTGGGTTGSCAGGATC | 179 |
| | | TCTAGCAGC | |
| VL_L50nnk | COMP | AAGCTGCTCATTTACNNKGCATCT | 180 |
| | | ACCCGGGAATC | |
| VL_V83EF | COVAR, | CTGCAGGCTGAAGATKWKGCAGTT | 181 |
| | CONS | TATTACTGTC | |

“Rationale” refers to the design method employed. Said methods are described in detail in U.S. Pat. Application No. 11/725,970.
Abbreviations are: “COMP”- Computational Analysis, “COVAR”-Covariation Analysis, “CONS”-Consensus Scoring, “DEGEN”- degenerate codons,
Ambiguous bases are abbreviated as follows: W = A or T, V = A or C or G, Y = C or T, S = C or G, M = A or C, N = A or C or G or T, R = A or G, K = G or T, B = C or G or T (J Biol Chem. 261(1):13-7 (1986)).

Mutagenesis reactions were performed and individual transformed colonies were picked into deep-well 96 well dishes, processed, and screened according to the methods detailed in U.S. patent application Ser. No. 11/725,970. Transformants were grown overnight in expression media consisting of SB (Teknova, Half Moon Bay, Calif. Cat. # S0140) supplemented with 0.6% glycine, 0.6% Triton X100, 0.02% arabinose, and 50 μg/ml carbenicillin at either 30° C. or 32° C.

Each library was screened in duplicate using a thermal challenge assay with supernatant from one replicate subjected to treatment conditions and the second supernatant serving as untreated reference. After thermal challenge, the aggregated material was removed by centrifugation and assayed in the soluble IGF-1R Fc DELFIA as described in Example 8c.

Assay data was processed using Spotfire DecisionSite software (Spotfire, Somerville, Mass.) and expressed as the ratio of the DELFIA counts observed at challenge temperature to the reference temperature for each clone. Clones that reproducibly gave ratios greater than or equal to twice what was observed for the parental plasmid were considered hits. Plasmid DNAs from these positive clones were isolated by mini-prep (Wizard Plus, Promega, Madison, Wis.) and retransformed back into E. coli W3110 for confirmation secondary thermal challenge assays as well as for DNA sequence determination.

Primary and confirmatory results from these assays are shown in Table 20. Many of the stabilized scFv molecules of the invention resulted in improvements in binding activity (T₅₀=50° C.) as compared with the conventional G11 scFv (pMJF060). In particular, the T₅₀ values of variant G11 scFvs from library position V_{L}50 (L50G, L50E, L50M, L50N), library position V_{L}83 (V83E), library position V_{H}6 (E6Q), and library position V_{H}50 (S49A, S49G), exhibited increases in thermal stability (□T₅₀ values) ranging from +2° C. to +6° C. relative to the conventional G11 scFv.

TABLE 20
G11 VH and VL library positions, library composition, and
screening results.

| Position | Library | Hit Seq. Observed | □T₅₀ (° C.) |
|---|---|---|---|
| VH6 | Q, S | E6Q | +2 |
| VH495 | A, G | S49A, S49G | +4, +3 |
| VL50 | All amino | L50G, L50M, L50N | +3, +3, +6 |
| | acids | | |
| VL83 | E, F | V83E | +3 |

Plasmids consisting of two permutations of stabilizing mutations were then constructed to identify combinations that further enhanced thermal stability. Individual transformed colonies were picked into deep-well 96 well dishes and processed for screening as described above. G11 scFv proteins containing individual and combined stabilizing mutations were tested for: thermal stability in the T₅₀ thermal challenge assay. Table 21 summarizes the results of the T₅₀ thermal challenge assay. Individual and combined stabilizing mutations were identified that exhibited increases in thermal stability ranging from +2° C. to +10° C. relative to the conventional G11 scFv. The T₅₀ values of combined G11 mutations at positions V_{L}50 and V_{H}6 (V_{L} L50N:V_{H} E6Q) and positions V_{L}83 and V_{H}6 (V_{L} V83E:V_{H} E6Q) enhanced the thermal stability (T₅₀) of the scFv by +4-10° C. relative to the conventional G11 scFv.

TABLE 21
Amino acid substitutions of stabilized G11 scFv proteins,
T₅₀ results from thermal challenge assay.

| Name | Mutation | T₅₀ (° C.) |
|---|---|---|
| MJF-060 | N/A | 50 |
| MJF-084 | VL L50G | 53 |
| MJF-085 | VL L50N | 56 |
| MJF-086 | VL L50M | 53 |
| MJF-087 | VL V83E | 53 |
| MJF-091 | VL L50N, VH E6Q | 60 |
| MJF-092 | VL V83E, VH E6Q | 54 |
| MJF-097 | VH S49G | 53 |

N/A = not applicable

Table 22 lists plasmid names and the positions of stabilizing VH and VL mutations in the G11 scFv according to the Kabat numbering system. Positions of VH and VL mutations are shown within a full-length heavy chain sequence for both C-anti-IGF-1R bispecific antibodies and N-anti-IGF-1R bispecific antibodies according to sequential numbering using FIGS. 33B (N-anti-IGF-1R bispecific antibody) and 34B (C-anti-IGF-1R bispecific antibody) as references sequences, respectively.

TABLE 22
Stabilizing amino acid residues in the anti-IGF-1R G116 scFv
used for constructing anti-IGF-1R bispecific antibodies. Positions
of stabilizing VH and VL mutations are shown according to the
Kabat numbering system (Kabat:). Positions of VH and VL
mutations are also shown within full-length heavy chain sequences
for both C-anti-IGF-1R bispecific antibodies (C:) and N-anti-IGF-1R
bispecific antibodies (N:) according to sequential numbering using
FIGS. 33B (N-anti-IGF-1R bispecific antibody) and 34B
(C-anti-IGF-1R bispecific antibody) as references sequences.

| | |
|---|---|
| VL | VH |

Kabat:

| | | | |
|---|---|---|---|
| 50 | 83 | 6 | 49 |

C:

| | | | |
|---|---|---|---|
| 522 | 555 | 604 | 648 |

N:

| Plasmid | 56 | 90 | 138 | 181 |
|---|---|---|---|---|
| wt | L | V | E | S |
| MJF-082 | | | Q | |
| MJF-084 | G | | | |
| MJF-085 | N | | | |
| MJF-086 | M | | | |
| MJF-087 | | E | | |
| MJF-091 | N | | Q | |
| MJF-092 | | E | Q | |
| MJF-097 | | | | G |

wt = wild-type.

h. Stable Expression of Anti-IGF-1R Bispecific Antibodies in CHO Cells, Antibody Purification, and Characterization

Plasmid DNAs pXWU135, pXWU136 and pXWU118 were used to transform DHFR-deficient CHO DG44 cells for stable production of antibody protein. Transfected cells were grown in alpha minus MEM medium containing 2 mM glutamine supplemented with 10% dialyzed fetal bovine serum (Invitrogen Corporation) and enriched as a stable bulk culture pool using fluorescently labeled antibodies and reiterative fluorescent-activated cell sorting (FACS) (Brezinsky, et al. J Immunol Methods. 277(1-2):141-55 (2003)). FACS was also used to generate individual cell lines. Cell pools or cell lines were adapted to serum-free conditions and scaled for antibody production.

50 L of C-anti-IGF-1R bispecific antibody (pXWU135/pXWU118) supernatant from a 10 day bioreactor run was harvested and precleared by ultrafiltration. The bispecific antibody was captured from the supernatant using Protein A Sepharose FF (GE Healthcare). The bispecific antibody was eluted from the Protein A using 0.1 M glycine at pH 3.0, neutralized with Tris base, and dialyzed into PBS without further purification. Endotoxin levels were assayed by kinetic quantitative chromogenic LAL Analysis using the EndoSafe® PTC kit (Charles River Labs). Purity and percentage of monomer tetravalent antibody product was assessed by 4-20% Tris-glycine SDS-PAGE and analytical size-exclusion HPLC, respectively. The process yielded 289 mg C-anti-IGF-1R bispecific antibody at a concentration of 5.4 mg/ml, 96.7% purity, with a residual endotoxin concentration of 0.67 EU/mg protein.

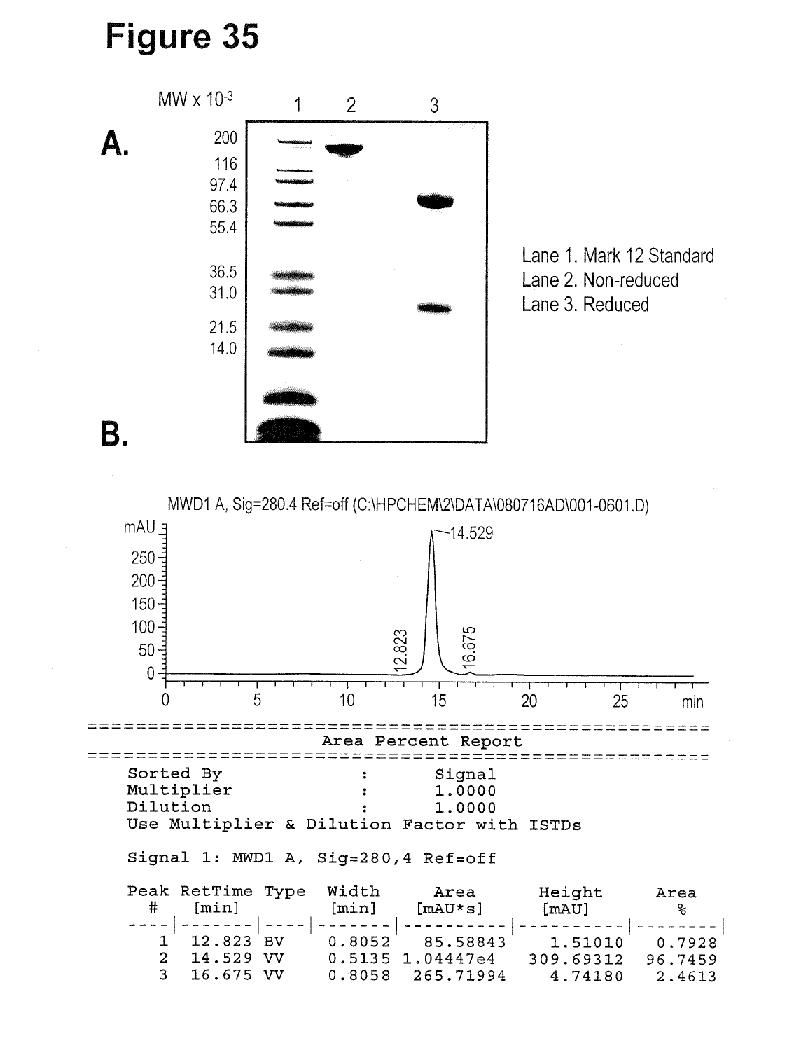

FIG. 35A shows an SDS-PAGE gel of purified stability-engineered C-anti-IGF-1R bispecific antibody (pXWU135/pXWU118). The reduced lane shows the expected sizes of the heavy and light chain proteins. Importantly, there is no significant level of degraded or unwanted lower molecular weight byproducts detected.

FIG. 35B shows an analytical SEC elution profile of purified stability-engineered C-anti-IGF-1R bispecific antibody (pXWU135/pXWU118). This analysis demonstrates that the stability-engineered C-anti-IGF-1R bispecific antibody is essentially >96.7% pure, monomeric, and free of higher order molecular weight species.

25 L of N-anti-IGF-1R bispecific antibody (pXWU136/pXWU118) supernatant from a 10 day bioreactor run was harvested and precleared by ultrafiltration. The N-anti-IGF-1R bispecific antibody was purified as described above for the C-anti-IGF-1R bispecific antibody. Purity and percentage of monomer tetravalent antibody product was assessed by 4-20% Tris-glycine SDS-PAGE and analytical size-exclusion HPLC, respectively. The process yielded 1401 mg N-anti-IGF-1R bispecific antibody at a concentration of 9.2 mg/ml, 97.3% purity, with a residual endotoxin concentration of 0.09 EU/mg protein.

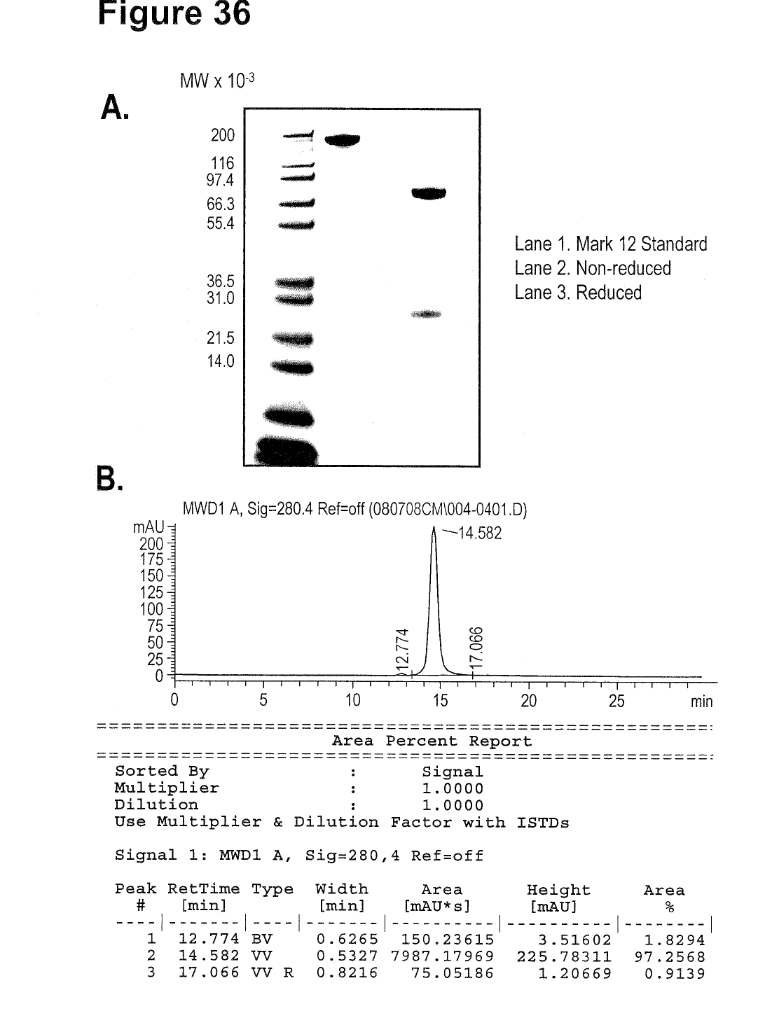

FIG. 36A shows an SDS-PAGE gel of purified stability-engineered N-anti-IGF-1R bispecific antibody (pXWU136/pXWU118). The reduced lane shows the expected sizes of the heavy and light chain proteins. Again, there is no significant level of degraded or unwanted lower molecular weight byproducts detected.

FIG. 36B shows an analytical SEC elution profile of purified stability-engineered N-anti-IGF-1R bispecific antibody (pXWU136/pXWU118). This analysis demonstrates that the stability-engineered N-anti-IGF-1R bispecific antibody is essentially 97.3% pure, monomeric, and free of higher order molecular weight species.

### Example 9

### Biochemical Characterization of Anti-IGF-1R Bispecifc Antibodies

a. Purification and Biochemical/Biophysical Characterization of the N- and C-Terminal Anti-IGF-1R BsAbs with a G11 Kappa Light Chain and G11 IgG1 Heavy Chain Backbone Fused at the N- or C-Terminus to the Stabilized C06 scFv.

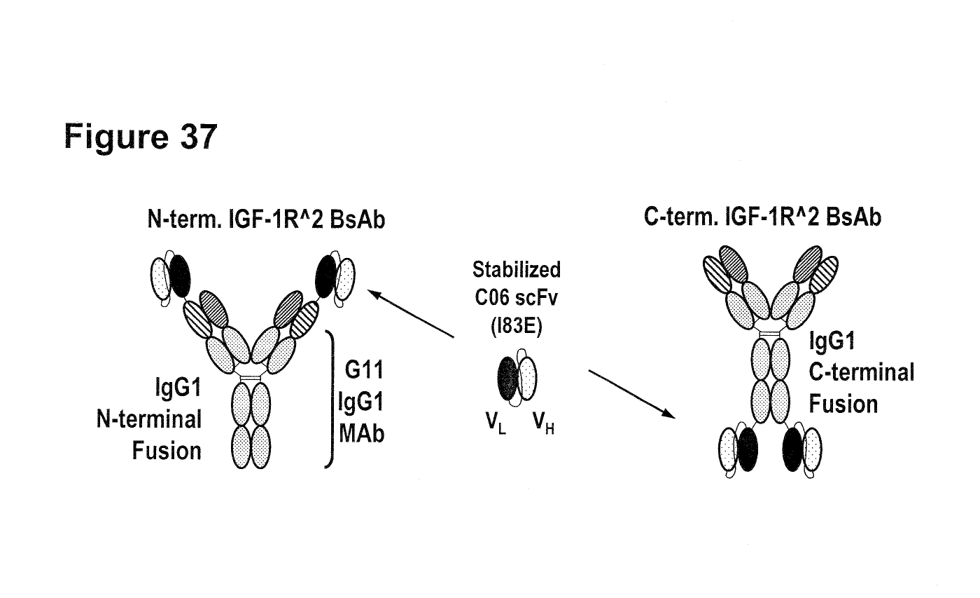

Bispecific IgG-like antibodies (“BsAbs”) have broadly met with product production and quality issues within the field of antibody engineering (Demarest, S.J., Glaser, S.M. (2008) Curr. Opin. Drug Discov. Devel. 5, In Press (September Issue)). Here, it is demonstrated that the use of a stabilized C06 scFv for the construction of N- and C-terminal G11 IgG1/C06 scFv IgG-like bispecific antibodies (denoted N- and C-term. IGF-1R bispecific antibodies, FIG. 37) results in recombinant protein with robust handling properties, high quality, and stability.

i. Methods

BsAb Expression: In-house mammalian expression vectors individually containing the BsAb heavy and light chains were transfected into DHFR-CHO DG44 as described in Example 8e above and in U.S. patent application Ser. No. 11/725,970. CHO cell lines producing the N- and C-terminal BsAbs were isolated as described previously (Brezinsky, S.C. G., Chiang, G. G., Szilvasi, A., Mohan, S., Shapiro, R. I., MacLean, A., Sisk, W., and Thill, G. (2003) J. Immunol. Methods 277, 141-155). The method for large-scale CHO cell cultures for the expression of both the N- and C-term. IGF-1R bispecific antibody proteins is described in Example 8e above and in U.S. patent application Ser. No. 11/725,970. The N- and C-terminal BsAbs were separated from CHO cell supernatants by capture onto MAbSelect protein A affinity resin preconditioned to neutral pH (GE Healthcare). Captured material was washed with 5 column volumes 0.5 M Tris-HCl, pH 8.5, 0.5 M NaCl, and protein was eluted with 0.1 M glycine, pH 3.0. Protein eluants were neutralized immediately using 1 M Tris-base and dialyzed against PBS. Protein concentrations were determined by UV-Vis using an extinction coefficient of 1.6 L cm g⁻¹. Endotoxin levels were determined using the Endosafe®-PTS LAL testing system (Charles River Labs). Plasmids for expressing the G11 IgG1 antibody were produced as a precursor to the production of the BsAbs. G11 IgG1 cell lines were selected and scaled for production as described for the BsAbs and the purification protocols were identical.

SDS-PAGE and Analytical Size Exclusion Chromatography (SEC): N- and C-term. IGF-1R bispecific antibodies were tested for purity using SDS-PAGE and analytical SEC chromatography. Each BsAb (5·g per well) was mixed with Novex® SDS loading buffer with and without ˜0.1 M β-mercaptoethanol for non-reducing and reducing conditions, respectively. Samples were heated ˜10 minutes at 95° C., applied to Novex® 4-20% Tris glycine gels using the XCell SureLock™ Mini Cell and Tris-glycine running buffer, and run according to the manufacturer's directions (Invitrogen).

Analytical SEC was performed on a BioSeptember 3000 300×7.8 mm (Phenomenex) SEC column equilibrated in 10 mM phosphate, 150 mM NaCl, 0.02% sodium Azide at pH 6.8 using an Agilent 1100 HPLC system. Between 30-100 μg of protein was applied to the column at 0.5 mL/min. Eluted protein was detected by UV absorption at 280 nm.

Circular Dichroism (CD) Spectroscopy: CD measurements were performed using a Jasco J-810 spectropolarimeter equipped with a thermoelectric peltier device for temperature control and an external water bath as a heat sink. Near and far UV scans were performed with 1 □M protein using 1 cm and 0.1 cm cuvettes, respectively. Spectral range was 197-260 nm and 250-320 nm for the near and far UV measurements, respectively. The scans were performed at 10° C. in the Continuous mode (100 nm/min) with a response time of 2 sec, data pitch of 0.1 nm and bandwidth of 2 nm and 1 nm for the near and far UV scans, respectively. All measurements were performed in the “High Sensitivity” mode. The baselines of all the protein spectra were corrected using background scans with PBS.

Differential Scanning Calorimetry (DSC): DSC scans were performed using an automated capillary DSC (capDSC, MicroCal, LLC). Protein and reference solutions were sampled automatically from 96-well plates using the robotic attachment. Prior to each protein scan, two buffer scans were performed to define the baseline for subtraction. All 96-well plates containing protein were stored within the instrument at 6° C. Concentrated G11 IgG1 protein (3.9 mg/mL) and the N- and C-term. IGF-1R bispecific antibodies (9.2 mg/mL and 5.4 mg/mL, respectively) were dialyzed against PBS. Subsequently the samples were diluted with PBS to 1.0 mg/mL G11 IgG1, and 1.3 mg/mL of both the BsAbs prior to the experiments to achieve an equimolar amount of protein in the calorimeter. Two background scans using PBS dialysate in both the sample and reference capillaries were performed prior to each protein scan using protein in the sample capillary and PBS dialysate in the reference capillary. Scans from 10-100° C. were performed at 2° C./min in the low feedback mode. Scans were analyzed using the Origin software supplied by the manufacturer. Subsequent to the subtraction of reference baseline scans, non-zero protein scan baselines were corrected using a third order polynomial.

ii. Results

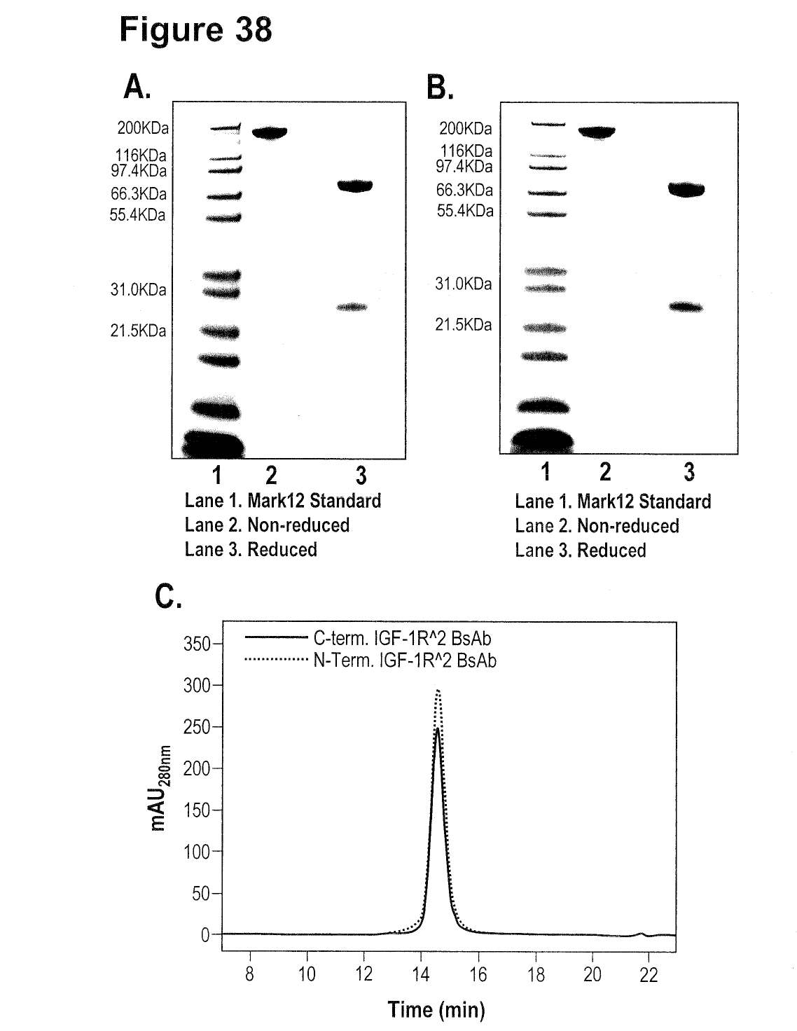

N- and C-terminal IGF-1R bispecific antibodies were produced in CHO cells using methods described in Example 8e above and in U.S. patent application Ser. No. 11/725,970. Two separate batches of each BsAb were produced, first from transfected stable bulk CHO cell pools and second from CHO cell lines selected to express the BsAbs. Material purified from the stable bulk pools yielded high quality N-terminal (20.4 mg from 5 L) and C-terminal (5.6 mg from 4 L) IGF-1R bispecific antibody material with >98% monomeric purity based on SDS-PAGE and analytical SEC chromatography (i.e. 200 kDa single band for non-reduced BsAb and 75 kDa and 25 kDa bands for the reduced and separated heavy and light chains, respectively, FIG. 38A,B). The BsAbs from the stable bulk cultures both eluted as single peaks with high purity off an analytical SEC column at a the expected time for an approximately 200 kDa protein based on protein molecular weight standards (FIG. 38C). The CHO cell lines selected to produce the N- and C-terminal IGF-1R bispecific antibodies were scaled up to 25 L and 50 L, respectively. Both production runs yielded material that was ˜97% pure with the correct molecular weight based on both non-reducing and reducing SDS PAGE analysis as well as analytical SEC. The C-terminal BsAb yielded 289 mg from a 50 L culture and the N-terminal BsAb yielded 1.4 g from a 25 L culture.

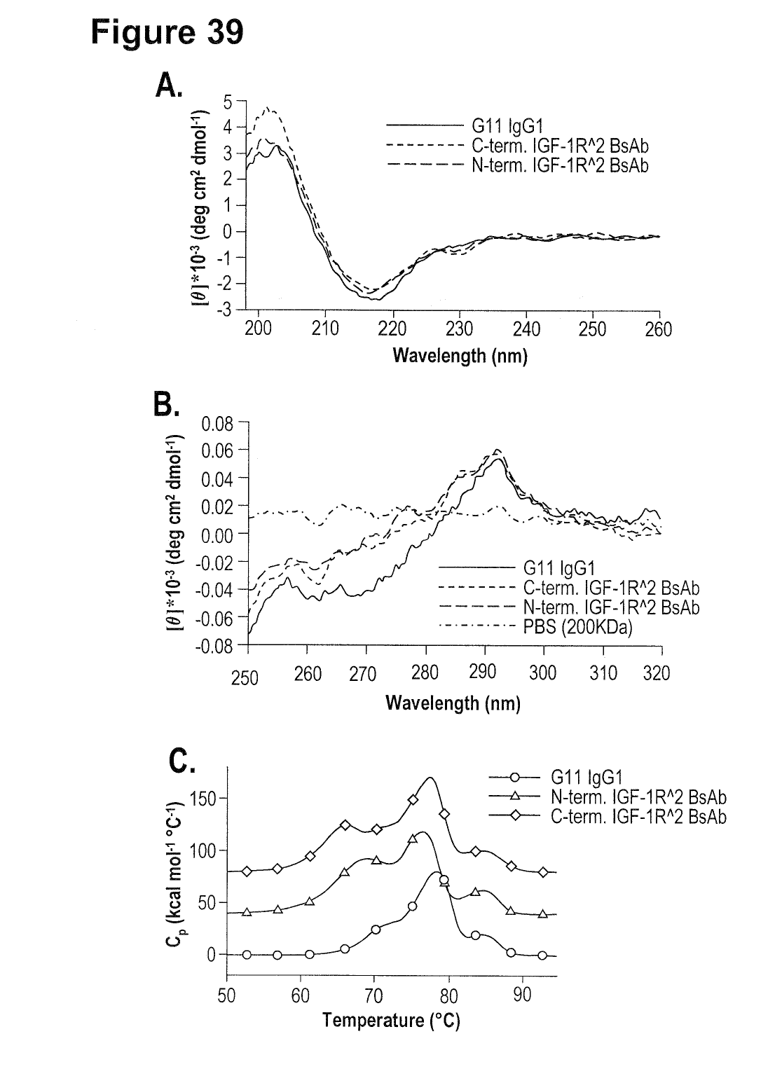

Circular dichroism (CD) measurements with the BsAbs demonstrate that the proteins are properly folded. The far UV CD spectra of the N- and C-terminal IGF-1R bispecific antibodies is very similar to what was observed for the G11 IgG1 protein that lacks the scFv (FIG. 39A). This is expected as the additional scFv simply adds additional Ig-fold protein domains that are of comparable □-sheet propensity to the domains of the parental G11 IgG1 molecule. Subtle differences in the spectra of the N- and C-terminal IGF-1R̂ at 217 nm likely reflect small differences in the overall average structure of the predominantly C-class Ig-folds of the G11 IgG1 and the additional V-class Ig-folds of the scFvs within the fusion proteins. While the signal-to-noise of the near UV CD spectra is weak, the spectra of the G11 IgG1 and the N- and C-terminal IGF-1R BsAbs are all very similar (FIG. 39B). Much of the NUV signal arises from the internal Trp residue that is canonical and invariant in both V-class and C-class Ig-folds and is buried in an identical position adjacent to the canonical Ig-fold internal disulfide bond. The NUV spectra of the G11 IgG1 and the N- and C-terminal IGF-1R bispecific antibodies are all similar as would be expected based on the properties of the protein domains within each molecule.

Differential scanning calorimetry (DSC) studies demonstrate that all domains within the N- and C-terminal IGF-1R bispecific antibodies are folded properly and have ideal (high temperature) thermal unfolding properties. Both the parental G11 IgG1 molecule and the N- and C-terminal IGF-1R bispecific antibodies are multi-domain proteins (FIG. 39C). Research has shown that IgG1 molecules often display 3 separate unfolding transitions, one each for the single C_{H}2 and C_{H}3 domains and a single large and apparently cooperative transition for the four domains of the Fab (V_{H}, V_{L}, C_{H}1, and C_{L}, Garber, E., Demarest, S.J. (2007) Biochem. Biophys. Res. Commun. 355, 751-757). The G11 IgG1 demonstrates the classical 3 transitions common for human IgG1s (FIG. 39C). Both the N- and C-terminal BsAbs also exhibit the 3 transitions for the C_{H}2, C_{H}3, and Fab domains plus one extra transition arising from the unfolding of the stabilized C06 scFv domains (FIG. 39C). The unfolding curves of the G11 IgG1 and the N- and C-terminal IGF-1R bispecific antibodies were fit to 3 and 4 transitions, respectively, and the data is provided in Table 23. The IgG transitions of the C-terminal IGF-1R bispecific antibody are nearly identical to what was observed for the G11 IgG1, suggesting very little interaction between the stabilized C06 scFv and the IgG1 portion of the fusion protein. Additionally, the stabilized C06 scFv demonstrated a single transition with a T_{M} of 66° C. and a large comparative enthalpy of unfolding (Table 23). The single unfolding transition measured for the scFv suggests the V_{H} and V_{L} domains either unfold at similar temperatures or unfold cooperatively. The high T_{M} values measured for the scFv suggest that thermal stability, in particular, of the scFv is unlikely to pose product quality issues. Data with the N-terminal IGF-1R bispecific antibody is similar to that observed for the C-terminal BsAb; however, the C06 scFv T_{M} is slightly higher while the G11 Fab T_{M} is slightly lower (Table 23). The data suggests there may be a very weak interaction between the scFv and the Fab regions of the fusion protein—although nothing that is likely to lead to product quality issues.

Purified N- and C-terminal IGF-1R̂2 proteins were monitored for the accumulation of aggregates in PBS at 2-8° C. over a 3 month period. Storage under these conditions is common for protein reagents. Many proteins—particularly those that are less stable or soluble—demonstrate the accumulation of soluble aggregates when kept in solution for any length of time. The data collected here demonstrate that the N- and C-terminal IGF-1R bispecific antibodies are highly stable and show virtually no sign of aggregation (within the limits of our detection) over the 3 month time course (Table 24).

TABLE 23
Thermodynamic unfolding parameters of the N- and C-term.
IGF-1R bispecific antibodies and the G11 IgG1 control protein
measured by DSC.

| | | | | C06 |
|---|---|---|---|---|
| | C_{H}2 T_{M} | Fab T_{M} | C_{H}3 T_{M} | scFv^{a} T_{M} |
| | (ΔH_{cal} | (ΔH_{cal} | (ΔH_{cal} | (ΔH_{cal} |
| Construct | kcal/mol) | kcal/mol) | kcal/mol) | kcal/mol) |
| G11 IgG1 | 71.6 (235) | 77.8 (415) | 85.0 (85) | — |
| C-term. IGF-1R BsAb | 71.0 (110) | 76.8 (555) | 85.1 (95) | 65.8 (360) |
| N-term. IGF-1R BsAb | 70.6 (110) | 76.1 (415) | 84.6 (115) | 67.3 (410) |

^{a}C06 scFv was stabilized using our BIIB platform design/screening technology - resulting in mutation at Ile 83 to Glu (I83E).

TABLE 24
Stability study of IGF-1R bispecific antibodies over 3 months at
2-8° C. in PBS as measured by analytical SEC. The N- and
C-term. IGF-1R bispecific antibodies were held at 4.5 mg/mL
and 1.7 mg/mL, respectively.

| Construct | % Monomer | % Aggregates |
|---|---|---|
| C-term. IGF-1R BsAb T = 0 | 100.0 | 0.0 |
| C-term. IGF-1R BsAb T = 1 week | 100.0 | 0.0 |
| C-term. IGF-1R BsAb T = 1 month | 99.2 | 0.8 |
| C-term. IGF-1R BsAb T = 3 months | 99.2 | 0.8 |
| N-term. IGF-1R BsAb T = 0 | 100.0 | 0.0 |
| N-term. IGF-1R BsAb T = 1 week | 99.7 | 0.3 |
| N-term. IGF-1R BsAb T = 1 month | 100.0 | 0.0 |
| N-term. IGF-1R BsAb T = 3 months | 99.6 | 0.4 |

b. IGF-1R Binding of (i) the C06 and G11 MAbs and (ii) the N- and C-Terminal IGF-1R Bispecific Antibodies Measured by Isothermal Titration Calorimetry (ITC)

The hypothesis that achieving enhanced IGF-1R inhibition by engaging multiple inhibitory epitopes on the receptor necessitates that each epitope can be engaged by an inhibitory anti-IGF-1R antibody without obstructing the other inhibitory epitope. Isothermal titration calorimetry (ITC) was used to measure IGF-1R binding of the inhibitory C06 and G1 MAbs that recognize different epitopes of the receptor. The experiments definitively demonstrate that the C06 and G11 MAbs can co-engage the receptor. Also, ITC experiments were performed to show that the C- and N-terminal BsAbs bind strongly to IGF-1R and saturate the receptor with the expected stoichiometry.

i. Methods

Antibodies. The C06 and G1 antibodies and the sIGF-1R(1-903) ectodomain protein were produced and purified as described in US Patent Application 2007/0243194.

Isothermal Titration Calorimetry (ITC). C06 MAb (100 □M), G11 MAb (67 □M), and sIGF-1R(1-903) (5 □M) were used for the antibody/receptor binding experiments. The reagents were co-dialyzed against PBS, pH 7.2. The protein solutions were adjusted to the concentrations listed above by dilution using PBS dialysate. Experiments with the BsAbs used 25 □M C-term. IGF-1R̂2, 30 μM N-term. IGF-1R̂2, and 2.5 □M sIGF-1R(1-903). The protein solutions were prepared as described above for the MAb binding experiments. All ITC experiments were performed on an ITC₂₀₀ microcalorimeter (MicroCal, LLC) at 25° C. For each reaction, the reaction cell was filled with hIGF-1R(1-903). To investigate MAb or BsAb binding to sIGF-1R(1-903), the syringe was filled with MAb or BsAb and titrated into the reaction cell using 15×1.5 □L injections for the C06 MAb, 18×2.0 μL injections for the G11 MAb, and 20×1.8 μL injections for both the N- and C-term. IGF-1R bispecific antibodies. A four minute equilibration period was used between all injections with an initial delay of 60 seconds. Numerical integration of the data was performed using the ITC data analysis software supplied by MicroCal (Origin). ΔH_{A}° (T) values were calculated based on the difference between the average heat liberated/absorbed during the binding phase of the injections and the average heat of dilution found once the receptor, IGF-1R, was saturated with MAb or BsAb. At 25° C., the binding affinity of both the C06 and G11 MAbs and the C-terminal IGF-1R bispecific antibody to IGF-1R was too high to be measured by ITC—as indicated by an absence of multiple transition points in the region of the titration where the MAbs and the C-term. BsAb saturate all receptor binding sites.

ii. Results

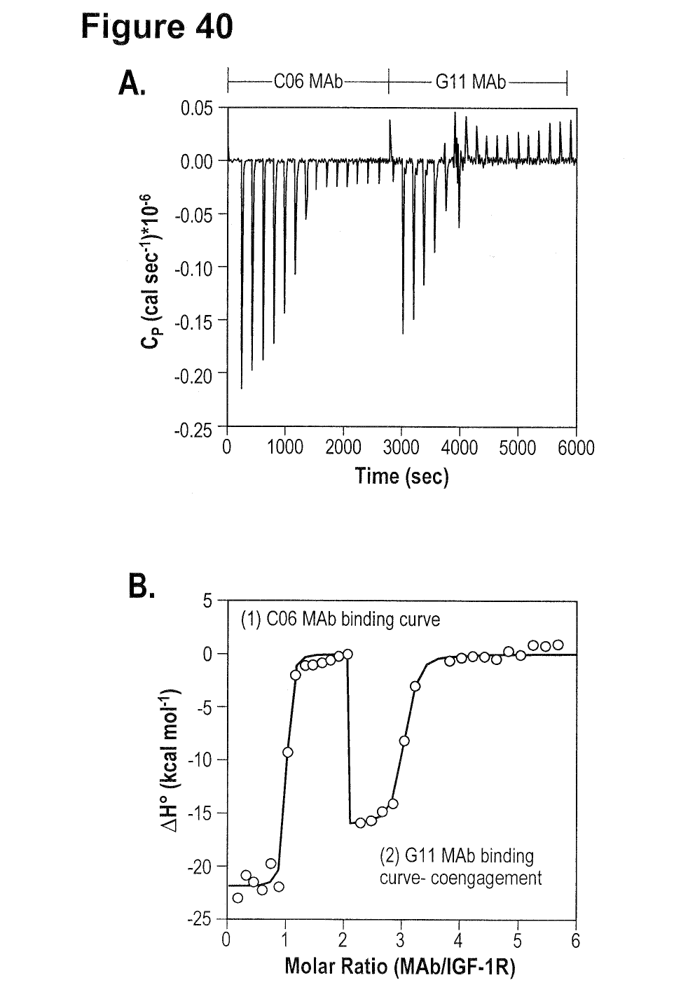

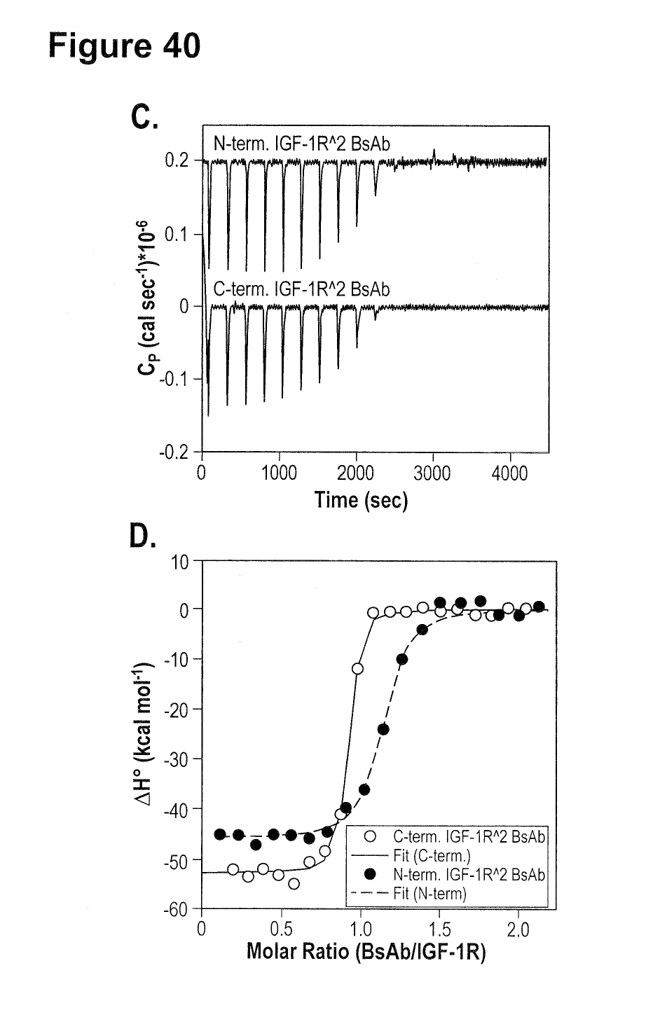

Isothermal titration calorimetry was used to demonstrate that the inhibitory anti-IG-1R MAbs, C06 and G11, could co-engage the receptor via their non-overlapping epitopes. First, C06 was titrated into a solution containing the recombinant soluble IGF-1R ectodomain, sIGF-1R(1-903) demonstrating a typical antibody binding curve (FIG. 40A,B). The sIGF-1R(1-903) protein has been shown to be dimeric by both SDS-PAGE and size exclusion chromatography/static light scattering (data not shown). Both the dimeric receptor and the bivalent MAbs contain two potential binding sites. The stoichiometry of binding was 1:1 as expected based on the equimolar number of binding sites between sIGF-1R(1-903) and the MAbs. Subsequently, the G11 MAb was titrated into solutions containing sIGF-1R(1-903) in the presence of a ˜2-fold excess of C06 MAb (FIG. 40A,B). The presence of the C06 MAb did not deter the G11 MAb from strongly engaging the receptor. The stoichiometry of the G11 MAb binding to sIGF-1R(1-903) was also 1:1. The experiments demonstrate the ability of the C06 and G11MAbs to co-engage IGF-1R.

Next, the C- and N-terminal IGF-1R bispecific antibodies were titrated into sIGF-1R(1-903) (FIG. 40C,D). Both BsAbs bound strongly and saturated the receptor at a 1:1 molar ratio. The C-term. IGF-1R bispecific antibody demonstrated an incredibly strong enthalpy of binding (−54 kcal/mol, Table 25), even greater than what would be expected based on the combination of the enthalpies measured for the C06 and G11 antibodies. Similar to the C06 and G11 MAbs, there were very few titration points in the transition region where saturation of the receptor occurs—suggestive of very tight binding (FIG. 40D). The N-term. IGF-1R bispecific antibody also showed a strong enthalpy (−43 kcal/mol, Table 25), albeit less than what was observed for the C-term. IGF-1R bispecific antibody. Additionally, there was more curvature (and more titration points) in the transition region for defining the affinity of the N-term. IGF-1R bispecific antibody to sIGF-1R(1-903). This enabled the measurement of an apparent equilibrium dissociation constant, K_{D}, of II nM (FIG. 40D, Table 25). These results suggest the binding affinity of the N-terminal IGF-1R bispecific antibody may be lower than that of the C-terminal IGF-1R bispecific antibody.

An interesting aspect of the ITC experiments is that they cannot distinguish whether the C06 scFv or the G11 Fv within the IgG1 portion of the BsAbs are capable of occluding each other for binding to the receptor. Even if they were 100% mutually exclusive, there would still be two active ends of the molecule enabling the 1:1 stoichiometry that is observed. However, the difference in the binding enthalpy of the two molecules combined with the weaker apparent affinity of the N-terminal IGF-1R bispecific antibody suggest the N-terminal BsAb may not be fully capable of binding the IGF-1R using all its binding sites. The binding moieties (i.e. the C06 scFv and the G11 antibody Fv) are identical within the two tetravalent formats; however, there relative position/spacing in the two formats is significantly different. It seems possible that steric hindrance between the scFv and the Fv may lead to subtle differences in the binding properties between the C- and N-term. IGF-1R bispecific antibodies.

TABLE 25
Thermodynamic parameters for the binding of IGF-1R to the
C06 and G11 Mabs ndividually and in combination as well as
the parameters for the binding of the C- and N-
terminal IGF-1R bispecific antibodies measured using ITC.

| | ΔH° | −TΔS | ΔG | K_{D} | |
|---|---|---|---|---|---|
| Construct | (kcal mol⁻¹) | (kcal mol⁻¹) | (kcal mol⁻¹) | (nM) | n |
| C06 MAb | −21.9 | 10.6^{a} | −11.3^{a} | 5^{a} | 0.95 |
| G11 MAb | −26.6 | 14.8^{a} | −11.2^{a} | 6^{a} | 0.90 |
| C06 MAb | −21.9 (C06) | —^{a} | —^{a} | —^{a} | 0.95 |
| then G11 MAb | | | | | |
| series titration | −16.2 (G11) | | | | 0.91 |
| C-term. | −53.9 | 42.3^{a} | −11.6^{a} | 3^{a} | 0.87 |
| IGF-1R BsAb^{b} | | | | | |
| N-term IGF-1R | −42.5 | 31.6 | −10.9 | 11 | 1.07 |
| BsAb^{b} | | | | | |

^{a}Affinity too high for accurate measurement.
^{b}Average of two separate measurements.

c. Determination of the Stoichiometry and Affinity of IGF-1R Binding to (i) the C06 and G11 Fabs, (ii) the C06 and G11 MAbs, and (iii) the N- and C-Terminal IGF-1R Bispecific Antibodies Using Solution Based Surface Plasmon Resonance

The C06 and G1 MAbs bind different and non-overlapping epitopes on IGF-1R; C06 on the surface of the first type-III fibronectin domain (FnIII-1) domain of the receptor and G1 on the surface of the cysteine rich region (CRR). Solution phase binding experiments were performed to investigate the ability of the N- and C-terminal BsAbs to bind IGF-1R using all 4 potential binding sites—2 sites to each epitope on IGF-1R.

i. Methods

Equilibrium Fab, MAb, and BsAb Binding to IGF-1R Using Surface Plasmon Resonance. All experiments were performed on a Biacore3000 instrument (Biacore). The C06 and G11 MAbs were separately immobilized to two different flow cell surfaces of a standard CM5 chip surface using standard amine chemistry protocols provided by the manufacturer. At these high immobilization levels of MAb, flowing low concentrations of sIGF-1R(1-903) (<50 nM) led to mass-transfer limited linear binding curves whose initial velocity of binding, Vᵢ (RU/s), depended linearly on the concentration of the sIGF-1R(1-903) solution flowed over the chip surface. Binding constants and stoichiometries of binding between sIGF-1R(1-903) (described in Example 9b) and the Fabs/MAbs/BsAbs could be determined by flowing mixtures of sIGF-1R(1-903) and antibody over the sensorchip surface containing C06 or G11. The C06 and G1 sensorchip surface measures the concentration of unbound sIGF-1R(1-903) in solutions containing sIGF-1R(1-903) and antibody. The equilibrium dissociation constant, K_{D}, and binding stoichiometry between the antibodies and sIGF-1R(1-903) is determined by the concentration of unbound sIGF-1R(1-903) using the equation below:

$$Vi = m \cdot [ {[ IGF - 1 R ]}_{T} - \frac{1}{2} \{ ( {n [ Ab ]}_{T} + {[ IGF - 1 R ]}_{T} + {K}_{D} ) - \sqrt{{( {n [ Ab ]}_{T} + {[ IGF - 1 R ]}_{T} + {K}_{D} )}^{2} - 4 {{n [ Ab ]}_{T} [ IGF - 1 R ]}_{T}} \} ]$$

where Vᵢ=initial rate of binding, m=slope of the sIGF-1R(1-903) concentration-dependent standard curve, [IGF-1R]_{f}=unbound IGF-1R concentration, [IGF-1R]ₜ=total IGF-1 concentration and [Ab]ₜ=total Fab/MAb/BsAb concentration (Day, E.S., Cachero, T.G., Qian, F., Sun, Y., Wen, D., Pelletier, M., Hsu, Y.-M., Whitty, A. (2005). “Selectivity of BAFF/BLyS and APRIL for Binding to the TNF Family Receptors BAFFR/BR3 and BCMA” Biochemistry 44: 1919-1931).

ii. Results

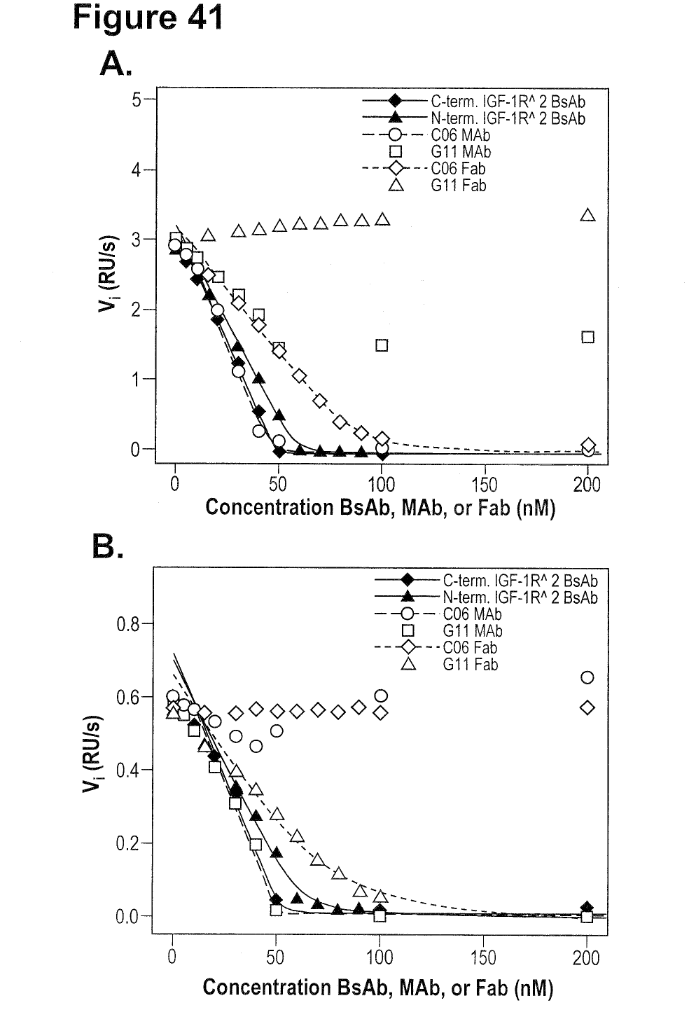

Equilibrium solution phase surface plasmon resonance experiments were performed to investigate the ability of all four binding sites of the tetravalent IGF-1R bispecific antibodies to bind to their appropriate epitopes. To test the binding to each epitope, C06 and G11 were immobilized to different sensor chip surfaces and used to probe whether the FnIII-1 and CRR epitopes on sIGF-1R(−1903), respectively, are unencumbered. The binding of the soluble IGF-1R ectodomain to the C06 surface and G11 surface was tested in the presence of varying amounts of N- and C-terminal IGF-1R bispecific antibodies (FIG. 41A,B). C06 and G11 MAbs and Fabs were used a controls for demonstrating a stoichiometry of binding of 1:1 and 2:1 antibody:receptor, respectively. It is important to note that the C06 MAb and Fab do not lead to inhibition of the G11 surface binding to sIGF-1R(1-903) and the G11 MAb and Fab do not lead to inhibition of the C06 surface binding to sIGF-1R(1-903)—demonstrating that each surface provides an exclusive measure of the accessibility of the epitope on sIGF-1R(1-903) to which it binds.

The results of the experiments clearly show that both the G11 Fab arms and both scFv arms of the C-terminal IGF-1R bispecific antibody are fully capable of binding their respective CRR and FnIII-1 epitopes with affinities identical to what was observed for the G11 and C06 MAbs (FIG. 41A,B). The inhibition curves of the C-term. IGF-1R bispecific antibody are identical to the inhibition curves observed for the C06 MAb versus the C06 surface and the G11 MAb versus the G11 surface. The binding stoichiometry of the MAbs and the C-terminal BsAb with respect to sIGF-1R(1-903) is 1:1 as expected based on the fact that the receptor is a homodimer (Table 26). The C06 and G11 Fabs demonstrate a 2:1 stoichiometry for binding to their respective epitopes, also as expected (Table 26). The Fabs also demonstrate lower apparent affinity—virtually identical to the affinities measured previously via kinetic biacore experiments (Table 26).

Unlike what was observed for the C-terminal IGF-1R bispecific antibody, results with the N-terminal IGF-1R bispecific antibody suggest that binding by the C06 scFvs and the G11 Fvs of the IgG1 portion of the molecule is not mutually exclusive. The N-terminal BsAb saturates the receptor (at both epitopes) with an apparent stoichiometry of 1.5:1—halfway between the values observed for the MAbs and the Fabs (FIG. 41A,B; Table 26).

In conclusion, the equilibrium solution-phase surface plasmon resonance experiments clearly demonstrate different binding capabilities between the N- and C-terminal BsAbs. While the C-terminal IGF-1R bispecific antibody behaves ideally with all four binding sites independently capable of engaging receptor without being encumbered by binding at the other binding sites of the BsAb. The N-terminal BsAb, alternately, appears to be unable to simultaneously engage all four of its binding moieties to IGF-1R in solution. One possibility is that the close proximity of the C06 scFv and G11 Fv in the N-terminal format leads to steric hindrance.

TABLE 26
Kinetic and equilibrium (solution phase) affinity measurements of the IGF-1R
bispecific antibodies along with control C06 and G11 MAbs and Fabs.

| | | | | | n (#C06 | | n (#G11 |
|---|---|---|---|---|---|---|---|
| | | | | K_{D} (nM | specific | K_{D} (nM | specific |
| | kₐ^{a} | k_{d}^{a} | K_{D}^{a} | vs. C06 | binding | vs. G11 | binding |
| Construct | (M⁻¹ s⁻¹) | (s⁻¹) | (nM) | surface) | sites) | surface) | sites) |
| C06 Fab | 8.8e5 | 1.1e−3 | 1.3 | 0.6 ± 0.4 | 2.0 ± 0.3 | — | — |
| G11 Fab | 3.0e5 | 1.2e−3 | 4.0 | — | — | 4.0 ± 2.0 | 1.7 ± 0.3 |
| C06 MAb | 1.9e6^{b} | 2.4e−4^{b} | 0.13^{b} | 0.1 ± 0.3 | 1.0 ± 0.1 | — | — |
| G11 MAb | 1.1e6^{b} | 1.1e−4^{b} | 0.11^{b} | — | — | 0.1 ± 0.1 | 1.1 ± 0.1 |
| CtermBsAb | _{—}^{b} | _{—}^{b} | _{—}^{b} | 0.03 ± 0.1 | 1.1 ± 0.1 | 0.1 ± 0.5 | 1.1 ± 0.1 |
| NtermBsAb | _{—}^{b} | _{—}^{b} | _{—}^{b} | 0.2 ± 0.2 | 1.5 ± 0.1 | 0.7 ± 0.7 | 1.5 ± 0.3 |

^{a}Described in US PCT 2007/0243194: Anti-IGF-1R antibodies and uses thereof.
^{b}Complex/multiphasic binding kinetics.

d. Determination of the Stoichiometry and Affinity of IGF-1R Binding to (i) the C06 and G11 Fabs, (ii) the C06 and G11 MAbs, and (iii) the N- and C-Terminal IGF-1R Bispecific Antibodies Solution Based Surface Plasmon Resonance

As described in Example 4 supra, combining multiple inhibitory anti-IGF-1R antibodies that recognize non-overlapping epitopes can lead to enhanced ligand blocking over what is achieved using single monoclonal antibodies. To combine the activities of two inhibitory anti-IGF-1R antibodies into a single protein construct, we generated tetravalent bispecific antibodies (BsAbs) that combine the competitive ligand blocking behavior of the G11 antibody with the allosteric blocking activity of the C06 antibody. The BsAbs consist of a stabilized scFv from the C06 antibody recombinantly fused to the N- or C-terminus of the G11 antibody in the IgG1 format (FIG. 37). As described in US. Patent Application 2008/0050370: “Stabilized polypeptide compositions,” stabilization of the scFv is an enabling step for producing high quality tetravalent IgG-like bispecific or multivalent antibodies. The data in this example shows that the N- and C-terminal IGF-1R bispecific antibodies exhibit enhanced ligand blockade over the single monoclonal antibodies.

i. Methods

Ligand Blocking Properties. The ability of the MAbs and BsAbs to block IGF-1 and IGF-2 was determined using the IGF-1 and IGF-2 blocking ELISA described in Example 4. IGF-1 and IGF-2 concentrations in the assay were 320 nM and 640 nM, respectively. To investigate the dependence of ligand concentration on the blocking ability of the C06 and G11 MAbs as well as the N- and C-terminal IGF-1R bispecific antibodies, the assays were performed at different ligand concentrations. Individual blocking ELISAs were performed using 20 nM, 80 nM, 320 nM, or 1300 nM IGF-1 or 40 nM, 160 nM, 640 nM, or 2600 nM IGF-2. IGF-2 concentrations were higher because IGF-2 has a slightly lower affinity for the receptor. To stay within the linear range of the ELISA curve, we needed to perform the assay at higher IGF-2 concentrations than the IGF-1 blocking ELISA to achieve comparable results.

ii. Results

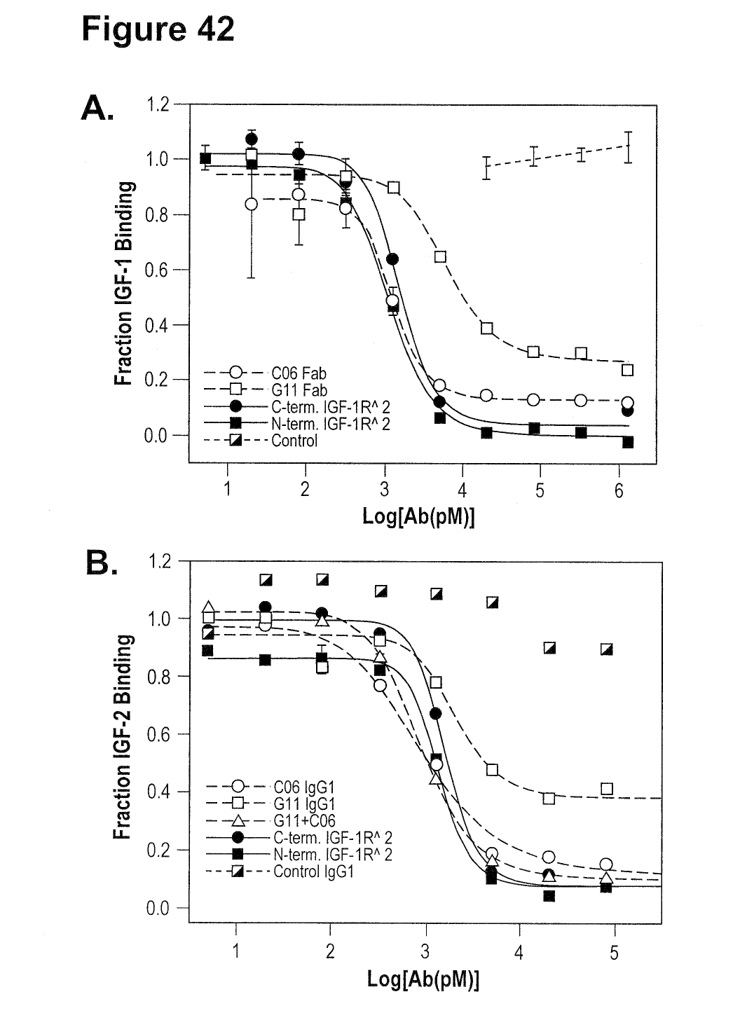

The C06 and G11 antibodies and the N- and C-terminal IGF-1R bispecific antibodies were tested side-by-side in the IGF-1 and IGF-2 blocking ELISAs (FIG. 42A-B) using 320 nM IGF-1 and 640 nM IGF-2. Both the N- and C-terminal BsAbs had ˜1 nM IC₅₀ values comparable to the higher affinity antibody, C06. Both the C06 and G11 antibodies could not completely abrogate IGF-1 or IGF-2 binding to IGF-1R at antibody concentrations <100 nM (FIG. 42A-B). Both the N- and C-term. IGF-1R bispecific antibodies were able to completely eliminate IGF-1 and IGF-2 binding to the receptor at low concentrations (<10 nM, FIG. 42A-B).

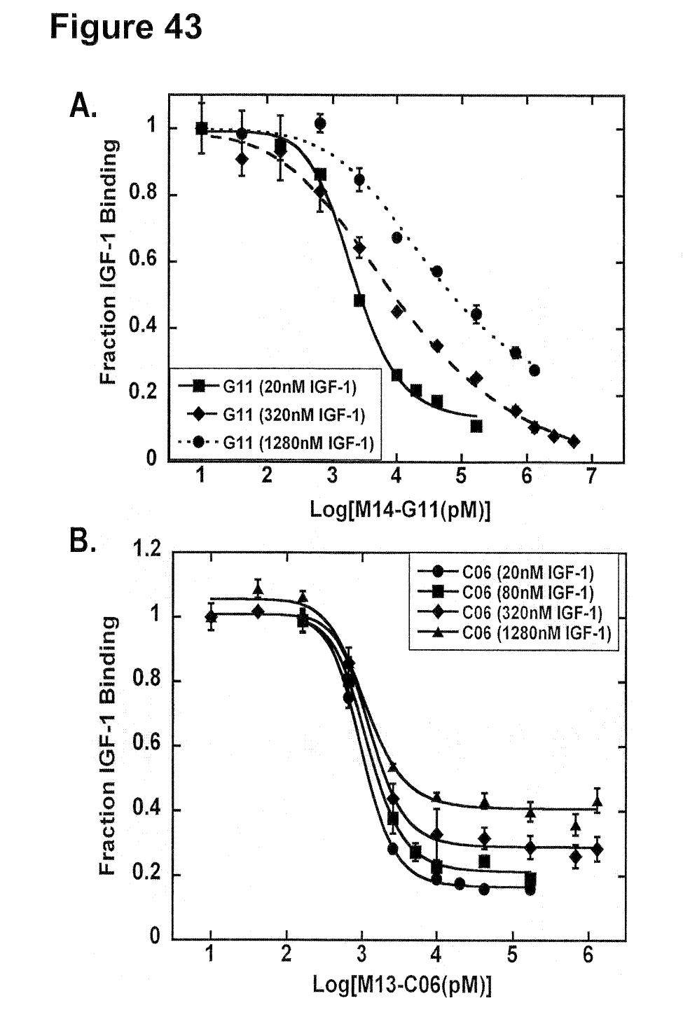

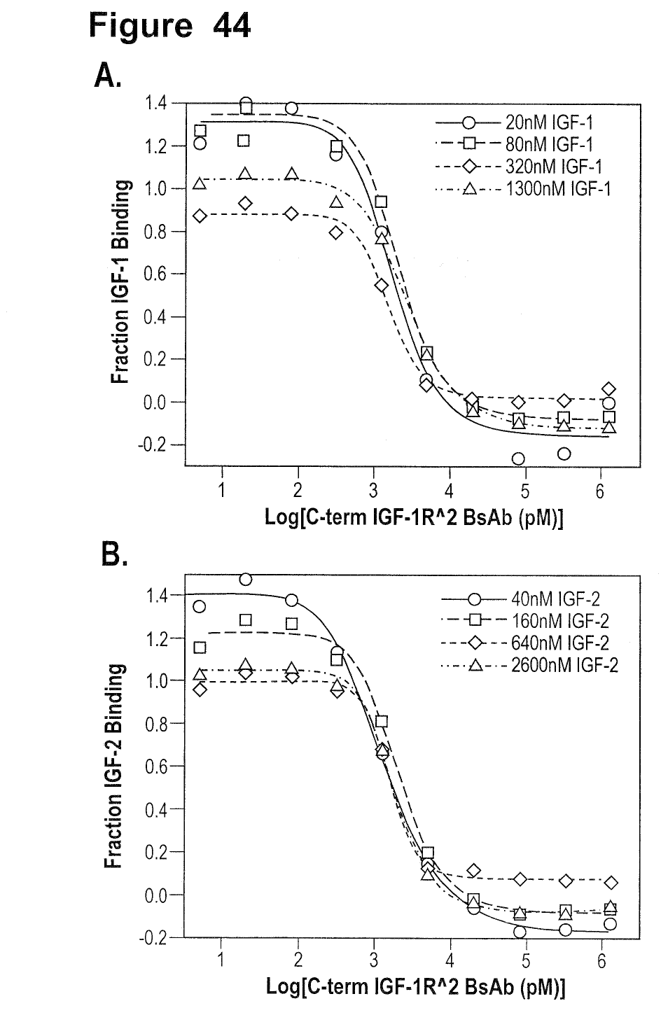

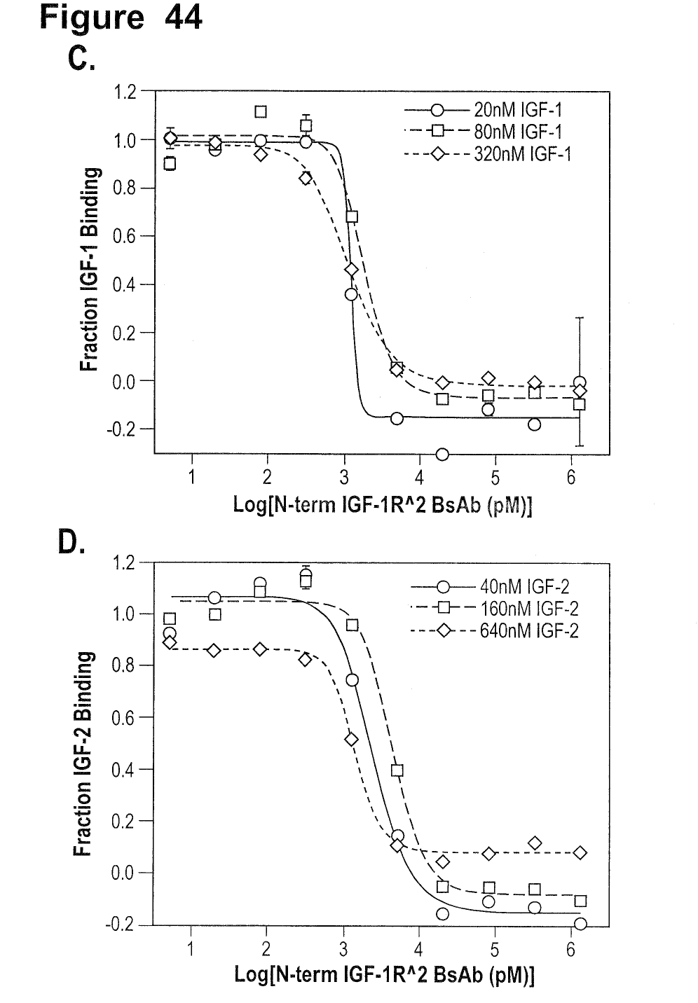

Upon increasing the ligand concentration used in the assay, the human antibody C06 loses some of its IGF-1 blocking activity (i.e., the percentage of IGF-1 capable of binding IGF-1R when the receptor is saturated with C06 goes up as ligand concentration goes up, FIG. 43A). This is a consequence of C06 being a purely allosteric inhibitor. The G11 antibody responds differently to increases in ligand concentration. As a competitive inhibitor, the human antibody G11 has to directly compete with ligand for binding the receptor. As ligand concentrations increase, the apparent potency of the G11 antibody decreases (FIG. 43B). By combining the ability to bind the receptor in the presence of ligand at the allosteric site and inhibit using both the allosteric and competitive mechanisms, the BsAbs are capable of completely abrogating both IGF-1 and IGF-2 from binding to the receptor—regardless of the ligand concentrations (FIG. 44A-D). Additionally, the IC₅₀ values measured for both the N- and C-term. IGF-1R̂2 antibodies are independent of the ligand concentrations used in the assay and equivalent to the IC₅₀ values observed for the C06 antibody in the assays:

C-terminal IGF-1R BsAb: IC₅₀ IGF-1 blocking=2.0±0.3

C-terminal IGF-1R BsAb: IC₅₀ IGF-2 blocking=1.7±0.2

N-terminal IGF-1R BsAb: IC₅₀ IGF-1 blocking=1.4±0.2

N-terminal IGF-1R BsAb: IC₅₀ IGF-2 blocking=2.7±0.9.

These results demonstrate that through their ability to recognize both the C06 and G11 inhibitory epitopes, both the N- and C-terminal IGF-1R bispecific antibodies demonstrate enhanced ligand blockade at all ligand concentrations over the standard human antibodies, C06 and G11.

e. Crosslinking of IGF-1R by the C06 and G11 MAbs and the N- and C-Terminal IGF-1R Bispecific Antibodies

One potentially discriminating feature of different inhibitory antibodies against IGF-1R is their ability to crosslink the receptor on the cell surface. Many receptor tyrosine kinase family members signal via ligand-mediated homo- or heterodimerization. IGF-1R (and the insulin receptor) does not signal via this mechanism, however. IGF-1R is a constitutive homodimer whose signaling depends on conformational changes induced by ligand binding that are not associated with dimerization.

Some antibodies to IGF-1R have demonstrated the ability to downregulate the receptor via internalization and degredation. Crosslinking is often associated with the efficiency of cell surface protein internalization (e.g. FcεRI is internalized by cross-linking). In such a way, crosslinking may have a significant impact on the activity of antibodies against cell surface proteins and in this case, IGF-1R. Additionally, the extent of antibody-mediated crosslinking of IGF-1R on the surface of a cell may influence the binding of the Fc-region of the antibodies to FcγR receptors or complement—influencing the activity of the antibodies with the host immune system.

Here we demonstrate that the C06 and G11 MAbs lead to different sized IGF-1R/MAb immune complexes in solution when incubated with a soluble version the IGF-1R ectodomain. We also investigate the resulting complexes formed by the introduction of both the C06 and G11 MAbs simultaneously to a solution with IGF-1R. Finally, we investigate the properties of the immune complexes formed by the N- and C-terminal IGF-1R bispecific antibodies.

i. Methods

Analytical size exclusion chromatography (SEC) with in-line static light scattering. SEC samples were prepared with the (i) C06 MAb, (ii) G11 MAb, and (iii) sIGF-1R(1-903); with the binary mixture of (iv) the C06 MAb and sIGF-1R(1-903) or (v) the G11 MAb and sIGF-1R(1-903); or with the ternary mixture of (vi) the C06 MAb, G11 MAb, and sIGF-1R(1-903). All samples contained 25 μg of each protein diluted to a final volume of 50 μL in PBS, pH 7.2, before injection onto the SEC column onto a TSKgel G3000SW XL, 5 mm, 250 Å Analytical SEC column (Tosoh Biosciences) equilibrated in 10 mM phosphate, 150 mM NaCl, 0.02% sodium Azide at pH 6.8 using an Agilent 1100 HPLC system. Light scattering data for material eluting from the SEC column were collected using a miniDAWN static light scattering detector coupled to an in-line refractive index meter (Wyatt Technologies). Light scattering data were analyzed using the ASTRA V software provided by the manufacturer.

Additional SEC samples were prepared using the (i) C-term. IGF-1R bispecific antibody (BsAb), (ii) the N-term. IGF-1R BsAb, and (iii) sIGF-1R(1-903); or complexes with the (iv) C-term. IGF-1R BsAb and sIGF-1R(1-903) or (v) N-term. IGF-1R BsAb and sIGF-1R(1-903). The amounts of sIGF-1R(1-903) and BsAb used in the experiments were 30 μg and 45 μg, respectively. All samples were diluted to a final volume of 50 μL in PBS, pH 7.2 before SEC/static light scattering analysis.

ii. Results

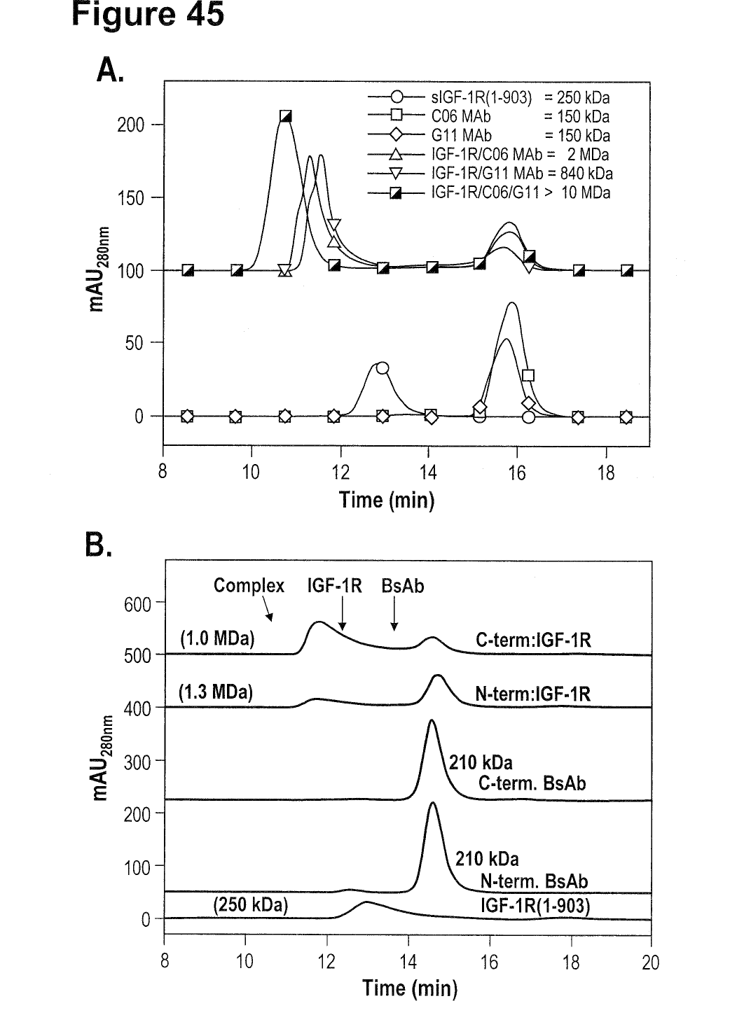

SEC/static light scattering results with the isolated C06 and G11 MAbs as well as with isolated sIGF-1R(1-903) demonstrated that the proteins all exhibit their expected molecular weights (150 kDa for the antibodies and ˜250 kDa for sIGF-1R(1-903), FIG. 45A). Complexes formed between the G11 MAb and sIGF-1R(1-903) appear to favor a 4 molecule species (2 molecules of G1 MAb and 2 molecules of sIGF-1R(1-903)) based on the observed molecular weight, 840 kDa (FIG. 45A). The C06 MAb forms much larger complexes with the soluble IGF-1R ectodomain, 2 MDa FIG. 45A. When both the C06 and G11 MAbs are introduced simultaneously to the soluble receptor ectodomain to form a ternary mixture, the average size of the immune complex(es) increases dramatically to >10 MDa outside our measureable range (FIG. 45A). This suggests, adding two antibodies that recognize non-overlapping epitopes to IGF-1R on the surface of a cell may lead to novel crosslinking and potentially downregulation behavior.

The N- and C-terminal IGF-1R bispecific antibody mixtures with sIGF-1R(1-903) were also examined by SEC/static light scattering (FIG. 45B). In isolation, the BsAbs and sIGF-1R(1-903) all exhibit their expected molecular weights (˜210 kDa for the BsAbs and ˜250 kDa for sIGF-1R(1-903)). Interestingly, when complexed with sIGF-1R(1-903), the BsAbs lead to similar complex sizes as was measured for the G11 MAb (˜1.0 MDa for the C-term. IGF-1R BsAb/sIGF-1R(1-903) complex and ˜1.3 MDa for the N-term. IGF-1R BsAb/sIGF-1R(1-903) complex). Due to the similar molecular weight of the BsAbs and sIGF-1R(1-903), we were not able to determine based on these studies alone whether the actual composition (i.e. number of BsAb vs. sIGF-1R(1-903 molecules) of the immune complexes are similar to what was observed for the G11 MAb.

In conclusion, we demonstrate that the C06 and G11 MAbs lead to the formation of very different immune complexes with the sIGF-1R(1-903) protein and that combination of the MAbs to form a ternary mixture leads to a very large increase in the size of the immune complexes. Conversion of the C06 and G11 antibodies into the bispecific format does not lead to a concomitant increase in the complexity and size of the immune complexes upon binding IGF-1R to the BsAbs. Interestingly, due perhaps to steric influences, the complexes formed by the BsAbs are small—similar to what was observed for the G11 MAb in isolation.

### Example 10

### Functional Activities of IGF-1R Bispecific Antibodies Targeting Two Distinct Epitopes on IGF-1R

The experimental results summarized in this example provide the biological rationale and the poor-of-concept that bispecific antibodies directed against two different epitopes of IGF-1R antibodies demonstrate enhanced biological activity over antibodies of single specificity and lead to a better anti-tumor activity in vivo.

C06 and G11 are two inhibitory anti-IGF-1R antibodies targeting two distinct epitopes on IGF-1R that block IGF-1 and IGF-2 binding through allosteric and competitive mechanisms respectively. Combination of C06 and G11 can enhance ligand blockade and inhibition of tumor cell growth. Therefore bispecific antibodies of two versions, N-IGF-1R bispecific antibody (N-terminal IGF-1R̂2 with the G11 IgG1 backbone using the C06 scFv with the I83E mutant) and C-IGF-1R bispecific antibody (C-terminal IGF-1R bispecific antibody with the G11 IgG1 backbone using the C06 scFv with the I83E mutant), were constructed as single bispecific molecules to target both C06 and G11 epitopes on IGF-1R. The biological effects of targeting two distinct inhibitory epitopes of IGF-1R with C06 and G1 combination, or single bispecific agents, N-IGF-1R bispecific antibody and C-IGF-1R bispecific antibody, have been evaluated in the following assays, summarized in the examples described herein: inhibition of IGF-1R phosphorylation; induction of IGF-1R downregulation; inhibition of AKT and MAPK inhibition; inhibition of cell growth in SFM and Serum, −/+IGF; cell cycle arrest; ADCC activity; inhibition of anchorage-independent growth; inhibition of SJSA-1 tumor growth in vivo; and PK study in mouse.

a. Targeting Two Distinct Inhibitory Epitopes of IGF-1R with Combination of C06 and G11 or a Single Agent Bispecific Antibody Inhibited IGF-1R Phosphorylation More Effectively than Single Monoclonal Antibodies

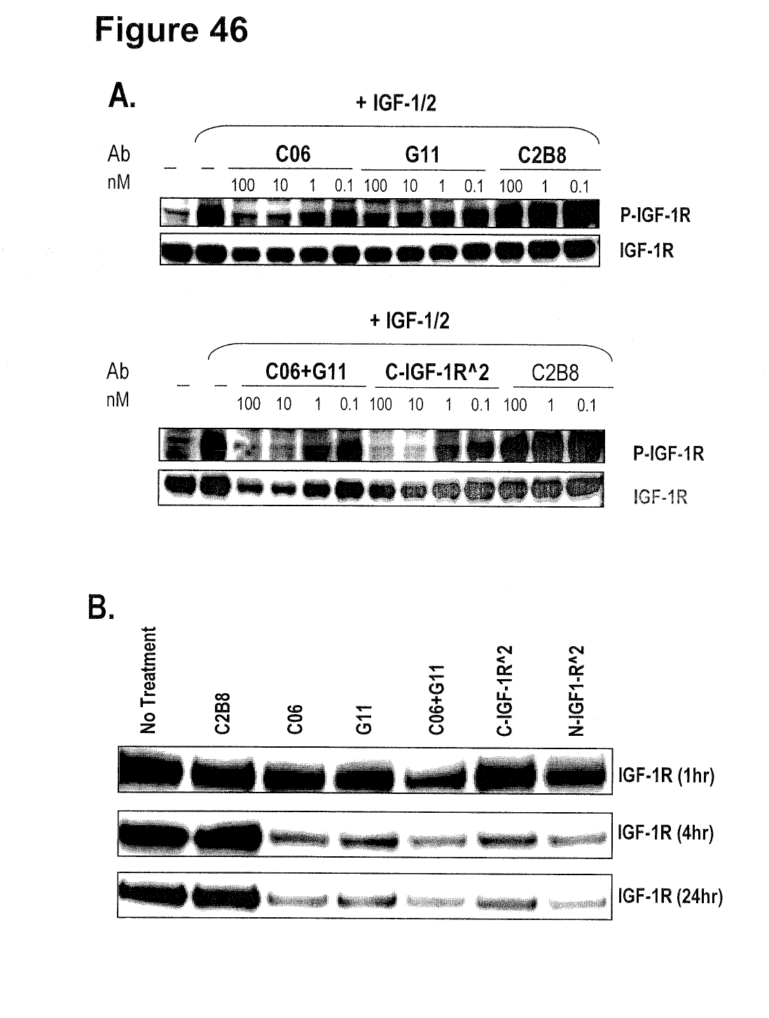

The effects of dual targeting two IGF-1R epitopes on IGF-1R phosphorylation were evaluated upon treatment of cells with C06/G11 combination or C-IGF-1R bispecific antibody. Briefly, H322M cells of human non-small cell lung carcinoma origin were seeded into 12-well culture plates and grown in RPMI-1640 medium containing 10% fetal bovine serum (FBS, Irvine Scientific, #3000A) overnight. Cells were serum starved for 24 hours and then treated with 0.1 nM, 1 nM, 10 nM, or 100 nM of C2B8 (anti-CD20, IgG1 isotype control antibody, Biogen Idec), C06, G11, combination of C06/G11, or C-IGF-1R bispecific antibody for 1 hour at 37° C. followed by stimulation with 100 ng/ml of IGF-1 and 100 ng/ml of IGF-2 (R & D Systems, #291-G1, #292-G2) for 20 minutes. Cellular proteins were extracted in cell lysis buffer (Meso Scale Discovery, cat# R60TX-3). Protein concentrations in lysates were measured using BCA protein assay kit (Pierce, cat# 23227) and equal amount of proteins was separated on a NUPAGE 4-12% Tris-Bis gel, and transferred to a Nitrocellulose membrane (0.45 μm pore). The blots were probed with anti-phospho-IGF-1R (Cell Signaling technology, cat# 3021 and 3024), and anti-total-IGF-1R (Cell Signaling technology, cat# 3027), then with detection antibody goat anti-Rabbit-IgG-HRP conjugate (Jackson ImmunoResearch, cat# 111-035-003). Blots were developed with Supersignal Western Substrate Kit (Pierce, cat# 34095) and chemoluminescence image captured on BioRad's VersaDoc 5000 imaging system. As shown in FIG. 46A, both C06 and G11 combination and the C-IGF-1R bispecific antibody demonstrated stronger inhibition of IGF-1R phosphorylation than C06 or G1 alone. Interestingly, combination of G11 and C06 more effectively downregulated total IGF-1R mass than single antibodies while C-IGF-1R bispecific antibody appeared to be less efficient at downregulating IGF-1R than the combination of C06 and G11. The fact that C-IGF-1R bispecific antibody can inhibit IGF-1R phosphorylation comparably to the combination without the same degree of receptor downregulation as detected by western blot indicates that the improved inhibitory activity of C-IGF-1R bispecific antibody on IGF-1R phosphorylation was mainly due to its increased IGF1/2 ligand blockade as previously shown. Similar results were seen with A549 non-small cell lung carcinoma cells and N-IGF-1R bispecific antibody (data not shown), suggesting that targeting two epitopes of IGF-1R using a combination of two single antibodies or a single bispecific antibody could inhibit IGF-1R activation more effectively than single monoclonal anti-IGF-1R antibodies in tumor cells.

b. Targeting Two Distinct Inhibitory Epitopes of IGF-1R with Combination of C06 and G11 or a Single Agent Bispecific Antibody Induced IGF-1R Downregulation Effectively

As demonstrated in Example 10a, when cells were treated with antibodies, in one hour the combination of G11 and C06 downregulated the total IGF-1R mass more effectively than single antibodies. We further investigated the ability of C06/G11 combination and BsAbs to degrade IGF-1R mass in a time course experiment. H322M cells were plated in 12-well culture plates and treated with 15 μg/ml of C06, G11, combination of C06 and G11, C-IGF-1R bispecific antibody or N-IGF-1R bispecific antibody for 1, 4, or 24 hours. Cellular proteins were extracted and separated on a NUPAGE 4-12% Tris-Bis gel, and transferred to a Nitrocellulose membrane as shown above in Example 10a. The blots were probed with anti-total-IGF-1R, and then with secondary antibody conjugate anti-Rabbit-IgG-HRP followed by development with Supersignal Western Substrate Kit from Pierce. FIG. 46B shows that after I-hour treatment, combination of C06 and G11 as well as N-IGF-1R bispecific antibody promoted more effective degradation of IGF-1R than single antibodies while C-IGF-1R bispecific antibody degraded IGF-1R to a similar degree as single antibody treatment. However, by 4 hours and 24 hours post addition of antibodies, the majority of IGF-1R in the cells was degraded by treatment with all antibodies, with little difference in the amount of remaining IGF-1R between cells treated single antibodies or C-BsAb, and cells treated with combination of C06 and G11 or N-IGF-1R.

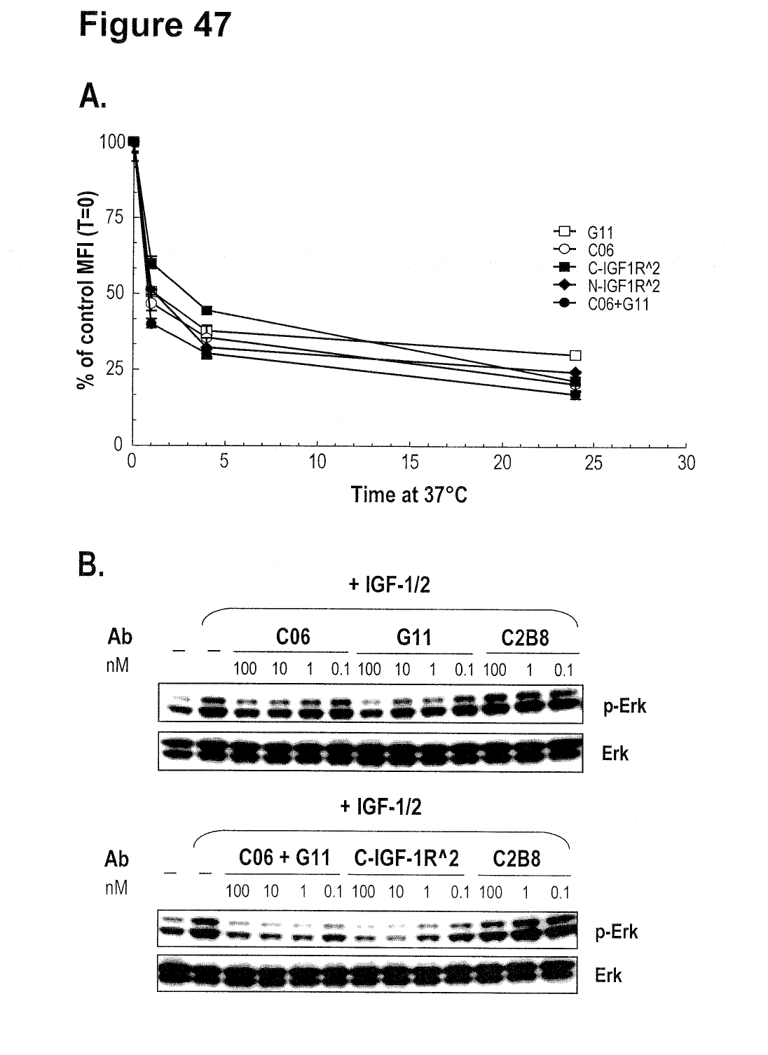

Furthermore, we examined the rates of IGF-1R internalization with various antibody treatment conditions by FACS (fluorescence-activated cell sorting). H322M cells were grown in 10% FBS containing RPMI-1640 medium for 48 hours. Cells were then incubated with 100 nM of C2B8 (isotype negative control), C06, G11, combination of C06 and G11, N- and C-IGF-1R bispecific antibody for 1 hour on ice to allow cell surface IGF-1R to be labeled with antibodies. A sample of cells stained with each antibody was kept on ice to prevent internalization and termed time zero (t=0). This was used as a 100% Ab bound control. The rest of antibody-stained cells were cultured in the growing medium for 1, 4, or 24 hours. At the end of each incubation time period, cells were lifted off the flask with cell dissociation buffer (Gibco, catalog #13151-014) and internalization was stopped with 0.1% sodium azide/1% BSA in PBS (FACS buffer) on ice. Cells were stained with PE-labeled secondary F(ab′)2 fragment goat anti-human IgG (H+L) (Jackson ImmunoResearch Lab, cat# 109-116-088; 2.5 μg/ml) in FACS buffer for 1 hour to detect antibody remaining on cell surface. Cells were fixed in 2% paraformadehyde (Electron Microscopy Sciences (EMS); cat#15710). Samples were then run on the FACS Calibur (BD) and fluorescence means (MFI) were determined. The fraction of IGF-1R labeled with each antibody remaining on cells surface after 1, 4, or 24 hours of treatment was calculated as a percentage of MFI at each time point over MFI at time zero. As shown in FIG. 47A, combination of C06 and G11 promoted a slightly higher rate of receptor internalization, while overall all IGF-1R antibodies can induce IGF-1R internalization quite efficiently, with reducing surface IGF-1R by more than 70% after 24 hours of antibody treatment. Taken together, these studies indicate that combination of G06 and G11 could downregulate IGF-1R more efficiently than monoclonal antibodies, however all antibodies including monoclonal antibodies and bispecific antibodies can eventually downregulate (internalize and degrade) IGF-1R quite effectively.

c. Targeting Two Distinct Inhibitory Epitopes of IGF-1R with Combination of C06 and G11 or a Single Agent Bispecific Antibody Inhibited Both AKT Survival and MAPK Proliferation Signaling Pathways More Effectively than Single Monoclonal Antibodies

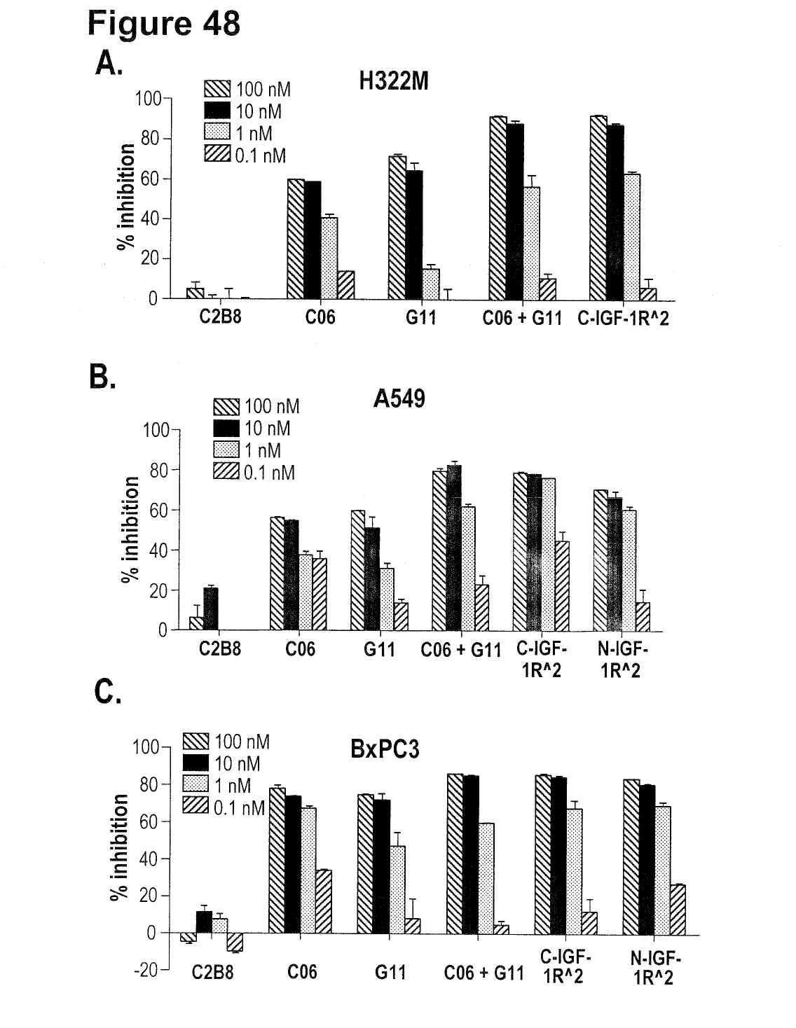

The effect of combining C06 and G11 or bispecific antibody targeting two epitopes of IGF-1R on downstream signaling events such as Akt and MAPK phosphorylation was examined. Cells were treated with 0.1 nM, 1 nM, 10 nM and 100 nM of C2B8, C06, G11, C06/G11, N- and C-IGF-1R bispecific antibody for 1 hour before stimulation with 100 ng/ml of IGF1 and IGF2 for 20 minutes as described in Example 10a. AKT phosphorylation was quantified using a p-AKT (Ser 473) MSD kit (Meso Scale Discovery, Cat #K15100D) according to the manufacturer's protocol. Briefly, cells were lysed in cell lysis buffer provided with the MSD kit, equal amount of proteins was added to the plate coated with anti-phospho-AKT antibody on electrodes, phosphorylated AKT was detected with an anti-total AKT antibody labeled with MSD SULFO-TAG reagent, and electrochemiluminescence was read out on SECTOR Imager 2400. The percentage of inhibition was calculated according to the formula [1−(Ab−SFM)/(IGF−SFM)]×100%. As shown in FIG. 48, C06 and G11 combination, C- and N-IGF-1R bispecific antibody all showed enhanced inhibition of AKT phosphorylation compared to C06 and G11 alone in H322M (FIG. 48A), A549 (FIG. 48B) and BxPC3 (FIG. 48C, pancreatic carcinoma) cells. In A549 cells, N-IGF-1R bispecific antibody showed slight less activity than C-IGF-1R bispecific antibody, suggesting that the two bispecific antibodies with different structures may have differentiated functions. For ERK phosphorylation determination, western blotting with anti-phospho-ERK (Cell signaling, Cat# 9101) and total ERK (Cell signaling, Cat# 9102) following a similar protocol as described in Example IOA was conducted. FIG. 47B shows that ERK phosphorylation was inhibited more effectively by C06 and G11 combination, and C-IGF-1R bispecific antibody than by C06 or G11 alone. Similar result that targeting two epitopes of IGF1-R with C06/G11 combination or C- and N-IGF-1R bispecific antibody led to enhanced inhibition of ERK phosphorylation was also observed in BxPC3 cells. These results support that targeting two distinct epitopes on IGF-1R with bispecific antibody or antibody combination will have stronger inhibition of IGF-1R downstream signaling such as AKT survival and/or ERK proliferation signaling in IGF-1R pathway sensitive tumors leading to enhanced anti-tumor activity.

d. Targeting Two Distinct Inhibitory Epitopes of IGF-1R with Combination of C06 and G1 or a Single Agent Bispecific Antibody Resulted in Enhanced Inhibition of Cell Growth of Various Tumors Compared to Single Monoclonal Antibodies

We examined the effect of C06/G11 combination, N- and C-IGF-1R bispecific antibody on cell growth of tumors of different origins in comparison to the effect of monoclonal antibodies C06 and G11 under different growth conditions, using a cell viability assay.

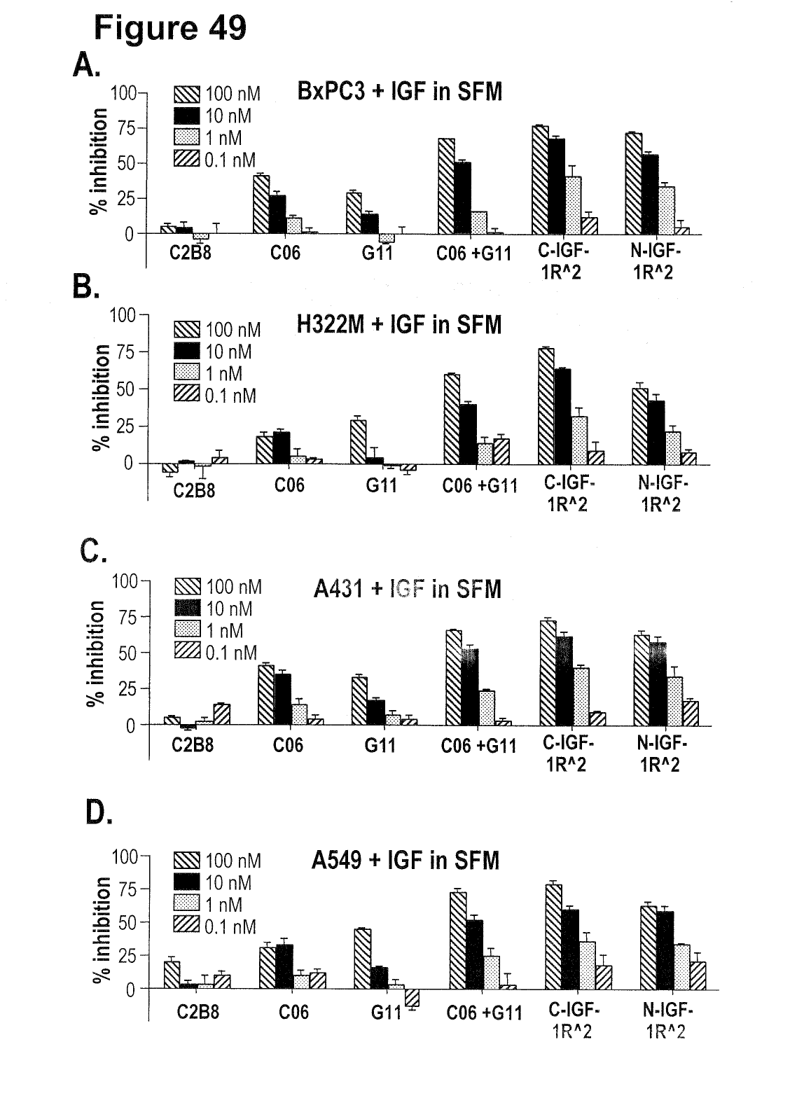

First, pancreatic cancer BxPC3 cells, non-small cell lung carcinoma H322M and A549 cells, and epidermoid carcinoma A431 cells were seeded at 5000-8000 cell per well in 96-well culture plates and grown in routine culture medium containing serum overnight, then cells were switched to serum-free medium and cultured in medium supplemented with 0.1 nM, 1 nM, 10 nM or 100 nM of C2B8, C06, G11, C06 and G11 combination, C- or N-IGF-1R bispecific antibody, and 100 ng/ml of IGF-1 and IGF-2 for additional three days. Cell viability was determined by measuring ATP levels with a Cell Titer Glo reagent (Promega, cat# G7571). FIG. 49 shows that N-IGF-1R bispecific antibody, C-IGF-1R bispecific antibody, combination of C06 and G11 all exhibited enhanced anti-tumor growth activity compared to C06 and G11 alone in BxPC3 (FIG. 49A), H322M (FIG. 49B), A431 (FIG. 49C) and A549 (FIG. 49D) cells. Interestingly, N-IGF-1R bispecific antibody appeared to be less effective than C-IGF-1R bispecific antibody in most of the cell lines tested, again highlighting the potential differences in structure-function relationship of the two bispecific molecules.

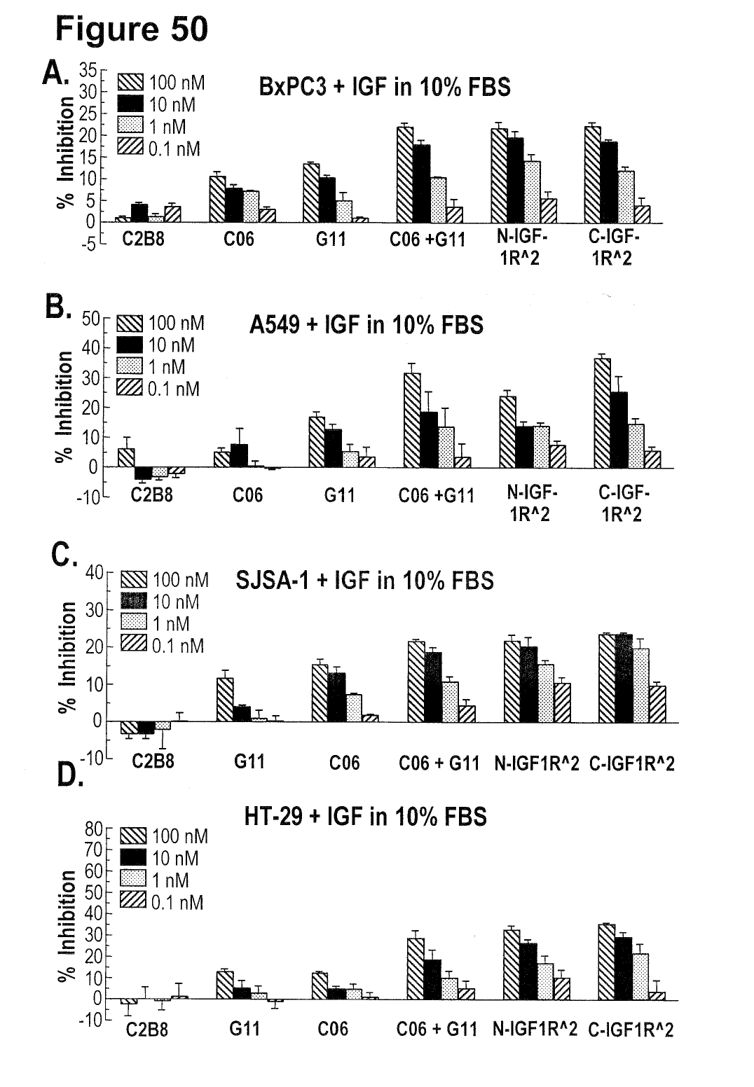

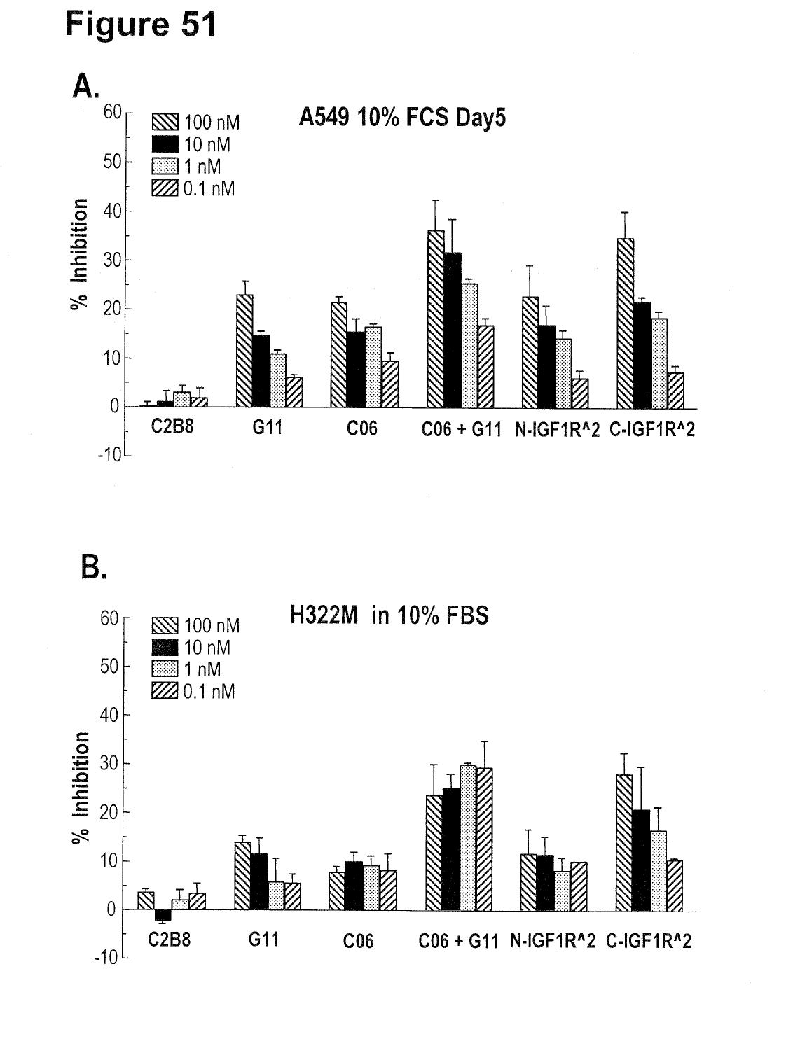

To further test the anti-tumor cell growth effect of antibodies under a more physiologically relevant condition, cells were grown in 10% serum (FBS, Hyclone cat# SH30071.03) containing medium supplemented with 0.1 nM, 1 nM, 10 nM or 100 nM of various antibodies and 200 ng/ml of IGF1 and IGF2 for 3 days prior to cell viability determination as described above. As shown in FIG. 50, similarly enhanced activity of IGF-1RA2 and C06/G11 combination were observed with BxPC3 (FIG. 50A), A549 (FIG. 50B), Osteosarcoma SJSA-1 (FIG. 50C) and Colon cancer HT-29 (FIG. 50D) cells. Additionally, antibodies were tested in the above serum containing cell growth conditions with no IGF1 or IGF2 supplementation in the cell culture medium. FIG. 51 shows that IGF-1R̂2 and C06/G11 combination demonstrated superior anti-tumor cell growth activity in serum-driven cell growth of A549 (FIG. 51A) and H322M (FIG. 51B) to C06 and G1 alone. Consistently, C-IGF-1R bispecific antibody displayed higher anti-tumor activity than N-IGF-1RA2 in several tumor cell lines tested, notably in H322M and A549 cells.

Overall these results indicate that targeting two distinct inhibitory epitopes of IGF-1R with C06 and G11 in combination or a single agent bispecific antibody can lead to enhanced anti-tumor cell growth activity compared to single monoclonal antibodies under various cell growth conditions.

e. Targeting Two Distinct Inhibitory Epitopes of IGF-1R with Combination of C06 and G11 or a Single Agent Bispecific Antibody Resulted in Tumor Cell Cycle Arrest

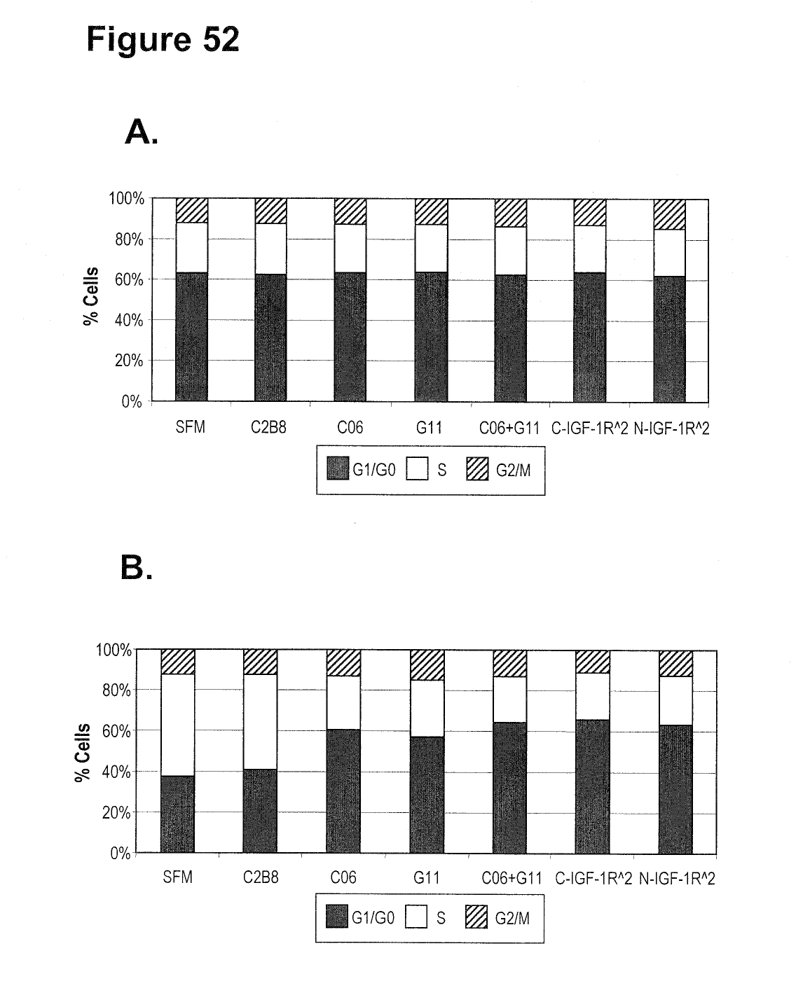

IGF-1R signaling is critical for cell survival and cell cycle progression. The effect of anti-IGF-1R antibodies on IGF-induced tumor cell cycle progression was evaluated using a FACS-based analysis. BxPC3 cells were seeded at 4×10⁵ cells per well into 6-well plates and cultured overnight in RPMI-1640 medium containing 5% FBS. Then cells were serum-starved for 24 hours before being treated with 100 ng/ml of IGF 1 and IGF-2 in the presence of 15 μg/ml of C2B8, C06, G11, C06/G11 combination, N- and C-IGF-1R bispecific for another 24 hours. Cells were lifted off the plates and then fixed in pre-chilled 70% ethanol. Fixed cells were stained with Propidium Iodide (PI, Molecular Probes, Cat# P3566) staining solution (20 μg/ml in PBS+0.1% BSA+Triton X-100) for 30 minutes at room temperature before FACS analysis of DNA contents. The relative percentage of cells in G0/G1, S, and G2/M phase was calculated from histograms using FlowJo 7.7.2 software. FIG. 52 shows that in serum-free conditions (FIG. 52A), more than 60% of cells were in G0/G1 phase, while IGF-1 and IGF-2 stimulation (FIG. 52B) dramatically decrease the percentage of cells in G0/G1 phase to approximately 40% and increased the percentage of cells in S phase by about two fold. All anti-IGF-1R antibodies were able to reverse the effect of IGF stimulation by increasing the percentage of cells in G0/G1 phase while reducing the percentage of cells in S phase. The combination of C06 and G11, and C-IGF-1R bispecific appeared to be most effective at blocking tumor cell cycle progression into S phase and inducing cell cycle arrest in G0/G1 phase.

f. Targeting Two Distinct Inhibitory Epitopes of IGF-1R with Combination of C06 and G11 or a Single Agent Bispecific Antibody do not Induce ADCC Activity

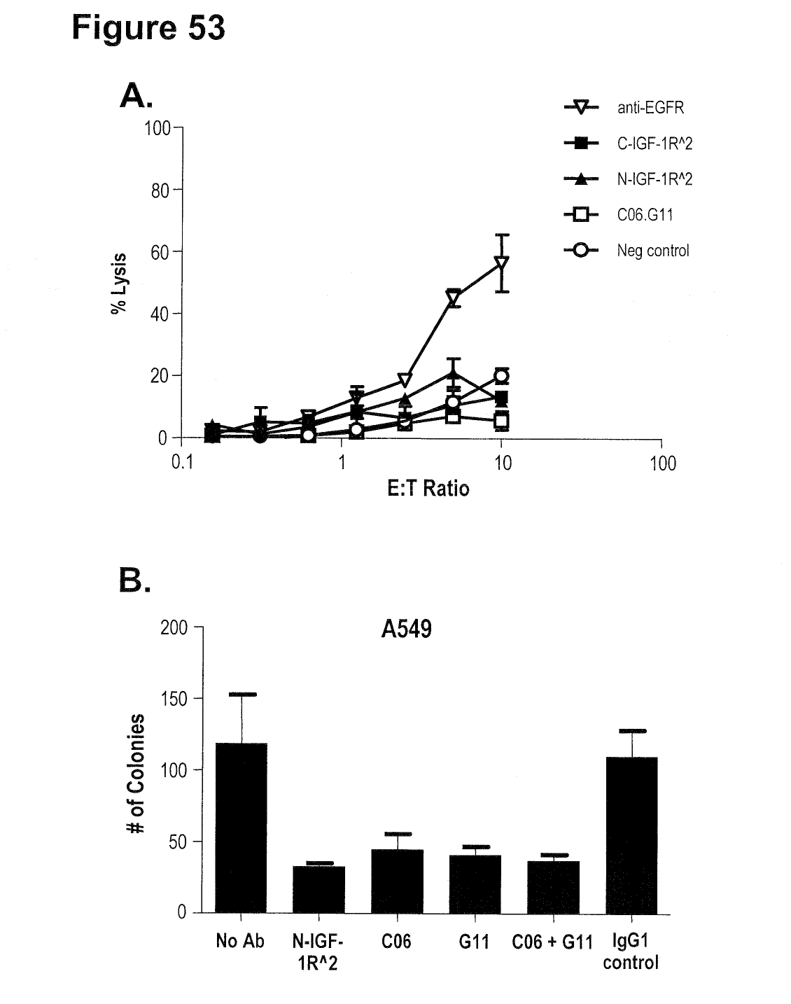

M13-C06 was shown to have no antibody-dependent cell-mediated cytotoxicity (ADCC). The ability of C06/G11 combination and bispecific anti-IGF-1R antibody to induce ADCC was tested in a ⁵¹Cr release ADCC assay. H322M cells expressing high IGF-1R were labeled with ⁵¹Cr for 1 hour, and then washed to remove the unincorporated ⁵¹Cr. Labeled target cells were added at 1×10⁴ cells per well to the wells containing 20 μg/ml of C06, G11, N- and C-IGF-1R bispecific antibodies. Effector cells were added for E:T ratios in the range of 0.1 to 10. After 4 hours of incubation at 37° C. in 5% C02 incubator, ⁵¹Cr released by target cells was measured using the Isodata Gamma Counter. Spontaneous release was from target cells with media only and maximum release was from targets in the presence of detergent Triton X-100. The percentage (%) of cell lysis was calculated using the formula (cpm_{sample release}−cpm_{spontanous release})/(cpm_{maximum release}−cpm_{spontanous release}). As shown in FIG. 53A, all anti-IGF-1R antibodies, C06, G11, N- and C-IGF-1R bispecific displayed similar activity as a negative control antibody with less than 20% cell lysis, whereas a positive control anti-EGFR antibody induced approximately 60% cell lysis at a E:T ratio of 10. The data indicates that targeting two epitopes of IGF-1R using bispecific antibodies or C06/G11 combination do not promote ADCC activity, similar to what was observed with the IgG1 versions of monoclonal antibodies C06 and G11. This result was consistent with the fact that all these anti-IGF-1R antibodies can efficiently induce IGF1-R downregulation, leading to inadequate surface receptors for an efficient antibody-NK cell engagement. These results clearly indicate that the anti-tumor activity of these anti-IGF-1R antibodies is dependent on their ability to block receptor signaling and biological functions and not on ADCC.

g. Targeting Two Distinct Inhibitory Epitopes of IGF-1R with Combination of C06 and G1 or a Single Agent Bispecific Antibody Resulted in Enhanced Inhibition of Anchorage-Independent Tumor Cell Growth Anchorage-independent growth is a hallmark of neoplastic transformation. To evaluate the ability of anti-IGF-1R antibodies to inhibit anchorage-independent growth, a soft agar colony formation assay was performed in 24-well culture plates. First, a bottom layer of 0.6% agar (Sigma, cat# A5431-250G) prepared with RPMI-1640 (Sigma, Cat# R1145) and 10% fetal bovine serum (Irvine Scientific, Cat# 3000A) was poured and allowed to solidify. Then, a top layer of 0.3% agar mixed with A549 cells at 50000 cells per well in medium containing 10% FBS and 15 μg/ml of C06, G11, N-IGF-1R bispecific, C06/G11 combination or CE9.1 (anti-CD4, IgG1 isotype negative control antibody, Biogen Idec) was poured at a controlled temperature between 38° C. to 42° C. The plates were incubated at 37° C. in 5% CO₂ incubator for 2-3 weeks, and colonies above 30 μm were counted with an automated mammalian cell colony counter (Oxford Optronix GELCOUNT). FIG. 53B shows the number of colonies formed under each antibody treatment conditions, indicating that all anti-IGF-1R antibodies strongly inhibited tumor cell anchorage-independent growth in soft agar, while N-IGF-1R bispecific and C06/G11 in combination displayed slightly enhanced inhibition compared to C06 or G11 alone. The result suggests combined targeting of two epitopes on IGF1-R may be more efficacious than monoclonal antibodies at inhibiting tumor formation and growth in vivo.

h. Targeting Two Distinct Inhibitory Epitopes of IGF-1R with Combination of C06 and G11 Led to Enhanced Inhibition of Tumor Growth In Vivo

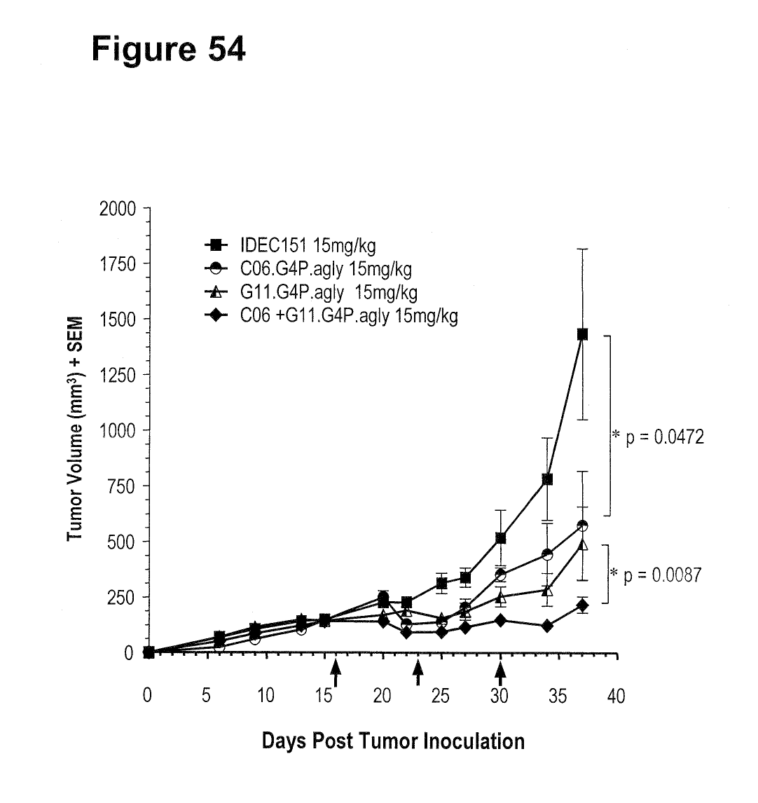

SJSA-1 osteosarcoma model was used to test the anti-tumor activity in vivo of M13-C06 and G11 in combination or as single agents. Nude mice (8-10 week old) were inoculated subcutaneously with 5×10⁶ SJSA-1 cells in 20% matrigel in the flank region. On day 15, mice with tumors were randomly sorted into 4 groups (n=8). The average tumor volume of the group at the initiation of treatment was approximately ˜150 mm³. Mice were injected intraperitoneally with M13-C06.G4P.agly (15 mg/kg), G11.G4P.agly (15 mg/kg), C06.G4P.agly plus G11.G4P.agly (15 mg/kg+15 mg/kg) or the control antibody IDEC151 (15 mg/kg) once a week for a total of 3 doses. Tumors were measured twice weekly, and tumor sizes were calculated using V=L*W²/2. FIG. 54 shows the tumor growth curves with different treatment regimens. Both M13-C06 and G11 were shown to significantly reduce tumor growth as single agent compared to control IDEC151 while combination of C06 and G11 resulted in significantly stronger inhibition of tumor growth than C06 or G11 alone. The anti-tumor activity of bispecifc antibodies is being tested in vivo in the SJSA-1 sarcoma model and other models, and it is expected to be comparable to that observed with combination of C06 and G11 while better than that of G11 and C06 alone.

i. Comparison of the Binding of the C06 and G11 Monoclonal Antibodies with the Binding of the N- and C-Terminal IGF-1R Bispecific Antibodies to Human Cancer Cells by Flow Cytometry

Cell-based flow cytometric analyses of antibody binding to human cancer cell lines were performed to extend analysis of the biophysical characteristics of the bispecific antibodies to a system with potentially greater biologic and therapeutic relevance. Analysis of the characteristics of binding of the bispecific antibodies, as compared to the relevant monoclonal antibodies, to the target antigen and epitopes presented on the complex surface of human cancer cell lines provides an opportunity to further define unique characteristics of the bispecific antibodies.

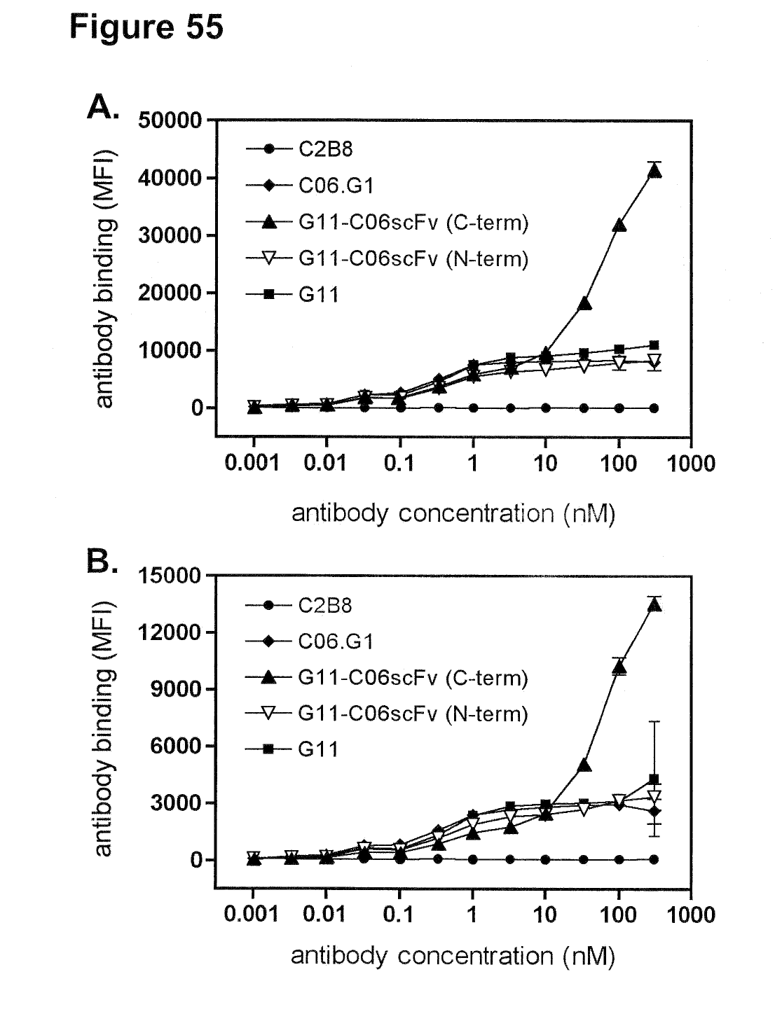

Cell lines. Cell lines analyzed in these studies included NCI-H322M (human non-small cell lung cancer cell line from NCI), NCI-MCF-7 (human breast adenocarcinoma cell line from NCI), and A431 (human epithelial carcinoma cell line from ATCC). Cells were routinely cultured to 80% confluence by passaging 24 hours prior to analysis. All cell lines were grown in complete growth medium containing RPMI-1640 (Gibco #11875) and 10% fetal bovine serum (Irvine Scientific Inc). Cell lines were routinely cultured no greater than 20 passages.
Flow cyotometric analysis of antibody binding to human cancer cells. Cells were lifted with cell dissociation buffer (Gibco catalog #13151-014), counted, washed and adjusted to 5×10⁶ cells/ml. Cells (2.5×10⁵; 50 uL) were added to each well of a 96-well round bottom plate (Costar #3799). Purified antibodies were tested at a starting concentration of 300 nM with serial half-log dilutions in FACS buffer. FACS buffer used throughout the assay was PBS (without Ca++/Mg++) containing 5% FBS. Samples were incubated on ice for 30 minutes, washed 3× with 200 uL FACS buffer and centrifuged at 1200 rpm for 3 minutes at 4° C. C2B8 (rituximab, IgG1) was used as a negative control. The supernatant was aspirated and 100 μl of PE-conjugated, affinity-purified F(ab′)₂ fragment-specific goat anti-human-IgG secondary detection antibody (Jackson ImmunoResearch Lab, catalog #109-116-097; used at 1:200 dilution) or PE-conjugated affinity-purified Fc-specific mouse anti-human-IgG secondary detection antibody (Leinco Technologies catalog #1-127; used at 5 uL/1×10⁶ cells dilution) was added to each corresponding well in FACS buffer. Samples were incubated for an additional 30 minutes on ice, shielded from light. Cells were washed as described above and resuspended in 100 μl FACS buffer containing propidium iodide (PI) for dead cell exclusion (BD Pharmingen, catalog# 51-66211E or 556463; use at 1:500 final in FACS buffer). Samples were run in triplicate using the FACSArray flow cytometer (Becton Dickinson) with 5000 live events collected per sample. Data analysis was performed using the GraphPad Prism version 5.0 software (GraphPad Software Inc., 11452 El Camino Real #215, San Diego, Calif. 92130).
Results. Analysis of binding of the anti-IGF-1R bispecific antibodies (C-terminal and N-terminal formats) and the relevant monoclonal antibodies to human cancer cell lines was performed by flow cytometry. Both the C06 and the G11 monoclonal antibodies demonstrated significant, dose-dependent binding to the non-small cell lung cancer cell line H322M (FIG. 55), with half-maximal binding for both antibodies observed at less than 1 nM (FIG. 55A: C06 EC₅₀ 0.20 nM, R² 0.98; G11 EC₅₀ 0.44 nM, R² 0.99; FIG. 55B: C06 EC₅₀ 0.25 nM, R² 0.97; G11 EC₅₀ 0.72 nM, R² 0.75). The N-terminal bispecific antibody, G11-C06scFv (N-term), exhibited cell-binding characteristics in these assays that was similar to both the single C06 and G11 monoclonal antibodies (FIG. 55A: G11-C06scFv (N-term) EC₅₀ 0.47 nM, R² 0.97; FIG. 55B: G11-C06scFv (N-term) EC₅₀ 0.48 nM, R² 0.96). However, the C-terminal bispecific antibody, G11-C06scFv (C-term), demonstrated significantly greater overall binding as compared to not only the single monoclonal antibodies, C06 and G11, but also as compared to the N-terminal bispecific antibody. Similar results were obtained in analyses of the MCF-7 human breast cancer cell line as well as the A431 human non-small cell lung cancer cell line (data not shown).

Assessment of binding of the monoclonal and the bispecific antibodies was performed using independent secondary reagents directed against either human Fab or human Fc gamma (FIGS. 55A and 55B, respectively) to exclude the possibility that the observed binding could reflect differences in the ability of the secondary reagent to bind to a monoclonal as compared to a bispecific antibody, or differences the ability of the secondary reagent to bind to the different bispecific antibody formats (e.g. N-terminal versus C-terminal fusions). The enhanced binding observed for the C-terminal bispecific antibody as compared to not only the corresponding monoclonal antibodies, but also as compared to the N-terminal bispecific antibody, was observed using both secondary reagents. These results therefore support the conclusion that the observed differences in binding reflect differences in binding of the antibodies to the tumor cells rather than differences in the binding of the secondary antibody reagent to the primary monoclonal and bispecific antibodies.

These results demonstrate that the G11-C06scFv (C-term) bispecific antibody binds to human cancer cells in a manner that is distinct from the binding of either of the monoclonal antibodies from which the bispecific antibody was derived. In addition, differences in the binding characteristics of the C-terminal bispecific antibody as compared to the N-terminal bispecific antibody further indicate that the specific conformation of the bispecific antibody influences binding to human cancer cells.

### Example 11

### Activity of IGF-1R Bispecific Antibodies In Vivo in Mouse

a. Pharmacological Kinetics

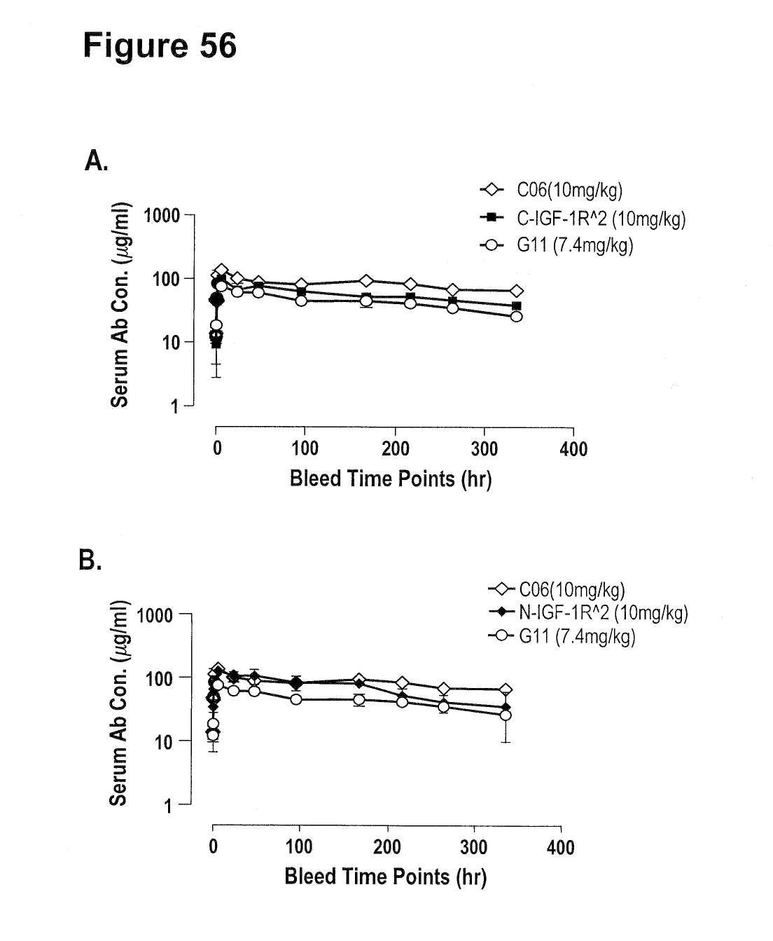

A single dose Pharmacological Kinetics (PK) study was conducted to evaluate the stability of bispecific Abs in non-tumor bearing mice to help dosing selection for efficacy studies in xenograft models. CB17 SCID female mice were dosed with 7.4 mg/kg of G11, 10 mg/kg of C06, N- or C-IGF-1R bispecific antibody. At various time points post dosing, mice were sacrificed and blood was collected by cardiac puncture and separated for serum recovery. Time points to be tested included pre dosing; at 0.25, 0.5, 1, 2, 6 and 24 hours, and at 2, 4, 7, 9, 11 and 14 days post dosing. Serum samples was frozen at time of collection and later tested by ELISA for the presence of antibodies. Briefly, ELISA plate (Terma Electron Corp., cat# 3455) was coated with goat anti-human IgG (Southern Biotech, Cat# 2040-01) overnight at 4° C. and then blocked with 1% nonfat milk and 0.05% Tween 20 in PBS at room temperature for 1 hour. Serial dilutions of sample serum were added to the coated plate and incubated for 1 hour, followed by additional incubation with detection antibody goat anti-human Kappa-HRP (Southern Biotech Cat# 2060-05) for 1 hour at room temperature. Control antibodies of known concentrations serially diluted in 1:25 normal mouse serum (Chemicon, Cat# S-25) were included to generate standard curves. Washes were performed between incubations. The plate was developed by addition of TMB substrate (3.3′, 5.5′-tetramethylbenzidine, KIRKEGAARD & PERRY LABS, cat# 50-76-00) and the reaction was stopped with H₂SO₄. OD (optical density) at 450 nm of each well was measured using a microplate reader (SpectraMax M2, Molecular Devices). The data was analyzed using SoftMax Pro and concentrations of human antibody in mouse serum were determined from the standard curves. FIG. 56 shows the concentrations of G11, C06, C-IGF-1R bispecific (FIG. 56A) and N-IGF-1R bispecific (FIG. 56B) in mouse serum over a 14-day period post single doing. The data indicates that both N- and C-IGF-1R BsAb molecules are quite stable in vivo and are cleared with rates similar to G11 and C06. Based on WinNonlin PK analysis, the half-lives (T_{1/2}) for C06, G11, C-IGF-1R bispecific antibody and N-IGF-1R bispecific antibody are approximately 18.8, 10.6, 11 and 7.5 days, respectively. The long half-lives suggest that the IGF-1R bispecific antibodies possess IgG-like pharmacokinectic properties suitable for in vivo efficacy studies, and potential therapeutic application in cancer treatment, with a dosing regimen similar to that of monoclonal antibodies.

b. Maintenance of Binding Activity

A number of studies have reported the production of IgG-like BsAbs using one or more scFv moieties as antigen recognition domains (Qu, Blood, 111: 2211-2219 (2008); Lu, J. Biol. Chem. 280: 19665-19672 (2006)). In these studies, the BsAb materials were shown to either lose activity towards the target(s) or to not recapitulate in vivo the activity of the molecules found using in vitro tumor cell proliferation/activity assays. Here we tested the ability of the anti-IGF-1R BsAbs containing a G11 IgG1 backbone fused to a stabilized C06 scFv to bind to both the C06 and G11 epitopes over the course of one week subsequent to IP injection in mice.

i. Methods

Equilibrium BsAb (in serum) binding to multiple epitopes of IGF-1R using surface plasmon resonance. All experiments were performed on a Biacore3000 instrument (Biacore). The C06 and G11 MAbs were separately immobilized to two different flow cell surfaces of a standard CM5 chip surface using standard amine chemistry protocols provided by the manufacturer. At these high immobilization levels of MAb, flowing low concentrations of sIGF-1R(1-903) (<50 nM) led to mass-transfer limited linear binding curves whose initial velocity of binding, Vᵢ (RU/s), depended linearly on the concentration of the sIGF-1R(1-903) solution flowed over the chip surface. The stoichiometry of binding between sIGF-1R(1-903) and the N- and C-terminal IGF-1R bispecific antibody was determined by flowing mixtures of sIGF-1R(1-903) and BsAb (serially diluted from serum) over the sensorchip surfaces containing C06 MAb, G11 MAb, or a blank surface blocked by ethanolamine after being activated using the standard NHC/EDC immobilization chemistry. The C06 and G11 sensorchip surfaces measure the concentration of unbound sIGF-1R(1-903) in solutions containing sIGF-1R(1-903) and the BsAbs. The binding stoichiometry, n, between the BsAbs and each sIGF-1R(1-903) epitope is determined by the concentration of unbound sIGF-1R(1-903) using the equation below:

$$Vi = m \cdot [ {[ IGF - 1 R ]}_{T} - \frac{1}{2} \{ ( {n [ BsAb ]}_{T} + {[ IGF - 1 R ]}_{T} + {K}_{D} ) - \sqrt{{( {n [ BsAb ]}_{T} + {[ IGF - 1 R ]}_{T} + {K}_{D} )}^{2} - 4 {{n [ Ab ]}_{T} [ IGF - 1 R ]}_{T}} \} ]$$

where Vᵢ=initial rate of binding, m=slope of the sIGF-1R(1-903) concentration-dependent standard curve, [IGF-1R]_{f}=unbound IGF-1R concentration=Vᵢ/m, [IGF-1R]ₜ=total IGF-1 concentration and [BsAb]ₜ total BsAb concentration (Day 2005). The stock concentrations of BsAb (within the serum) were determined by ELISA as described in the BsAb PK example.

ii. Results

The amount of intact/active C- and N-terminal IGF-1R bispecific antibody in serum collected from mice that were injected with 10 mg/kg of either BsAb after 1, 6, 24, and 168 hrs was assessed using solution Biacore measurements. The activity of the BsAb was measured as its ability to bind with full capability to both the G11 and C06 epitopes. Each serum sample doped with sIGF-1R(1-903) (30 nM, in-house ectodomain reagent) was serial diluted with a solution of 30 nM sIGF-1R(1-903). The samples were run over both C06 and G11 immobilized sensor chips to detect the ability of the BsAbs to block the ability of the sIGF-1R(1-903) from binding to the sensorchip surfaces. Time points at the 2 week period of the PK study were not measured for activity because the serum levels were expected to drop below the level where we could accurately determine the binding activity/stoichiometry using our Biacore method.

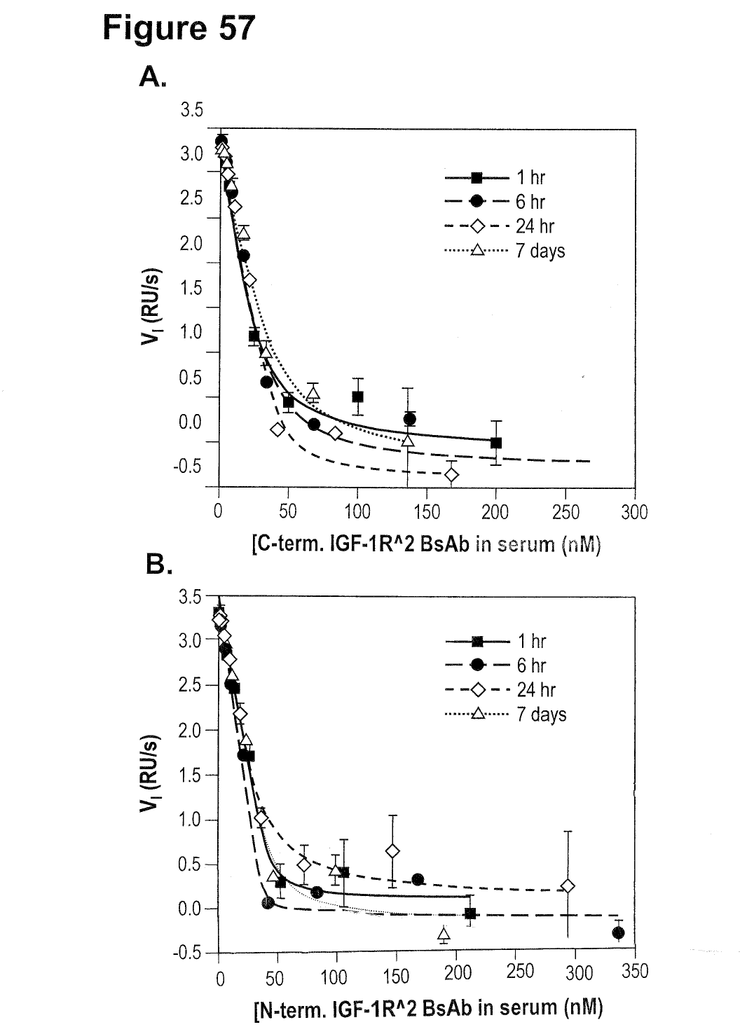

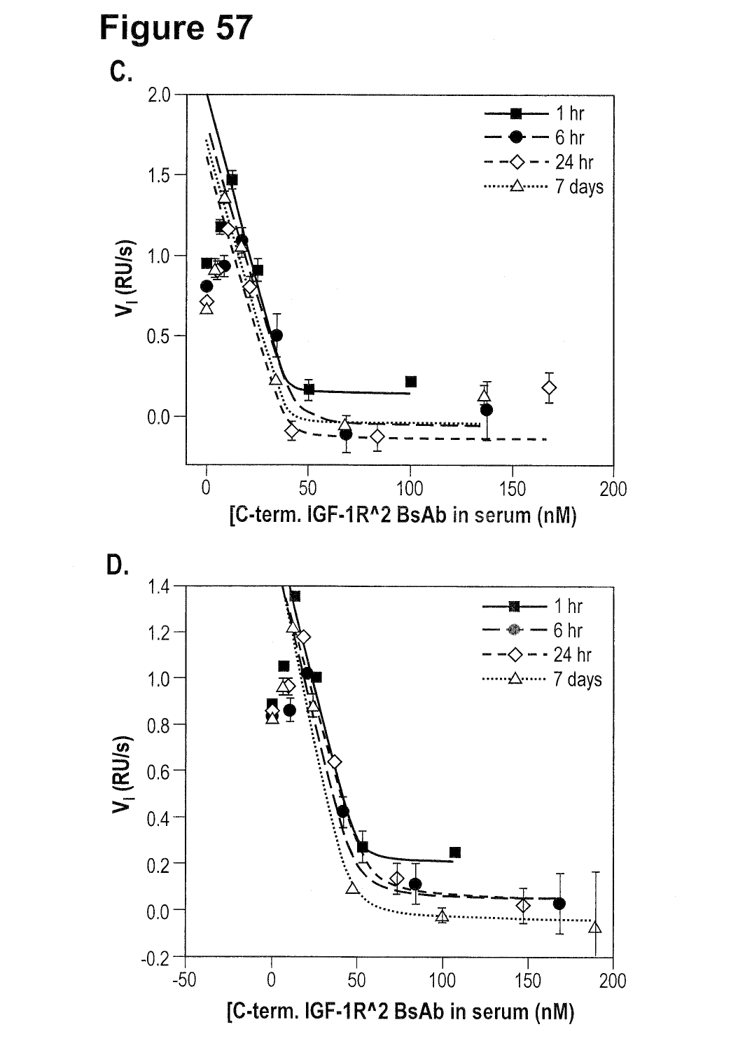

Both the C- and N-terminal IGF-1R bispecific antibodies appeared to retain full activity after being circulated in serum over the course of 1 week. As shown in FIG. 57, both the C- and N-term. IGF-1R bispecific antibodies demonstrate similar inhibition curves at all time points—similar to the curves measured in the previous example investigating the stoichiometry and affinity of the purified bispecific antibodies for IGF-1R. The slopes of the curves were all similar within the experimental error and no trends towards product degradation were observed (Table 26). The C-terminal BsAb appeared to be fully active (i.e. was capable of inhibiting sIGF-1R(1-903) at lower concentrations—concentrations that suggest all 4 binding sites of the C-term. tetravalent molecule are active, Table 26). The N-terminal BsAb did not demonstrate 1:1 stoichiometry (i.e., n=1.0), but instead had n=1.3 and 1.6 against the C06 and G11 surfaces, respectively (Table 26). Interestingly, this result was a recapitulation of the results observed for purified protein in the previous example suggesting that even purified N-term. IGF-1R bispecific antibody is not fully capable of engaging all its binding sites. Therefore, based on the activity of the purified protein, the N-terminal BsAb molecule exhibits no detectable loss of activity in vivo over the timecourse of the study. In conclusion, the N- and C-terminal IGF-1R bispecific antibody containing a stabilized C06 scFv appear to retain full activity in mice after 1 week in serum.

TABLE 26
Equilibrium (solution phase) stoichiometry measurements of the IGF-
1Rbispecific antibodies binding to either the C06 or G11 epitope.

| | n (C06 surface, | n (G11 surface, |
|---|---|---|
| Construct (time in | sIGF- | sIGF- |
| serum) | 1R/BsAb) | 1R/BsAb) |
| C-term. IGF-1R BsAb | | |
| 1 hr | 0.8 ± 0.2 | 1.3 ± 0.5 |
| 6 hr | 1.3 ± 0.2 | 1.4 ± 0.4 |
| 24 hr | 1.1 ± 0.2 | 1.3 ± 0.4 |
| 7 days | 1.1 ± 0.2 | 1.3 ± 0.4 |
| Average Stoichiometry | 1.1 ± 0.1 | 1.3 ± 0.1 |
| N-term. IGF-1R BsAb | | |
| 1 hr | 1.4 ± 0.2 | 1.7 ± 0.7 |
| 6 hr | 1.3 ± 0.2 | 1.6 ± 0.6 |
| 24 hr | 1.0 ± 0.3 | 1.4 ± 0.4 |
| 7 days | 1.4 ± 0.2 | 1.5 ± 0.6 |
| Average Stoichiometry | 1.3 ± 0.1 | 1.6 ± 0.1 |

### Example 12

### Treatment of Human Cancer Using Bispecific IGF-1R Bispecific Antibodies

This example describes methods for treating cancer using bispecific anti-IGF-1R antibodies (also referred to as IGF-1R̂2) to target epithelial and non-epithelial (e.g., sarcoma, lymphomas) malignant cells, for example hyperproliferative disorders where IGF-1R expression is detectable at the mRNA or protein level.

Treatment of tumor bearing mice with a mixture of two anti-IGF1-1R antibodies, C06 and G11 shows enhanced anti-tumor activity compared to C06 or G11 alone. Based on this observation and the enhanced biological activity demonstrated in vitro, it is expected that anti-IGF-1R bispecific antibodies (N- and C-versions) described in this embodiment will demonstrate greater anti-tumor activity in multiple epithelial, non-epithelial (e.g., sarcomas) and hematological malignancies (e.g., lymphoma, multiple myeloma), compared to any single anti-IGF-1R antibodies, including the C06 and G11.

Enhancement of the anti-tumor activity of IGF-1R bispecific antibody is expected in IGF-1R pathway sensitive tumors, including ones that are dependent on the IGF-2/IGF-1R autocrine axis for tumor growth and survival. Examples of IGF-1R pathway sensitive tumors are breast, non-small cell lung cancer (adeno carcinoma and non-adeno carcinoma), small cell lung cancer, prostate cancer, colon cancer, head and neck cancer (squamous), hepatocellular carcinoma, pancreatic carcinoma, intestinal and gastric carcinoma, renal cell carcinoma, Wilms tumor, bladder cancer, melanoma, adrenocortical carcinoma, glioblastoma multiforme, sarcomas (more than 70 different types, including Ewing's sarcoma, liposarcoma, synovial sarcoma, leiomyosarcoma, rhabdomyosarcoma, osteosarcoma, fibrosarcoma, neuroblastoma, craniopharyngioma), gastro intestinal stromal tumors (GIST), multiple myeloma, non-Hodgkin's lymphoma (B and T cell), acute lymphocytic leukemia (ALL) and other leukemias (B and T cell) and Hodgkin's diseases. It is also expected that the bispecific IGF-1R bispeicific antibodies will demonstrate greater anti-tumor activity not only among tumor subtypes that are known to be highly responsive to single anti-IGF-1R antibodies but also in tumor types that are marginally or less responsive to monoclonal IGF-1R antibody therapy. The ability of the bispecific IGF-1R bispecific antibodies to efficiently inhibit Akt survival pathway in a number of tumors indicates that the IGF-1R bispecific antibodies described in this example are potent Akt inhibitors and may become a preferred antagonistic over other modes of Akt antagonisms in treating cancer.

In certain embodiments the bispecific IGF-1R bispecific antibody (−C and −N terminal) is purified and formulated with a suitable pharmaceutical vehicle for injection. A human patient with a hyperproliferative disorder is given multiple doses of bispecific IGF-1R bispecific by intravenous infusion at a range of 1 mg/kg body weight to about 100 kg/mg body weight. Dosing is performed once a week or once in 2, 3 or 4 weeks until clinical evidence of disease progression or death is observed. The dosing interval can vary depending on the prognostic indicators measured during the course of the treatment. Antibodies can be administered prior to, concurrently with, or after standard of care for each indication (chemotherapy, radiotherapy, other targeted therapies, surgical resection). The patient is monitored to determine whether treatment has resulted in an anti-tumor response by evaluating one or more of the following parameters; tumor regression/reduction in the incidences of new lesions (tumors) as measured by CT scan, effect on glucose uptake (metabolic response) as measured by FDG-PET scan, and modulation on prognostic biomarkers used for evaluating disease prognosis.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed:

1. A method of inhibiting proliferation of a tumor cell expressing IGF-1R comprising contacting the tumor cell with a first binding moiety that binds to a first epitope of IGF-1R and blocks the binding of at least one of IGF-1 and IGF-2 to IGF-1R and a second binding moiety that binds to a second, different epitope of IGF-1R and blocks the binding of at least one of IGF-1 and IGF-2 to IGF-1R, wherein the binding of the first and second moiety to IGF-1R block IGF-1R-mediated signaling to a greater extent than the binding of the first or second moiety alone, to thereby inhibit survival or growth of a tumor cell expressing IGF-1R.

2. The method of claim 1, wherein the first and the second binding moiety block the binding of at least one of IGF-1 and IGF-2 to IGF-1R by different mechanisms.

3. The method of claim 1, wherein the first and the second binding moiety are present in the same binding molecule or separate binding molecules.

4.-5. (canceled)

6. A multispecific IGF-1R binding molecule comprising a first IGF-1R binding moiety that binds to a first epitope of IGF-1R and blocks the binding of at least one of IGF-1 and IGF-2 to IGF-1R and a second binding moiety that binds to a second, different epitope of IGF-1R and blocks the binding of at least one of IGF-1 and IGF-2 to IGF-1R.

7. A multispecific IGF-1R binding molecule said molecule comprising:
a) at least a first allosteric IGF-1R binding moiety which specifically binds a first allosteric IGF-1R epitope thereby allosterically blocking binding of IGF-1 and IGF-2 to IGF-1R; and
b) at least a second IGF-1R binding moiety wherein said second binding moiety specifically binds (i) a competitive IGF-1R epitope thereby competitively blocking binding of IGF-1 and IGF-2 to IGF-1R; or (ii) a second allosteric IGF-1R epitope thereby allosterically blocking binding of IGF-1 and not IGF-2 to IGF-1R.

8. The binding molecule of claim 7, wherein the first allosteric epitope is located within a region spanning the FnIII-1 domain of IGF-1R and comprising amino acids 437-586 of IGF-1R.

9. The binding molecule of claim 7, wherein the first allosteric epitope comprises at least 3 contiguous or non contiguous amino acids wherein at least one of the amino acids of the epitope is selected from the group consisting of amino acid positions 437, 438, 459, 460, 461, 462, 464, 466, 467, 469, 470, 471, 472, 474, 476, 477, 478, 479, 480, 482, 483, 488, 490, 492, 493, 495, 496, 509, 513, 514, 515, 513, 544, 545, 546, 547, 548, 551, 564, 565, 568, 570, 571, 572, 573, 577, 578, 579, 582, 584, 585, 586, and 587 of IGF-1R.

10. The binding molecule of claim 7, wherein the first allosteric epitope comprises at least one of amino acids 461, 462, and 464 of IGF-1R.

11. The binding molecule of claim 7, wherein the competitive epitope is located within a region encompassing a portion of the CRR domain and which region encompasses amino acid residues 248-303 of IGF-1R.

12. The binding molecule of claim 7, wherein the competitive epitope comprises at least 3 contiguous or non-contiguous amino acids wherein at least one of the amino acids of the epitope is selected from the group consisting of amino acids 248, 250, 254, 257, 259, 260, 263, 265, 301, and 303 of IGF-1R.

13. The binding molecule of claim 7, wherein the competitive epitope comprises amino acids 248, 250, and 254 of IGF-1R.

14. The binding molecule of claim 7, wherein the second allosteric epitope is located within a region that includes both the CRR and L2 domains of IGF-1R and which region encompasses residues 241-379 of IGF-1R.

15. The binding molecule of claim 7, wherein the second allosteric epitope comprises at least 3 contiguous or non-contiguous amino acids wherein at least one of the amino acids is selected from the group consisting of amino acids 241, 248, 250, 251, 254, 257, 263, 265, 266, 301, 303, 308, 327, and 379 of IGF-1R.

16. The binding molecule of claim 7, wherein the second allosteric epitope comprises at least one of amino acids 241, 242, 251, 257, 265, and 266 of IGF-1R.

17. The binding molecule of claim 7, wherein said first allosteric binding moiety is derived from a M13-C06 antibody (ATCC Accession No. PTA-7444) or a M14-C03 antibody (ATCC Accession No. PTA-7445) or wherein said first allosteric binding moiety competes for binding with said M-13-C06 or M15-C03 antibody.

18. The binding molecule of claim 17, wherein the first alloesteric binding moiety is an antigen binding site comprising CDRs 1-6 of the M13-C06 antibody (ATCC Accession No. PTA-7444) or the M14-C03 antibody (ATCC Accession No. PTA-7445).

19. (canceled)

20. The binding molecule of claim 7, wherein said competitive binding moiety is derived from a M14-G11 antibody (ATCC Accession No. PTA-7855) or competes for binding to IGF-1R with said M14-G11 antibody.

21. The binding molecule of claim 14, wherein the competitive binding moiety is an antigen binding site comprising CDRs 1-6 of the M14-G11 antibody (ATCC Accession No. PTA-7855).

22. (canceled)

23. The binding molecule of claim 7, wherein said second allosteric binding moiety is derived from a P1E2 antibody (ATCC Accession No. PTA-7730) or a αIR3 antibody or an antibody which competes with said P1E2 antibody or IR3 antibody.

24. The binding molecule of claim 23, wherein the second allosteric binding moiety is an antigen binding site comprising CDRs 1-6 of the P1E2 antibody (ATCC Accession No. PTA-7730) or the αIR3 antibody.

25. (canceled)

26. The binding molecule of claim 7, which is bispecific.

27. The binding molecule of claim 7, wherein the binding molecule is multivalent for the first or second binding specificity.

28. (canceled)

29. The binding molecule of claim 7, wherein the binding molecule comprises four binding moieties.

30. The binding molecule of claim 7, wherein at least one of the binding moieties is an scFv molecule.

31. The binding molecule of claim 7, wherein the binding molecule is a tetravalent antibody molecule comprising two scFv molecules.

32. The binding molecule of claim 31, wherein said scFv molecules are fused to the C-termini or N-termini of the heavy chains of the tetravalent antibody molecule or the N-termini or said heavy chains.

33.-34. (canceled)

35. The binding molecule of claim 7, which comprises a stabilized scFv molecule.

36. The binding molecule of claim 7, wherein the binding molecule is fully human, humanized, or chimeric.

37.-38. (canceled)

39. The binding molecule of claim 7, which comprises a heavy chain constant region or fragment thereof.

40. The binding molecule of claim 39, wherein said heavy chain constant region or fragment thereof is human IgG4.

41. The binding molecule of claim 40, wherein said IgG4 constant region lacks glycosylation.

42. The binding molecule of claim 41, wherein said IgG4 constant regions comprises a S228P and T299A mutation as compared to a wild-type IgG4 constant region, numbering according to the EU numbering system.

43. A bispecific IGF-1R antibody molecule comprising two allosteric binding moieties derived from a M13-C06 antibody (ATCC Accession No. PTA-7444) and two competitive binding moieties derived from a M14-G11 antibody (ATCC Accession No. PTA-7855).

44. The antibody molecule of claim 43, wherein said competitive binding moieties are provided by an IgG antibody and said allosteric binding moieties are provided by two scFv molecules that are linked or fused to said IgG antibody.

45. The antibody molecule of claim 44, wherein said IgG antibody comprises the light chain (VL) and heavy chain (VH) variable domains from the M14-G11 antibody.

46. The antibody molecule of claim 45, wherein said VL domain of said IgG antibody comprises the amino acid sequence of SEQ ID NO:93 and said VH domain of said IgG antibody comprises the amino acid sequence of SEQ ID NO:32.

47. The antibody molecule of claim 44, wherein one or both of said scFv molecules of said allosteric binding moieties comprise a light chain (VL) and a heavy chain (VH) variable domain derived from the M13-C06 antibody.

48. The antibody molecule of claim 47, wherein one or both of said scFv molecules is a stabilized C06 scFv molecule having a T50 of greater than 60-61° C.

49. The antibody molecule of claim 47, wherein one or both of said scFv molecules is a stabilized scFv molecule having a T50 that is at least 2° C.-10° C. higher than that of a conventional C06 scFv molecule (pWXU092 or pWXU090).

50. The antibody molecule of claim 48, wherein the variable light domain (VL) of said stabilized scFv is identical to the VL domain of the M13-CO6 antibody (SEQ ID NO:78) but for the presence of one or more stabilizing mutations at amino acid positions within the VL domain selected from the group consisting of: (i) M4, (ii) L11; (iii) V15, (iv) T20, (v) Q24, (vi) R30, (vii) T47, (viii) A51, (ix) G63, (x) D70, (xi) S72, (xii) T74, (xiii) S77 and (xiv) 183 (Kabat numbering convention).

51. The antibody molecule of claim 50, said stabilizing mutations are selected from the group consisting of: M4L, L11G, V15A, V15D, V15E, V15G, V15I, V15N, V15P, V15R, V15S, T20R, Q24K, R30N, R30T, R30Y, A51G, G63S, D70E, S72N, S72Y, T74S, S77G, I83D, I83E, I83G, I83M, I83R, I83S and I83V.

52. The antibody molecule of claim 48, wherein the variable heavy domain (VH) of said stabilized scFv is identical to the VH domain of the M13-CO6 antibody (SEQ ID NO: 14) but for the presence one or more stabilizing mutations at amino acid positions selected from the group consisting of: (i) S21, (ii) W47, (iii) R83 and (iv) T110 (Kabat numbering convention).

53. The antibody molecule of claim 52, wherein said stabilizing mutations are selected from the group consisting of: S21E, W47F, R83K, R83T and T110V.

54. The antibody molecule of claim 48, wherein said stabilized scFv molecule comprises a combination of mutations selected from the group consisting of VL L15S: VH T110V or VL S77G: VL I83Q.

55. (canceled)

56. The antibody molecule of claim 48, wherein said stabilized scFv molecule is a stabilized CO6 scFv molecule is selected from the group consisting of MJF-014, MJF-015, MJF-016, MJF-017, MJF-018, MJF-019, MJF-020, MJF-021, MJF-022, MJF-023, MJF-024, MJF-025, MJF-026, MJF-027, MJF-028, MJF-029, MJF-030, MJF-031, MJF-032, MJF-033, MJF-034, MJF-035, MJF-036, MJF-037, MJF-038, MJF-039, MJF-040, MJF-041, MJF-042, MJF-043, MJF-044, MJF-045, MJF-046, MJF-047, MJF-048, MJF-049, MJF-050 and MJF-051.

57. The antibody molecule of claim 48, wherein said stabilized scFv molecule is a stabilized C06 VH/VL (I83E) scFv molecule comprising the amino acid sequence of MJF-045 (SEQ ID NO:128).

58. The antibody molecule of claim 44, wherein one or both of said scFv molecules is linked to said IgG antibody by a Gly/Ser linker.

59. The antibody molecule of claim 58, wherein said Gly/Ser linker is a (Gly₄Ser)₅ or Ser(Gly₄Ser)₃ linker.

60. The antibody molecule of claim 44, wherein said scFv molecules are linked or fused to said IgG antibody via the VL domain or VH domain of said scFv molecules.

61. The antibody molecule of claim 60, wherein the scFv molecule is of the orientation VH→(Gly4Ser)ₙ linker →VL or VL→(Gly4Ser)ₙ linker →VH, and wherein n is 3, 4, 5, or 6.

62.-63. (canceled)

64. The antibody molecule of claim 44, wherein one or both of said scFv molecules is linked or fused to a heavy chain of said IgG antibody to form a heavy chain of said bispecific antibody.

65. The antibody molecule of claim 64, wherein one of said scFv molecules is linked or fused to a first heavy chain of said IgG antibody and one of said scFv molecules is linked or fused to a second heavy chain of said IgG antibody.

66. The antibody molecule of claim 65, wherein said scFv molecules are linked or fused to the N-terminus of said first and second heavy chains of said IgG antibody.

67. The antibody molecule of claim 66, wherein the light chains of said IgG antibody comprise the G1 light chain sequence of SEQ ID NO: 130 (pXWU118); and wherein the heavy chains of said bispecific antibody comprise the amino acid sequence of SEQ ID NO:133 (pXWU136).

68. The antibody molecule of claim 66, wherein said binding molecule is produced by the cell line deposited as ATCC Deposit No. PTA-9440.

69. The antibody molecule of claim 65, wherein said scFv molecules are linked or fused to the C-terminus of said first and second heavy chains of said IgG antibody to form the heavy chains of said bispecific antibody molecule.

70. The antibody molecule of claim 69, wherein the light chains of said IgG antibody comprise the G11 light chain sequence of SEQ ID NO: 130 (pXWU118) and wherein the scFv molecule when linked to the N-terminus of said heavy chain comprises the sequence of SEQ ID NO:137 (pXWU135).

71. The antibody molecule of claim 69, wherein said binding molecule is produced by the cell line deposited as ATCC Deposit No. PTA-9439.

72. The antibody molecule of claim 44, wherein one or both of said scFv molecules is linked or fused to a light chain of said IgG antibody.

73. The antibody molecule of claim 72, wherein one of said scFv molecules is linked or fused to a first light chain of said IgG antibody and one of said scFv molecules is linked or fused to a second light chain of said IgG antibody.

74. The antibody molecule of claim 72, wherein said scFv molecules are linked or fused to the N-terminus of said first and second light chains of said IgG antibody.

75. The antibody molecule of claim 43, wherein said allosteric binding moieties are provided by a IgG antibody and said competitive binding moieties are provided by two scFv molecules that are linked or fused to said IgG antibody.

76. The antibody molecule of claim 75, wherein said IgG antibody comprises the light chain (VL) and heavy chain (VH) variable domains from the M13-C06 antibody or M14-G11 antibody.

77. The antibody molecule of claim 76, wherein said VL domain of said IgG antibody comprises the amino acid sequence of SEQ ID NO:78 and said VH domain of said IgG antibody comprises the amino acid sequence of SEQ ID NO:14.

78. (canceled)

79. The antibody molecule of claim 78, wherein one or both of said scFv molecules is a stabilized G11 scFv molecule having a T50 of greater than 50-51° C.

80. The antibody molecule of claim 78, wherein one or both of said scFv molecules is a stabilized scFv molecule having a T50 that is at least 2° C.-10° C. higher than that of a conventional G11 (VL/GS4/VH) scFv molecule (pMJF060).

81. The antibody molecule of claim 79, wherein the variable light domain (VL) of said stabilized scFv is identical to the VL domain of the M14-G11 antibody (SEQ ID NO:93) but for the presence of one or more stabilizing mutations at amino acid positions L50 and/or V83 (Kabat numbering convention).

82. The antibody molecule of claim 81, said stabilizing mutations are selected from the group consisting of: L50G, L50M, L50N and V83E.

83. The antibody molecule of claim 79, wherein the variable heavy domain (VH) of said stabilized scFv is identical to the VH domain of the M14-G11 antibody (SEQ ID NO:32) but for the presence one or more stabilizing mutations at amino acid positions E6 and/or S49 (Kabat numbering convention).

84. The antibody molecule of claim 83, wherein said stabilizing mutations are selected from the group consisting of: E6Q, S49A and S49G.

85. The antibody molecule of claim 79, wherein said stabilized scFv molecule comprises a combination of mutations selected from the group consisting of VL L50N: VH E6Q or VL V83E: VH E6Q.

86. (canceled)

87. The antibody molecule of claim 79, wherein said stabilized scFv molecule is a stabilized G11 scFv molecule is selected from the group consisting of MJF-060, MJF-084, MJF-085, MJF-086, MJF-087, MJF-091, MJF-092 and MJF-097.

88. The antibody molecule of claim 75, wherein one or both of said scFv molecules is linked to said IgG antibody by a Gly/Ser linker.

89. The antibody molecule of claim 88, wherein said Gly/Ser linker is a (Gly₄Ser)₅ or Ser(Gly₄Ser)₃ linker.

90. The antibody molecule of claim 75, wherein said scFv molecules are linked or fused to said IgG antibody via the VL domain or VH domain of said scFv molecules.

91. The antibody molecule of claim 90, wherein the scFv molecule is of the orientation VH→(Gly4Ser)ₙ linker →VL or VL→(Gly4Ser)ₙ linker →VH, and wherein n is 3, 4, 5, or 6.

92.-93. (canceled)

94. The antibody molecule of claim 75, wherein one or both of said scFv molecules is linked or fused to a heavy chain of said IgG antibody.

95. The antibody molecule of claim 94, wherein one of said scFv molecules is linked or fused to a first heavy chain of said IgG antibody and one of said scFv molecules is linked or fused to a second heavy chain of said IgG antibody.

96. The antibody molecule of claim 95, wherein said scFv molecules are linked or fused to the N-terminus of said first and second heavy chains of said IgG antibody.

97. The antibody molecule of claim 96, wherein the light chains of said IgG antibody comprise the C06 light chain sequence of SEQ ID NO:140 and wherein the scFv molecule when linked to the N-terminus of said heavy chain comprises the sequence of SEQ ID NO:144.

98. (canceled)

99. The antibody molecule of claim 95, wherein said scFv molecules are linked or fused to the C-terminus of said first and second heavy chains of said IgG antibody.

100. The antibody molecule of claim 99, wherein the light chains of said IgG antibody comprise the C06 light chain sequence of SEQ ID NO:140 and wherein the scFv molecule when linked to the N-terminus of said heavy chain comprises the sequence of SEQ ID NO:144.

101. (canceled)

102. The antibody molecule of claim 95, wherein one or both of said scFv molecules is linked or fused to a light chain of said IgG antibody.

103. The antibody molecule of claim 102, wherein one of said scFv molecules is linked or fused to a first light chain of said IgG antibody and one of said scFv molecules is linked or fused to a second light chain of said IgG antibody.

104. The antibody molecule of claim 103, wherein said scFv molecules are linked or fused to the N-terminus of said first and second light chains of said IgG antibody.

105. The antibody molecule of claim 43, wherein said IgG antibody comprises heavy chain constant domains of the human IgG4 or IgG1 isotype.

106. (canceled)

107. The antibody molecule of claim 43, wherein said IgG antibody is a chimeric of heavy chain constant domain portions from two or more human antibody isotypes.

108. The antibody molecule of claim 107, wherein the IgG antibody comprises a Fc region wherein residues 233-236 and 327-331 of the Fc region are from a human IgG2 antibody and the remaining residues of the Fc region are from a human IgG4 antibody.

109-110. (canceled)

111. The binding molecule of claim 7, which (i) is essentially resistant to aggregation when produced at commercial scale, (ii) inhibits IGF-1R-mediated cell proliferation, (iii) inhibits IGF-1 or IGF-2-mediated IGF-1R phosphorylation, (iv) inhibits IGF-1 or IGF-2-mediated AKT phosphorylation, (v) inhibits AKT mediated survival signalling, (vi) inhibits tumor growth in vivo, or (vi) induces IGF-1R internalization.

112.-117. (canceled)

118. The binding molecule of claim 7, wherein said binding molecule is conjugated to an agent selected from the group consisting of cytotoxic agent, a therapeutic agent, cytostatic agent, a biological toxin, a prodrug, a peptide, a protein, an enzyme, a virus, a lipid, a biological response modifier, pharmaceutical agent, a lymphokine, a heterologous antibody or fragment thereof, a detectable label, polyethylene glycol (PEG), and a combination of two or more of any said agents.

119. (canceled)

120. A composition comprising the binding molecule of claim 7, and a carrier.

121. A method of treating a subject suffering from a hyperproliferative disorder comprising administering a binding molecule of claim 7, to the subject such that treatment occurs.

122.-123. (canceled)

124. A nucleic acid molecule encoding the binding molecule of claim 7 or a heavy chain or a light chain thereof.

125. The nucleic acid molecule of claim 124, which is in a vector.

126. A host cell comprising the vector of claim 125.

127. A method of producing the binding molecule of claim 7, comprising
(i) culturing the host cell of claim 126 such that the binding molecule is secreted in host cell culture media and (ii) isolating the binding molecule from the media.

128. A stabilized scFv molecule, wherein the stabilized scFv molecule has a T50 that is at least 2° C.-10° C. higher than that of a conventional scFv molecule.

129. The scFv molecule of claim 128, wherein said molecule has a T50 of greater than 50° C. or 60° C.

130. (canceled)

131. The scFv molecule of claim 128, which comprises one or more stabilizing mutations as compared to a conventional scFv molecule, wherein said mutations are present at VL amino acid positions selected from the group of VL amino acid positions consisting of: (i) 4, (ii) 11; (iii) 15, (iv) 20, (v) 24, (vi) 30, (vii) 47, (viii) 50, (ix) 51, (x) 63, (xi) 70, (xii) 72, (xiii) 74, (xiv) 77 and (xv) 83 (Kabat numbering convention).

132. The scFv molecule of claim 131, wherein said stabilizing mutations are selected from the group consisting of: 4L, 11G, 15A, 15D, 15E, 15G, 15I, 15N, 15P, 15R, 15S, 20R, 24K, 30N, 30T, 30Y, 50G, 50M, 50N, 51G, 63S, 70E, 72N, 72Y, 74S, 77G, 83D, 83E, 83G, 83M, 83R, 83S and 83V.

133. The scFv molecule of claim 128, which comprises one or more stabilizing mutations as compared to a conventional scFv molecule, wherein said mutations are present at VH amino acid positions selected from the group of VH amino acid positions consisting of: (i) 6, (ii) 21, (iii) 47, (iv) 49 and (v) 110 (Kabat numbering convention).

134. The scFv molecule of claim 133, wherein said stabilizing mutations are selected from the group consisting of: 6Q, 21E, 47F, 49A, 49G, 83K, 83T and 110V.

135. The scFv molecule of claim 128, which comprises one or more stabilizing mutations as compared to a conventional scFv molecule, wherein said mutations are present at amino acid positions selected consisting of: (i) VL amino acid position 50, (ii) VL amino acid position 83; (iii) VH amino acid position 6 and (iv) VH amino acid position 49 (Kabat numbering convention).

136. The scFv molecule of claim 128, which comprises stabilizing mutations as compared to a conventional scFv molecule, wherein said mutations are present at: (i) VL amino acid position 50, (ii) VL amino acid position 83; (iii) VH amino acid position 6 and (iv) VH amino acid position 49 (Kabat numbering convention).

137. The scFv molecule of claim 135 or 136, wherein said stabilizing mutations are selected from the group consisting of: VL 50G, VL 50M, VL 50N, VL 83D, VL 83E, VL 83G, VL 83M, VL 83R, VL 83S, VL 83V, VH 6Q, VH 49A and VH 49G.

138. The scFv molecule of claim 128, wherein said stabilized scFv molecule has a T50 that is at least 2° C.-10° C. higher than that of a conventional C06 scFv molecule (pWXU092 or pWXU090).

139. The scFv molecule of claim 138, wherein the variable light domain (VL) of said stabilized scFv is identical to the VL domain of the M13-CO6 antibody (SEQ ID NO:78) but for the presence of one or more stabilizing mutations at amino acid positions within the VL domain selected from the group consisting of: (i) M4, (ii) L11; (iii) V15, (iv) T20, (v) □24, (vi) R30, (vii) T47, (viii) A51, (ix) G63, (x) D70, (xi) S72, (xii) T74, (xiii) S77 and (xiv) 183 (Kabat numbering convention).

140. The scFv molecule of claim 139, said stabilizing mutations are selected from the group consisting of: M4L, L11G, V15A, V15D, V15E, V15G, V15I, V15N, V15P, V15R, V15S, T20R, Q24K, R30N, R30T, R30Y, A51G, G63S, D70E, S72N, S72Y, T74S, S77G, I83D, I83E, I83G, I83M, I83R, I83S and I83V.

141. The scFv molecule of claim 138, wherein the variable heavy domain (VH) of said stabilized scFv is identical to the VH domain of the M13-C06 antibody (SEQ ID NO:14) but for the presence one or more stabilizing mutations at amino acid positions selected from the group consisting of: (i) S21, (ii) W47, (iii) R83 and (iv) T110 (Kabat numbering convention).

142. The scFv molecule of claim 141, wherein said stabilizing mutations are selected from the group consisting of: S21E, W47F, R83K, R83T and T110V.

143. The scFv molecule of claim 138, wherein said stabilized scFv molecule comprises a combination of mutations selected from the group consisting of VL L15S: VH T110V or VL S77G: VL 183Q.

144. (canceled)

145. The scFv molecule of claim 138, wherein said stabilized scFv molecule is a stabilized C06 scFv molecule is selected from the group consisting of MJF-014, MJF-015, MJF-016, MJF-017, MJF-018, MJF-019, MJF-020, MJF-021, MJF-022, MJF-023, MJF-024, MJF-025, MJF-026, MJF-027, MJF-028, MJF-029, MJF-030, MJF-031, MJF-032, MJF-033, MJF-034, MJF-035, MJF-036, MJF-037, MJF-038, MJF-039, MJF-040, MJF-041, MJF-042, MJF-043, MJF-044, MJF-045, MJF-046, MJF-047, MJF-048, MJF-049, MJF-050 and MJF-051

146. The scFv molecule of claim 128, which is stabilized scFv molecule having a T50 that is at least 2° C.-10° C. higher than that of a conventional G11 (VL/GS4/VH) scFv molecule (pMJF060).

147. The scFv molecule of claim 146, wherein the variable light domain (VL) of said stabilized scFv is identical to the VL domain of the M14-G11 antibody (SEQ ID NO:93) but for the presence of one or more stabilizing mutations at amino acid positions L50 and/or V83 (Kabat numbering convention).

148. The scFv molecule of claim 147, said stabilizing mutations are selected from the group consisting of: L50G, L50M, L50N and V83E.

149. The scFv molecule of claim 146, wherein the variable heavy domain (VH) of said stabilized scFv is identical to the VH domain of the M14-G11 antibody (SEQ ID NO:32) but for the presence one or more stabilizing mutations at amino acid positions E6 and/or S49 (Kabat numbering convention).

150. The scFv molecule of claim 149, wherein said stabilizing mutations are selected from the group consisting of: E6Q, S49A and S49G.

151. The scFv molecule of claim 146, wherein said stabilized scFv molecule comprises a combination of mutations VL L50N: VH E6Q or VL V83E: VH E6Q.

152. (canceled)

153. The scFv molecule of claim 146, wherein said stabilized scFv molecule is a stabilized G11 scFv molecule is selected from the group consisting of MJF-060, MJF-084, MJF-085, MJF-086, MJF-087, MJF-091, MJF-092 and MJF-097.

154. The scFv molecule of claim 128, wherein said scFv molecule has binding specificity for IGF-1R.

155. A multivalent binding molecule comprising the stabilized scFv molecule of claim 128.

156. The multivalent binding molecule of claim 155, which is essentially free of aggregates when produced at a commercial scale or following incubation in a buffering system (e.g., PBS) for at least 3 months.

157. (canceled)

158. The multivalent binding molecule of claim 155, having a melting temperature (Tm) of at least 60° C.

159. A method of making a stabilized multivalent binding molecule, the method comprising genetically fusing the stabilized scFv molecule of claim 128, to an amino terminus or a carboxy terminus of a light or heavy chain of an antibody molecule.

160. A nucleic acid molecule comprising a nucleotide sequence which encodes the stabilized scFv molecule of claim 128.

161. The nucleic acid molecule of claim 160 which is in a vector.

162. A host cell comprising the vector of claim 161.

163. A method of producing a stabilized binding molecule, comprising culturing the host cell of claim 162 under conditions such that the stabilized binding molecule is produced.

164. The method of claim 163, wherein the host cell is cultured at commercial scale (e.g., 50 L) and wherein at least 5 mg or at least 50 mg of the stabilized binding molecule is produced for every liter of the host cell culture medium.

165. (canceled)

166. The method of claim 163, wherein the host cell is cultured at commercial scale and wherein not more than 10% of the binding molecule is present in aggregate form.

167. The multispecific IGF-1R binding molecule of claim 6,
wherein binding of the multispecific IGF-1R binding molecule to IGF-1R inhibits IGF-1R mediated tumor cell growth in vitro to a greater extent than a (i) a first monospecific binding molecule comprising said first binding moiety, (ii) a second monospecific binding molecule comprising said second moiety, or (iii) a combination of said first and second monospecific binding molecules.

168. The multispecific IGF-1R binding molecule of claim 6,
wherein binding of the multispecific IGF-1R binding molecule to IGF-1R inhibits IGF-1R mediated tumor cell growth in vivo to a greater extent than a (i) a first monospecific binding molecule comprising said first binding moiety, (ii) a second monospecific binding molecule comprising said second moiety, or (iii) a combination of said first and second monospecific binding molecules.

169. The multispecific IGF-1R binding molecule of claim 6,
wherein binding of the multispecific IGF-1R binding molecule to IGF-1R blocks IGF-1R-mediated signaling to a greater extent than a (i) a first monospecific binding molecule comprising said first binding moiety, (ii) a second monospecific binding molecule comprising said second moiety, or (iii) a combination of said first and second monospecific binding molecules.

170. The multispecific IGF-1R binding molecule of claim 6,
wherein the multispecific IGF-1R binding molecule binds to IGF-1R with a higher binding affinity than a (i) a first monospecific binding molecule comprising said first binding moiety, (ii) a second monospecific binding molecule comprising said second moiety, or (iii) a combination of said first and second monospecific binding molecules.

171. The multispecific IGF-1R binding molecule of claim 6,
wherein binding of the multispecific IGF-1R binding molecule to IGF-1R blocks binding of IGF-1 and/or IGF-2 to IGF-1R to a greater extent than a (i) a first monospecific binding molecule comprising said first binding moiety, (ii) a second monospecific binding molecule comprising said second moiety, or (iii) a combination of said first and second monospecific binding molecules.

172. The multispecific IGF-1R binding molecule of claim 6,
wherein the multi specific IGF-1R binding molecule has a longer serum half-life than a (i) a first monospecific binding molecule comprising said first binding moiety, (ii) a second monospecific binding molecule comprising said second moiety, or (iii) a combination of said first and second monospecific binding molecules.

173. The multispecific IGF-1R binding molecule of claim 6,
wherein binding of the multispecific IGF-1R binding molecule to IGF-1R inhibits IGF-1 or IGF-2-mediated IGF-1R phosphorylation to a greater extent than a (i) a first monospecific binding molecule comprising said first binding moiety, (ii) a second monospecific binding molecule comprising said second moiety, or (iii) a combination of said first and second monospecific binding molecules.

174. The multispecific IGF-1R binding molecule of claim 6,
wherein binding of the multispecific IGF-1R binding molecule to IGF-1R inhibits IGF-1 or IGF-2-mediated AKT and/or MAPK phosphorylation to a greater extent than a (i) a first monospecific binding molecule comprising said first binding moiety, (ii) a second monospecific binding molecule comprising said second moiety, or (iii) a combination of said first and second monospecific binding molecules.

175. The multispecific IGF-1R binding molecule of claim 6,
wherein the multispecific IGF-1R binding molecule cross-links IGF-1R receptors to a greater extent than a (i) a first monospecific binding molecule comprising said first binding moiety, (ii) a second monospecific binding molecule comprising said second moiety, or (iii) a combination of said first and second monospecific binding molecules.

176. The multispecific IGF-1R binding molecule of claim 6,
wherein binding of the multispecific IGF-1R binding molecule to IGF-1R induces IGF-1R receptor internalization to a greater extent than a (i) a first monospecific binding molecule comprising said first binding moiety, (ii) a second monospecific binding molecule comprising said second moiety, or (iii) a combination of said first and second monospecific binding molecules.

177. The multispecific IGF-1R binding molecule of claim 6,
wherein binding of the multispecific IGF-1R binding molecule to IGF-1R induces tumor cell cycle arrest to a greater extent than a (i) a first monospecific binding molecule comprising said first binding moiety, (ii) a second monospecific binding molecule comprising said second moiety, or (iii) a combination of said first and second monospecific binding molecules.

178. The multispecific IGF-1R binding molecule of claim 6,
wherein binding of the multispecific IGF-1R binding molecule to IGF-1R inhibits IGF-1R mediated tumor cell growth to a greater extent than a (i) a first monospecific binding molecule comprising said first binding moiety, (ii) a second monospecific binding molecule comprising said second moiety, or (iii) a combination of said first and second monospecific binding molecules.
